(19) United States
(12) Patent Application Publication
(10) Pub. No.: US 2025/0186594 A1
(43) Pub. Date: Jun. 12, 2025

(54) IL-18 POLYPEPTIDES MODIFIED WITH POLYMERS

(71) Applicant: BRIGHT PEAK THERAPEUTICS AG, Allschwil (CH)

(72) Inventors: Vijaya Raghavan PATTABIRAMAN, Volketswil (CH); Bertolt KREFT, Kleinmachnow (DE); Jean-Philippe CARRALOT, Saint-Louis (FR); Arnaud GOEPFERT, Sierentz (FR); Philipp MOOSMANN, Basel (CH); Camille DELON, Mulhouse (FR)

(21) Appl. No.: 18/813,101

(22) Filed: Aug. 23, 2024

Related U.S. Application Data
(60) Provisional application No. 63/534,297, filed on Aug. 23, 2023.

(51) Int. Cl. A61K 47/60; A61K 38/00; A61K 45/06; A61P 35/00; C07K 14/54

(52) U.S. Cl. CPC A61K 47/60; A61K 45/06; A61P 35/00; C07K 14/54; A61K 38/00

(57) ABSTRACT

The present disclosure relates a modified interleukin 18 (IL-18) polypeptide comprising a polymer, methods of making the same, and methods of using the modified IL-18 polypeptides for treatment of diseases including cancer. The modified IL-18 polypeptide can exhibit an half-a maximal effective concentration (EC50) to induce IFNγ production is a cell lower than the EC50 of wild type IL-18 (WT IL-18). The modified IL-18 polypeptide can exhibit superior tumor growth inhibition properties and increased half-life in plasma compared to an identical IL-18 polypeptide with no polymer attached. The modified IL-18 polypeptide can also include an optimally sized polymer attached for enhanced efficacy.

### CROSS REFERENCE

This application claims the benefit of U.S. Provisional Application No. 63/534,297 filed Aug. 23, 2023, which applications is incorporated herein by reference in their entirety

### SEQUENCE LISTING

The instant application contains a Sequence Listing which has been submitted electronically in XML format and is hereby incorporated by reference in its entirety. Said XML copy, created on Aug. 8, 2024, is named 94917-0159_735201US_SL.xml and is 137,545 bytes in size.

### BACKGROUND

Immunotherapies utilize the immune system of a subject to aid in the treatment of ailments. Immunotherapies can be designed to activate or suppress the immune system depending on the nature of the disease being treated. The goal of immunotherapies for the treatment of cancer is to stimulate the immune system so that it recognizes and destroys tumors or other cancerous tissue. One method of activating the immune system to attack cancer cells in the body of a subject is cytokine therapy. Cytokines are proteins produced in the body that are important in cell signaling and in modulating the immune system. Some cytokine therapy utilizes these properties of cytokines to enhance the immune system of a subject to kill cancer cells.

### BRIEF SUMMARY

In one aspect, provided herein, is a modified interleukin 18 (IL-18) polypeptide. The modified IL-18 polypeptide can comprise a polymer. The polymer can be covalently attached to an amino acid residue of the modified IL-18 polypeptide. The modified IL-18 polypeptide exhibits an ability to induce interferon gamma (IFNγ) production when in contact with a cell. In some embodiments, the modified IL-18 polypeptide can exhibit a half-maximal effective concentration (EC50) to induce IFNγ production in a cell which is lower than the EC50 for wild type IL-18 (WT IL-18). In some embodiments, the modified IL-18 polypeptide exhibits an enhanced ability for signaling through an IL-18 receptor compared to WT IL-18. In some embodiments, the modified IL-18 polypeptide exhibits a diminished ability to be inhibited by IL-18 binding protein (IL-18BP) compared to WT IL-18. In certain embodiments, the modified IL-18 polypeptide exhibits i) an enhanced ability to induce IFNγ production in a cell compared to WT IL-18, and ii) an enhanced ability for signaling through an IL-18 receptor compared to WT IL-18. In certain embodiments, the modified IL-18 polypeptide exhibits i) an enhanced ability to induce IFNγ production in a cell compared to WT IL-18, ii) an enhanced ability for signaling through an IL-18 receptor compared to WT IL-18, and iii) a diminished ability to be inhibited by IL-18BP compared to WT IL-18. In certain embodiments, the modified IL-18 polypeptide exhibits an enhanced half-life in plasma compared to an identical IL-18 polypeptide without the polymer attached. In certain embodiments, the modified IL-18 polypeptide exhibits superior tumor growth inhibition properties compared to an identical IL-18 polypeptide without the polymer attached. In certain embodiments, the modified IL-18 polypeptide exhibits an ability to activate NK cells in a tumor and/or tumor microenvironment. In certain embodiments, the modified IL-18 polypeptide exhibits an ability to induce IFNγ production in tumor and/or tumor microenvironment. In certain embodiments, the modified IL-18 polypeptide exhibits an ability to induce TNFα production in tumor and/or tumor microenvironment.

In some embodiments, the polymer is attached to a natural amino acid residue. In some embodiments, the natural amino acid residue is selected from asparagine, aspartic acid, cysteine, glutamic acid, glutamine, lysine, and tyrosine. In some embodiments, the polymer is attached to a cysteine residue of the modified IL-18 polypeptide. In some embodiments, the polymer is attached to an unnatural amino acid residue. The polymer can be attached at residue 68, 69, or 70 of the modified IL-18 polypeptide, wherein the residue position numbering is based on SEQ ID NO: 1 as the reference sequence. Unless specifically mentioned otherwise, the residue position numbering is provided in this paragraph, and elsewhere in this disclosure, is based on SEQ ID NO: 1, as a reference sequence. In some embodiments, the polymer is attached at residue 68 of the modified IL-18 polypeptide. In some embodiments, the polymer is attached covalent attached to residue C68 of the modified IL-18 polypeptide. In some embodiments, the polymer is attached at residue 69 of the modified IL-18 polypeptide. In some embodiments, the polymer is attached at residue 70 of the modified IL-18 polypeptide. In some embodiments, one of the residues 69 or 70 is substituted for a cysteine. The polymer attachment may increase plasma half-life and/or increase tumor growth inhibition properties of the modified IL-18 polypeptide compared to an identical IL-18 polypeptide (e.g., having the same amino acid sequence as of the modified IL-18 polypeptide), without the polymer attached.

In some embodiments, the polymer has a MW of about 10 kDa. In some embodiments, the polymer has a Mw of about 20 kDa. In some embodiments, the polymer has a Mw of about 25 kDa. In some embodiments, the polymer has a MW of about 30 kDa. In some embodiments, the polymer has a Mw of about 35 kDa. In some embodiments, the polymer has a Mw of about 40 kDa. In some embodiments, the polymer has a MW of about 50 kDa. In some embodiments, the polymer comprises a water-soluble polymer. In some embodiments, the water-soluble polymer comprises poly(alkylene oxide), polysaccharide, poly(vinyl pyrrolidone), poly(vinyl alcohol), polyoxazoline, poly(acryloylmorpholine), or a combination thereof. In some embodiments, the water-soluble polymer comprises poly(alkylene oxide). In some embodiments, the poly(alkylene oxide) is polyethylene glycol (PEG).

In some embodiments, the PEG has a MW of at most about 50 kDa. In some embodiments, the PEG has a Mw of at most about 40 kDa. In some embodiments, the PEG has a MW of at most about 35 kDa. In some embodiments, the PEG has a MW of about 10 kDa. In some embodiments, the PEG has a MW of about 20 kDa. In some embodiments, the PEG has a MW of about 25 kDa. In some embodiments, the PEG has a MW of about 30 kDa. In some embodiments, the PEG has a MW of about 35 kDa. In some embodiments, the PEG has a MW of about 40 kDa. In some embodiments, the PEG has a MW of about 50 kDa. In some embodiments, the PEG has a MW of about 0.1 kDa to about 50 kDa. In some embodiments, the PEG has a MW of about 0.1 kDa to about 0.5 kDa, about 0.1 kDa to about 1 kDa, about 0.1 kDa to about 5 kDa, about 0.1 kDa to about 10 kDa, about 0.1 kDa to about 20 kDa, about 0.1 kDa to about 25 kDa, about 0.1 kDa to about 30 kDa, about 0.1 kDa to about 35 kDa, about 0.1 kDa to about 40 kDa, about 0.1 kDa to about 45 kDa, about 0.1 kDa to about 50 kDa, about 0.5 kDa to about 1 kDa, about 0.5 kDa to about 5 kDa, about 0.5 kDa to about 10 kDa, about 0.5 kDa to about 20 kDa, about 0.5 kDa to about 25 kDa, about 0.5 kDa to about 30 kDa, about 0.5 kDa to about 35 kDa, about 0.5 kDa to about 40 kDa, about 0.5 kDa to about 45 kDa, about 0.5 kDa to about 50 kDa, about 1 kDa to about 5 kDa, about 1 kDa to about 10 kDa, about 1 kDa to about 20 kDa, about 1 kDa to about 25 kDa, about 1 kDa to about 30 kDa, about 1 kDa to about 35 kDa, about 1 kDa to about 40 kDa, about 1 kDa to about 45 kDa, about 1 kDa to about 50 kDa, about 5 kDa to about 10 kDa, about 5 kDa to about 20 kDa, about 5 kDa to about 25 kDa, about 5 kDa to about 30 kDa, about 5 kDa to about 35 kDa, about 5 kDa to about 40 kDa, about 5 kDa to about 45 kDa, about 5 kDa to about 50 kDa, about 10 kDa to about 20 kDa, about 10 kDa to about 25 kDa, about 10 kDa to about 30 kDa, about 10 kDa to about 35 kDa, about 10 kDa to about 40 kDa, about 10 kDa to about 45 kDa, about 10 kDa to about 50 kDa, about 20 kDa to about 25 kDa, about 20 kDa to about 30 kDa, about 20 kDa to about 35 kDa, about 20 kDa to about 40 kDa, about 20 kDa to about 45 kDa, about 20 kDa to about 50 kDa, about 25 kDa to about 30 kDa, about 25 kDa to about 35 kDa, about 25 kDa to about 40 kDa, about 25 kDa to about 45 kDa, about 25 kDa to about 50 kDa, about 30 kDa to about 35 kDa, about 30 kDa to about 40 kDa, about 30 kDa to about 45 kDa, about 30 kDa to about 50 kDa, about 35 kDa to about 40 kDa, about 35 kDa to about 45 kDa, about 35 kDa to about 50 kDa, about 40 kDa to about 45 kDa, about 40 kDa to about 50 kDa, or about 45 kDa to about 50 kDa. In some embodiments, the PEG has a Mw of about 0.1 kDa, about 0.5 kDa, about 1 kDa, about 5 kDa, about 10 kDa, about 20 kDa, about 25 kDa, about 30 kDa, about 35 kDa, about 40 kDa, about 45 kDa, or about 50 kDa. In some embodiments, the PEG has a MW of at least about 0.1 kDa, about 0.5 kDa, about 1 kDa, about 5 kDa, about 10 kDa, about 20 kDa, about 25 kDa, about 30 kDa, about 35 kDa, about 40 kDa, or about 45 kDa. In some embodiments, the PEG has a Mw of at most about 0.5 kDa, about 1 kDa, about 5 kDa, about 10 kDa, about 20 kDa, about 25 kDa, about 30 kDa, about 35 kDa, about 40 kDa, about 45 kDa, or about 50 kDa. In some embodiments, the PEG has a MW of about 25 kDa to about 35 kDa. In some embodiments, the PEG has a MW of about 25 kDa to about 26 kDa, about 25 kDa to about 27 kDa, about 25 kDa to about 28 kDa, about 25 kDa to about 29 kDa, about 25 kDa to about 30 kDa, about 25 kDa to about 31 kDa, about 25 kDa to about 32 kDa, about 25 kDa to about 33 kDa, about 25 kDa to about 34 kDa, about 25 kDa to about 35 kDa, about 26 kDa to about 27 kDa, about 26 kDa to about 28 kDa, about 26 kDa to about 29 kDa, about 26 kDa to about 30 kDa, about 26 kDa to about 31 kDa, about 26 kDa to about 32 kDa, about 26 kDa to about 33 kDa, about 26 kDa to about 34 kDa, about 26 kDa to about 35 kDa, about 27 kDa to about 28 kDa, about 27 kDa to about 29 kDa, about 27 kDa to about 30 kDa, about 27 kDa to about 31 kDa, about 27 kDa to about 32 kDa, about 27 kDa to about 33 kDa, about 27 kDa to about 34 kDa, about 27 kDa to about 35 kDa, about 28 kDa to about 29 kDa, about 28 kDa to about 30 kDa, about 28 kDa to about 31 kDa, about 28 kDa to about 32 kDa, about 28 kDa to about 33 kDa, about 28 kDa to about 34 kDa, about 28 kDa to about 35 kDa, about 29 kDa to about 30 kDa, about 29 kDa to about 31 kDa, about 29 kDa to about 32 kDa, about 29 kDa to about 33 kDa, about 29 kDa to about 34 kDa, about 29 kDa to about 35 kDa, about 30 kDa to about 31 kDa, about 30 kDa to about 32 kDa, about 30 kDa to about 33 kDa, about 30 kDa to about 34 kDa, about 30 kDa to about 35 kDa, about 31 kDa to about 32 kDa, about 31 kDa to about 33 kDa, about 31 kDa to about 34 kDa, about 31 kDa to about 35 kDa, about 32 kDa to about 33 kDa, about 32 kDa to about 34 kDa, about 32 kDa to about 35 kDa, about 33 kDa to about 34 kDa, about 33 kDa to about 35 kDa, or about 34 kDa to about 35 kDa. In some embodiments, the PEG has a MW of about 25 kDa, about 26 kDa, about 27 kDa, about 28 kDa, about 29 kDa, about 30 kDa, about 31 kDa, about 32 kDa, about 33 kDa, about 34 kDa, or about 35 kDa. In some embodiments, the PEG has a MW of at least about 25 kDa, about 26 kDa, about 27 kDa, about 28 kDa, about 29 kDa, about 30 kDa, about 31 kDa, about 32 kDa, about 33 kDa, or about 34 kDa. In some embodiments, the PEG has a MW of at most about 26 kDa, about 27 kDa, about 28 kDa, about 29 kDa, about 30 kDa, about 31 kDa, about 32 kDa, about 33 kDa, about 34 kDa, or about 35 kDa.

In certain embodiments, the modified IL-18 polypeptide comprises a substitution at residue V11. In certain embodiments, the modified IL-18 polypeptide comprises V11I substitution. Unless specifically mentioned otherwise, the residue position numbering is provided in this paragraph, and elsewhere in this disclosure is based on SEQ ID NO: 1, as a reference sequence. Unless specifically mentioned otherwise, the amino acid substitutions provided in this paragraph, and elsewhere in this disclosure is with respect to SEQ ID NO: 1, as a reference sequence. In certain embodiments, the modified IL-18 polypeptide comprises a substitution at a residue selected from Y1, F2, E6, K8, S10, D17, T34, D35, S36, D37, D40, N41, I49, M51, K53, D54, S55, Q103, S105, G108, H109, D110, and D132. In certain embodiments, the modified IL-18 polypeptide comprises a substitution at residue E6. In certain embodiments, the modified IL-18 polypeptide comprises E6K substitution. In certain embodiments, the modified IL-18 polypeptide comprises a substitution at residue M51. In certain embodiments, the modified IL-18 polypeptide comprises M51G substitution. In certain embodiments, the modified IL-18 polypeptide comprises a substitution at residue K53. In certain embodiments, the modified IL-18 polypeptide comprises K53A substitution. In certain embodiments, the modified IL-18 polypeptide comprises a substitution at residue T63. In certain embodiments, the modified IL-18 polypeptide comprises a T63A substitution. In certain embodiments, the modified IL-18 polypeptide comprises i) substitution at residue V11, and ii) substitution at residue E6, M51 and/or K53. In certain embodiments, the modified IL-18 polypeptide comprises i) V11I substitution, and ii) E6K, M51G, and/or K53A substitution. In certain embodiments, the modified IL-18 polypeptide comprises substitutions at residues E6, V11, K53 and T63. In certain embodiments, the modified IL-18 polypeptide comprises E6K, V11I, K53A and T63A substitutions. In certain embodiments, the modified IL-18 polypeptide comprises substitutions at residues V11, M51, and K53. In certain embodiments, the modified IL-18 polypeptide comprises V11I, M51G, and K53A substitutions. In certain embodiments, the modified IL-18 polypeptide comprises substitutions at residues V11, M51, K53 and T63. In certain embodiments, the modified IL-18 polypeptide comprises V11I, M51G, K53A, and T63A substitutions. In certain embodiments, the modified IL-18 polypeptide comprises substitutions at residues E6, V11, M51, K53 and T63. In certain embodiments, the modified IL-18 polypeptide comprises E6K, V11I, M51G, K53A and T63A substitutions. In certain embodiments, the modified IL-18 polypeptide comprises a substitution at residue C38, C68, C76, and/or C127. In certain embodiments, the modified IL-18 polypeptide comprises a substitution at residue C38. In certain embodiments, the modified IL-18 polypeptide comprises a substitution at residue C68. In certain embodiments, the modified IL-18 polypeptide comprises a substitution at residue C76. In certain embodiments, the modified IL-18 polypeptide comprises a substitution at residue C127. In certain embodiments, the modified IL-18 polypeptide comprises a C38A, C38S, C68A, C68S, C76A, C76S, C127A, and/or C127S substitution. In certain embodiments, the modified IL-18 polypeptide comprises a C38A substitution. In certain embodiments, the modified IL-18 polypeptide comprises C38S. In certain embodiments, the modified IL-18 polypeptide comprises a C68A substitution. In certain embodiments, the modified IL-18 polypeptide comprises C68S. In certain embodiments, the modified IL-18 polypeptide comprises a C76A substitution. In certain embodiments, the modified IL-18 polypeptide comprises C76S. In certain embodiments, the modified IL-18 polypeptide comprises a C127A substitution. In certain embodiments, the modified IL-18 polypeptide comprises C127S. In certain embodiments, the modified IL-18 polypeptide comprises substitutions at each of residues C38, C76, and C127, wherein each of the substitutions at residues C38, C76, and C127 is for a serine or alanine. In certain embodiments, the modified IL-18 polypeptide comprises C38A, C76A, and C127A substitutions. In certain embodiments, the modified IL-18 polypeptide comprises C38S, C76S and C127S substitutions. In certain embodiments, the modified IL-18 polypeptide comprises substitutions at residues E6, V11, C38, K53, T63, C76, and C127. In certain embodiments, the modified IL-18 polypeptide comprises E6K, V11I, C38A, K53A, T63A, C76A, and C127A substitutions. In certain embodiments, the modified IL-18 polypeptide comprises substitutions at residues V11, C38, M51, K53, T63, C76, and C127. In certain embodiments, the modified IL-18 polypeptide comprises V11I, C38A, M51G, K53A, T63A, C76A, and C127A substitutions. In certain embodiments, the modified IL-18 polypeptide comprises substitutions at residues V11, C38, M51, K53, C76, and C127. In certain embodiments, the modified IL-18 polypeptide comprises V11, C38A, M51G, K53A, C76A, and C127A substitutions. In certain embodiments, the modified IL-18 polypeptide comprises substitutions at residues E6, V1, C38, M51, K53, T63, C76, and C127. In certain embodiments, the modified Il-18 polypeptide comprises E6K, V11I, C38A, M51G, K53A, T63A, C76A, and C127A substitutions. In certain embodiments, the modified IL-18 polypeptide comprises at least one glycine residue attached to the N-terminus of the IL-18 polypeptide. In certain embodiments, the modified IL-18 polypeptide comprises a chain of 1 to 10 glycine residues attached to the N-terminus of the IL-18 polypeptide. In certain embodiments, the modified IL-18 polypeptide comprises a chain of 1, 2, 3, 4, 5, 6, 7, 8, 9, or 10, or any range therebetween glycine residues attached to the N-terminus of the IL-18 polypeptide. In certain embodiments, the modified IL-18 polypeptide comprises a glycine residue attached to the N-terminus of the IL-18 polypeptide. In certain embodiments, the modified IL-18 polypeptide comprises a chain of 2 glycine residues attached to the N-terminus of the IL-18 polypeptide. In certain embodiments, the modified IL-18 polypeptide comprises a chain of 3 glycine residues attached to the N-terminus of the IL-18 polypeptide. In certain embodiments, the modified IL-18 polypeptide comprises a chain of 4 glycine residues attached to the N-terminus of the IL-18 polypeptide. In certain embodiments, the modified IL-18 polypeptide comprises a chain of 5 glycine residues attached to the N-terminus of the IL-18 polypeptide. In certain embodiments, the modified IL-18 polypeptide comprises i) a chain of 1, 2, 3, 4, 5, 6, 7, 8, 9, or 10, or any range therebetween glycine residues attached to the N-terminus of the IL-18 polypeptide, and ii) substitutions at residues E6, V11, C38, K53, T63, C76, and C127. In certain embodiments, the modified IL-18 polypeptide comprises i) a chain of 1, 2, 3, 4, 5, 6, 7, 8, 9, or 10, or any range therebetween glycine residues attached to the N-terminus of the IL-18 polypeptide, and ii) E6K, V11I, C38A, K53A, T63A, C76A, and C127A substitutions. In certain embodiments, the modified IL-18 polypeptide comprises i) a glycine residue attached to the N-terminus of the IL-18 polypeptide, and ii) substitutions at residues E6, V11, C38, K53, T63, C76, and C127. In certain embodiments, the modified IL-18 polypeptide comprises i) a glycine residue attached to the N-terminus of the IL-18 polypeptide, and ii) E6K, V11I, C38A, K53A, T63A, C76A, and C127A substitutions. In certain embodiments, the modified IL-18 polypeptide comprises i) a chain of 4 glycine residues attached to the N-terminus of the IL-18 polypeptide, and ii) substitutions at residues E6, V11, C38, K53, T63, C76, and C127. In certain embodiments, the modified IL-18 polypeptide comprises i) a chain of 4 glycine residues attached to the N-terminus of the IL-18 polypeptide, and ii) E6K, V11, C38A, K53A, T63A, C76A, and C127A substitutions. In certain embodiments, the modified IL-18 polypeptide comprises i) a chain of 1, 2, 3, 4, 5, 6, 7, 8, 9, or 10, or any range therebetween glycine residues attached to the N-terminus of the IL-18 polypeptide, and ii) substitutions at residues E6, V11, C38, M51, K53, T63, C76, and C127. In certain embodiments, the modified IL-18 polypeptide comprises i) a chain of 1, 2, 3, 4, 5, 6, 7, 8, 9, or 10, or any range therebetween glycine residues attached to the N-terminus of the IL-18 polypeptide, and ii) E6K, V11I, C38A, M51G, K53A, T63A, C76A, and C127A substitutions. In certain embodiments, the modified IL-18 polypeptide comprises i) a glycine residue attached to the N-terminus of the IL-18 polypeptide, and ii) substitutions at residues E6, V11, C38, M51, K53, T63, C76, and C127. In certain embodiments, the modified IL-18 polypeptide comprises i) a glycine residue attached to the N-terminus of the IL-18 polypeptide, and ii) E6K, V11I, C38A, M51G, K53A, T63A, C76A, and C127A substitutions.

In certain embodiments, the modified IL-18 polypeptide comprises an amino acid sequence having at least 80%, 85%, 90%, 91%, 92%, 93%, 94%, or 95% sequence identity with the sequence set forth in SEQ ID NO: 1. In certain embodiments, the modified IL-18 polypeptide comprises an amino acid sequence having at least 80% sequence identity with the sequence set forth in SEQ ID NO: 1. In certain embodiments, the modified IL-18 polypeptide comprises an amino acid sequence having at least 90% sequence identity with the sequence set forth in SEQ ID NO: 1. In certain embodiments, the modified IL-18 polypeptide comprises an amino acid sequence having at least 95% sequence identity with the sequence set forth in SEQ ID NO: 1. In certain embodiments, the modified IL-18 polypeptide comprises i) E6K, V11I, K53A, and T63A, substitutions, and ii) a polymer comprising a PEG attached to a residue of the modified IL-18 polypeptide, wherein the PEG has a Mw of about 10 kDa, 20 kDa, 30 kDa, 40 kDa, or 50 kDa. In certain embodiments, the modified IL-18 polypeptide comprises i) E6K, V11I, C38A, K53A, T63A, C76A, and C127A substitutions, and ii) a polymer comprising a PEG attached to a residue of the modified IL-18 polypeptide, wherein the PEG has a Mw of about 10 kDa, 20 kDa, 30 kDa, 40 kDa, or 50 kDa. In certain embodiments, the modified IL-18 polypeptide comprises i) E6K, V11I, C38A, K53A, T63A, C76A, and C127A substitutions, ii) a polymer comprising a PEG attached to a residue of the modified IL-18 polypeptide, wherein the PEG has a Mw of about 10 kDa, 20 kDa, 30 kDa, 40 kDa, or 50 kDa, and iii) a chain of 1, 2, 3, 4, 5, 6, 7, 8, 9, or 10, or any range therebetween glycine residues attached to the N-terminus of the modified IL-18 polypeptide. In certain embodiments, the modified IL-18 polypeptide comprises i) E6K, V11I, C38A, K53A, T63A, C76A, and C127A substitutions, and ii) a polymer comprising a PEG attached to residue 68 of the modified IL-18 polypeptide, wherein the PEG has a Mw of about 30 kDa. In certain embodiments, the modified IL-18 polypeptide comprises i) E6K, V11, C38A, K53A, T63A, C76A, and C127A substitutions, ii) a polymer comprising a PEG attached to residue 68 of the modified IL-18 polypeptide, wherein the PEG has a Mw of about 30 kDa, and iii) a chain of 1, 2, 3, 4, 5, 6, 7, 8, 9, or 10, or any range therebetween glycine residues attached to the N-terminus of the modified IL-18 polypeptide. In certain embodiments, the modified IL-18 polypeptide comprises i) E6K, V11I, C38A, K53A, T63A, C76A, and C127A substitutions, ii) a polymer comprising a PEG attached to residue 68 of the modified IL-18 polypeptide, wherein the PEG has a Mw of about 30 kDa, and iii) a chain of 4 glycine residues attached to the N-terminus of the modified IL-18 polypeptide. In certain embodiments, the modified IL-18 polypeptide comprises i) E6K, V11I, C38A, K53A, T63A, C76A, and C127A substitutions, ii) a polymer comprising a PEG attached to residue 68 of the modified IL-18 polypeptide, wherein the PEG has a Mw of about 30 kDa, and iii) a glycine residue attached to the N-terminus of the modified IL-18 polypeptide. In certain embodiments, the modified IL-18 polypeptide comprises i) the amino acid sequence set forth in SEQ ID NO: 30, and ii) a polymer comprising PEG attached to residue 68 of the modified IL-18 polypeptide, wherein the PEG has a Mw of about 30 kDa. In certain embodiments, the modified IL-18 polypeptide comprises i) the amino acid sequence set forth in SEQ ID NO: 241, and ii) a polymer comprising PEG attached to residue 68 of the modified IL-18 polypeptide, wherein the PEG has a Mw of about 30 kDa. In certain embodiments, the modified IL-18 polypeptide comprises i) the amino acid sequence set forth in SEQ ID NO: 242, and ii) a polymer comprising PEG attached to residue 68 of the modified IL-18 polypeptide, wherein the PEG has a Mw of about 30 kDa.

In certain embodiments, the modified IL-18 polypeptide comprises i) V11I, M51G, and K53A substitutions, and ii) a polymer comprising a PEG attached to a residue of the modified IL-18 polypeptide, wherein the PEG has a Mw of about 10 kDa, 20 kDa, 30 kDa, 40 kDa, or 50 kDa. In certain embodiments, the modified IL-18 polypeptide comprises i) V11I, C38A, M51G, K53A, C76A, and C127A substitutions, and ii) a polymer comprising a PEG attached to a residue of the modified IL-18 polypeptide, wherein the PEG has a Mw of about 10 kDa, 20 kDa, 30 kDa, 40 kDa, or 50 kDa, and iii) a chain of 1, 2, 3, 4, 5, 6, 7, 8, 9, or 10, or any range therebetween glycine residues attached to the N-terminus of the modified IL-18 polypeptide. In certain embodiments, the modified IL-18 polypeptide comprises i) V11I, C38A, M51G, K53A, C76A, and C127A substitutions, and ii) a polymer comprising a PEG attached to a residue 68 of the modified IL-18 polypeptide, wherein the PEG has a Mw of about 10 kDa, 20 kDa, 30 kDa, 40 kDa, or 50 kDa. In certain embodiments, the modified IL-18 polypeptide comprises i) V11I, C38A, M51G, K53A, C76A, and C127A substitutions, and ii) a polymer comprising a PEG attached to a residue of the modified IL-18 polypeptide, wherein the PEG has a Mw of about 30 kDa. In certain embodiments, the modified IL-18 polypeptide comprises i) V11I, C38A, M51G, K53A, C76A, and C127A substitutions, and ii) a polymer comprising a PEG attached to residue 68 of the modified IL-18 polypeptide, wherein the PEG has a Mw of about 30 kDa. In certain embodiments, the modified IL-18 polypeptide comprises i) the amino acid sequence set forth in SEQ ID NO: 207, and ii) a polymer comprising PEG attached to residue 68 of the modified IL-18 polypeptide, wherein the PEG has a Mw of about 30 kDa.

In certain embodiments, the modified IL-18 polypeptide comprises i) E6K, V11I, M51G, K53A, and T63A substitutions, and ii) a polymer comprising a PEG attached to a residue of the modified IL-18 polypeptide, wherein the PEG has a Mw of about 10 kDa, 20 kDa, 30 kDa, 40 kDa, or 50 kDa. In certain embodiments, the modified IL-18 polypeptide comprises i) E6K, V11I, C38A, M51G, K53A, T63A, C76A, and C127A substitutions, and ii) a polymer comprising a PEG attached to a residue of the modified IL-18 polypeptide, wherein the PEG has a Mw of about 10 kDa, 20 kDa, 30 kDa, 40 kDa, or 50 kDa. In certain embodiments, the modified IL-18 polypeptide comprises i) E6K, V11I, C38A, M51G, K53A, T63A, C76A, and C127A substitutions, ii) a polymer comprising a PEG attached to a residue of the modified IL-18 polypeptide, wherein the PEG has a Mw of about 10 kDa, 20 kDa, 30 kDa, 40 kDa, or 50 kDa, and iii) a chain of 1, 2, 3, 4, 5, 6, 7, 8, 9, or 10, or any range therebetween glycine residues attached to the N-terminus of the modified IL-18 polypeptide. In certain embodiments, the modified IL-18 polypeptide comprises i) E6K, V11I, C38A, M51G, K53A, T63A, C76A, and C127A substitutions, and ii) a polymer comprising a PEG attached to a residue 68 of the modified IL-18 polypeptide, wherein the PEG has a Mw of about 10 kDa, 20 kDa, 30 kDa, 40 kDa, or 50 kDa. In certain embodiments, the modified IL-18 polypeptide comprises i) E6K, V11I, C38A, M51G, K53A, T63A, C76A, and C127A substitutions, and ii) a polymer comprising a PEG attached to a residue of the modified IL-18 polypeptide, wherein the PEG has a Mw of about 30 kDa. In certain embodiments, the modified IL-18 polypeptide comprises i) E6K, V11I, C38A, M51G, K53A, T63A, C76A, and C127A substitutions, and ii) a polymer comprising a PEG attached to residue 68 of the modified IL-18 polypeptide, wherein the PEG has a Mw of about 30 kDa. In certain embodiments, the modified IL-18 polypeptide comprises i) E6K, V11, C38A, K53A, T63A, C76A, and C127A substitutions, ii) a polymer comprising a PEG attached to residue 68 of the modified IL-18 polypeptide, wherein the PEG has a Mw of about 30 kDa, and iii) a glycine residue attached to the N-terminus of the modified IL-18 polypeptide. In certain embodiments, the modified IL-18 polypeptide comprises i) the amino acid sequence set forth in SEQ ID NO: 239, and ii) a polymer comprising PEG attached to residue 68 of the modified IL-18 polypeptide, wherein the PEG has a Mw of about 30 kDa. In certain embodiments, the modified IL-18 polypeptide comprises i) the amino acid sequence set forth in SEQ ID NO: 244, and ii) a polymer comprising PEG attached to residue 68 of the modified IL-18 polypeptide, wherein the PEG has a Mw of about 30 kDa.

In some embodiments, the modified IL-18 polypeptide is recombinant.

In some embodiments, the modified IL-18 polypeptide exhibits a half-maximal effective concentration (EC50) to induce IFNγ production in a cell which is at least 2-fold lower than the EC50 for wild type IL-18 (WT IL-18). In certain embodiments, the EC50 to induce IFNγ production in a cell for the modified IL-18 polypeptide is at least 3-fold lower, 4-fold, 5-fold, 6-fold, 7-fold, 8-fold, 9-fold, 10-fold, 15 fold, 20-fold, 25 fold, 30-fold, 35 fold, 40-fold, 45 fold, or 50-fold lower than the EC50 for WT IL-18. In certain embodiments, the EC50 to induce IFNγ production in a cell for the modified IL-18 polypeptide is at least 3-fold lower than the EC50 for WT IL-18. In certain embodiments, the EC50 to induce IFNγ production in a cell for the modified IL-18 polypeptide is at least 4-fold lower than the EC50 for WT IL-18. In certain embodiments, the EC50 to induce IFNγ production in a cell for the modified IL-18 polypeptide is at least 5-fold lower than the EC50 for WT IL-18. In certain embodiments, the EC50 to induce IFNγ production in a cell for the modified IL-18 polypeptide is at least 6-fold lower than the EC50 for WT IL-18. In certain embodiments, the EC50 to induce IFNγ production in a cell for the modified IL-18 polypeptide is at least 7-fold lower than the EC50 for WT IL-18. In certain embodiments, the EC50 to induce IFNγ production in a cell for the modified IL-18 polypeptide is at least 8-fold lower than the EC50 for WT IL-18. In certain embodiments, the EC50 to induce IFNγ production in a cell for the modified IL-18 polypeptide is at least 9-fold lower than the EC50 for WT IL-18. In certain embodiments, the EC50 to induce IFNγ production in a cell for the modified IL-18 polypeptide is at least 10-fold lower than the EC50 for WT IL-18. In certain embodiments, the EC50 to induce IFNγ production in a cell for the modified TL-18 polypeptide is at least 20-fold lower than the EC50 for WT IL-18. In certain embodiments, the EC50 to induce IFN7 production in a cell for the modified IL-18 polypeptide is at least 30-fold lower than the EC50 for WT IL-18. In certain embodiments, the EC50 to induce IFNγ production in a cell for the modified IL-18 polypeptide is at least 40-fold lower than the EC50 for WT IL-18. In certain embodiments, the EC50 to induce IFNγ production in a cell for the modified TL-18 polypeptide is at least 50-fold lower than the EC50 for WT IL-18. The cell can be an immune cell. In certain embodiments, the cell is NK cell. In certain embodiments, the cell is PBMC. In certain embodiments, the EC50 to induce IFN7 production in PBMCs for the modified IL-18 polypeptide is at least 4-fold lower than the EC50 for WT IL-18. In certain embodiments, the EC50 to induce IFNγ production in NK cells can be measured using IFNγ Induction NK-92 Cellular Assay (e.g., as provided in the Examples herein). In certain embodiments, the EC50 to induce IFNγ production in PBMCs can be measured using IFNγ secretion assay in PBMCs (e.g., as provided in the Examples herein).

In some embodiments, the modified IL-18 polypeptide exhibits a half-maximal effective concentration (EC50) to induce IFNγ production in a cell which is at most 5-fold higher than an identical IL-18 polypeptide with no polymer attached. In some embodiments, the modified IL-18 polypeptide exhibits a half-maximal effective concentration (EC50) to induce IFNγ production in a cell which is at most 5-fold, 10-fold, 15-fold, 20-fold, 25-fold, 30-fold, 35-fold, 40-fold, 45-fold, 50-fold, 55-fold, 60-fold, 70-fold, 80-fold, 90-fold or 100-fold higher than an identical IL-18 polypeptide with no polymer attached. In some embodiments, the modified IL-18 polypeptide exhibits a half-maximal effective concentration (EC50) to induce IFNγ production in a cell which is at most 10-fold higher than an identical IL-18 polypeptide with no polymer attached. In some embodiments, the modified IL-18 polypeptide exhibits a half-maximal effective concentration (EC50) to induce IFNγ production in a cell which is at most 25-fold higher than an identical IL-18 polypeptide with no polymer attached. In some embodiments, the modified IL-18 polypeptide exhibits a half-maximal effective concentration (EC50) to induce IFNγ production in a cell which is at most 50-fold higher than an identical IL-18 polypeptide with no polymer attached. In some embodiments, the modified IL-18 polypeptide exhibits a half-maximal effective concentration (EC50) to induce IFNγ production in a cell which is at most 100-fold higher than an identical IL-18 polypeptide with no polymer attached. The cell can be an immune cell. In certain embodiments, the cell is NK cell. In certain embodiments, the cell is PBMC. In certain embodiments, the EC50 to induce IFNγ production in NK cells can be measured using IFNγ Induction NK-92 Cellular Assay (e.g., as provided in the Examples herein). In certain embodiments, the EC50 to induce IFNγ production in PBMC can be measured using IFNγ secretion assay in PBMCs (e.g., as provided in the Examples herein).

In some embodiments, the modified IL-18 polypeptide exhibits a ratio of half-maximal inhibitory concentration (IC50) by IL-18BP to half-maximal effective concentration (EC50) to induce IFNγ production which is enhanced relative to WT IL-18. In some embodiments, the ratio is at least 10, at least 20, at least 30, at least 40, at least 50, at least 100, at least 200, at least 500, or at least 1000. EC50 to induce IFNγ production can be measured using an IFNγ Induction NK-92 Cellular Assay (e.g., as provided herein in the Examples) and IC50 by IL-18BP can be measured by IL-18 Binding Protein-mediated Inhibition of IFNγ secretion in NK-92 Cellular Assay (e.g., as provided herein in the Examples).

In some embodiments, the modified IL-18 polypeptide exhibits a half maximal effective concentration (EC50) for signaling through an IL-18 receptor which is at least 4.6-fold lower than the EC50 for WT IL-18. In certain embodiments, the EC50 for signaling through the IL-18 receptor for the modified IL-18 polypeptide is at least 5-fold, 6-fold, 7-fold, 8-fold, 9-fold, or 10-fold lower than the EC50 for WT IL-18, wherein the modified IL-18 polypeptide comprises the polymer attached and the WT IL-18 has no polymer attached. In certain embodiments, the EC50 for signaling through the IL-18 receptor for the modified IL-18 polypeptide is at least 40-fold lower than the EC50 for WT IL-18. In certain embodiments, the EC50 for signaling through the IL-18 receptor for the modified IL-18 polypeptide is at least 50-fold lower than the EC50 for WT IL-18. EC50 for signaling through the IL-18 receptor can be measured using HEK-Blue IL18R reporter assay (e.g., as provided in the Examples herein).

The modified IL-18 polypeptide exhibits a half-maximal inhibitory concentration (IC50) by IL-18BP higher than IC50 of WT IL-18. In some embodiments, the modified IL-18 polypeptide exhibits a half-maximal inhibitory concentration (IC50) by IL-18BP at least 50-fold higher than IC50 of WT IL-18. In some embodiments, the modified IL-18 polypeptide exhibits a half-maximal inhibitory concentration (IC50) by IL-18BP at higher than IC50 of an identical IL-18 polypeptide with no polymer attached. In some embodiments, the modified IL-18 polypeptide exhibits a half-maximal inhibitory concentration (IC50) by IL-18BP at least X-fold higher than IC50 of an identical IL-18 polypeptide with no polymer attached.

In one aspect, provided herein, is a host cell comprising a modified IL-18 polypeptide provided herein. In some embodiments, a method of producing a modified IL-18 polypeptide is provided herein, comprising expressing the modified IL-18 polypeptide in a host cell. In some embodiments, the host cell is a prokaryotic cell or a eukaryotic cell. In some embodiments, the host cell is a mammalian cell, an avian cell, a fungal cell, or an insect cell. In some embodiments, the host cell is a CHO cell, a COS cell, or a yeast cell.

In another aspect, provided herein, is a pharmaceutical composition comprising a modified IL-18 polypeptide provided herein and a pharmaceutically acceptable carrier or excipient. In some embodiments, the pharmaceutical composition comprising the modified IL-18 polypeptide, is in a lyophilized form.

In another aspect is a method of treating cancer in a subject in need thereof comprising administering to the subject a pharmaceutically effective amount of, a modified IL-18 polypeptide, or a pharmaceutical composition comprising a modified IL-18 polypeptide, provided herein. The effective amount of the modified IL-18 polypeptide administered can be 1 μg/kg/administration to about 30 mg/kg/administration. In some embodiments, the effective amount of the modified IL-18 polypeptide administered is about 1 μg/kg/administration to about 100 μg/kg/administration. In some embodiments, the effective amount of the modified IL-18 polypeptide administered is about about 1 μg/kg/administration to about 100 μg/kg/administration, about 5 μg/kg/administration to about 100 μg/kg/administration, about 10 μg/kg/administration to about 20 μg/kg/administration, about 10 μg/kg/administration to about 30 μg/kg/administration, about 10 μg/kg/administration to about 40 μg/kg/administration, about 10 μg/kg/administration to about 50 μg/kg/administration, about 10 μg/kg/administration to about 100 μg/kg/administration, about 20 μg/kg/administration to about 30 μg/kg/administration, about 20 μg/kg/administration to about 40 μg/kg/administration, about 20 μg/kg/administration to about 50 μg/kg/administration, about 20 μg/kg/administration to about 100 μg/kg/administration, about 30 μg/kg/administration to about 40 μg/kg/administration, about 30 μg/kg/administration to about 50 μg/kg/administration, about 30 μg/kg/administration to about 100 μg/kg/administration, about 40 μg/kg/administration to about 50 μg/kg/administration, about 40 μg/kg/administration to about 100 μg/kg/administration, or about 50 μg/kg/administration to about 100 μg/kg/administration, about. In some embodiments, the effective amount of the modified IL-18 polypeptide administered is about 1 μg/kg/administration, about 5 μg/kg/administration, about 10 μg/kg/administration, about 20 μg/kg/administration, about 30 μg/kg/administration, about 40 μg/kg/administration, about 50 μg/kg/administration, about 60 μg/kg/administration, about 70 μg/kg/administration, about 80 μg/kg/administration, about 90 μg/kg/administration, or about 100 μg/kg/administration. In some embodiments, the effective amount of the modified IL-18 polypeptide administered is at least about 1 μg/kg/administration, about 5 μg/kg/administration, about 10 μg/kg/administration, about 20 μg/kg/administration, about 30 μg/kg/administration, about 40 μg/kg/administration, about 50 μg/kg/administration, about 60 μg/kg/administration, about 70 μg/kg/administration, about 80 μg/kg/administration, or about 90 μg/kg/administration. In some embodiments, the effective amount of the modified IL-18 polypeptide administered is at most about 5 μg/kg/administration, about 10 μg/kg/administration, about 20 μg/kg/administration, about 30 μg/kg/administration, about 40 μg/kg/administration, about 50 μg/kg/administration, about 60 μg/kg/administration, about 70 μg/kg/administration, about 80 μg/kg/administration, about 90 μg/kg/administration, or about 100 μg/kg/administration.

In some embodiments, the effective amount of the modified IL-18 polypeptide administered is about 0.1 mg/kg/administration to about 10 mg/kg/administration. In such embodiments, these higher doses are generally preferred in non-human mammalian subjects, such mice and other smaller mammals. In some embodiments, the effective amount of the modified IL-18 polypeptide administered is about 0.1 mg/kg/administration to about 0.5 mg/kg/administration, about 0.1 mg/kg/administration to about 1 mg/kg/administration, about 0.1 mg/kg/administration to about 5 mg/kg/administration, about 0.5 mg/kg/administration to about 1 mg/kg/administration, about 0.5 mg/kg/administration to about 5 mg/kg/administration, about 0.5 mg/kg/administration to about 10 mg/kg/administration, about 1 mg/kg/administration to about 2 mg/kg/administration, about 1 mg/kg/administration to about 3 mg/kg/administration, about 1 mg/kg/administration to about 5 mg/kg/administration, about 1 mg/kg/administration to about 8 mg/kg/administration, a about 1 mg/kg/administration to about 10 mg/kg/administration, about 2 mg/kg/administration to about 5 mg/kg/administration, about 2 mg/kg/administration to about 10 mg/kg/administration, about 3 mg/kg/administration to about 5 mg/kg/administration, about 3 mg/kg/administration to about 10 mg/kg/administration, about 4 mg/kg/administration to about 10 mg/kg/administration, about 5 mg/kg/administration to about 10 mg/kg/administration, about 6 mg/kg/administration to about 10 mg/kg/administration, or about 7 mg/kg/administration to about 10 mg/kg/administration. In some embodiments, the effective amount of the modified IL-18 polypeptide administered is about 0.1 mg/kg/administration, about 0.5 mg/kg/administration, about 1 mg/kg/administration, about 2 mg/kg/administration, about 3 mg/kg/administration, about 4 mg/kg/administration, about 5 mg/kg/administration, about 6 mg/kg/administration, about 7 mg/kg/administration, about 8 mg/kg/administration, about 9 mg/kg/administration, or about 10 mg/kg/administration. In some embodiments, the effective amount of the modified IL-18 polypeptide administered is at least about 0.1 mg/kg/administration, about 0.5 mg/kg/administration, about 1 mg/kg/administration, about 2 mg/kg/administration, about 3 mg/kg/administration, about 4 mg/kg/administration, about 5 mg/kg/administration, about 6 mg/kg/administration, about 7 mg/kg/administration, about 8 mg/kg/administration, or about 9 mg/kg/administration. In some embodiments, the effective amount of the modified IL-18 polypeptide administered is at most about 0.5 mg/kg/administration, about 1 mg/kg/administration, about 2 mg/kg/administration, about 3 mg/kg/administration, about 4 mg/kg/administration, about 5 mg/kg/administration, about 6 mg/kg/administration, about 7 mg/kg/administration, about 8 mg/kg/administration, about 9 mg/kg/administration, or about 10 mg/kg/administration.

In some embodiments, the cancer is a solid cancer. In some embodiments, the solid cancer is adrenal cancer, anal cancer, bile duct cancer, bladder cancer, bone cancer, brain cancer, breast cancer, carcinoid cancer, cervical cancer, colorectal cancer, esophageal cancer, eye cancer, gallbladder cancer, gastrointestinal stromal tumor, germ cell cancer, head and neck cancer, kidney cancer, liver cancer, lung cancer, nasal cavity and paranasal sinus cancer, nasopharyngeal cancer, neuroblastoma, neuroendocrine cancer, oral cancer, oropharyngeal cancer, ovarian cancer, pancreatic cancer, pediatric cancer, penile cancer, pituitary cancer, prostate cancer, skin cancer, soft tissue cancer, spinal cord cancer, stomach cancer, testicular cancer, thymus cancer, thyroid cancer, ureteral cancer, uterine cancer, vaginal cancer, or vulvar cancer. In some embodiments, the solid cancer is a carcinoma or a sarcoma. In some embodiments, the cancer is a blood cancer. In some embodiments, the blood cancer is leukemia, non-Hodgkin lymphoma, Hodgkin lymphoma, an AIDS-related lymphoma, multiple myeloma, plasmacytoma, post-transplantation lymphoproliferative disorder, or Waldenstrom macroglobulinemia. In some embodiments, the method comprises reconstituting a lyophilized form of the modified IL-18 polypeptide or the pharmaceutical composition.

Another aspect provides a method of making a modified IL-18 polypeptide provided herein comprising synthesizing two or more fragments of the modified IL-18 polypeptide, ligating the fragments, folding the ligated fragments, and attaching a polymer to the ligated folded fragments by a reaction with a conjugation handle, wherein at least one of the fragments of the IL-18 polypeptide comprises the conjugation handle. In some embodiments, one of the fragments comprises the polymer.

Another aspect provides a modified IL-18 polypeptide having at least about 80%, at least about 85%, at least about 90%, at least about 95%, at least about 96%, at least about 97%, at least about 98%, or at least about 99% sequence identity to the sequence set forth in SEQ ID NO: 30, and comprising a covalently attached polymer. In some embodiments, the polymer is covalently attached at residue 68, 69, or 70. In some embodiments, the polymer is covalently attached at residue 68. In some embodiments, the modified IL-18 polypeptide comprises each of the substitution present in SEQ ID NO: 30 relative to SEQ ID NO: 1.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawing, of which:

FIG. 1 illustrates the mechanism of action of IL-18 on IFNγ and IL-18BP production, and IL-18 inhibitory activity by IL-18BP.

FIG. 2 illustrates the coupling of a dibenzocyclooctyne (DBCO) polyethylene glycol (PEG) with a modified IL-18 polypeptide comprising an azide.

FIG. 3 illustrates the binding of a modified IL-18 polypeptide comprising a polymer with IL-18Rα.

FIG. 4A shows the IFNγ induction ability of a modified IL-18 polypeptide compared to a wild type IL-18 polypeptide.

FIG. 4B shows IL-18BP inhibition of a modified IL-18 polypeptide compared to a wild type IL-18 polypeptide.

FIG. 5 shows a schematic representation of coupling of a bifunctional probe to an IL-18 polypeptide provided herein.

FIG. 6 shows a schematic representation of coupling of a poly(ethylene glycol) moiety to an IL-18 polypeptide activated with a bifunctional probe.

FIG. 7 shows plots measuring the binding activity of wild type IL-18 and of modified IL-18 polypeptides to human IL-18 receptor alpha (CD218a) in surface plasmon resonance experiments, where the x-axis is time, and the y-axis is relative response. The modified IL-18 polypeptides tested in these experiments are native IL-18 wild-type (SEQ ID NO: 01), SEQ ID NO: 57, SEQ ID NO: 59, and SEQ ID NO: 30.

FIG. 8 shows plots measuring the binding activity of wild type IL-18 and of modified IL-18 polypeptides to heterodimeric human IL-18 receptor alpha (CD218a) and IL-18R accessory protein (IL-18RAP/CDw218b) in surface plasmon resonance experiments, where the x-axis is time, and the y-axis is relative response. The modified IL-18 polypeptides tested in these experiments are native IL-18 wild-type (SEQ ID NO: 01), SEQ ID NO: 57, SEQ ID NO: 59, and SEQ ID NO: 30.

FIG. 9 shows plots measuring the binding activity of wild type IL-18 and of modified IL-18 polypeptides to human IL-18 binding protein (IL-18BP) in surface plasmon resonance experiments, where the x-axis is time, and the y-axis is relative response. The modified IL-18 polypeptides tested in these experiments are native IL-18 wild-type (SEQ ID NO: 01), SEQ ID NO: 57, SEQ ID NO: 59, and SEQ ID NO: 30.

FIG. 10 shows plots measuring the ability of wild type IL-18 and of modified IL-18 polypeptides to bind to the human IL-18 Binding Protein (IL-18BP). The figure shows mean free IL-18BP AlphaLISA signal on the y-axis, and dosage of wild type IL-18 and of modified IL-18 polypeptides on the x-axis. The unconjugated IL-18 variants are native IL-18 wild-type (SEQ ID NO: 01), SEQ ID NO: 57, SEQ ID NO: 59, SEQ ID NO: 30, SEQ ID NO: 207, SEQ ID NO: 239, SEQ ID NO: 241, SEQ ID NO: 242, and SEQ ID NO: 244.

FIG. 11 shows plots measuring ability of wild type IL-18 and of modified IL-18 polypeptides to stimulate the secretion of IFNgamma by NK-92 cells. The figure shows mean IFNg alphaLISA signal on the y-axis and dosage of the IL-18 polypeptides on the x-axis. The IL-18 polypeptides are native IL-18 wild-type (SEQ ID NO: 01), SEQ ID NO: 57, SEQ ID NO: 59, and SEQ ID NO: 30.

FIG. 12 shows plots measuring the ability of the human IL-18 Binding Protein to inhibit the secretion of IFNgamma by NK92 cells stimulated with 2 nM of wild type IL-18 and of modified IL-18 polypeptides. The figure shows mean IFNg alphaLISA signal on the y-axis, and dosage of the human IL-18 Binding Protein on the x-axis. The IL-18 polypeptides are native IL-18 wild-type (SEQ ID NO: 01), SEQ ID NO: 57, SEQ ID NO: 59, and SEQ ID NO: 30.

FIG. 13 shows plots measuring ability of wild type IL-18 and of modified IL-18 polypeptides to induce the NF-κB/AP-1-inducible secreted embryonic alkaline phosphatase (SEAP) reporter gene in HEK Blue cells expressing the IL-18 receptor. The figure shows mean SEAP reporter signal (OD 620 nm) on the y-axis, and dosage of the IL-18 polypeptides on the x-axis. The IL-18 polypeptides are native IL-18 wild-type (SEQ ID NO: 01), SEQ ID NO: 57, SEQ ID NO: 59, SEQ ID NO: 207, SEQ ID NO: 239, SEQ ID NO: 241, SEQ ID NO: 242, and SEQ ID NO: 244.

FIG. 14 shows plots measuring ability of wild type IL-18 and of modified IL-18 polypeptides to stimulate the secretion of IFNgamma by human Peripheral Blood Mononuclear Cells (PBMCs). The figure shows mean IFNg signal on the y-axis and dosage of the IL-18 polypeptides on the x-axis. The IL-18 polypeptides are native IL-18 wild-type (SEQ ID NO: 01), SEQ ID NO: 57, SEQ ID NO: 59, and SEQ ID NO: 30.

FIGS. 15A-B show effect of attaching 30 kDa PEG to IL-18 variants on IL-18 mediated IFNγ secretion in human peripheral blood mononuclear cells. EC50 for IL-18 mediated IFNγ secretion for IL-18 of SEQ ID NO: 59, 4 and 9 (with and without 30 kD PEG) is shown in FIG. 15A; and of SEQ ID NO: 30, SEQ ID NO: 30+30 kD PEG, SEQ ID NO: 5, SEQ ID NO: 5+30 kD PEG, SEQ ID NO: 6, and SEQ ID NO: 6+30 kD PEG, is shown in FIG. 15B.

FIG. 16A shows a plot describing plasma exposure of modified IL-18 polypeptides in C57BL/6 mice implanted with MC38 syngeneic colon carcinoma tumors. The figure shows plasma concentrations on the y-axis, and time on the x-axis. The IL-18 polypeptides tested in this figure are SEQ ID NO: 57 and SEQ ID NO: 57 conjugated to a 30 kDa PEG tested as a single agent at 3 mg/kg as a single i.v. injection. (n=3 per time point; mean±SEM).

FIG. 16B shows a plot describing the effect of modified IL-18 polypeptides on levels of IFNgamma and TNFalpha in blood of C57BL/6 mice implanted with MC38 syngeneic colon carcinoma tumors. The figure shows plasma concentrations of IFNg and TNFα on the y-axis, and time on the x-axis. The IL-18 polypeptides tested in this figure are SEQ ID NO: 57 and SEQ ID NO: 57 conjugated to a 30 kDa PEG tested as a single agent at 3 mg/kg as a single i.v. injection. (n=3 per time point; mean±SEM).

FIG. 17A shows a plot describing plasma exposure of modified IL-18 polypeptides in C57BL/6 mice implanted with MC38 syngeneic colon carcinoma tumors. The figure shows plasma concentrations on the y-axis, and time on the x-axis. The IL-18 polypeptides tested in this figure are SEQ ID NO: 59 and SEQ ID NO: 59 conjugated to a 10, 20 and 30 kDa PEG tested as a single agent at 3 mg/kg as a single i.v. injection. (n=3 per time point; mean±SEM).

FIG. 17B shows a plot describing the effect of modified IL-18 polypeptides on levels of IFNgamma and TNFalpha in blood of C57BL/6 mice implanted with MC38 syngeneic colon carcinoma tumors. The figure shows plasma concentrations of IFNg and TNFα on the y-axis, and time on the x-axis. The IL-18 polypeptides tested in this figure are SEQ ID NO: 59 and SEQ ID NO: 59 conjugated to a 10, 20 and 30 kDa PEG tested as a single agent at 3 mg/kg as a single i.v. injection. (n=3 per time point; mean±SEM).

FIG. 18A shows a plot describing plasma exposure of modified IL-18 polypeptides in C57BL/6 mice implanted with MC38 syngeneic colon carcinoma tumors. The figure shows plasma concentrations on the y-axis, and time on the x-axis. The IL-18 polypeptides tested in this figure are SEQ ID NO: 30 and SEQ ID NO: 30 conjugated to a 05, 10 and 30 kDa PEG tested as a single agent at 3 mg/kg as a single i.v. injection. (n=3 per time point; mean±SEM).

FIG. 18B shows a plot describing the effect of modified IL-18 polypeptides on levels of IFNgamma in blood (left panel) and tumors (right panel) of C57BL/6 mice implanted with MC38 syngeneic colon carcinoma tumors. The figure shows plasma concentrations of IFNg and TNFα on the y-axis, and time on the x-axis. The IL-18 polypeptides tested in this figure are SEQ ID NO: 30 and SEQ ID NO: 30 conjugated to a 05, 10 and 30 kDa PEG tested as a single agent at 3 mg/kg as a single i.v. injection. (n=3 per time point; mean±SEM).

FIG. 19 shows a plot describing the effect of modified IL-18 polypeptides on activation status of NK and CD8+ T-cells in blood (upper panel) and tumors (bottom panel) of C57BL/6 mice implanted with MC38 syngeneic colon carcinoma tumors. The figure surface expression levels (Median Fluorescence Intensity) on the y-axis, and time on the x-axis. The IL-18 polypeptides tested in this figure are SEQ ID NO: 30 and SEQ ID NO: 30 conjugated to a 05, 10 and 30 kDa PEG tested as a single agent at 3 mg/kg as a single i.v. injection. (n=3 per time point; mean±SEM).

FIG. 20 shows tumor growth inhibition effects of IL-18 variant SEQ ID NO: 30 with 10, 20, and 30 kDa PEG attached to residue C68. The IL-18 variants were administered via single intravenous injections, at dosage 3 mg/kg to mice having MC38 tumor.

FIG. 21A shows a plot describing the effect of modified IL-18 polypeptides on the growth of MC38 syngeneic colon carcinoma tumors in C57BL/6 mice. The figure shows tumor volume of each individual mouse on the y-axis and time on the x-axis. The IL-18 polypeptide tested in this figure is SEQ ID NO: 59 conjugated to a 30 kDa PEG tested as a single agent at 3 and 7 mg/kg as two weekly i.v. injections. (n=6; mean±SEM).

FIG. 21B shows a plot describing the effect of modified IL-18 polypeptides on the weight C57BL/6 mice engrafted with MC38 syngeneic colon carcinoma tumors. The figure shows weight of each individual mouse on the y-axis and time on the x-axis. The IL-18 polypeptide tested in this figure is SEQ ID NO: 59 conjugated to a 30 kDa PEG tested as a single agent at 3 and 7 mg/kg as two weekly i.v. injections. (n=6; mean±SEM).

FIG. 22A shows a plot describing the effect of modified IL-18 polypeptide on the growth of MC38 syngeneic colon carcinoma tumors in C57BL/6 mice. The figure shows the mean tumor volume on day 12 post treatment initiation on the y-axis. The modified IL-18 polypeptide tested in this figure is SEQ ID NO: 59 conjugated to a 30 kDa PEG tested as a single agent at 3 and 7 mg/kg as two weekly i.v. injections. (n=6; mean±SEM; TGI: Tumor Growth Inhibition).

FIG. 22B shows a plot describing the effect of modified IL-18 polypeptide on the survival of MC38 syngeneic colon carcinoma tumor-bearing C57BL/6 mice the modified IL-18 polypeptide tested in this figure is SEQ ID NO: 59 conjugated to a 30 kDa PEG tested as a single agent at 3 and 7 mg/kg as two weekly i.v. injections. (n=6; CR: Complete response).

FIG. 23A shows a plot describing the effect of modified IL-18 polypeptides on the growth of MC38 syngeneic colon carcinoma tumors in C57BL/6 mice. The figure shows mean tumor volume on the y-axis and time on the x-axis. The IL-18 polypeptide tested in this figure is SEQ ID NO: 30 conjugated to a 30 kDa PEG tested as a single agent at 0.5, 2.5, and 10 mg/kg as two weekly i.v. injections. As a control, anti PD-1 was applied at 2 mg/kg as a single agent as six biweekly i.v. injections for two weeks. SEQ ID NO: 30 conjugated to a 30 kDa PEG was also tested at 2.5 mg/kg in combination with anti-PD1 antibody at 2 mg/kg (n=15; mean±SEM).

FIG. 23B shows a plot describing the effect of modified IL-18 polypeptides on the growth of MC38 syngeneic colon carcinoma tumors in C57BL/6 mice. The figure shows the mean tumor volume on day 15 post treatment initiation on the y-axis. The IL-18 polypeptide tested in this figure is SEQ ID NO: 30 conjugated to a 30 kDa PEG tested as a single agent at 0.5, 2.5, and 10 mg/kg as two weekly i.v. injections. As a control, anti PD-1 was applied at 2 mg/kg as a single agent as six biweekly i.v. injections for two weeks. SEQ ID NO: 30 conjugated to a 30 kDa PEG was also tested at 2.5 mg/kg in combination with anti-PD1 antibody at 2 mg/kg (n=15; One-way Anova test P-value<0.001, ** P-value<0.01, * P-value<0.1, ns non-significant, TGI: Tumor Growth Inhibition).

FIG. 24 shows a plot describing the effect of modified IL-18 polypeptide on the survival of MC38 syngeneic colon carcinoma tumor-bearing C57BL/6 mice. The IL-18 polypeptide tested in this figure is SEQ ID NO: 30 conjugated to a 30 kDa PEG tested as a single agent at 0.5, 2.5, and 10 mg/kg as two weekly i.v. injections. As a control, anti PD-1 was applied at 2 mg/kg as a single agent as six biweekly i.v. injections for two weeks. SEQ ID NO: 30 conjugated to a 30 kDa PEG was also tested at 2.5 mg/kg in combination with anti-PD1 antibody at 2 mg/kg (n=15; CR: Complete response).

FIG. 25 shows a plot describing the effect of modified IL-18 polypeptides on the growth of MC38 syngeneic colon carcinoma tumors in C57BL/6 mice. The figure shows tumor volume of each individual mouse on the y-axis and time on the x-axis. The IL-18 polypeptide tested in this figure is SEQ ID NO: 30 conjugated to a 30 kDa PEG tested as a single agent at 0.5, 2.5, and 10 mg/kg as two weekly i.v. injections. As a control, anti PD-1 was applied at 2 mg/kg as a single agent as six biweekly i.v. injections for two weeks. SEQ ID NO: 30 conjugated to a 30 kDa PEG was also tested at 2.5 mg/kg in combination with anti-PD1 antibody at 2 mg/kg.

FIGS. 26A-B show plasma levels of the IL-18 variant of SEQ ID NO: 30+30 kDa PEG, from non-human primates (NHPs) administered with the IL-18 variant at dosage 10 μg/kg, 30 μg/kg, and 100 μg/kg. FIG. 26A shows individual PK, and FIG. 26B shows dose normalized average PK over 1 to 120 hrs post administration

FIGS. 27A-C show the IL-18 variant of SEQ ID NO: 30 w/30 kDa PEG is resistant to IL-18 Binding protein in NHPs. Plasma levels of free IL-18 variant and total IL-18 variant (free IL-18+IL-18complexed with IL18BP), of NHPs administered with the variant at dosage 10 μg/kg (FIG. 27A), 30 μg/kg (FIG. 27B), and 100 μg/kg (FIG. 27C) is shown.

FIGS. 28A-C show the degree to which IL-18 variant of SEQ ID NO: 30 w/30 kDa PEG induces dose dependent cytokine release in blood in NHPs. Mice were administered with the IL-18 variant at dosage 10 μg/kg, 30 μg/kg, and 100 μg/kg. Post administration plasma levels of IFNγ (FIG. 28A), IL-6 (FIG. 28B), and TNFα (FIG. 28C) are shown.

### DETAILED DESCRIPTION

Immune responses to tumors are primarily the function of T helper type 1 (Th1) lymphocytes. Th1 responses include the secretion of cytokines IL-2, IL-12, IL-18, IFNγ, and the generation of specific cytotoxic T lymphocytes that recognize specific tumor antigens. The Th1 response is a vital arm of host defense against many microorganisms. However, the Th1 response is also associated with autoimmune diseases and organ transplant rejection.

Interleukin 18 (IL-18) is a pro-inflammatory cytokine that elicits biological activities that initiate or promote host defense and inflammation following infection or injury. IL-18 has been implicated in autoimmune diseases, myocardial function, emphysema, metabolic syndromes, psoriasis, inflammatory bowel disease, hemophagocytic syndromes, macrophage activation syndrome, sepsis, and acute kidney injury. In some models of disease, IL-18 plays a protective role.

IL-18 also plays a major role in the production of IFNγ from T-cells and natural killer cells. IFNγ is a Th1 cytokine mainly produced by T cells, NK cells, and macrophages and is critical for innate and adaptive immunity against viral, some bacterial, and protozoal infections. IFNγ is also an important activator of macrophages and inducer of Class II major histocompatibility complex (MHC) molecule expression.

IL-18 forms a signaling complex by binding to the IL-18 alpha chain (IL-18Rα), which is the ligand binding chain for mature IL-18. However, the binding affinity of IL-18 to IL-18Rα is low. In cells that express the co-receptor, IL-18 receptor beta chain (IL-18Rβ), a high affinity heterodimer complex is formed, which then activates cell signaling.

In order to improve the therapeutic potential of IL-18, addition of a half-life-extending polymer (e.g., polyethylene glycol (PEG)) can be desirous in order to improve pharmacokinetic (PK) or pharmacodynamics (PD) properties of IL-18. However, addition of a polymer to certain residues of IL-18 has been observed to result in reduced affinity to one or more subunits of the IL-18 receptor (e.g., the alpha subunit, the beta subunit, or the alpha/beta heterodimer) and result in a concomitant reduced bioactivity of the IL-18 (e.g., reduced potency in inducing production of IFN□ compared to WT IL-18). For example, PCT Publication Number WO2004091517, which is hereby incorporated by reference as if set forth in its entirety, shows that PEGylation of cysteine residues (natural or substituted) at certain positions of IL-18 in some instances may result in substantial loss of affinity to IL-18RD and ability to induce IFN□ production in immune cells compared to both WT IL-18 and to the non-PEGylated version of the modified IL-18. Thus, identification of modified IL-18 polypeptides that have extended half-life, enhanced ability to induce IFNγ production in immune cells, and superior in vivo tumor growth inhibition efficacy would provide substantial advantages.

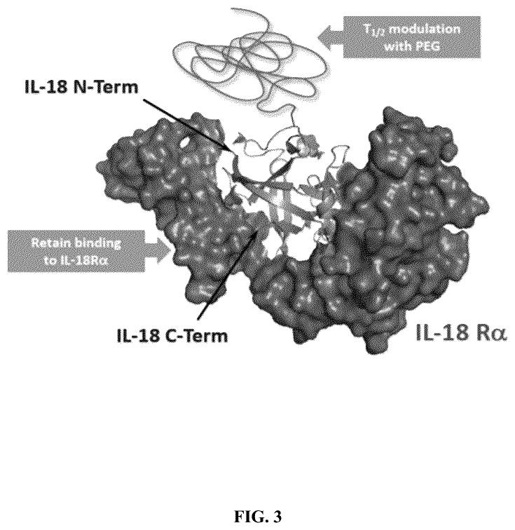

In order to overcome these deficiencies, provided herein are IL-18 polypeptides modified with polymers that substantially retain binding to IL-18R and bioactivity. In some embodiments, the addition of polymers to residues of the IL-18 polypeptide as provided herein allows for a half-life extended IL-18 with binding to IL-18R and/or bioactivity similar to or only slightly reduced compared to WT IL-18. In some embodiments, the addition of polymers to residues of the IL-18 polypeptide as provided herein allows for a half-life extended IL-18 with binding to IL-18R and/or bioactivity similar to or only slightly reduced compared to the IL-18 with no polymer attached. An exemplary picture of a modified IL-18 polypeptide with a polymer provided herein binding to an IL-18 receptor alpha subunit is shown in FIG. 3.

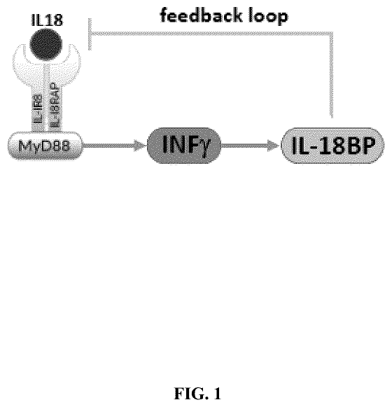

While extending the half-life of an IL-18 polypeptide with a polymer in a way that does not substantially diminish binding to IL-18R can offer advantages over WT and other variant IL-18s, other considerations can also affect the therapeutic potential of modified IL-18 polypeptides. For example, the activity of IL-18 is balanced by the presence of a high affinity, naturally occurring IL-18 binding protein (IL-18BP). IL-18BP binds IL-18 and neutralizes the biological activity of IL-18. Cell surface IL-18Rα competes with IL-18BP for IL-18 binding. Increased disease severity can be associated with an imbalance of IL-18 to IL-18BP such that levels of free IL-18 are elevated in the circulation. FIG. 1 illustrates the mechanism of action of IL-18, IFNγ production, IL-18BP production, and inhibition of IL-18 activity by IL-18BP. IL-18 induces IFNγ production, which in turn induces IL-18BP production. IL-18BP then competes with IL-18Rα to inhibit TL-18 activity. This feedback loop of IL-18BP production after stimulation of IFN□ production has limited the effectiveness of IL-18 as a treatment modality in previous efforts.

In some embodiments, a polymer modified IL-18 polypeptide described herein exhibits enhanced activity (e.g., interferon gamma stimulating activity, or another IL-18 related activity described herein) as compared to WT IL-18 with no polymer attached. In some embodiments, the polymer modified IL-18 polypeptide will exhibit an IL-18 related activity in vitro which inversely correlates with polymer molecular weight (i.e., the polymer modified IL-18 polypeptide exhibits less in vitro potency as the molecular weight increases). However, in some instances, it has been surprisingly found that such polymer modified IL-18 polypeptides with higher molecular weight polymers which show less in vitro potency (e.g., are observed to have greater potency in vivo (e.g., increased tumor growth inhibition properties). Thus, in some embodiments, a critical size of polymer attached to an IL-18 polypeptide which optimizes half-life extension and minimally reduces IL-18 activity is described herein which provides an optimized polymer bearing IL-18 therapeutic with better in vivo efficacy (e.g., tumor growth inhibition) despite apparent lower in vitro activity (e.g., interferon gamma stimulating activity).

In some embodiments, IL-18 polypeptides modified with polymers provided herein also exhibit reduced binding affinity to IL-18BP compared to WT IL-18. In some embodiments, the presence of the polymer results in reduced binding of the modified IL-18 polypeptide to IL-18 BP. In some embodiments, the modified IL-18 polypeptide contains one or more amino acid substitutions that reduce the interaction of the modified IL-18 with IL-18BP. In some embodiments, the modified IL-18 polypeptide contains one or more amino acid substitutions that reduce the ability of the IL-18 to be neutralized by IL-18BP and which enhance the IL-18's ability to bind to IL-18R. In some embodiments, IL-18 polypeptides modified with polymers provided herein contain one or more amino acid substitutions that allow the IL-18 polypeptide to have an optimal mix of high half-life, reduced IL-18BP neutralization, high affinity for IL-18R, and high in vivo tumor growth inhibition efficacy.

Thus, in certain embodiments, a modified IL-18 polypeptides of the instant disclosure shows sufficient potency to maximize the therapeutic benefit, sufficient half-life to allow for optimal in vivo response.

The following description and examples illustrate embodiments of the present disclosure in detail. It is to be understood that this present disclosure is not limited to the particular embodiments described herein and as such can vary. Those of skill in the art will recognize that there are numerous variations and modifications of this present disclosure, which are encompassed within its scope.

Although various features of the present disclosure may be described in the context of a single embodiment, the features may also be provided separately or in any suitable combination. Conversely, although the present disclosure may be described herein in the context of separate embodiments for clarity, the present disclosure may also be implemented in a single embodiment.

The section headings used herein are for organizational purposes only and are not to be construed as limiting the subject matter described.

### I. Definitions

All terms are intended to be understood as they would be understood by a person skilled in the art. Unless defined otherwise, all technical and scientific terms used herein have the same meaning as commonly understood by one of ordinary skill in the art to which the disclosure pertains.

The following definitions supplement those in the art and are directed to the current application and are not to be imputed to any related or unrelated case, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only and is not intended to be limiting.

In this application, the use of the singular includes the plural unless specifically stated otherwise. As used herein, the singular forms “a”, “an” and “the” are intended to include the plural forms as well, unless the context clearly indicates otherwise.

In this application, the use of “or” means “and/of” unless stated otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 15%, up to 10%, up to 5%, or up to 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value should be assumed.

Throughout the instant description, certain numerical or other similar values may be described as, for example, “at least” or “at most” a set of values indicated in a list form (e.g., “at least 2, 3, 4, 5, or 6”). In such cases, unless context clearly indicates otherwise, it is intended that the phrase “at least,” “at most,” or other similar term is applied individually to each value in the list. For example, the phrase “at least 2, 3, 4, 5, or 6” is to be interpreted as “at least 2, at least 3, at least 4, at least 5, or at least 6.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures. To facilitate an understanding of the present disclosure, a number of terms and phrases are defined below.

Referred to herein are polymers which are “attached” or “covalently attached” to residues of IL-18 polypeptides. As used herein, “attached” or “covalently attached” means that the polymer is tethered to the indicated reside, and such tethering can include a linking group (i.e., a linker). Thus, for a polymer “attached” or “covalently attached” to a residue, it is expressly contemplated that such linking groups are also encompassed.

Binding affinity refers to the strength of a binding interaction between a single molecule and its ligand/binding partner. A higher binding affinity refers to a higher strength bond than a lower binding affinity. In some instances, binding affinity is measured by the dissociation constant (KD) between the two relevant molecules. When comparing KD values, a binding interaction with a lower value will have a higher binding affinity than a binding interaction with a higher value. For a protein-ligand interaction, KD is calculated according to the following formula:

$${K}_{D} = \frac{[ L ] [ P ]}{[ L P ]}$$

where [L] is the concentration of the ligand, [P] is the concentration of the protein, and [LP] is the concentration of the ligand/protein complex.

Referred to herein are certain amino acid sequences (e.g., polypeptide sequences) which have a certain percent sequence identity to a reference sequence or refer to a residue at a position corresponding to a position of a reference sequence. Sequence identity is measured by protein-protein BLAST algorithm using parameters of Matrix BLOSUM62, Gap Costs Existence:11, Extension:1, and Compositional Adjustments Conditional Compositional Score Matrix Adjustment. This alignment algorithm is also used to assess if a residue is at a “corresponding” position through an analysis of the alignment of the two sequences being compared.

Referred to herein are polymers which are “attached” or “covalently attached” to residues of IL-18 polypeptides. As used herein, “attached” or “covalently attached” means that the polymer is tethered to the indicated reside, and such tethering can include a linking group (i.e., a linker). Thus, for a polymer “attached” or “covalently attached” to a residue, it is expressly contemplated that such linking groups are also encompassed.

In some instances, peptides provided herein may be depsipeptides. For example, a depsipeptide linkage result from certain ligation reactions described herein (e.g., KAHA ligations) during the synthesis of synthetic IL-18s and relevant precursor peptides. In particular, hydroxyl containing amino acids (e.g., serine, threonine, and homoserine) form depsipeptide linkages with the adjacent amino acid on the N-terminal side. Thus, when a sequence ID lists an amino acid sequence, it is also contemplated that a depsipeptide version of the sequence is also encompassed, particularly at homoserine residues.

The term “pharmaceutically acceptable” refers to approved or approvable by a regulatory agency of the Federal or a state government or listed in the U.S. Pharmacopeia or other generally recognized pharmacopeia for use in animals, including humans.

A “pharmaceutically acceptable excipient, carrier or diluent” refers to an excipient, carrier or diluent that can be administered to a subject, together with an agent, and which does not destroy the pharmacological activity thereof and is nontoxic when administered in doses sufficient to deliver a therapeutic amount of the agent.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

The term “subject” or “patient” refers to an animal which is the object of treatment, observation, or experiment. By way of example only, a subject or patient includes, but is not limited to, a mammal, including, but not limited to, a human or a non-human mammal, such as a non-human primate, bovine, equine, canine, ovine, or feline.

Certain formulas and other illustrations provided herein depict triazole reaction products resulting from azide-alkyne cycloaddition reactions. While such formulas generally depict only a single regioisomer of the resulting triazole formed in the reaction, it is intended that the formulas encompass both resulting regioisomers. Thus, while the formulas depict only a single regioisomer

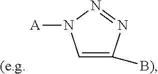

it is intended that the other regioisomer

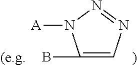

is also encompassed.

The term “optional” or “optionally” denotes that a subsequently described event or circumstance can but need not occur, and that the description includes instances where the event or circumstance occurs and instances in which it does not.

The term “moiety” refers to a specific segment or functional group of a molecule. Chemical moieties are often recognized chemical entities embedded in or appended to a molecule.

As used herein, “conjugation handle” refers to a reactive group capable of forming a bond upon contacting a complementary reactive group. In some instances, a conjugation handle preferably does not have a substantial reactivity with other molecules which do not comprise the intended complementary reactive group. Non-limiting examples of conjugation handles, their respective complementary conjugation handles, and corresponding reaction products can be found in the table below. While table headings place certain reactive groups under the title “conjugation handle” or “complementary conjugation handle,” it is intended that any reference to a conjugation handle can instead encompass the complementary conjugation handles listed in the table (e.g., a trans-cyclooctene can be a conjugation handle, in which case tetrazine would be the complementary conjugation handle). In some instances, amine conjugation handles and conjugation handles complementary to amines are less preferable for use in biological systems owing to the ubiquitous presence of amines in biological systems and the increased likelihood for off-target conjugation.

Table of Conjugation Handles

| Conjugation | | Reaction |
|---|---|---|
| Handle | Complementary Conjugation Handle | Product |
| Sulfhydryl | alpha-halo-carbonyl (e.g., bromoacet- | thioether |
| | amide), alpha-beta unsaturated carbonyl | |
| | (e.g., maleimide, acrylamide) | |
| Azide | alkyne (e.g., terminal alkyne, substituted | triazole |
| | cyclooctyne (e.g., dibenzocycloocytne | |
| | (DBCO), difluorocyclooctyne, | |
| | bicyclo[6.1.0]nonyne, etc.)) | |
| Phosphine | Azide/ester pair | amide |
| Tetrazine | trans-cyoclooctene | dihydropy- |
| | | ridazine |
| Amine | Activated ester (e.g., N-hydroxysuccin- | amide |
| | imide ester, pentaflurophenyl ester) | |
| isocyanate | Amine | urea |
| epoxide | Amine | alkyl-amine |
| hydroxyl amine | aldehyde, ketone | oxime |
| hydrazide | aldehyde, ketone | hydrazone |
| potassium acyl | O-substituted hydroxylamine (e.g., O- | amide |
| trifluoroborate | carbamoylhydroxylamine) | |

Throughout the instant application, prefixes are used before the term “conjugation handle” to denote the functionality to which the conjugation handle is linked. For example, a “protein conjugation handle” is a conjugation handle attached to a protein (either directly or through a linker), an “antibody conjugation handle” is a conjugation handle attached to an antibody (either directly or through a linker), and a “linker conjugation handle” is a conjugation handle attached to a linker group (e.g., a bifunctional linker used to link a synthetic protein and an antibody)

As used herein, the term “number average molecular weight” (Mn) means the statistical average molecular weight of all the individual units in a sample, and is defined by Formula (1):

$$M n = \frac{\sum {N}_{i} {M}_{i}}{\sum {N}_{i}} \qquad Formula ( 1 )$$

where Mᵢ is the molecular weight of a unit and Nᵢ is the number of units of that molecular weight.

As used herein, the term “weight average molecular weight” (Mw) means the number defined by Formula (2):

$$M w = \frac{\sum {N}_{i} {M}_{i}^{2}}{\sum {N}_{i} {M}_{i}} \qquad Formula ( 2 )$$

where Mᵢ is the molecular weight of a unit and Nᵢ is the number of units of that molecular weight.

As used herein, “peak molecular weight” (Mp) means the molecular weight of the highest peak in a given analytical method (e.g. mass spectrometry, size exclusion chromatography, dynamic light scattering, analytical centrifugation, etc.).

The term “alkyl” refers to a straight or branched hydrocarbon chain radical, having from one to twenty carbon atoms, and which is attached to the rest of the molecule by a single bond. An alkyl comprising up to 10 carbon atoms is referred to as a C₁-C₁₀ alkyl, likewise, for example, an alkyl comprising up to 6 carbon atoms is a C₁-C₆ alkyl. Alkyls (and other moieties defined herein) comprising other numbers of carbon atoms are represented similarly. Alkyl groups include, but are not limited to, C₁-C₁₀ alkyl, C₁-C₉ alkyl, C₁-C₈ alkyl, C₁-C₇ alkyl, C₁-C₆ alkyl, C₁-C₅ alkyl, C₁-C₄ alkyl, C₁-C₃ alkyl, C₁-C₂ alkyl, C₂-C₈ alkyl, C₃-C₈ alkyl and C₄-C₈ alkyl. Representative alkyl groups include, but are not limited to, methyl, ethyl, «-propyl, 1-methyl ethyl (/-propyl), «-butyl, /-butyl, 5-butyl, «-pentyl, 1,1-dimethyl ethyl (/-butyl), 3-methylhexyl, 2-methylhexyl, 1-ethyl-propyl, and the like. In some embodiments, the alkyl is methyl or ethyl. In some embodiments, the alkyl is —CH(CH₃)₂ or —C(CH₃)₃. Unless stated otherwise specifically in the specification, an alkyl group may be optionally substituted. “Alkylene” or “alkylene chain” refers to a straight or branched divalent hydrocarbon chain linking the rest of the molecule to a radical group. In some embodiments, the alkylene is —CFF—, —CH₂CH₂—, or —CH₂CH₂CH₂—. In some embodiments, the alkylene is —CH₂—. In some embodiments, the alkylene is —CH₂CH₂—. In some embodiments, the alkylene is —CH₂CH₂CH₂—. Unless stated otherwise specifically in the specification, an alkylene group may be optionally substituted.

The term “alkenylene” or “alkenylene chain” refers to a straight or branched divalent hydrocarbon chain in which at least one carbon-carbon double bond is present linking the rest of the molecule to a radical group. In some embodiments, the alkenylene is —CH═CH—, —CH₂CH═CH—, or —CH═CHCH₂—. In some embodiments, the alkenylene is —CH═CH—. In some embodiments, the alkenylene is —CH₂CH═CH—. In some embodiments, the alkenylene is —CH═CHCH₂—.

The term “alkynyl” refers to a type of alkyl group in which at least one carbon-carbon triple bond is present. In one embodiment, an alkenyl group has the formula —C□C—R^{X}, wherein R^{x} refers to the remaining portions of the alkynyl group. In some embodiments, R^{x} is H or an alkyl. In some embodiments, an alkynyl is selected from ethynyl, propynyl, butynyl, pentynyl, hexynyl, and the like. Non-limiting examples of an alkynyl group include —C□CH, —C□CCH₃, —C□CCH₂CH₃, and —CH₂C□CH.

The term “alkynyl” refers to a type of alkyl group in which at least one carbon-carbon triple bond is present. In one embodiment, an alkenyl group has the formula —C□C—R^{X}, wherein R^{X} refers to the remaining portions of the alkynyl group. In some embodiments, R^{X} is H or an alkyl. In some embodiments, an alkynyl is selected from ethynyl, propynyl, butynyl, pentynyl, hexynyl, and the like. Non-limiting examples of an alkynyl group include —C□CH, —C□CCH₃, —C□CCH₂CH, and —CH₂C□CH.

The term “aryl” refers to a radical comprising at least one aromatic ring wherein each of the atoms forming the ring is a carbon atom. Aryl groups can be optionally substituted. Examples of aryl groups include, but are not limited to phenyl, and naphthyl. In some embodiments, the aryl is phenyl. Depending on the structure, an aryl group can be a monoradical or a diradical (i.e., an arylene group). Unless stated otherwise specifically in the specification, the term “aryl” or the prefix “ar-” (such as in “aralkyl”) is meant to include aryl radicals that are optionally substituted. In some embodiments, an aryl group comprises a partially reduced cycloalkyl group defined herein (e.g., 1,2-dihydronaphthalene). In some embodiments, an aryl group comprises a fully reduced cycloalkyl group defined herein (e.g., 1,2,3,4-tetrahydronaphthalene). When aryl comprises a cycloalkyl group, the aryl is bonded to the rest of the molecule through an aromatic ring carbon atom. An aryl radical can be a monocyclic or polycyclic (e.g., bicyclic, tricyclic, or tetracyclic) ring system, which may include fused, spiro or bridged ring systems.

The term “cycloalkyl” refers to a monocyclic or polycyclic non-aromatic radical, wherein each of the atoms forming the ring (i.e. skeletal atoms) is a carbon atom. In some embodiments, cycloalkyls are saturated or partially unsaturated. In some embodiments, cycloalkyls are spirocyclic or bridged compounds. In some embodiments, cycloalkyls are fused with an aromatic ring (in which case the cycloalkyl is bonded through a non-aromatic ring carbon atom). Cycloalkyl groups include groups having from 3 to 10 ring atoms. Representative cycloalkyls include, but are not limited to, cycloalkyls having from three to ten carbon atoms, from three to eight carbon atoms, from three to six carbon atoms, or from three to five carbon atoms. Monocyclic cycloalkyl radicals include, for example, cyclopropyl, cyclobutyl, cyclopentyl, cyclohexyl, cycloheptyl, and cyclooctyl. In some embodiments, the monocyclic cycloalkyl is cyclopentyl. In some embodiments, the monocyclic cycloalkyl is cyclopentenyl or cyclohexenyl. In some embodiments, the monocyclic cycloalkyl is cyclopentenyl. Polycyclic radicals include, for example, adamantyl, 1,2-dihydronaphthalenyl, 1,4-dihydronaphthalenyl, tetrainyl, decalinyl, 3,4-dihydronaphthalenyl-1(2H)-one, spiro[2.2]pentyl, norbornyl and bicycle[1.1.1]pentyl. Unless otherwise stated specifically in the specification, a cycloalkyl group may be optionally substituted.

The term “heteroalkylene” or “heteroalkylene chain” refers to a straight or branched divalent heteroalkyl chain linking the rest of the molecule to a radical group. Unless stated otherwise specifically in the specification, the heteroalkyl or heteroalkylene group may be optionally substituted as described below. Representative heteroalkylene groups include, but are not limited to —CH₂—O—CH₂—, —CH₂—N(alkyl)-CH₂—, —CH₂—N(aryl)-CH₂—, —OCH₂CH₂O—, —OCH₂CH₂OCH₂CH₂O—, or —OCH₂CH₂OCH₂CH₂OCH₂CH₂O—.

The term “heteocycloalkyl” refers to a cycloalkyl group that includes at least one heteroatom selected from nitrogen, oxygen, and sulfur. Unless stated otherwise specifically in the specification, the heterocycloalkyl radical may be a monocyclic, or bicyclic ring system, which may include fused (when fused with an aryl or a heteroaryl ring, the heterocycloalkyl is bonded through a non-aromatic ring atom) or bridged ring systems. The nitrogen, carbon or sulfur atoms in the heterocyclyl radical may be optionally oxidized. The nitrogen atom may be optionally quaternized. The heterocycloalkyl radical is partially or fully saturated. Examples of heterocycloalkyl radicals include, but are not limited to, dioxolanyl, thienyl[1,3]dithianyl, tetrahydroquinolyl, tetrahydroisoquinolyl, decahydroquinolyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl. The term heterocycloalkyl also includes all ring forms of carbohydrates, including but not limited to monosaccharides, disaccharides and oligosaccharides. Unless otherwise noted, heterocycloalkyls have from 2 to 12 carbons in the ring. In some embodiments, heterocycloalkyls have from 2 to 10 carbons in the ring. In some embodiments, heterocycloalkyls have from 2 to 10 carbons in the ring and 1 or 2 N atoms. In some embodiments, heterocycloalkyls have from 2 to 10 carbons in the ring and 3 or 4 N atoms. In some embodiments, heterocycloalkyls have from 2 to 12 carbons, 0-2 N atoms, 0-2 O atoms, 0-2 P atoms, and 0-1 S atoms in the ring. In some embodiments, heterocycloalkyls have from 2 to 12 carbons, 1-3 N atoms, 0-1 O atoms, and 0-1 S atoms in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Unless stated otherwise specifically in the specification, a heterocycloalkyl group may be optionally substituted.

The term “heteroaryl” refers to an aryl group that includes one or more ring heteroatoms selected from nitrogen, oxygen, and sulfur. In some embodiments, heteroaryl is monocyclic or bicyclic. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, furazanyl, indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Illustrative examples of bicyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, heteroaryl is pyridinyl, pyrazinyl, pyrimidinyl, thiazolyl, thienyl, thiadiazolyl or furyl. In some embodiments, a heteroaryl contains 0-6 N atoms in the ring. In some embodiments, a heteroaryl contains 1-4 N atoms in the ring. In some embodiments, a heteroaryl contains 4-6 N atoms in the ring. In some embodiments, a heteroaryl contains 0-4 N atoms, 0-1 O atoms, 0-1 P atoms, and 0-1 S atoms in the ring. In some embodiments, a heteroaryl contains 1-4 N atoms, 0-1 O atoms, and 0-1 S atoms in the ring. In some embodiments, heteroaryl is a C₁-C₉ heteroaryl. In some embodiments, monocyclic heteroaryl is a C₁-C₅ heteroaryl. In some embodiments, monocyclic heteroaryl is a 5-membered or 6-membered heteroaryl. In some embodiments, a bicyclic heteroaryl is a C₆-C₉ heteroaryl. In some embodiments, a heteroaryl group comprises a partially reduced cycloalkyl or heterocycloalkyl group defined herein (e.g., 7,8-dihydroquinoline). In some embodiments, a heteroaryl group comprises a fully reduced cycloalkyl or heterocycloalkyl group defined herein (e.g., 5,6,7,8-tetrahydroquinoline). When heteroaryl comprises a cycloalkyl or heterocycloalkyl group, the heteroaryl is bonded to the rest of the molecule through a heteroaromatic ring carbon or hetero atom. A heteroaryl radical can be a monocyclic or polycyclic (e.g., bicyclic, tricyclic, or tetracyclic) ring system, which may include fused, spiro or bridged ring systems.

The term “optionally substituted” or “substituted” means that the referenced group is optionally substituted with one or more additional group(s) individually and independently selected from D, halogen, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from D, halogen, —CN, —NH₂, —NH(CH₃), —N(CH₃)₂, —OH, —CO₂H, —CO₂(C₁-C₄alkyl), —C(═O)NH₂, —C(═O)NH(C₁-C₄alkyl), —C(═O)N(C₁-C₄alkyl)₂, —S(═O)₂NH₂, —S(═O)₂NH(C₁-C₄alkyl), —S(═O)₂N(C₁-C₄alkyl)₂, C₁-C₄alkyl, C₃-C₆cycloalkyl, C₁-C₄fluoroalkyl, C₁-C₄heteroalkyl, C₁-C₄alkoxy, C₁-C₄fluoroalkoxy, —SC₁-C₄alkyl, —S(═O)C₁-C₄alkyl, and —S(═O)₂C₁-C₄alkyl. In some embodiments, optional substituents are independently selected from D, halogen, —CN, —NH₂, —OH, —NH(CH₃), —N(CH₃)₂, —NH(cyclopropyl), —CH₃, —CH₂CH₃, —CF₃, —OCH₃, and —OCF₃. In some embodiments, substituted groups are substituted with one or two of the preceding groups. In some embodiments, an optional substituent on an aliphatic carbon atom (acyclic or cyclic) includes oxo (═O).

### II. Modified IL-18 Polypeptides

Certain aspects of the present disclosure relate to modified IL-18 polypeptides useful as therapeutic agents. The modified IL-18 polypeptides provided herein can be used as immunotherapies or as parts of other immunotherapy regimens. The modified IL-18 polypeptide comprises a polymer covalently attached to an amino acid residue of the modified IL-18 polypeptide. In some embodiments, the modified IL-18 polypeptides provided herein exhibit an improved to ability to induce IFNγ production in a cell, compared to WT IL-18, even in the presence of the polymer. In certain embodiments, the modified IL-18 polypeptides provided herein exhibit a reduced binding to IL-18 binding protein (IL-18BP) compared to WT IL-18, and/or an improved activity of IL-18 receptor (IL-18R) signaling in the presence of IL-18BP compared to WT IL-18. In certain embodiments, the modified IL-18 polypeptides provided herein exhibit a reduced binding to IL-18 binding protein (IL-18BP) compared to WT IL-18, and an improved activity of IL-18 receptor (IL-18R) signaling in the presence of IL-18BP compared to WT IL-18.

IIa. Site Specific Modification of the Modified IL-18 Polypeptide

In some embodiments, a modified IL-18 polypeptide described herein comprises one or more modifications at one or more amino acid residues. As used herein, “modifications” of the modified IL-18 polypeptide are relative to SEQ ID NO: 1. In some embodiments, the residue position numbering of the modified IL-18 polypeptide is based on SEQ ID NO: 1 as a reference sequence.

Modifications to the polypeptides described herein encompass mutations, addition of various functionalities, deletion of amino acids, addition of amino acids, or any other alteration of the wild-type version of the protein or protein fragment. Functionalities which may be added to polypeptides include polymers, linkers, alkyl groups, detectable molecules such as chromophores or fluorophores, reactive functional groups, or any combination thereof. In some embodiments, functionalities are added to individual amino acids of the polypeptides. In some embodiments, functionalities are added site-specifically to the polypeptides.

In some embodiments, the modified IL-18 polypeptides described herein contain 1, 2, 3, 4, 5, 6, 7, 8, 9, or 10 modified amino acid residues.

In some embodiments, a modified IL-18 polypeptide described herein comprises at least 1, at least 2, at least 3, at least 4, at least 5, at least 6, at least 7, or at least 9 amino acid substitutions, wherein the amino acid substitutions are relative to SEQ ID NO: 1. In some embodiments, the modified IL-18 polypeptide comprises 1 to 9 amino acid substitutions. In some embodiments, the modified IL-18 polypeptide comprises 1 or 2 amino acid substitutions, 1 to 3 amino acid substitutions, 1 to 4 amino acid substitutions, 1 to 5 amino acid substitutions, 1 to 6 amino acid substitutions, 1 to 7 amino acid substitutions, 1 to 8 amino acid substitutions, 2 to 3 amino acid substitutions, 2 to 4 amino acid substitutions, 2 to 5 amino acid substitutions, 2 to 6 amino acid substitutions, 2 to 7 amino acid substitutions, 2 to 8 amino acid substitutions, 2 to 9 amino acid substitutions 3 or 4 amino acid substitutions, 3 to 5 amino acid substitutions, 3 to 6 amino acid substitutions, 3 to 7 amino acid substitutions, 3 to 9 amino acid substitutions, 4 or 5 amino acid substitutions, 4 to 6 amino acid substitutions, 4 to 7 amino acid substitutions, 4 to 9 amino acid substitutions, 5 or 6 amino acid substitutions, 5 to 7 amino acid substitutions, 5 to 9 amino acid substitutions, 6 or 7 amino acid substitutions, 6 to 9 amino acid substitutions, or 7 to 9 amino acid substitutions. In some embodiments, the modified IL-18 polypeptide comprises 3 amino acid substitutions, 4 amino acid substitutions, 5 amino acid substitutions, 6 amino acid substitutions, 7 amino acid substitutions, or 9 amino acid substitutions. In some embodiments, the modified IL-18 polypeptide comprises at most 4 amino acid substitutions, 5 amino acid substitutions, 6 amino acid substitutions, 7 amino acid substitutions, or 9 amino acid substitutions.

In certain embodiments, the modified IL-18 polypeptide comprises at least one glycine residue attached to the N-terminus of the IL-18 polypeptide. In certain embodiments, the modified IL-18 polypeptide comprises a chain of glycine residues attached to the N-terminus of the polypeptide, wherein the chain of glycine residues comprises 1 to 15 glycine residues. In certain embodiments, the modified IL-18 polypeptide comprises a chain of 1 to 10 glycine residues attached to the N-terminus of the IL-18 polypeptide. In certain embodiments, the modified IL-18 polypeptide comprises a chain of 1, 2, 3, 4, 5, 6, 7, 8, 9, or 10, or any range therebetween glycine residues attached to the N-terminus of the IL-18 polypeptide. In certain embodiments, the modified IL-18 polypeptide comprises a glycine residue attached to the N-terminus of the IL-18 polypeptide. In certain embodiments, the modified IL-18 polypeptide comprises a chain of 2 glycine residues attached to the N-terminus of the IL-18 polypeptide. In certain embodiments, the modified IL-18 polypeptide comprises a chain of 3 glycine residues attached to the N-terminus of the IL-18 polypeptide. In certain embodiments, the modified IL-18 polypeptide comprises a chain of 4 glycine residues attached to the N-terminus of the IL-18 polypeptide. In certain embodiments, the modified IL-18 polypeptide comprises a chain of 5 glycine residues attached to the N-terminus of the IL-18 polypeptide. In certain embodiments, the modified IL-18 polypeptide comprises a chain of glycine residues attached to the N-terminus of the polypeptide, wherein the chain of glycine residues comprises 1 to 2, 1 to 3, 1 to 4, 1 to 5, 1 to 6, 1 to 7, 1 to 8, 1 to 9, 1 to 10, 1 to 12, 1 to 15, 2 to 3, 2 to 4, 2 to 5, 2 to 6, 2 to 7, 2 to 8, 2 to 9, 2 to 10, 2 to 12, 2 to 15, 3 to 4, 3 to 5, 3 to 6, 3 to 7, 3 to 8, 3 to 9, 3 to 10, 3 to 12, 3 to 15, 4 to 5, 4 to 6, 4 to 7, 4 to 8, 4 to 9, 4 to 10, 4 to 12, 4 to 15, 5 to 6, 5 to 7, 5 to 8, 5 to 9, 5 to 10, 5 to 12, 5 to 15, 6 to 7, 6 to 8, 6 to 9, 6 to 10, 6 to 12, 6 to 15, 7 to 8, 7 to 9, 7 to 10, 7 to 12, 7 to 15, 8 to 9, 8 to 10, 8 to 12, 8 to 15, 9 to 10, 9 to 12, 9 to 15, 10 to 12, 10 to 15, or 12 to 15 glycine residues. In certain embodiments, the modified IL-18 polypeptide comprises a chain of glycine residues attached to the N-terminus of the polypeptide, wherein the chain of glycine residues comprises 1, 2, 3, 4, 5, 6, 7, 8, 9, 10, 12, or 15 glycine residues. In certain embodiments, the modified IL-18 polypeptide comprises a chain of glycine residues attached to the N-terminus of the polypeptide, wherein the chain of glycine residues comprises at least 1, 2, 3, 4, 5, 6, 7, 8, 9, 10, or 12 glycine residues. In certain embodiments, the modified IL-18 polypeptide comprises a chain of glycine residues attached to the N-terminus of the polypeptide, wherein the chain of glycine residues comprises at most 2, 3, 4, 5, 6, 7, 8, 9, 10, 12, or 15 glycine residues.

In certain embodiments, the modified IL-18 polypeptide comprises a substitution at residue Y1. In certain embodiments, the modified IL-18 polypeptide can comprises Y1M substitution. Unless specifically mentioned otherwise, the residue position numbering is provided in this paragraph, and elsewhere in this disclosure is based on SEQ ID NO: 1, as a reference sequence. Unless specifically mentioned otherwise, the amino acid substitutions provided in this paragraph, and elsewhere in this disclosure is with respect to SEQ ID NO: 1, as a reference sequence. In certain embodiments, the modified IL-18 polypeptide comprises a substitution at residue F2. In certain embodiments, the modified IL-18 polypeptide can comprises F2A substitution. In certain embodiments, the modified IL-18 polypeptide comprises a substitution at residue E6. In certain embodiments, the modified IL-18 polypeptide comprises E6K substitution. In certain embodiments, the modified IL-18 polypeptide comprises E6R substitution. In certain embodiments, the modified IL-18 polypeptide comprises a substitution at residue K8. In certain embodiments, the modified IL-18 polypeptide comprises K8L substitution. In certain embodiments, the modified IL-18 polypeptide comprises K8E substitution. In certain embodiments, the modified IL-18 polypeptide comprises K8R substitution. In certain embodiments, the modified IL-18 polypeptide comprises a substitution at residue V11. In certain embodiments, the modified IL-18 polypeptide can comprises V11I substitution. In certain embodiments, the modified IL-18 polypeptide comprises a substitution at residue E31. In certain embodiments, the modified IL-18 polypeptide comprises E31A substitution. In certain embodiments, the modified IL-18 polypeptide comprises a substitution at residue T34. In certain embodiments, the modified IL-18 polypeptide comprises T34A substitution. In certain embodiments, the modified IL-18 polypeptide comprises a substitution at residue D35. In certain embodiments, the modified IL-18 polypeptide comprises D35A substitution. In certain embodiments, the modified IL-18 polypeptide comprises a substitution at residue S36. In certain embodiments, the modified IL-18 polypeptide comprises S36A substitution. In certain embodiments, the modified IL-18 polypeptide comprises a substitution at residue D37. In certain embodiments, the modified IL-18 polypeptide comprises D37A substitution. In certain embodiments, the modified IL-18 polypeptide comprises a substitution at residue D40. In certain embodiments, the modified IL-18 polypeptide comprises D40A substitution. In certain embodiments, the modified IL-18 polypeptide comprises a substitution at residue N41. In certain embodiments, the modified IL-18 polypeptide comprises N41A substitution. In certain embodiments, the modified IL-18 polypeptide comprises a substitution at residue 149. In certain embodiments, the modified IL-18 polypeptide comprises 149E substitution. In certain embodiments, the modified IL-18 polypeptide comprises I49M substitution. In certain embodiments, the modified IL-18 polypeptide comprises I49R substitution. In certain embodiments, the modified IL-18 polypeptide comprises a substitution at residue K53. In certain embodiments, the modified IL-18 polypeptide comprises K53A substitution. In certain embodiments, the modified IL-18 polypeptide comprises a substitution at residue D54. In certain embodiments, the modified IL-18 polypeptide comprises D54A substitution. In certain embodiments, the modified IL-18 polypeptide comprises a substitution at residue S55. In certain embodiments, the modified IL-18 polypeptide comprises S55A substitution. In certain embodiments, the modified IL-18 polypeptide comprises S55T substitution. In certain embodiments, the modified IL-18 polypeptide comprises S55H substitution. In certain embodiments, the modified IL-18 polypeptide comprises S55R substitution. In certain embodiments, the modified IL-18 polypeptide comprises a substitution at residue T63. In certain embodiments, the modified IL-18 polypeptide comprises T63A substitution. In certain embodiments, the modified IL-18 polypeptide comprises a substitution at residue Q103. In certain embodiments, the modified IL-18 polypeptide comprises Q103R substitution. In certain embodiments, the modified IL-18 polypeptide comprises Q103E substitution. In certain embodiments, the modified IL-18 polypeptide comprises Q103K substitution. In certain embodiments, the modified IL-18 polypeptide comprises a substitution at residue G108. In certain embodiments, the modified IL-18 polypeptide comprises G108A substitution. In certain embodiments, the modified IL-18 polypeptide comprises a substitution at residue H109. In certain embodiments, the modified IL-18 polypeptide comprises H109A substitution. In certain embodiments, the modified IL-18 polypeptide comprises a substitution at residue D110. In certain embodiments, the modified IL-18 polypeptide comprises D110A substitution. In certain embodiments, the modified IL-18 polypeptide comprises a substitution at residue D132. In certain embodiments, the modified IL-18 polypeptide comprises D132A substitution. In certain embodiments, the modified IL-18 polypeptide comprises a substitution at residue V153. In certain embodiments, the modified IL-18 polypeptide comprises V153R substitution. In certain embodiments, the modified IL-18 polypeptide comprises V153E substitution. In certain embodiments, the modified IL-18 polypeptide comprises V153Y substitution. In certain embodiments, the modified IL-18 polypeptide comprises a substitution at residue C38. In certain embodiments, the modified IL-18 polypeptide comprises C38A substitution. In certain embodiments, the modified IL-18 polypeptide comprises C38S substitution. In certain embodiments, the modified IL-18 polypeptide comprises a substitution at residue C68. In certain embodiments, the modified IL-18 polypeptide comprises C68A substitution. In certain embodiments, the modified IL-18 polypeptide comprises C68S substitution. In certain embodiments, the modified IL-18 polypeptide comprises a substitution at residue C76. In certain embodiments, the modified IL-18 polypeptide comprises C76A substitution. In certain embodiments, the modified IL-18 polypeptide comprises C76S substitution. In certain embodiments, the modified IL-18 polypeptide comprises a substitution at residue C127. In certain embodiments, the modified IL-18 polypeptide comprises C127A substitution. In certain embodiments, the modified IL-18 polypeptide comprises C127S substitution. In certain embodiments, the modified IL-18 polypeptide comprises a substitution at residue C38, C68, C76, and/or C127. In certain embodiments, the modified IL-18 polypeptide comprises a C38A, C38S, C68A, C68S, C76A, C76S, C127A, and/or C127S substitution. In certain embodiments, the modified IL-18 polypeptide comprises C38A, C76A, and C127A substitutions. In certain embodiments, the modified IL-18 polypeptide comprises C38S, C76S and C127S substitutions.

In certain embodiments, the modified IL-18 polypeptide comprises i) a substitution at residue V11 and ii) at least one additional substitution at a residue selected from Y1, F2, E6, K8, S10, D17, T34, D35, S36, D37, D40, N41, I49, M51, K53, D54, S55, Q103, S105, G108, H109, D110, and D132. In certain embodiments, the modified IL-18 polypeptide comprises i) a substitution at residue V11 and ii) at least one additional substitution at a residue selected from Y1, F2, E6, K8, S10, D17, T34, D35, S36, D37, D40, N41, I49, M51, K53, D54, S55, Q103, S105, G108, H109, D110, D132 and V153. In certain embodiments, the modified IL-18 polypeptide comprises i) V11I substitution and ii) at least one additional substitution selected from Y1M, F2A, E6K, E6R, K8L, K8E, K8R, T34A, D35A, S36A, D37A, D40A, N41A, 149E, I49M, I49R, M51G, K53A, D54A, S55A, S55T, S55H, S55R, S55H, Q103R, Q103E, Q103K, S105K, S105I, G108A, H109A, D110A, and D132A. In certain embodiments, the modified IL-18 polypeptide comprises i) V11I substitution and ii) at least one additional substitution selected from Y1M, F2A, E6K, E6R, K8L, K8E, K8R, T34A, D35A, S36A, D37A, D40A, N41A, 149E, I49M, I49R, M51G, K53A, D54A, S55A, S55T, S55H, S55R, S55H, Q103R, Q103E, Q103K, S105K, S105I, G108A, H109A, D110A, D132A, V153R, V153E and V153Y. In certain embodiments, the modified IL-18 polypeptide comprises i) substitution at residue V11, and ii) substitution at residue E6, M51 and/or K53. In certain embodiments, the modified IL-18 polypeptide comprises i) V11I substitution, and ii) E6K, M51G, and/or K53A substitution. In certain embodiments, the modified IL-18 polypeptide comprises substitutions at residues E6, V11, K53 and T63. In certain embodiments, the modified IL-18 polypeptide comprises E6K, V11, K53A and T63A substitutions. In certain embodiments, the modified IL-18 polypeptide comprises substitutions at residues V11, M51, K53. In certain embodiments, the modified IL-18 polypeptide comprises V11I, M51G, and K53A substitutions. In certain embodiments, the modified IL-18 polypeptide comprises substitutions at residues E6, V11, M51, K53 and T63. In certain embodiments, the modified IL-18 polypeptide comprises E6K, V11I, M51G, K53A and T63A substitutions. In certain embodiments, the modified IL-18 polypeptide comprises substitutions at residues E6, V11, C38, K53, T63, C76, and C127. In certain embodiments, the modified Il-18 polypeptide comprises E6K, V11, C38A, K53A, T63A, C76A, and C127A substitutions. In certain embodiments, the modified IL-18 polypeptide comprises substitutions at residues V11, C38, M51, K53, T63, C76, and C127. In certain embodiments, the modified 11-18 polypeptide comprises V11, C38A, M51G, K53A, T63A, C76A, and C127A substitutions. In certain embodiments, the modified IL-18 polypeptide comprises substitutions at residues V11, C38, M51, K53, C76, and C127. In certain embodiments, the modified Il-18 polypeptide comprises V11, C38A, M51G, K53A, C76A, and C127A substitutions. In certain embodiments, the modified IL-18 polypeptide comprises substitutions at residues E6, V11, C38, M51, K53, T63, C76, and C127. In certain embodiments, the modified IL-18 polypeptide comprises E6K, V11I, C38A, M51G, K53A, T63A, C76A, and C127A substitutions. In certain embodiments, the modified IL-18 polypeptide comprises i) a chain of 1, 2, 3, 4, 5, 6, 7, 8, 9, or 10, or any range therebetween glycine residues attached to the N-terminus of the IL-18 polypeptide, and ii) substitutions at residues E6, V11, C38, K53, T63, C76, and C127. In certain embodiments, the modified IL-18 polypeptide comprises i) a chain of 1, 2, 3, 4, 5, 6, 7, 8, 9, or 10, or any range therebetween glycine residues attached to the N-terminus of the IL-18 polypeptide, and ii) E6K, V11, C38A, K53A, T63A, C76A, and C127A substitutions. In certain embodiments, the modified IL-18 polypeptide comprises i) a glycine residue attached to the N-terminus of the IL-18 polypeptide, and ii) substitutions at residues E6, V11, C38, K53, T63, C76, and C127. In certain embodiments, the modified 11-18 polypeptide comprises i) a glycine residue attached to the N-terminus of the IL-18 polypeptide, and ii) E6K, V11, C38A, K53A, T63A, C76A, and C127A substitutions. In certain embodiments, the modified IL-18 polypeptide comprises i) a chain of 4 glycine residues attached to the N-terminus of the IL-18 polypeptide, and ii) substitutions at residues E6, V11, C38, K53, T63, C76, and C127. In certain embodiments, the modified IL-18 polypeptide comprises i) a chain of 4 glycine residues attached to the N-terminus of the IL-18 polypeptide, and ii) E6K, V11, C38A, K53A, T63A, C76A, and C127A substitutions. In certain embodiments, the modified IL-18 polypeptide comprises i) a chain of 1, 2, 3, 4, 5, 6, 7, 8, 9, or 10, or any range therebetween glycine residues attached to the N-terminus of the IL-18 polypeptide, and ii) substitutions at residues E6, V11, C38, M51, K53, T63, C76, and C127. In certain embodiments, the modified IL-18 polypeptide comprises i) a chain of 1, 2, 3, 4, 5, 6, 7, 8, 9, or 10, or any range therebetween glycine residues attached to the N-terminus of the IL-18 polypeptide, and ii) E6K, V11I, C38A, M51G, K53A, T63A, C76A, and C127A substitutions. In certain embodiments, the modified IL-18 polypeptide comprises i) a glycine residue attached to the N-terminus of the IL-18 polypeptide, and ii) substitutions at residues E6, V11, C38, M51, K53, T63, C76, and C127. In certain embodiments, the modified Il-18 polypeptide comprises i) a glycine residue attached to the N-terminus of the IL-18 polypeptide, and ii) E6K, V11I, C38A, M51G, K53A, T63A, C76A, and C127A substitutions. In certain embodiments, the modified IL-18 polypeptide comprises substitutions at residues V11, C38, K53, C76, and C127. In certain embodiments, the modified IL-18 polypeptide comprises V11I, C38A, K53A, C76A, and C127A substitutions. In certain embodiments, the modified IL-18 polypeptide comprises substitutions at residues V11, C38, K53, T63, C76, and C127. In certain embodiments, the modified IL-18 polypeptide comprises V11I, C38A, K53A, T63A, C76A, and C127A substitutions. In certain embodiments, the modified IL-18 polypeptide comprises substitutions at residues V11, C38, K53, S55, C76, and C127. In certain embodiments, the modified IL-18 polypeptide comprises V11I, C38A, K53A, S55A, C76A, and C127A substitutions. In certain embodiments, the modified IL-18 polypeptide comprises substitutions at residues V11, C38, K53, M51, C76, and C127. In certain embodiments, the modified IL-18 polypeptide comprises V11I, C38A, K53A, M51G, C76A, and C127A substitutions. In certain embodiments, the modified IL-18 polypeptide comprises substitutions at residues V11, C38, K53, D54, C76, and C127. In certain embodiments, the modified IL-18 polypeptide comprises V11I, C38A, K53A, D54A, C76A, and C127A substitutions. In certain embodiments, the modified IL-18 polypeptide comprises substitutions at residues F2, V11, C38, K53, C76, and C127. In certain embodiments, the modified IL-18 polypeptide comprises F2A, V11I, C38A, K53A, C76A, and C127A substitutions. In certain embodiments, the modified IL-18 polypeptide comprises substitutions at residues V11, E31, C38, K53, C76, and C127. In certain embodiments, the modified IL-18 polypeptide comprises V11I, E31A, C38A, K53A, C76A, and C127A substitutions. In certain embodiments, the modified IL-18 polypeptide comprises substitutions at residues V11, T34, C38, K53, C76, and C127. In certain embodiments, the modified IL-18 polypeptide comprises V11I, T34A, C38A, K53A, C76A, and C127A substitutions. In certain embodiments, the modified IL-18 polypeptide comprises substitutions at residues V11, D35, C38, K53, C76, and C127. In certain embodiments, the modified IL-18 polypeptide comprises V11I, D35A, C38A, K53A, C76A, and C127A substitutions. In certain embodiments, the modified IL-18 polypeptide comprises substitutions at residues V11, S36, C38, K53, C76, and C127. In certain embodiments, the modified IL-18 polypeptide comprises V11I, S36A, C38A, K53A, C76A, and C127A substitutions. In certain embodiments, the modified IL-18 polypeptide comprises substitutions at residues V11, D37, C38, K53, C76, and C127. In certain embodiments, the modified IL-18 polypeptide comprises V11I, D37A, C38A, K53A, C76A, and C127A substitutions. In certain embodiments, the modified IL-18 polypeptide comprises substitutions at residues V11, E31, D37, C38, K53, C76, and C127. In certain embodiments, the modified IL-18 polypeptide comprises V11I, E31A, D37A, C38A, K53A, C76A, and C127A substitutions. In certain embodiments, the modified IL-18 polypeptide comprises substitutions at residues V11, C38, D40, K53, C76, and C127. In certain embodiments, the modified IL-18 polypeptide comprises V11I, C38A, D40A, K53A, C76A, and C127A substitutions. In certain embodiments, the modified IL-18 polypeptide comprises substitutions at residues V11, C38, N41, K53, C76, and C127. In certain embodiments, the modified IL-18 polypeptide comprises V11I, C38A, N41A, K53A, C76A, and C127A substitutions. In certain embodiments, the modified IL-18 polypeptide comprises substitutions at residues V11, C38, K53, C76, D132, and C127. In certain embodiments, the modified IL-18 polypeptide comprises V11, C38A, K53A, C76A, D132A, and C127A substitutions. In certain embodiments, the modified IL-18 polypeptide comprises substitutions at residues V11, C38, K53, C76, G108, and C127. In certain embodiments, the modified IL-18 polypeptide comprises V11I, C38A, K53A, C76A, G108A, and C127A substitutions. In certain embodiments, the modified IL-18 polypeptide comprises substitutions at residues V11, C38, K53, C76, H109 and C127. In certain embodiments, the modified IL-18 polypeptide comprises V11I, C38A, K53A, C76A, H109A and C127A substitutions. In certain embodiments, the modified IL-18 polypeptide comprises substitutions at residues V11, C38, K53, C76, D110 and C127. In certain embodiments, the modified IL-18 polypeptide comprises V11I, C38A, K53A, C76A, D110A and C127A substitutions. In certain embodiments, the modified IL-18 polypeptide comprises substitutions at residues K8, V11, C38, C76, Q103, and C127. In certain embodiments, the modified IL-18 polypeptide comprises K8R, V11, C38A, C76A, Q103E and C127A substitutions. In certain embodiments, the modified IL-18 polypeptide comprises K8E, V11I, C38A, C76A, Q103R and C127A substitutions. In certain embodiments, the modified IL-18 polypeptide comprises substitutions at residues V11, C38, C76, Q103, and C127. In certain embodiments, the modified IL-18 polypeptide comprises V11I, C38A, C76A, Q103K and C127A substitutions. In certain embodiments, the modified IL-18 polypeptide comprises substitutions at residues V11, C38, S55, C76, and C127. In certain embodiments, the modified IL-18 polypeptide comprises V11I, C38A, S55H, C76A, and C127A substitutions. In certain embodiments, the modified IL-18 polypeptide comprises V11, C38A, S55R, C76A, and C127A substitutions. In certain embodiments, the modified IL-18 polypeptide comprises V11I, C38A, S55T, C76A, and C127A substitutions. In certain embodiments, the modified IL-18 polypeptide comprises substitutions at residues V11, C38, C76, S105, and C127. In certain embodiments, the modified IL-18 polypeptide comprises V11, C38A, C76A, S105I and C127A substitutions. In certain embodiments, the modified IL-18 polypeptide comprises V11I, C38A, C76A, S105K, and C127A substitutions. In certain embodiments, the modified IL-18 polypeptide comprises substitutions at residues E6, V11, C38, K53, T63, C76, and C127. In certain embodiments, the modified IL-18 polypeptide comprises E6K, V11I, C38A, K53A, T63A, C76A, and C127A substitutions. In certain embodiments, the modified IL-18 polypeptide comprises substitutions at residues E6, K8, V11, C38, K53, T63, C76, and C127. In certain embodiments, the modified IL-18 polypeptide comprises E6K, K8L, V11I, C38A, K53A, T63A, C76A, and C127A substitutions. In certain embodiments, the modified IL-18 polypeptide comprises substitutions at residues E6, V11, C38, I49, K53, T63, C76, and C127. In certain embodiments, the modified IL-18 polypeptide comprises E6K, V11I, C38A, 149E, K53A, T63A, C76A, and C127A substitutions. In certain embodiments, the modified IL-18 polypeptide comprises E6K, V11I, C38A, I49M, K53A, T63A, C76A, and C127A substitutions. In certain embodiments, the modified IL-18 polypeptide comprises E6K, V11I, C38A, I49R, K53A, T63A, C76A, and C127A substitutions. In certain embodiments, the modified IL-18 polypeptide comprises substitutions at residues E6, V11, C38, K53, T63, C76, Q103, and C127. In certain embodiments, the modified IL-18 polypeptide comprises E6K, V11I, C38A, K53A, T63A, C76A, Q103R, and C127A substitutions. In certain embodiments, the modified IL-18 polypeptide comprises substitutions at residues E6, K8, V11, C38, K53, T63, C76, Q103, and C127. In certain embodiments, the modified IL-18 polypeptide comprises E6K, K8E, V11I, C38A, K53A, T63A, C76A, Q103R, and C127A substitutions. In certain embodiments, the modified IL-18 polypeptide comprises substitutions at residues E6, V11, C38, K53, T63, C76, V153, and C127. In some embodiments, the modified IL-18 polypeptide comprises at least seven modifications to the sequence of SEQ ID NO: 1, wherein the seven modifications comprise E6K, V11, C38A, K53A, T63A, C76A, and C127A.

In certain embodiments, the modified IL-18 polypeptide comprises E6K, V11I, C38A, K53A, T63A, C76A, V153R and C127A substitutions. In certain embodiments, the modified IL-18 polypeptide comprises E6K, V11I, C38A, K53A, T63A, C76A, V153E and C127A substitutions. In certain embodiments, the modified IL-18 polypeptide comprises E6K, V11I, C38A, K53A, T63A, C76A, V153Y, and C127A substitutions. In certain embodiments, the modified IL-18 polypeptide comprises substitutions at residues E6, V11, C38, M51, K53, T63, C76, and C127. In certain embodiments, the modified IL-18 polypeptide comprises E6K, V11I, C38A, M51G, K53A, T63A, C76A, and C127A substitutions. In certain embodiments, the modified IL-18 polypeptide comprises E6R, V11I, C38A, K53A, T63A, C76A, and C127A substitutions. In certain embodiments, the modified IL-18 polypeptide comprises substitutions at residues Y1, E6, V11, C38, K53, T63, C76, and C127. In certain embodiments, the modified IL-18 polypeptide comprises Y1M, E6K, V11, C38A, K53A, T63A, C76A, and C127A substitutions.

In some embodiments, the modified IL-18 polypeptide has an amino acid sequence at least 80%, at least 85%, at least 90%, at last 91%, at least 92%, at least 93%, at least 94%, or at least 95% identical to the sequence set forth in SEQ ID NO: 1. In some embodiments, the modified IL-18 polypeptide comprises the sequence set forth in SEQ ID NO: 30.

In certain embodiments, the modified IL-18 polypeptide comprises an amino acid sequence having at least 80%, 85%, 90%, 91%, 92%, 93%, 94% or 95% sequence identity to the sequence set forth in SEQ ID NO: 1. In certain embodiments, the modified IL-18 polypeptide comprises an amino acid sequence having at least 80% sequence identity to the sequence set forth in SEQ ID NO: 1. In certain embodiments, the modified IL-18 polypeptide comprises an amino acid sequence having at least 85% sequence identity to the sequence set forth in SEQ ID NO: 1. In certain embodiments, the modified IL-18 polypeptide comprises an amino acid sequence having at least 90% sequence identity to the sequence set forth in SEQ ID NO: 1. In certain embodiments, the modified IL-18 polypeptide comprises an amino acid sequence having at least 91% sequence identity to the sequence set forth in SEQ ID NO: 1. In certain embodiments, the modified IL-18 polypeptide comprises an amino acid sequence having at least 92% sequence identity to the sequence set forth in SEQ ID NO: 1. In certain embodiments, the modified IL-18 polypeptide comprises an amino acid sequence having at least 93% sequence identity to the sequence set forth in SEQ ID NO: 1. In certain embodiments, the modified IL-18 polypeptide comprises an amino acid sequence having at least 94% sequence identity to the sequence set forth in SEQ ID NO: 1. In certain embodiments, the modified IL-18 polypeptide comprises an amino acid sequence having at least 95% sequence identity to the sequence set forth in SEQ ID NO: 1.

In certain embodiments, the modified IL-18 polypeptide comprises an amino acid sequence having at least 80%, 85%, 90%, 95%, 98%, 99%, or 100% sequence identity to the sequence set forth in SEQ ID NO: 204. In certain embodiments, the modified IL-18 polypeptide comprises the amino acid sequence set forth in SEQ ID NO: 204. In certain embodiments, the modified IL-18 polypeptide comprises an amino acid sequence having at least 80%, 85%, 90%, 95%, 98%, 99%, or 100% sequence identity to the sequence set forth in SEQ ID NO: 205. In certain embodiments, the modified IL-18 polypeptide comprises the amino acid sequence set forth in SEQ ID NO: 205. In certain embodiments, the modified IL-18 polypeptide comprises an amino acid sequence having at least 80%, 85%, 90%, 95%, 98%, 99%, or 100% sequence identity to the sequence set forth in SEQ ID NO: 206. In certain embodiments, the modified IL-18 polypeptide comprises the amino acid sequence set forth in SEQ ID NO: 206. In certain embodiments, the modified IL-18 polypeptide comprises an amino acid sequence having at least 80%, 85%, 90%, 95%, 98%, 99%, or 100% sequence identity to the sequence set forth in SEQ ID NO: 207. In certain embodiments, the modified IL-18 polypeptide comprises an amino acid sequence having at least 80% sequence identity to the sequence set forth in SEQ ID NO: 207. In certain embodiments, the modified IL-18 polypeptide comprises an amino acid sequence having at least 85% sequence identity to the sequence set forth in SEQ ID NO: 207. In certain embodiments, the modified IL-18 polypeptide comprises an amino acid sequence having at least 90% sequence identity to the sequence set forth in SEQ ID NO: 207. In certain embodiments, the modified IL-18 polypeptide comprises an amino acid sequence having at least 95% sequence identity to the sequence set forth in SEQ ID NO: 207. In certain embodiments, the modified IL-18 polypeptide comprises an amino acid sequence having at least 98% sequence identity to the sequence set forth in SEQ ID NO: 207. In certain embodiments, the modified IL-18 polypeptide comprises an amino acid sequence having at least 99% sequence identity to the sequence set forth in SEQ ID NO: 207. In certain embodiments, the modified IL-18 polypeptide comprises the amino acid sequence set forth in SEQ ID NO: 207. In certain embodiments, the modified IL-18 polypeptide comprises an amino acid sequence having at least 80%, 85%, 90%, 95%, 98%, 99%, or 100% sequence identity to the sequence set forth in SEQ ID NO: 208. In certain embodiments, the modified IL-18 polypeptide comprises the amino acid sequence set forth in SEQ ID NO: 208. In certain embodiments, the modified IL-18 polypeptide comprises an amino acid sequence having at least 80%, 85%, 90%, 95%, 98%, 99%, or 100% sequence identity to the sequence set forth in SEQ ID NO: 209. In certain embodiments, the modified IL-18 polypeptide comprises the amino acid sequence set forth in SEQ ID NO: 209. In certain embodiments, the modified IL-18 polypeptide comprises an amino acid sequence having at least 80%, 85%, 90%, 95%, 98%, 99%, or 100% sequence identity to the sequence set forth in SEQ ID NO: 210. In certain embodiments, the modified IL-18 polypeptide comprises the amino acid sequence set forth in SEQ ID NO: 210. In certain embodiments, the modified IL-18 polypeptide comprises an amino acid sequence having at least 80%, 85%, 90%, 95%, 98%, 99%, or 100% sequence identity to the sequence set forth in SEQ ID NO: 211. In certain embodiments, the modified IL-18 polypeptide comprises the amino acid sequence set forth in SEQ ID NO: 211. In certain embodiments, the modified IL-18 polypeptide comprises an amino acid sequence having at least 80%, 85%, 90%, 95%, 98%, 99%, or 100% sequence identity to the sequence set forth in SEQ ID NO: 212. In certain embodiments, the modified IL-18 polypeptide comprises the amino acid sequence set forth in SEQ ID NO: 212. In certain embodiments, the modified IL-18 polypeptide comprises an amino acid sequence having at least 80%, 85%, 90%, 95%, 98%, 99%, or 100% sequence identity to the sequence set forth in SEQ ID NO: 213. In certain embodiments, the modified IL-18 polypeptide comprises the amino acid sequence set forth in SEQ ID NO: 213. In certain embodiments, the modified IL-18 polypeptide comprises an amino acid sequence having at least 80%, 85%, 90%, 95%, 98%, 99%, or 100% sequence identity to the sequence set forth in SEQ ID NO: 214. In certain embodiments, the modified IL-18 polypeptide comprises the amino acid sequence set forth in SEQ ID NO: 214. In certain embodiments, the modified IL-18 polypeptide comprises an amino acid sequence having at least 80%, 85%, 90%, 95%, 98%, 99%, or 100% sequence identity to the sequence set forth in SEQ ID NO: 215. In certain embodiments, the modified IL-18 polypeptide comprises the amino acid sequence set forth in SEQ ID NO: 215. In certain embodiments, the modified IL-18 polypeptide comprises an amino acid sequence having at least 80%, 85%, 90%, 95%, 98%, 99%, or 100% sequence identity to the sequence set forth in SEQ ID NO: 216. In certain embodiments, the modified IL-18 polypeptide comprises the amino acid sequence set forth in SEQ ID NO: 216. In certain embodiments, the modified IL-18 polypeptide comprises an amino acid sequence having at least 80%, 85%, 90%, 95%, 98%, 99%, or 100% sequence identity to the sequence set forth in SEQ ID NO: 217. In certain embodiments, the modified IL-18 polypeptide comprises the amino acid sequence set forth in SEQ ID NO: 217. In certain embodiments, the modified IL-18 polypeptide comprises an amino acid sequence having at least 80%, 85%, 90%, 95%, 98%, 99%, or 100% sequence identity to the sequence set forth in SEQ ID NO: 218. In certain embodiments, the modified IL-18 polypeptide comprises the amino acid sequence set forth in SEQ ID NO: 218. In certain embodiments, the modified IL-18 polypeptide comprises an amino acid sequence having at least 80%, 85%, 90%, 95%, 98%, 99%, or 100% sequence identity to the sequence set forth in SEQ ID NO: 219. In certain embodiments, the modified IL-18 polypeptide comprises the amino acid sequence set forth in SEQ ID NO: 219. In certain embodiments, the modified IL-18 polypeptide comprises an amino acid sequence having at least 80%, 85%, 90%, 95%, 98%, 99%, or 100% sequence identity to the sequence set forth in SEQ ID NO: 220. In certain embodiments, the modified IL-18 polypeptide comprises the amino acid sequence set forth in SEQ ID NO: 220. In certain embodiments, the modified IL-18 polypeptide comprises an amino acid sequence having at least 80%, 85%, 90%, 95%, 98%, 99%, or 100% sequence identity to the sequence set forth in SEQ ID NO: 221. In certain embodiments, the modified IL-18 polypeptide comprises the amino acid sequence set forth in SEQ ID NO: 221. In certain embodiments, the modified IL-18 polypeptide comprises an amino acid sequence having at least 80%, 85%, 90%, 95%, 98%, 99%, or 100% sequence identity to the sequence set forth in SEQ ID NO: 222. In certain embodiments, the modified IL-18 polypeptide comprises the amino acid sequence set forth in SEQ ID NO: 222. In certain embodiments, the modified IL-18 polypeptide comprises an amino acid sequence having at least 80%, 85%, 90%, 95%, 98%, 99%, or 100% sequence identity to the sequence set forth in SEQ ID NO: 223. In certain embodiments, the modified IL-18 polypeptide comprises the amino acid sequence set forth in SEQ ID NO: 223. In certain embodiments, the modified IL-18 polypeptide comprises an amino acid sequence having at least 80%, 85%, 90%, 95%, 98%, 99%, or 100% sequence identity to the sequence set forth in SEQ ID NO: 224. In certain embodiments, the modified IL-18 polypeptide comprises the amino acid sequence set forth in SEQ ID NO: 224. In certain embodiments, the modified IL-18 polypeptide comprises an amino acid sequence having at least 80%, 85%, 90%, 95%, 98%, 99%, or 100% sequence identity to the sequence set forth in SEQ ID NO: 225. In certain embodiments, the modified IL-18 polypeptide comprises the amino acid sequence set forth in SEQ ID NO: 225. In certain embodiments, the modified IL-18 polypeptide comprises an amino acid sequence having at least 80%, 85%, 90%, 95%, 98%, 99%, or 100% sequence identity to the sequence set forth in SEQ ID NO: 226. In certain embodiments, the modified IL-18 polypeptide comprises the amino acid sequence set forth in SEQ ID NO: 226. In certain embodiments, the modified IL-18 polypeptide comprises an amino acid sequence having at least 80%, 85%, 90%, 95%, 98%, 99%, or 100% sequence identity to the sequence set forth in SEQ ID NO: 227. In certain embodiments, the modified IL-18 polypeptide comprises the amino acid sequence set forth in SEQ ID NO: 227. In certain embodiments, the modified IL-18 polypeptide comprises an amino acid sequence having at least 80%, 85%, 90%, 95%, 98%, 99%, or 100% sequence identity to the sequence set forth in SEQ ID NO: 228. In certain embodiments, the modified IL-18 polypeptide comprises the amino acid sequence set forth in SEQ ID NO: 228. In certain embodiments, the modified IL-18 polypeptide comprises an amino acid sequence having at least 80%, 85%, 90%, 95%, 98%, 99%, or 100% sequence identity to the sequence set forth in SEQ ID NO: 229. In certain embodiments, the modified IL-18 polypeptide comprises the amino acid sequence set forth in SEQ ID NO: 229. In certain embodiments, the modified IL-18 polypeptide comprises an amino acid sequence having at least 80%, 85%, 90%, 95%, 98%, 99%, or 100% sequence identity to the sequence set forth in SEQ ID NO: 230. In certain embodiments, the modified IL-18 polypeptide comprises the amino acid sequence set forth in SEQ ID NO: 230. In certain embodiments, the modified IL-18 polypeptide comprises an amino acid sequence having at least 80%, 85%, 90%, 95%, 98%, 99%, or 100% sequence identity to the sequence set forth in SEQ ID NO: 231. In certain embodiments, the modified IL-18 polypeptide comprises the amino acid sequence set forth in SEQ ID NO: 231. In certain embodiments, the modified IL-18 polypeptide comprises an amino acid sequence having at least 80%, 85%, 90%, 95%, 98%, 99%, or 100% sequence identity to the sequence set forth in SEQ ID NO: 232. In certain embodiments, the modified IL-18 polypeptide comprises the amino acid sequence set forth in SEQ ID NO: 232. In certain embodiments, the modified IL-18 polypeptide comprises an amino acid sequence having at least 80%, 85%, 90%, 95%, 98%, 99%, or 100% sequence identity to the sequence set forth in SEQ ID NO: 233. In certain embodiments, the modified IL-18 polypeptide comprises the amino acid sequence set forth in SEQ ID NO: 233. In certain embodiments, the modified IL-18 polypeptide comprises an amino acid sequence having at least 80%, 85%, 90%, 95%, 98%, 99%, or 100% sequence identity to the sequence set forth in SEQ ID NO: 234. In certain embodiments, the modified IL-18 polypeptide comprises the amino acid sequence set forth in SEQ ID NO: 234. In certain embodiments, the modified IL-18 polypeptide comprises an amino acid sequence having at least 80%, 85%, 90%, 95%, 98%, 99%, or 100% sequence identity to the sequence set forth in SEQ ID NO: 235. In certain embodiments, the modified IL-18 polypeptide comprises the amino acid sequence set forth in SEQ ID NO: 235. In certain embodiments, the modified IL-18 polypeptide comprises an amino acid sequence having at least 80%, 85%, 90%, 95%, 98%, 99%, or 100% sequence identity to the sequence set forth in SEQ ID NO: 236. In certain embodiments, the modified IL-18 polypeptide comprises the amino acid sequence set forth in SEQ ID NO: 236. In certain embodiments, the modified IL-18 polypeptide comprises an amino acid sequence having at least 80%, 85%, 90%, 95%, 98%, 99%, or 100% sequence identity to the sequence set forth in SEQ ID NO: 237. In certain embodiments, the modified IL-18 polypeptide comprises the amino acid sequence set forth in SEQ ID NO: 237. In certain embodiments, the modified IL-18 polypeptide comprises an amino acid sequence having at least 80%, 85%, 90%, 95%, 98%, 99%, or 100% sequence identity to the sequence set forth in SEQ ID NO: 238. In certain embodiments, the modified IL-18 polypeptide comprises the amino acid sequence set forth in SEQ ID NO: 238. In certain embodiments, the modified IL-18 polypeptide comprises an amino acid sequence having at least 80%, 85%, 90%, 95%, 98%, 99%, or 100% sequence identity to the sequence set forth in SEQ ID NO: 239. In certain embodiments, the modified IL-18 polypeptide comprises an amino acid sequence having at least 80% sequence identity to the sequence set forth in SEQ ID NO: 239. In certain embodiments, the modified IL-18 polypeptide comprises an amino acid sequence having at least 85% sequence identity to the sequence set forth in SEQ ID NO: 239. In certain embodiments, the modified IL-18 polypeptide comprises an amino acid sequence having at least 90% sequence identity to the sequence set forth in SEQ ID NO: 239. In certain embodiments, the modified IL-18 polypeptide comprises an amino acid sequence having at least 95% sequence identity to the sequence set forth in SEQ ID NO: 239. In certain embodiments, the modified IL-18 polypeptide comprises an amino acid sequence having at least 98% sequence identity to the sequence set forth in SEQ ID NO: 239. In certain embodiments, the modified IL-18 polypeptide comprises an amino acid sequence having at least 99% sequence identity to the sequence set forth in SEQ ID NO: 239. In certain embodiments, the modified IL-18 polypeptide comprises the amino acid sequence set forth in SEQ ID NO: 239. In certain embodiments, the modified IL-18 polypeptide comprises an amino acid sequence having at least 80%, 85%, 90%, 95%, 98%, 99%, or 100% sequence identity to the sequence set forth in SEQ ID NO: 240. In certain embodiments, the modified IL-18 polypeptide comprises the amino acid sequence set forth in SEQ ID NO: 240. In certain embodiments, the modified IL-18 polypeptide comprises an amino acid sequence having at least 80%, 85%, 90%, 95%, 98%, 99%, or 100% sequence identity to the sequence set forth in SEQ ID NO: 241. In certain embodiments, the modified IL-18 polypeptide comprises an amino acid sequence having at least 80% sequence identity to the sequence set forth in SEQ ID NO: 241. In certain embodiments, the modified IL-18 polypeptide comprises an amino acid sequence having at least 85% sequence identity to the sequence set forth in SEQ ID NO: 241. In certain embodiments, the modified IL-18 polypeptide comprises an amino acid sequence having at least 90% sequence identity to the sequence set forth in SEQ ID NO: 241. In certain embodiments, the modified IL-18 polypeptide comprises an amino acid sequence having at least 95% sequence identity to the sequence set forth in SEQ ID NO: 241. In certain embodiments, the modified IL-18 polypeptide comprises an amino acid sequence having at least 98% sequence identity to the sequence set forth in SEQ ID NO: 241. In certain embodiments, the modified IL-18 polypeptide comprises an amino acid sequence having at least 99% sequence identity to the sequence set forth in SEQ ID NO: 241. In certain embodiments, the modified IL-18 polypeptide comprises the amino acid sequence set forth in SEQ ID NO: 241. In certain embodiments, the modified IL-18 polypeptide comprises an amino acid sequence having at least 80%, 85%, 90%, 95%, 98%, 99%, or 100% sequence identity to the sequence set forth in SEQ ID NO: 242. In certain embodiments, the modified IL-18 polypeptide comprises an amino acid sequence having at least 80% sequence identity to the sequence set forth in SEQ ID NO: 242. In certain embodiments, the modified IL-18 polypeptide comprises an amino acid sequence having at least 85% sequence identity to the sequence set forth in SEQ ID NO: 242. In certain embodiments, the modified IL-18 polypeptide comprises an amino acid sequence having at least 90% sequence identity to the sequence set forth in SEQ ID NO: 242. In certain embodiments, the modified IL-18 polypeptide comprises an amino acid sequence having at least 95% sequence identity to the sequence set forth in SEQ ID NO: 242. In certain embodiments, the modified IL-18 polypeptide comprises an amino acid sequence having at least 98% sequence identity to the sequence set forth in SEQ ID NO: 242. In certain embodiments, the modified IL-18 polypeptide comprises an amino acid sequence having at least 99% sequence identity to the sequence set forth in SEQ ID NO: 242. In certain embodiments, the modified IL-18 polypeptide comprises the amino acid sequence set forth in SEQ ID NO: 242. In certain embodiments, the modified IL-18 polypeptide comprises an amino acid sequence having at least 80%, 85%, 90%, 95%, 98%, 99%, or 100% sequence identity to the sequence set forth in SEQ ID NO: 243. In certain embodiments, the modified IL-18 polypeptide comprises the amino acid sequence set forth in SEQ ID NO: 243. In certain embodiments, the modified IL-18 polypeptide comprises an amino acid sequence having at least 80%, 85%, 90%, 95%, 98%, 99%, or 100% sequence identity to the sequence set forth in SEQ ID NO: 244. In certain embodiments, the modified IL-18 polypeptide comprises an amino acid sequence having at least 80% sequence identity to the sequence set forth in SEQ ID NO: 244. In certain embodiments, the modified IL-18 polypeptide comprises an amino acid sequence having at least 85% sequence identity to the sequence set forth in SEQ ID NO: 244. In certain embodiments, the modified IL-18 polypeptide comprises an amino acid sequence having at least 90% sequence identity to the sequence set forth in SEQ ID NO: 244. In certain embodiments, the modified IL-18 polypeptide comprises an amino acid sequence having at least 95% sequence identity to the sequence set forth in SEQ ID NO: 244. In certain embodiments, the modified IL-18 polypeptide comprises an amino acid sequence having at least 98% sequence identity to the sequence set forth in SEQ ID NO: 244. In certain embodiments, the modified IL-18 polypeptide comprises an amino acid sequence having at least 99% sequence identity to the sequence set forth in SEQ ID NO: 244. In certain embodiments, the modified IL-18 polypeptide comprises the amino acid sequence set forth in SEQ ID NO: 244.

A modified IL-18 polypeptide as described herein can comprise one or more unnatural amino acids. “Unnatural” amino acids can refer to amino acid residues in D- or L-form that are not among the 20 canonical amino acids generally incorporated into naturally occurring proteins. In some embodiments, one or more amino acids of the modified IL-18 polypeptides are substituted with one or more unnatural amino acids. Unnatural amino acids include, but are not limited to L-azidolysine, p-azido-L-phenylalanine, and L-biphenylalanine.

Exemplary unnatural amino acids also include p-acetyl-L-phenylalanine, p-iodo-L-phenylalanine, p-propargyloxyphenylalanine, p-propargyl-phenylalanine, L-3-(2-naphthyl) alanine, 3-methyl-phenylalanine, tri-O-acetyl-GlcNAcp-serine, L-Dopa, fluorinated phenylalanine, isopropyl-L-phenylalanine, p-azido-L-phenylalanine, p-acyl-L-phenylalanine, p-benzoyl-L-phenylalanine, p-Boronophenylalanine, p-bromophenylalanine, p-amino-L-phenylalanine, isopropyl-L-phenylalanine, an analogue of a tyrosine amino acid; an analogue of a glutamine amino acid; an analogue of a phenylalanine amino acid; an analogue of a serine amino acid; an analogue of a threonine amino acid; a β-amino acid; a cyclic amino acid other than proline or histidine; an aromatic amino acid other than phenylalanine, tyrosine or tryptophan; or a combination thereof. In some embodiments, the unnatural amino acids are selected from β-amino acids, homoamino acids, and cyclic amino acids. In some embodiments, the unnatural amino acids comprise β-alanine, β-aminopropionic acid, piperidinic acid, aminocaprioic acid, aminoheptanoic acid, aminopimelic acid, desmosine, diaminopimelic acid, N^{α}-ethylglycine, N^{α}-ethylaspargine, isodesmosine, allo-isoleucine, N^{α}-methylglycine, N^{α}-methylisoleucine, N^{α}-methylvaline, γ-carboxyglutamate, N^{α}-acetylserine, N^{α}-formylmethionine, 3-methylhistidine, and/or other similar amino acids.

IIb. Points of Attachment of Polymer Moieties to Modified IL-18 Polypeptides

The modified IL-18 polypeptides described herein contain one or more polymers. The modified IL-18 polypeptides can comprise a polymer covalently attached thereto. The modified IL-18 polypeptides can comprise an amino acid sequence described herein, and a polymer covalently attached to an amino acid residue of the polypeptide. In some embodiments, the modified IL-18 polypeptide is conjugated to one polymer moiety.

In some embodiments, the residue to which the polymer attached is a natural amino acid residue. In some embodiments, the residue to which the polymer is covalently attached is selected from cysteine, aspartate, asparagine, glutamate, glutamine, serine, threonine, lysine, and tyrosine. In some embodiments, the residue to which the polymer is covalently attached is selected from asparagine, aspartic acid, cysteine, glutamic acid, glutamine, lysine, and tyrosine. In some embodiments, the polymer is covalently attached to a cysteine. In some embodiments, the polymer is covalently attached to a lysine. In some embodiments, the polymer is covalently attached to a glutamine. In some embodiments, the polymer is covalently attached to an asparagine. In some embodiments, the residue to which the polymer is attached is the natural amino acid in that position in SEQ ID NO: 1. In some embodiments, the polymer is attached to a different natural amino acid which is substituted at the relevant position. In some embodiments, the polymer is covalently attached to site-specifically to a natural amino acid.

In some embodiments, the polymer is attached at an unnatural amino acid residue. In some embodiments, the unnatural amino acid residue comprises a conjugation handle. In some embodiments, the conjugation handle facilitates the addition of the polymer to the modified IL-18 polypeptide. The conjugation handle can be any of the conjugation handles provided herein. In some embodiments, the polymer is covalently attached site-specifically to the unnatural amino acid.

In some embodiments, the polymer is attached at residue 68, 69, or 70 of the modified IL-18 polypeptide, wherein the residue position numbering is based on SEQ ID NO: 1 as the reference sequence.

In some embodiments, the polymer is covalently attached at residue 68. In some embodiments, the polymer is covalently attached at residue C68, C68D, C68Q, C68K, C68N, C68E, or C68Y. In some embodiments, the polymer is covalently attached at residue C68. In some embodiments, the polymer is covalently attached to an unnatural amino acid at residue 68.

In some embodiments, the polymer is covalently attached at residue 69. In some embodiments, the polymer is covalently attached at residue E69, E69C, E69D, E69Q, E69K, E69N, or E69Y. In some embodiments, the polymer is covalently attached at residue E69. In some embodiments, the polymer is covalently attached at residue E69C. In some embodiments, the polymer is covalently attached to an unnatural amino acid at residue 69.

In some embodiments, the polymer is covalently attached at residue 70. In some embodiments, the polymer is covalently attached at residue K70, K70C, K70E, K70D, K70Q, K70N, or K70Y. In some embodiments, the polymer is covalently attached at residue K70. In some embodiments, the polymer is covalently attached at residue K70C. In some embodiments, the polymer is covalently attached to an unnatural amino acid at residue 70.

In some embodiments, the polymer is covalently attached at residue 85. In some embodiments, the polymer is covalently attached at residue E85, E85C, E85D, E85Q, E85K, E85N, or E85Y. In some embodiments, the polymer is covalently attached at residue E85. In some embodiments, the polymer is covalently attached residue E85C. In some embodiments, the polymer is covalently attached to an unnatural amino acid at residue 85.

In some embodiments, the polymer is covalently attached at residue 86. In some embodiments, the polymer is covalently attached at residue M86C, M86D, M86Q, M86K, M86N, M86E, or M86Y. In some embodiments, the polymer is covalently attached M86C. In some embodiments, the polymer is covalently attached to an unnatural amino acid at residue 86.

In some embodiments, the polymer is covalently attached at residue 95. In some embodiments, the polymer is covalently attached at residue T95, T95C, T95D, T95Q, T95K, T95N, T95E, or T95Y. In some embodiments, the polymer is covalently attached at residue T95C, T95D, T95Q, T95K, T95N, T95E, or T95Y. In some embodiments, the polymer is covalently attached at residue T95C. In some embodiments, the polymer is covalently attached to an unnatural amino acid at residue 95.

In some embodiments, the polymer is covalently attached at residue 98. In some embodiments, the polymer is covalently attached at residue D98, D98C, D98Q, D98K, D98N, D98E, or D98Y. In some embodiments, the polymer is covalently attached at residue D98C. In some embodiments, the polymer is covalently attached to an unnatural amino acid at residue 98.

In some embodiments, the polymer is covalently attached through a modified amino acid. In some embodiments, the modified amino acid is an amino-acid-PEG-azide group. In some embodiments, the modified amino acid is a glutamate, aspartate, lysine, cysteine, or tyrosine modified to incorporate an azide group linked to the amino acid through a PEG spacer. In some embodiments, the modified amino acid a has a structure selected from:

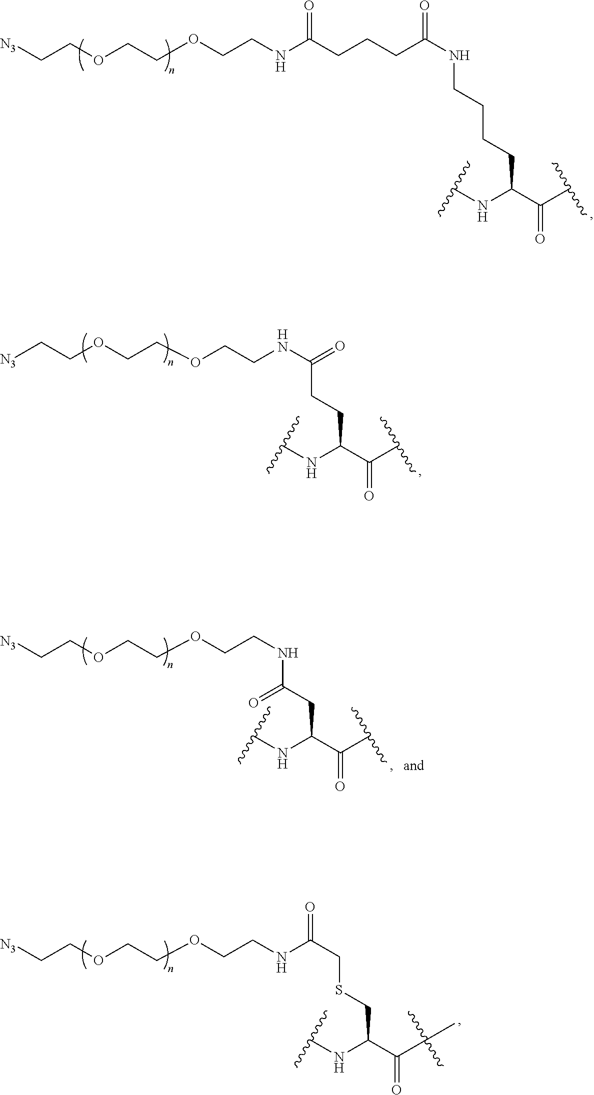

wherein each n is independently an integer from 1-30. In some embodiments, n is an integer from 1-20, 1-10, 2-30, 2-20, 2-10, 5-30, 5-20, or 5-10. In some embodiments, n is 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30. In some embodiments, n is 10. In some embodiments, n is 8. In some embodiments, n is 6. In some embodiments, n is 12.

In some embodiments, the modified IL-18 polypeptide comprises a polymer covalently attached to a modified lysine residue. In some embodiments, the modified lysine residue comprises a conjugation handle. In some embodiments, the modified lysine residue comprises an azide. In some embodiments, the modified lysine residue has a structure of Structure B, wherein Structure B is

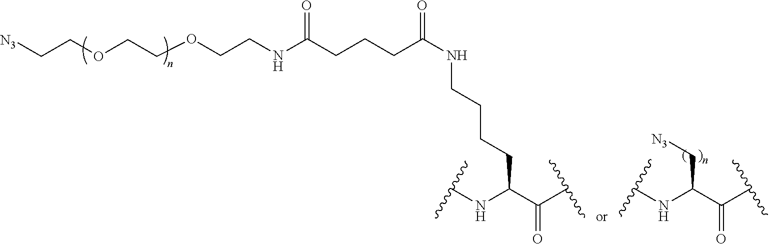

wherein each n is independently an integer from 1-30. In some embodiments, n is an integer from 1-20, 1-10, 2-30, 2-20, 2-10, 5-30, 5-20, or 5-10. In some embodiments, n is 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30. In some embodiments, n is 1, 2, 3, 4, 5, 6, 7, or 8. In some embodiments, n is 3. In some embodiments, n is 4. In some embodiments, n is 5. In some embodiments, n is 6. In some embodiments, n is 8. In some embodiments, n is 10. In some embodiments, n is 12.

In some embodiments, the modified lysine of Structure B is located at a position on the modified IL-18 polypeptide in the region of residues 79-120. In some embodiments, the modified lysine of Structure B is located at a position on the modified IL-18 polypeptide selected from residue 85, residue 86, and residue 98. In some embodiments, the modified lysine of Structure B is located at residue 85 of the modified IL-18 polypeptide. In some embodiments, the modified lysine of Structure B is located at residue 86 of the modified IL-18 polypeptide. In some embodiments, the modified lysine of Structure B is located at residue 95 of the modified IL-18 polypeptide. In some embodiments, the modified lysine of Structure B is located at residue 98 of the modified IL-18 polypeptide.

In some embodiments, the modified IL-18 polypeptide comprises more than one polymer covalently attached thereto. In some embodiments, the modified IL-18 polypeptide comprises a second polymer covalently attached. In some embodiments, the modified IL-18 polypeptide comprises a second polymer covalently attached at a residue provided herein.

IIc. Conjugation Handle

In some embodiments, amino acid residues of the modified IL-18 polypeptides are modified to comprise or substituted with a conjugation handle. In some embodiments, the amino acid residues comprise an amino, azide, allyl, ester, and/or amide functional groups. In some embodiments, the conjugation handles can serve as useful anchor points to attach a polymer and/or additional moieties to the modified IL-18 polypeptides. In some embodiments, the amino acid residues have a structure built from precursors Structure 1, Structure 2, Structure 3, or Structure 4:

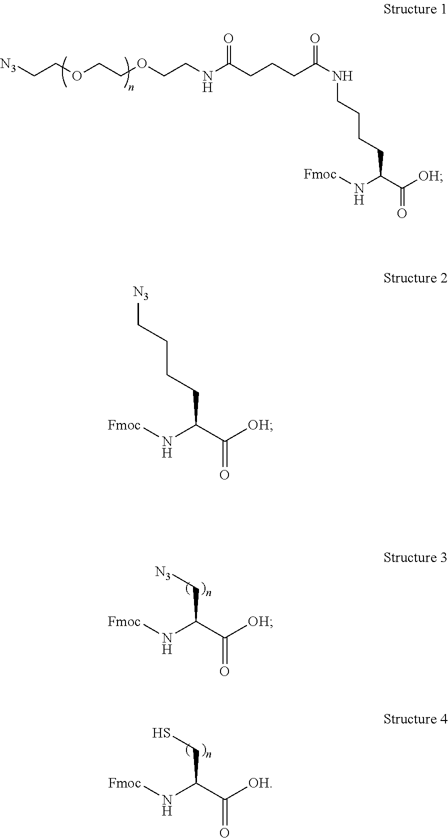

In some embodiments, the modified IL-18 polypeptide contains a substitution for modified natural amino acid residues which can be used for attachment of a polymer and/or additional functional groups which can be used to facilitate conjugation reaction or attachment of various payloads to the modified IL-18 polypeptide (e.g., polymers). The substitution can be for a naturally occurring amino acid which is more amenable to attachment of additional functional groups (e.g., aspartic acid/asparagine cysteine, glutamic acid/glutamine, lysine, serine, threonine, or tyrosine), a derivative of a modified version of any naturally occurring amino acid, or any unnatural amino acid (e.g., an amino acid containing a desired reactive group, such as a CLICK chemistry reagent such as an azide, alkyne, etc.). Non-limiting examples of modified natural amino acid residues include the modified lysine, glutamic acid, aspartic acid, and cysteine provided below:

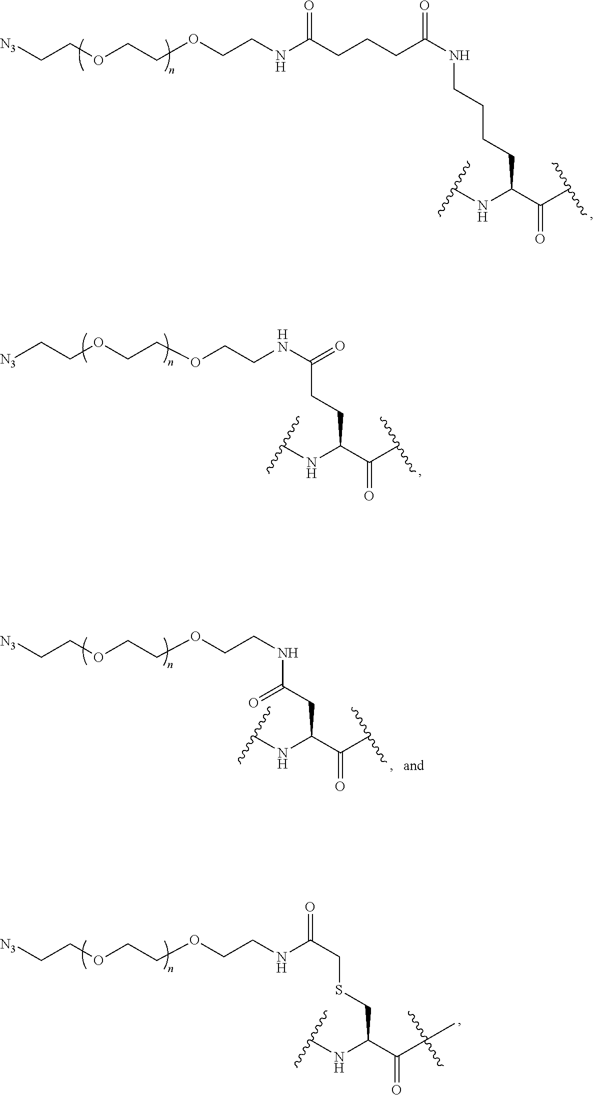

wherein each n is an integer from 1-30. Other examples of natural amino acids which can be similarly modified include those with heteroatoms capable of easily forming a bond with a suitable group to link the polymeric group to the amino acid (e.g., tyrosine, serine, threonine). These non-limiting examples of modified amino acid residues can be used at any location at which it is desirable to add an additional functionality (e.g., a polymer) to the modified IL-18 polypeptide.

In some embodiments, any of structures 1-4, the modified lysine, the modified glutamic acid, the modified aspartic acid, or the modified cysteine provided above can be substituted for a different residue of the modified IL-18 polypeptide to allow for conjugation at a different site of the IL-18 polypeptide. The azide functionality may also be replaced with another suitable conjugation handle. The conjugation handles provided herein can be any suitable reactive group capable of reacting with a complementary reactive group. In some embodiments, the conjugation handle comprises a reagent for a Cu(I)-catalyzed or “copper-free” alkyne-azide triazole-forming reaction (e.g., strain promoted cycloadditions), the Staudinger ligation, inverse-electron-demand Diels-Alder (IEDDA) reaction, “photo-click” chemistry, tetrazine cycloadditions with trans-cyclooctenes, or a metal-mediated process such as olefin metathesis and Suzuki-Miyaura, O-substituted hydroxylamine, potassium acyltrifluoroborate or Sonogashira cross-coupling.

In some embodiments, the conjugation handle comprises a reagent for a “copper-free” alkyne azide triazole-forming reaction. Non-limiting examples of alkynes for said alkyne azide triazole forming reaction include cyclooctyne reagents (e.g., (1R,8S,9s)-Bicyclo[6.1.0]non-4-yn-9-ylmethanol containing reagents, dibenzocyclooctyne-amine reagents, difluorocyclooctynes, or derivatives thereof).

In some embodiments, the conjugation handle comprises a reactive group selected from azide, alkyne, tetrazine, halide, sulfhydryl, disulfide, maleimide, activated ester, alkene, aldehyde, ketone, imine, hydrazine, acyltrifluoroborate, hydroxylamine (e.g., 0-substituted hydroxylamine), phosphine, trans-cyclooctene, and hydrazide. In some embodiments, the conjugation handle and complementary conjugation handle comprise “CLICK” chemistry reagents. Exemplary groups of click chemistry residue are shown in Hein et al., “Click Chemistry, A Powerful Tool for Pharmaceutical Sciences,” Pharmaceutical Research, volume 25, pages 2216-2230 (2008); Thirumurugan et al., “Click Chemistry for Drug Development and Diverse Chemical-Biology Applications,” Chem. Rev. 2013, 113, 7, 4905-4979; US20160107999A1; U.S. Ser. No. 10/266,502B2; and US20190204330A1, each of which is incorporated by reference in its entirety.

In some embodiments, a group attached to the modified IL-18 polypeptide (e.g., a polymer moiety or an additional polypeptide) comprises a conjugation handle or a reaction product of a conjugation handle with a complementary conjugation handle. In some embodiments, the reaction product of the conjugation handle with the complementary conjugation handle results from a KAT ligation (reaction of potassium acyltrifluoroborate with hydroxylamine), a Staudinger ligation (reaction of an azide with a phosphine), a tetrazine cycloaddition (reaction of a tetrazine with a trans-cyclooctene), or a Huisgen cycloaddition (reaction of an alkyne with an azide). In some embodiments, the group attached to the IL-18 polypeptide (e.g., the polymer or the additional polypeptide) will comprise a reaction product of a conjugation handle with a complementary conjugation handle which was used to attach the group to the modified IL-18 polypeptide.

IId. Polymers Attached to Modified IL-18 Polypeptides

The modified IL-18 polypeptide comprises a covalently attached polymer. In some embodiments, described herein is a modified IL-18 polypeptide comprising one or more polymers covalently attached. In some embodiments, the modified IL-18 polypeptide comprises 1, 2, 3, 4, 5, 6, 7, 8, 9, 10, or more polymers covalently attached.

In some embodiments, the polymer comprises a conjugation handle which can be used to further attach an additional moiety to the polymer modified IL-18 polypeptide (e.g., the addition of an additional polypeptide, such as an antibody). Any suitable reactive group capable of reacting with a complementary reactive group attached to another moiety can be used as the conjugation handle. In some embodiments, the conjugation handle comprises a reagent for a Cu(I)-catalyzed or “copper-free” alkyne-azide triazole-forming reaction (e.g., strain promoted cycloadditions), the Staudinger ligation, inverse-electron-demand Diels-Alder (IEDDA) reaction, “photo-click” chemistry, tetrazine cycloadditions with trans-cyclooctenes, potassium acyltrifluoroborate ligations (e.g., with O-substituted hydroxylamines) or a metal-mediated process such as olefin metathesis and Suzuki-Miyaura or Sonogashira cross-coupling.

In some embodiments, the conjugation handle comprises a reagent for a “copper-free” alkyne azide triazole-forming reaction. Non-limiting examples of alkynes for said alkyne azide triazole forming reaction include cyclooctyne reagents (e.g., (1R,8S,9s)-Bicyclo[6.1.0]non-4-yn-9-ylmethanol containing reagents, dibenzocyclooctyne-amine reagents, difluorocyclooctynes, or derivatives thereof).

In some embodiments, the conjugation handle comprises a reactive group selected from azide, alkyne, tetrazine, halide, sulfhydryl, disulfide, maleimide, activated ester, alkene, aldehyde, ketone, imine, hydrazine, acyltrifluoroborate, hydroxylamine, phosphine, trans-cyclooctene, and hydrazide. In some embodiments, the conjugation handle and the complementary conjugation handle comprise “CLICK” chemistry reagents. Exemplary groups of click chemistry residue are shown in Hein et al., “Click Chemistry, A Powerful Tool for Pharmaceutical Sciences,” Pharmaceutical Research, volume 25, pages 2216-2230 (2008); Thirumurugan et al., “Click Chemistry for Drug Development and Diverse Chemical-Biology Applications,” Chem. Rev. 2013, 113, 7, 4905-4979; US20160107999A1; U.S. Ser. No. 10/266,502B2; and US20190204330A1, each of which is incorporated by reference in its entirety.

In some embodiments, the polymer comprises a conjugation handle or a reaction product of a conjugation handle with a complementary conjugation handle. In some embodiments, the reaction product of the conjugation handle with the complementary conjugation handle results from a KAT ligation (reaction of potassium acyltrifluoroborate with hydroxylamine), a Staudinger ligation (reaction of an azide with a phosphine), a tetrazine cycloaddition (reaction of a tetrazine with a trans-cyclooctene), or a Huisgen cycloaddition (reaction of an alkyne with an azide). In some embodiments, the polymer comprises a reaction product of a conjugation handle with a complementary conjugation handle which was used to attach the polymer to the modified IL-18 polypeptide.

In some embodiments, the polymer comprises an azide moiety. In some embodiments, the polymer comprises an alkyne moiety. In some embodiments, the polymer comprises an azide moiety, an alkyne moiety, or reaction product of an azide-alkyne cycloaddition reaction. In some embodiments, the reaction product of the azide-alkyne cycloaddition reaction is a 1,2,3-triazole.

In some embodiments, the polymer is attached to the modified IL-18 polypeptide through use of a bifunctional linker. In some embodiments, the bifunctional linker reacts with a reactive group of an amino acid residue on the modified IL-18 polypeptide (e.g., a cysteine sulfhydryl) to form a covalent bond. In some embodiments, in a second step, the second reactive group of the bifunctional a linker (e.g., a conjugation handle such as an azide or alkyne) is then used to attach a second moiety, such as the polymer.

In some embodiments, the polymer is a water-soluble polymer. In some embodiments, the water-soluble polymer comprises poly(alkylene oxide), polysaccharide, poly(vinyl pyrrolidone), poly(vinyl alcohol), polyoxazoline, poly(acryloylmorpholine), or a combination thereof. In some embodiments, the water-soluble polymer is a polysaccharide. In some embodiments, the water-soluble polymer comprises poly(alkylene oxide). In some embodiments, the poly(alkylene oxide) is polyethylene glycol (PEG).

In some embodiments, the polyethylene glycol has a weight average molecular weight of about 0.1 kDa to about 50 kDa. In some embodiments, the polyethylene glycol has a weight average molecular weight of about 0.5 kDa to about 50 kDa. In some embodiments, the polyethylene glycol has a weight average molecular weight of about 10 kDa, about 20 kDa, or about 30 kDa. In some embodiments, the polyethylene glycol has a weight average molecular weight of about 30 kDa. In some embodiments, the polyethylene glycol has a weight average molecular weight of about 20 kDa to about 40 kDa, 25 kDa to about 35 kDa, about 20 kDa to about 50 kDa, or about 40 kDa to about 50 kDa. In some embodiments, the polyethylene glycol has a weight average molecular weight of about 0.5 kDa, about 1 kDa, about 5 kDa, about 10 kDa, about 20 kDa, about 40 kDa, or about 50 kDa. In some embodiments, the polyethylene glycol has a weight average molecular weight of at least about 0.1 kDa, about 0.5 kDa, about 1 kDa, about 5 kDa, about 10 kDa, about 20 kDa, or about 40 kDa. In some embodiments, the polyethylene glycol has a weight average molecular weight of at most about 5 kDa, about 10 kDa, about 20 kDa, about 40 kDa, or about 50 kDa. In some embodiments, the PEG has a Mw of at most about 50 kDa. In some embodiments, the PEG has a Mw of at most about 40 kDa. In some embodiments, the PEG has a Mw of at most about 35 kDa. In some embodiments, the PEG has a Mw of about 10 kDa. In some embodiments, the PEG has a Mw of about 20 kDa. In some embodiments, the PEG has a Mw of about 25 kDa.

In some embodiments, the PEG has a Mw of about 30 kDa. In some embodiments, the PEG has a Mw of about 35 kDa. In some embodiments, the PEG has a Mw of about 40 kDa. In some embodiments, the PEG has a Mw of about 50 kDa. In some embodiments, the PEG has a Mw of about 25 kDa to about 35 kDa. In some embodiments, the PEG has a Mw of about 25 kDa to about 26 kDa, about 25 kDa to about 27 kDa, about 25 kDa to about 28 kDa, about 25 kDa to about 29 kDa, about 25 kDa to about 30 kDa, about 25 kDa to about 31 kDa, about 25 kDa to about 32 kDa, about 25 kDa to about 33 kDa, about 25 kDa to about 34 kDa, about 25 kDa to about 35 kDa, about 26 kDa to about 27 kDa, about 26 kDa to about 28 kDa, about 26 kDa to about 29 kDa, about 26 kDa to about 30 kDa, about 26 kDa to about 31 kDa, about 26 kDa to about 32 kDa, about 26 kDa to about 33 kDa, about 26 kDa to about 34 kDa, about 26 kDa to about 35 kDa, about 27 kDa to about 28 kDa, about 27 kDa to about 29 kDa, about 27 kDa to about 30 kDa, about 27 kDa to about 31 kDa, about 27 kDa to about 32 kDa, about 27 kDa to about 33 kDa, about 27 kDa to about 34 kDa, about 27 kDa to about 35 kDa, about 28 kDa to about 29 kDa, about 28 kDa to about 30 kDa, about 28 kDa to about 31 kDa, about 28 kDa to about 32 kDa, about 28 kDa to about 33 kDa, about 28 kDa to about 34 kDa, about 28 kDa to about 35 kDa, about 29 kDa to about 30 kDa, about 29 kDa to about 31 kDa, about 29 kDa to about 32 kDa, about 29 kDa to about 33 kDa, about 29 kDa to about 34 kDa, about 29 kDa to about 35 kDa, about 30 kDa to about 31 kDa, about 30 kDa to about 32 kDa, about 30 kDa to about 33 kDa, about 30 kDa to about 34 kDa, about 30 kDa to about 35 kDa, about 31 kDa to about 32 kDa, about 31 kDa to about 33 kDa, about 31 kDa to about 34 kDa, about 31 kDa to about 35 kDa, about 32 kDa to about 33 kDa, about 32 kDa to about 34 kDa, about 32 kDa to about 35 kDa, about 33 kDa to about 34 kDa, about 33 kDa to about 35 kDa, or about 34 kDa to about 35 kDa. In some embodiments, the PEG has a Mw of about 25 kDa, about 26 kDa, about 27 kDa, about 28 kDa, about 29 kDa, about 30 kDa, about 31 kDa, about 32 kDa, about 33 kDa, about 34 kDa, or about 35 kDa. In some embodiments, the PEG has a Mw of at least about 25 kDa, about 26 kDa, about 27 kDa, about 28 kDa, about 29 kDa, about 30 kDa, about 31 kDa, about 32 kDa, about 33 kDa, or about 34 kDa. In some embodiments, the PEG has a Mw of at most about 26 kDa, about 27 kDa, about 28 kDa, about 29 kDa, about 30 kDa, about 31 kDa, about 32 kDa, about 33 kDa, about 34 kDa, or about 35 kDa.

In some embodiments, the attached polymer has a weight average molecular weight of about 6,000 Daltons to about 50,000 Daltons. In some embodiments, the polymer has a weight average molecular weight of about 6,000 Daltons to about 10,000 Daltons, about 6,000 Daltons to about 25,000 Daltons, about 6,000 Daltons to about 50,000 Daltons, about 10,000 Daltons to about 25,000 Daltons, about 10,000 Daltons to about 50,000 Daltons, or about 25,000 Daltons to about 50,000 Daltons. In some embodiments, the polymer has a weight average molecular weight of about 6,000 Daltons, about 10,000 Daltons, about 25,000 Daltons, or about 50,000 Daltons. In some embodiments, the polymer has a weight average molecular weight of at least about 6,000 Daltons, about 10,000 Daltons, or about 25,000 Daltons. In some embodiments, the polymer has a weight average molecular weight of at most about 10,000 Daltons, about 25,000 Daltons, or about 50,000 Daltons.

In some embodiments, the attached polymer has a weight average molecular weight of about 120 Daltons to about 1,000 Daltons. In some embodiments, the polymer has a weight average molecular weight of about 100 Daltons to about 250 Daltons, about 100 Daltons to about 300 Daltons, about 100 Daltons to about 400 Daltons, about 100 Daltons to about 500 Daltons, about 100 Daltons to about 1,000 Daltons, about 250 Daltons to about 300 Daltons, about 250 Daltons to about 400 Daltons, about 250 Daltons to about 500 Daltons, about 250 Daltons to about 1,000 Daltons, about 300 Daltons to about 400 Daltons, about 300 Daltons to about 500 Daltons, about 300 Daltons to about 1,000 Daltons, about 400 Daltons to about 500 Daltons, about 400 Daltons to about 1,000 Daltons, or about 500 Daltons to about 1,000 Daltons. In some embodiments, the polymer has a weight average molecular weight of about 100 Daltons, about 250 Daltons, about 300 Daltons, about 400 Daltons, about 500 Daltons, or about 1,000 Daltons. In some embodiments, the polymer has a weight average molecular weight of at least about 100 Daltons, about 250 Daltons, about 300 Daltons, about 400 Daltons, or about 500 Daltons. In some embodiments, the polymer has a weight average molecular weight of at most about 250 Daltons, about 300 Daltons, about 400 Daltons, about 500 Daltons, or about 1,000 Daltons.

In some embodiments, the polymer comprises one or more linker groups. In some embodiments, the linker groups used to attach the polymeric portion of the polymer to the modified IL-18 polypeptide. In some embodiments, the one or more linker groups comprise bifunctional linkers such as an amide group, an ester group, an ether group, a thioether group, a carbonyl group and alike. In some embodiments, the one or more linker groups comprise an amide linker group. In some embodiments, the modified poly(alkylene oxide) comprises one or more spacer groups. In some embodiments, the spacer groups comprise a substituted or unsubstituted C1-C6 alkylene group. In some embodiments, the spacer groups comprise —CH₂—, —CH₂CH₂—, or —CH₂CH₂CH₂—. In some embodiments, the linker group is the product of a biorthogonal reaction (e.g., biocompatible and selective reactions). In some embodiments, the bioorthogonal reaction is a Cu(I)-catalyzed or “copper-free” alkyne-azide triazole-forming reaction, the Staudinger ligation, inverse-electron-demand Diels-Alder (IEDDA) reaction, alkyne-nitrone cycloaddition chemistry, or a metal-mediated process such as olefin metathesis and Suzuki-Miyaura or Sonogashira cross-coupling. In some embodiments, the first polymer is attached to the IL-18 polypeptide via click chemistry.

In some embodiments, the polymer comprises a linker comprising a structure of Formula (X)

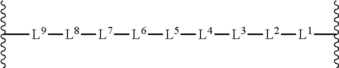

wherein each of L¹, L², L³, L⁴, L⁵, L⁶, L⁷, L⁸, and L⁹ is independently —O—, —NR^{L}—, —(C₁-C₆ alkylene)NR^{L}—, —NR^{L}(C₁-C₆ alkylene)-, —N(R^{L})₂⁺—, —(C₁-C₆ alkylene)N(R^{L})₂⁺—, —N(R^{L})₂⁺—(C₁-C₆ alkylene)-, —OP(═O)(OR^{L})O—, —S—, —(C₁-C₆ alkylene)S—, —S(C₁-C₆ alkylene)-, —S(═O)—, —S(═O)₂—, —C(═O)—, —(C₁-C₆ alkylene)C(═O)—, —C(═O) (C₁-C₆ alkylene)-, —C(═O)O—, —OC(═O)—, —OC(═O)O—, —C(═O)NR^{L}—, —C(═O)NR^{L}(C₁-C₆ alkylene)-, —(C₁-C₆ alkylene)C(═O)NR^{L}—, —NR^{L}C(═O)—, —(C₁-C₆ alkylene)NR^{L}C(═O)—, —NR^{L}C(_O)(C₁-C₆ alkylene)-, —OC(═O)NR^{L}—, —NR^{L}C(═O)O—, —NR^{L}C(═O)NR^{L}—, —NR^{L}C(═S)NR^{L}—, —CR^{L}=N—, —N═CR^{L}, —NR^{L}S(═O)₂—, —S(═O)₂NR^{L}—, —C(═O)NR^{L}S(═O)₂—, —S(═O)₂NR^{L}C(═O)—, substituted or unsubstituted C₁-C₆ alkylene, substituted or unsubstituted C₁-C₆ heteroalkylene, substituted or unsubstituted C₂-C₆ alkenylene, substituted or unsubstituted C₂-C₆ alkynylene, substituted or unsubstituted C₆-C₂₀ arylene, substituted or unsubstituted C₂-C₂₀ heteroarylene, —(CH₂—CH₂—O)_{qa}—, —(O—CH₂—CH₂)_{qb}—, —(CH₂—CH(CH₃)—O)_{qc}—, —(O—CH(CH₃)—CH₂)_{qd}—, a reaction product of a conjugation handle and a complementary conjugation handle, or absent; (C₁-C₆ alkylene)
each R^{L} is independently hydrogen, substituted or unsubstituted C₁-C₄ alkyl, substituted or unsubstituted C₁-C₄ heteroalkyl, substituted or unsubstituted C₂-C₆ alkenyl, substituted or unsubstituted C₂-C₅ alkynyl, substituted or unsubstituted C₃-C₈ cycloalkyl, substituted or unsubstituted C₂-C₇ heterocycloalkyl, substituted or unsubstituted aryl, or substituted or unsubstituted heteroaryl; and
each of qa, qb, qc and qd is independently an integer from 1-100,
wherein each

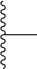

is a point of attachment to the modified IL-18 polypeptide or the polymeric portion of the polymer.

In some embodiments, the polymer comprises a linker comprising a structure of Formula (X′)

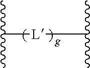

wherein each L′ is independently —O—, —NR^{L}—, —(C₁-C₆ alkylene)NR^{L}—, —NR^{L}(C₁-C₆ alkylene)-, —N(R^{L})₂⁺—, —(C₁-C₆ alkylene)N(R^{L})₂⁺-, —N(R^{L})₂⁺—(C₁-C₆ alkylene)-, —OP(═O)(OR^{L})O—, —S—, —(C₁-C₆ alkylene)S—, —S(C₁-C₆ alkylene)-, —S(═O)—, —S(═O)₂—, —C(═O)—, —(C₁-C₆ alkylene)C(═O)—, —C(═O) (C₁-C₆ alkylene)-, —C(═O)O—, —OC(═O)—, —OC(═O)O—, —C(═O)NR^{L}—, —C(═O)NR^{L}(C₁-C₆ alkylene)-, —(C₁-C₆ alkylene)C(═O)NR^{L}—, —NR^{L}C(═O)—, —(C₁-C₆ alkylene)NR^{L}C(═O)—, —NR^{L}C(═O)(C₁-C₆ alkylene)-, —OC(═O)NR^{L}—, —NR^{L}C(═O)O—, —NR^{L}C(═O)NR^{L}—, —NR^{L}C(═S)NR^{L}—, —CR^{L}=N—, —N=CR^{L}, —NR^{L}S(═O)₂—, —S(═O)₂NR^{L}—, —C(═O)NR^{L}S(═O)₂—, —S(═O)₂NR^{L}C(═O)—, substituted or unsubstituted C₁-C₆ alkylene, substituted or unsubstituted C₁-C₆ heteroalkylene, substituted or unsubstituted C₂-C₆ alkenylene, substituted or unsubstituted C₂-C₆ alkynylene, substituted or unsubstituted C₆-C₂₀ arylene, substituted or unsubstituted C₂-C₂₀ heteroarylene, —(CH₂—CH₂—O)_{qa}, —(O—CH₂—CH₂)_{qb}—, —(CH₂—CH(CH₃)—O)_{qc}—, —(O—CH(CH₃)—CH₂)_{qd}—, a reaction product of a conjugation handle and a complementary conjugation handle, or absent; (C₁-C₆ alkylene)
each R^{L} is independently hydrogen, substituted or unsubstituted C₁-C₄ alkyl, substituted or unsubstituted C₁-C₄ heteroalkyl, substituted or unsubstituted C₂-C₆ alkenyl, substituted or unsubstituted C₂-C₅ alkynyl, substituted or unsubstituted C₃-C₈ cycloalkyl, substituted or unsubstituted C₂-C₇ heterocycloalkyl, substituted or unsubstituted aryl, or substituted or unsubstituted heteroaryl; and
each of qa, qb, qc and qd is independently an integer from 1-100,
g is an integer from 1-100,
wherein each

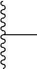

is a point of attachment to the modified IL-18 polypeptide or the polymeric portion of the polymer.

In some embodiments, a polymer modified IL-18 polypeptide provided herein comprises a polymer which includes a linker selected from Table 1. In Table 1, each

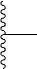

is a point of attachment to either the modified IL-18 polypeptide (e.g., an amino group of the modified IL-18 polypeptide) or to the polymeric portion of the polymer.

TABLE 1
Exemplary linker structures for modified IL-18 polypeptides

| Linker | |
|---|---|
| Identifier | Linker Structure |
| Formula A | 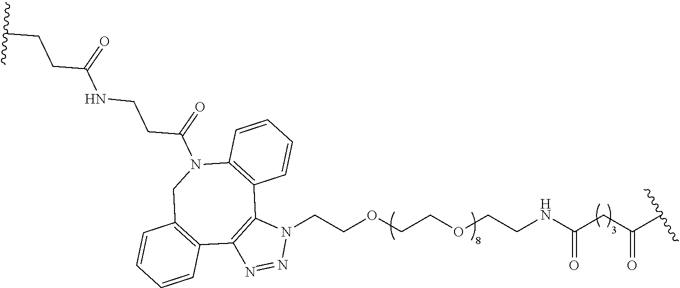 |
| Formula B | 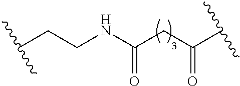 |
| Formula C | 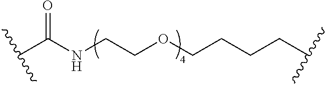 |
| Formula D | 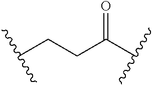 [SMILES: *CCC(*)=O] |
| Formula E | 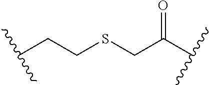 [SMILES: *CCSCC(*)=O] |
| Formula F | 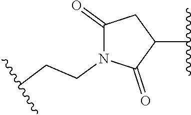 [SMILES: *CCN1C(=O)CC(*)C1=O] |
| Formula G | 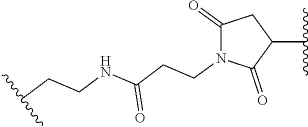 [SMILES: *CCNC(=O)CCN1C(=O)CC(*)C1=O] |
| Formula H | 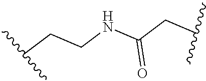 [SMILES: *CCNC(=O)C*] |
| Formula I | 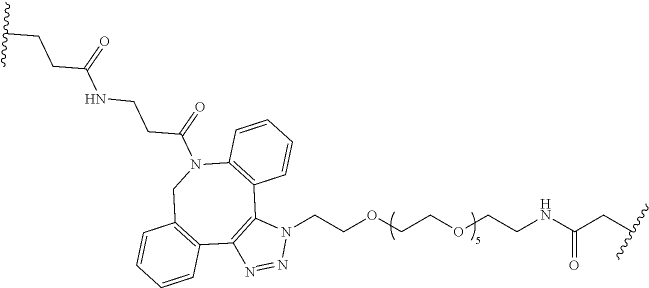 |

In some embodiments, the water-soluble polymer is linear or branched. In some embodiments, the water-soluble polymer is branched and includes a plurality of polyethylene glycol chains. In some embodiments, the water-soluble polymer comprises from 1 to 10 polyethylene glycol chains. In some embodiments, the water-soluble polymer comprises 1 polyethylene glycol chains to 2 polyethylene glycol chains, 1 polyethylene glycol chains to 4 polyethylene glycol chains, 1 polyethylene glycol chains to 6 polyethylene glycol chains, 1 polyethylene glycol chains to 10 polyethylene glycol chains, 2 polyethylene glycol chains to 4 polyethylene glycol chains, 2 polyethylene glycol chains to 6 polyethylene glycol chains, 2 polyethylene glycol chains to 10 polyethylene glycol chains, 4 polyethylene glycol chains to 6 polyethylene glycol chains, 4 polyethylene glycol chains to 10 polyethylene glycol chains, or 6 polyethylene glycol chains to 10 polyethylene glycol chains. In some preferred embodiments, the polyethylene glycol is a linear, unbranched polyethylene glycol.

In some embodiments, addition of the polymer to the modified IL-18 polypeptide increases the stability of the modified IL-18 polypeptide in vivo or in vitro. In some embodiments, addition of the polymer increases one or more pharmacokinetic (PK) properties of the modified IL-18 polypeptide. In some embodiments, the modified IL-18 polypeptide of the disclosure comprises a covalently attached polymer for plasma or serum half-life extension. In some embodiments, a plasma or serum half-life of the polymer modified IL-18 polypeptide of the disclosure is at least 1.5-fold, 2-fold, 3-fold, 4-fold, 5-fold, 6-fold, 7-fold, 8-fold, 9-fold, or 10-fold longer compared to a plasma or serum half-life of a wild-type IL-18 polypeptide. In some embodiments, a plasma or serum half-life of the polymer modified IL-18 polypeptide of the disclosure is 1.5-fold to 10-fold longer compared to a plasma or serum half-life of a wild-type IL-18 polypeptide. In some embodiments, a half-life of the polymer modified IL-18 polypeptide is at least 10% longer than a half-life of a wild-type IL-18 polypeptide. In some embodiments, the half-life of the polymer modified IL-18 polypeptide is at least 30% longer than the half-life of wild-type IL-18 polypeptide.

In some embodiments, a plasma or serum half-life of a modified IL-18 polypeptide described herein is at least 1.5-fold, 2-fold, 3-fold, 4-fold, 5-fold, 6-fold, 7-fold, 8-fold, 9-fold, or 10-fold longer compared to a plasma or serum half-life of an identical IL-18 polypeptide without the polymer attached. In some embodiments, a plasma or serum half-life of the modified IL-18 polypeptide of the disclosure is 1.5-fold to 10-fold longer compared to a plasma or serum half-life of an identical IL-18 polypeptide without the polymer attached. In some embodiments, a half-life of the modified IL-18 polypeptide is at least 10% longer than a half-life of an identical IL-18 polypeptide without the polymer. In some embodiments, the half-life of the modified IL-18 polypeptide is at least 30% longer than the half-life of the an identical IL-18 polypeptide without the polymer.

In some embodiments, the plasma or serum half-life of the modified IL-18 polypeptide described herein is at least about 12 hours, at least about 14 hours, at least about 16 hours, at least about 18 hours, at least about 20 hours, at least about 22 hours, or at least about 24 hours.

### III. Biological Activity of Modified IL-18 Polypeptides

The modified IL-18 polypeptides provided herein can be used as immunotherapies or as parts of other immunotherapy regimens. In some embodiments, the modified IL-18 polypeptides provided herein exhibit an improved serum half-life compared to wild type IL-18 (WT IL-18), reduced binding to IL-18 binding protein (IL-18BP) compared to WT IL-18, and/or an improved activity of IL-18 receptor (IL-18R) signaling in the presence of IL-18BP compared to WT IL-18.

In some embodiments, the modified IL-18 polypeptide exhibits a binding affinity to IL-18R no more than 10-fold reduced compared to an identical IL-18 polypeptide without the polymer (e.g., at most 10-fold, 9-fold, 8-fold, 7-fold, 6-fold, 5-fold, 4-fold, 3-fold, 2-fold reduced affinity or substantially the same affinity). In some embodiments, the modified IL-18 polypeptide displays an enhanced binding affinity to IL-18R as compared do WT IL-18. In some embodiments, the modified IL-18 polypeptides have an increased affinity for the IL-18R□/□ heterodimer compared to WT IL-18. In one aspect, the modified IL-18 polypeptides described herein have no significant decrease in affinity for the IL-18R□/□ heterodimer compared to WT IL-18. In some embodiments, the modified IL-18 polypeptide retains activity against the IL-18 receptor despite an apparent loss of affinity to the IL-18 receptor.

The modified IL-18 polypeptides described herein may display binding characteristics for IL-18 receptor subunits that differ from wild-type IL-18 (e.g., a higher affinity or modestly lower affinity for the IL-18 receptor alpha subunit (IL-18R□) or the IL-18 receptor beta subunit (IL-18R□)). In some embodiments, the binding affinity between the modified IL-18 polypeptides and IL-18R□ is the same as or higher than the binding affinity between a wild-type IL-18 and IL-18R□. In some embodiments, the binding affinity between the modified IL-18 polypeptides and IL-18R□ is the same as or only moderately lower than the binding affinity between a wild type IL-18 and IL-18R□)). In some embodiments, the binding affinity between the modified IL-18 polypeptides and IL-18R□ is the same as or higher than the binding affinity between a wild-type IL-18 and IL-18R□. In some embodiments, the binding affinity between the modified IL-18 polypeptides and IL-18R□ is the same as or only moderately lower than the binding affinity between a wild type IL-18 and IL-18R□.

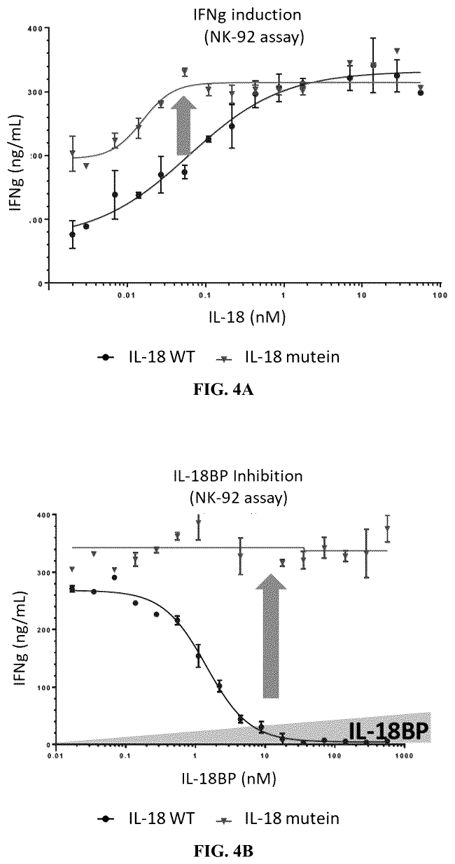

In some embodiments, a modified IL-18 polypeptide provided herein displays an ability to induce interferon gamma (IFN□) production after administration to a subject or to a population of cells. In some embodiments, the ability to induce IFN□□ of the modified IL-18 polypeptide is higher than that of a wild type IL-18 (e.g., displays an EC50 for IFN□□ induction lower than that of a wild type IL-18). An exemplary IL-18 polypeptide provided herein displaying this characteristic is shown in FIG. 4A, which shows a comparison of IFN□ production (ng/mL, γ-axis) as a function of concentration of a wild type versus modified IL-18 polypeptide (mutein) (nM, x-axis).

In some embodiments, the modified interleukin-18 (IL-18) polypeptide, wherein the modified IL-18 polypeptide displays a dissociation constant (K_{D}) for an IL-18 receptor alpha/beta heterodimer (IL-18R□□□) which is at most eight fold greater than the binding affinity displayed by an identical IL-18 polypeptide with no polymer attached. In some embodiments, the dissociation constant for the IL-18R□□□ interaction for the modified IL-18 polypeptide is at most two fold, four fold, or six fold greater than the dissociation constant displayed by an identical IL-18 polypeptide with no polymer attached.

In some embodiments, the modified interleukin-18 (IL-18) polypeptide, wherein the modified IL-18 polypeptide displays a dissociation constant (K_{D}) for an IL-18 receptor alpha (IL-18R□) which is at most 9-fold greater than the dissociation constant displayed by an identical modified IL-18 polypeptide with no polymer attached. In some embodiments, the dissociation constant for the IL-18R□□ interaction of the modified IL-18 polypeptide is at most two fold, four fold, or six fold greater than the dissociation constant displayed by an identical IL-18 polypeptide with no polymer attached.

In some embodiments, a modified IL-18 polypeptide provided herein also display a reduced binding IL-18 binding protein (IL-18BP), as compared to WT IL-18. In some embodiments, the reduced binding to IL-18BP is the result of the attachment polymer and/or additional modifications to the modified IL-18 polypeptide (e.g., additional amino acid substitutions). In some embodiments, a modified IL-18 polypeptide provided herein can induce IFN□□ even in the presence of IL-18BP (e.g., the ability of the modified IL-18 polypeptide to induce IFN□□ is not substantially inhibited by the presence of IL-18BP□□□ nM, x-axis)□□ An example of an IL-18 polypeptide with this property compared to wild type IL-18 is shown in FIG. 4B, which shows IFN□ production (ng/mL, y-axis) as a function of IL-18BP concentration (nM, x-axis) in a sample treated with the same level of wild type IL-18 (circles) or a modified IL-18 polypeptide provided herein (inverted triangles). Notably, the modified IL-18 polypeptide provided herein showed no inhibition in its ability to induce IFN□□ in the presence of IL-18BP, whereas the wild type IL-18 displayed substantial reduction in this ability as the concentration of IL-18BP increased□

### IIIa. Binding Affinity

### Affinity for IL-18 Receptor Alpha/Beta Heterodimer (Il-18R□□)

In some embodiments, the modified IL-18 polypeptide displays at most an only slightly diminished affinity for IL-18R□□ compared to WT IL-18 (SEQ ID NO: 1). In some embodiments, the modified IL-18 polypeptide exhibits at most a 2-fold lower, at most a 3-fold lower, at most a 4-fold lower, at most 5-fold lower, at most a 10-fold lower, at most a 15-fold lower, at most a 20-fold lower, at most a 30-fold lower, at most a 40-fold lower, at most a 50-fold lower, at most a 75-fold lower, or at most a 100-fold lower affinity for IL-18 R□□ as compared to the affinity of WT IL-18 for IL-18R□□. In some embodiments, the modified IL-18 polypeptide exhibits at most at most a 10-fold lower affinity for IL-18R□□ as compared to the affinity of WT IL-18 for IL-18R□□. In some embodiments, the polymer modified IL-18 polypeptide exhibits at most at most a 20-fold lower affinity for IL-18R□□ as compared to the affinity of WT IL-18 for IL-18R□□. In some embodiments, the polymer modified IL-18 polypeptide exhibits at most at most a 50-fold lower affinity for IL-18R□□ as compared to the affinity of WT IL-18 for IL-18R□□. In some embodiments, the polymer modified IL-18 polypeptide exhibits at most at most a 100-fold lower affinity for IL-18R□□ as compared to the affinity of WT IL-18 for IL-18R□□□

In some embodiments, the modified IL-18 polypeptide provided herein exhibits at most only a slight reduction in binding to IL-18R□□ as measured by K_{D}. In some embodiments, the modified IL-18 polypeptide exhibits a K_{D} with IL-18R□□ of at most about 20 nM, at most about 30 nM, at most about 50 nM, at most about 75 nM, at most about 100 nM, or at most about 200 nM. In some embodiments, the modified IL-18 polypeptide exhibits a K_{D} with IL-18R□□ of at most about 20 nM. In some embodiments, the modified IL-18 polypeptide exhibits a K_{D} with IL-18R□□ of at most about 30 nM. In some embodiments, the modified IL-18 polypeptide exhibits a K_{D} with IL-18R□□ of at most about 40 nM. In some embodiments, the modified IL-18 polypeptide exhibits a K_{D} with IL-18R□□ of at most about 50 nM.

In some embodiments, the modified IL-18 polypeptide displays at most an only slightly diminished affinity for IL-18R□□ compared to an identical IL-18 polypeptide without the polymer attached. In some embodiments, the modified IL-18 polypeptide exhibits at most a 2-fold lower, at most a 3-fold lower, at most a 4-fold lower, at most 5-fold lower, at most a 10-fold lower, at most a 15-fold lower, at most a 20-fold lower, at most a 30-fold lower, at most a 40-fold lower, at most a 50-fold lower, at most a 75-fold lower, or at most a 100-fold lower affinity for IL-18R□□ as compared to the affinity of an identical IL-18 polypeptide without the polymer attached for IL-18R□□. In some embodiments, the modified IL-18 polypeptide exhibits at most a 2-fold lower affinity for IL-18R□□ as compared to the affinity of an identical IL-18 polypeptide without the polymer attached. In some embodiments, the modified IL-18 polypeptide exhibits at most a 3-fold lower affinity for IL-18R□□ as compared to the affinity of an identical IL-18 polypeptide without the polymer attached. In some embodiments, the modified IL-18 polypeptide exhibits at most a 4-fold lower affinity for IL-18R□□ as compared to the affinity of an identical IL-18 polypeptide without the polymer attached. In some embodiments, the modified IL-18 polypeptide exhibits at most a 5-fold lower affinity for IL-18R□□ as compared to the affinity of an identical IL-18 polypeptide without the polymer attached. In some embodiments, the modified IL-18 polypeptide exhibits at most a 10-fold lower affinity for IL-18R□□ as compared to the affinity of an identical IL-18 polypeptide without the polymer attached.

### Affinity for IL-18BP

In some embodiments, the modified IL-18 polypeptide provided herein exhibits reduced affinity for IL-18 binding protein (IL-18BP) compared to WT IL-18 (SEQ ID NO: 1). In some embodiments, the modified IL-18 polypeptide exhibits at least 2-fold, at least 5-fold, at least 10-fold, at least 20-fold, at least 30-fold, at least 40-fold, at least 50-fold, at least 60-fold, at least 70-fold, at least 80-fold, at least 90-fold, at least 100-fold, or at least 200-fold, lower affinity for IL-18BP compared to WT IL-18. In some embodiments, the modified IL-18 polypeptide exhibits at least 2-fold lower affinity for IL-18BP compared to WT IL-18. In some embodiments, the modified IL-18 polypeptide exhibits at least 2-fold lower affinity for IL-18BP compared to WT IL-18. In some embodiments, the modified IL-18 polypeptide exhibits at least 5-fold lower affinity for IL-18BP compared to WT IL-18. In some embodiments, the modified IL-18 polypeptide exhibits at least 10-fold lower affinity for IL-18BP compared to WT IL-18. In some embodiments, the modified IL-18 polypeptide exhibits at least 20-fold lower affinity for IL-18BP compared to WT IL-18. In some embodiments, the modified IL-18 polypeptide exhibits at least 30-fold lower affinity for IL-18BP compared to WT IL-18. In some embodiments, the modified IL-18 polypeptide exhibits at least 40-fold lower affinity for IL-18BP compared to WT IL-18. In some embodiments, the modified IL-18 polypeptide exhibits at least 50-fold lower affinity for IL-18BP compared to WT IL-18. In some embodiments, the modified IL-18 polypeptide exhibits at least 60-fold lower affinity for IL-18BP compared to WT IL-18. In some embodiments, the modified IL-18 polypeptide exhibits at least 70-fold lower affinity for IL-18BP compared to WT IL-18. In some embodiments, the modified IL-18 polypeptide exhibits at least 80-fold lower affinity for IL-18BP compared to WT IL-18. In some embodiments, the modified IL-18 polypeptide exhibits at least 90-fold lower affinity for IL-18BP compared to WT IL-18. In some embodiments, the modified IL-18 polypeptide exhibits at least 100-fold lower affinity for IL-18BP compared to WT IL-18. In some embodiments, the modified IL-18 polypeptide exhibits at least 200-fold lower affinity for IL-18BP compared to WT IL-18.

### IIIb. Functional Activity

The modified IL-18 polypeptide provided herein exhibits a higher ability to signal through the IL-18 receptor (IL-18R) as compared to WT IL-18. The modified IL-18 polypeptide also displays a reduced ability to be inhibited by IL-18BP compared to WT IL-18.

In some embodiments, a modified IL-18 polypeptide provided herein display one or more activities associated with WT IL-18. In some embodiments, the modified IL-18 polypeptide exhibits a similar ability to signal through the IL-18 receptor (IL-18R) as compared to an identical IL-18 polypeptide without the polymer attached (e.g., substantially the same or only slightly reduced ability (e.g., reduced by 20-fold or lower).

In some embodiments, the modified IL-18 polypeptide also displays a reduced ability to be inhibited by IL-18BP compared to WT IL-18. In some embodiments, the modified IL-18 polypeptide with polymer attached displays a reduced ability to be inhibited by IL-18BP compared to an identical IL-18 polypeptide without the polymer attached.

### IFN□ Production Stimulation Activity (EC₅₀)

In some embodiments, the modified IL-18 polypeptide modulates IFN□ production when in contact with a cell (e.g., an immune cell, such as an PBMC, NK cell). In some embodiments, the modified IL-18 polypeptide's ability to modulate IFN□ production is measured as a half-maximal effective concentration (EC₅₀). The modified IL-18 polypeptide exhibits higher ability to induce IFNγ production in a cell compared to WT IL-18. In certain embodiments, the EC50 to induce IFNγ production in a cell for the modified IL-18 polypeptide is lower than the EC50 for WT IL-18 by at least 2-fold.

In certain embodiments, the EC50 to induce IFNγ production in a cell for the modified IL-18 polypeptide is lower than the EC50 for WT IL-18 by at least 2-fold. In certain embodiments, the EC50 to induce IFNγ production in a cell for the modified IL-18 polypeptide is lower than the EC50 for WT IL-18 by at least 2-fold, 3-fold, 4-fold, 5-fold, 7-fold, or 10-fold. In certain embodiments, the EC50 to induce IFNγ production in a cell for the modified IL-18 polypeptide is lower than the EC50 for WT IL-18 by at least 2-fold. In certain embodiments, the EC50 to induce IFNγ production in a cell for the modified IL-18 polypeptide is lower than the EC50 for WT IL-18 by at least 3-fold. In certain embodiments, the EC50 to induce IFNγ production in a cell for the modified IL-18 polypeptide is lower than the EC50 for WT IL-18 by at least 4-fold. In certain embodiments, the EC50 to induce IFNγ production in a cell for the modified IL-18 polypeptide is lower than the EC50 for WT IL-18 by at least 5-fold. In certain embodiments, the EC50 to induce IFNγ production in a cell for the modified IL-18 polypeptide is lower than the EC50 for WT IL-18 by at least 6-fold. In certain embodiments, the EC50 to induce IFNγ production in a cell for the modified IL-18 polypeptide is lower than the EC50 for WT IL-18 by at least 7-fold. In certain embodiments, the EC50 to induce IFNγ production in a cell for the modified IL-18 polypeptide is lower than the EC50 for WT IL-18 by at least 8-fold. In certain embodiments, the EC50 to induce IFNγ production in a cell for the modified IL-18 polypeptide is lower than the EC50 for WT IL-18 by at least 9-fold. In certain embodiments, the EC50 to induce IFNγ production in a cell for the modified IL-18 polypeptide is lower than the EC50 for WT IL-18 by at least 10-fold.

In certain embodiments, the EC50 for signaling through the IL-18 receptor, for the modified IL-18 polypeptide is lower than the EC50 for WT IL-18 by at least 5-fold. In certain embodiments, the EC50 for signaling through the IL-18 receptor, for the modified IL-18 polypeptide is lower than the EC50 for WT IL-18 by 5-fold to 50-fold, 5-fold to 25-fold, or 5-fold to 20-fold. In certain embodiments, the EC50 for signaling through the IL-18 receptor, for the modified IL-18 polypeptide is lower than the EC50 for WT IL-18 by at least 5-fold, 6-fold, 7-fold, 8-fold, 9-fold, or 10-foldIn certain embodiments, the EC50 for signaling through the IL-18 receptor, for the modified IL-18 polypeptide is at least 5-fold lower than the EC50 for WT IL-18. In certain embodiments, the EC50 for signaling through the IL-18 receptor, for the modified IL-18 polypeptide is at least 10-fold lower than the EC50 for WT IL-18. EC50 for signaling through the IL-18 receptor can be measured using HEK-Blue IL18R Reporter Assay (e.g., as provided herein in the Examples).

In some embodiments, an EC₅₀ (nM) of the modified IL-18 polypeptide's ability to induce IFNγ is less than 10-fold higher than, less than 5-fold higher than, or less than an EC₅₀ (nM) of an IL-18 polypeptide of SEQ ID NO: 1. In some embodiments, the EC₅₀ of the polymer modified IL-18 polypeptide's ability to induce IFNγ is less than 10-fold higher than an EC₅₀ (nM) of an IL-18 polypeptide of SEQ ID NO: 1. In some embodiments, the EC₅₀ of the polymer modified IL-18 polypeptide's ability to induce IFNγ is less than 5-fold higher than an EC₅₀ (nM) of an IL-18 polypeptide of SEQ ID NO: 1. In some embodiments, the EC₅₀ of the polymer modified IL-18 polypeptide's ability to induce IFNγ is less than an EC₅₀ (nM) of an IL-18 polypeptide of SEQ ID NO: 1. In some embodiments, the EC₅₀ of the modified IL-18 polypeptide's ability to induce IFNγ is less than 10-fold higher than, less than 8-fold higher than, less than 6-fold higher than, less than 5-fold higher than, less than 4-fold higher than, less than 3-fold higher than, or less than 2-fold higher than an EC₅₀ (nM) of an IL-18 polypeptide of SEQ ID NO: 1. In some embodiments, the EC₅₀ of the polymer modified IL-18 polypeptide to induce IFNg is lower than that of an IL-18 polypeptide of SEQ ID NO: 1 (e.g., at least 2-fold, 3-fold, 4-fold, 5-fold, or 6-fold lower). In some embodiments, the EC₅₀ of the polymer modified IL-18 polypeptide's ability to induce IFN□□ is □ measured by an IFN□ induction cellular assay. In some embodiments, the ability is measured in peripheral blood mononuclear cells. In some embodiments, the ability is measured in NK92 cells.

In some embodiments, an EC₅₀ of the polymer modified IL-18 polypeptide's ability to induce IFN□□ production is less than about 100 nM, less than about 75 nM, less than about 50 nM, less than about 40 nM, less than about 30 nM, less than about 20 nM, less than about 15 nM, less than about 10 nM, less than about 0.1 nM, less than about 0.09 nM, less than about 0.08 nM, less than about 0.07 nM, less than about 0.06 nM, or less than about 0.05 nM. In some embodiments, an EC₅₀ of the polymer modified IL-18 polypeptide's ability to induce IFN□□ production is less than about 100 nM. In some embodiments, an EC₅₀ of the polymer modified IL-18 polypeptide's ability to induce IFN□□ production is less than about 80 nM. In some embodiments, an EC₅₀ of the polymer modified IL-18 polypeptide's ability to induce IFN□□ production is less than about 50 nM. In some embodiments, an EC₅₀ of the polymer modified IL-18 polypeptide's ability to induce IFN□□ production is less than about 10 nM. In some embodiments, an EC₅₀ of the polymer modified IL-18 polypeptide's ability to induce IFNg production is less than about 1 nM. In some embodiments, an EC₅₀ of the polymer modified IL-18 polypeptide's ability to induce IFNg production is less than about 0.1 nM. In some embodiments, an EC₅₀ of the polymer modified IL-18 polypeptide's ability to induce IFNg production is less than about 0.09 nM. In some embodiments, an EC₅₀ of the polymer modified IL-18 polypeptide's ability to induce IFNg production is less than about 0.08 nM. In some embodiments, an EC₅₀ of the polymer modified IL-18 polypeptide's ability to induce IFNg production is less than about 0.07 nM. In some embodiments, an EC₅₀ of the polymer modified IL-18 polypeptide's ability to induce IFNg production is less than about 0.06 nM. In some embodiments, an EC₅₀ of the polymer modified IL-18 polypeptide's ability to induce IFNg production is less than about 0.05 nM.

In some embodiments, the modified IL-18 polypeptide displays a similar or only slightly reduced ability to induce IFN D production compared to an identical IL-18 polypeptide without the polymer attached. In some embodiments, an EC₅₀ (nM) of IFN□ production of the modified IL-18 polypeptide is at most about 100-fold higher than, at most about 90-fold higher than, at most about 80-fold higher than, at most about 70-fold higher than, at most about 60-fold higher than, at most about 50-fold higher than, at most about 40-fold higher than, at most about 50-fold higher than, at most about 40-fold higher than, at most about 50-fold higher than, at most about 40-fold higher than, at most about 30-fold higher than, at most about 20-fold higher than, at most about 10-fold higher than, at most about 9-fold higher than, at most about 8-fold, higher than at most about 7-fold higher than, at most about 6-fold higher than, at most about 5-fold higher than, at most about 4-fold higher than, at most about 3-fold higher than, or at most about 2-fold higher than an EC₅₀ of an identical IL-18 polypeptide without the polymer attached. In some embodiments, EC₅₀ of IFNγ production of the modified IL-18 polypeptide is at most about 5-fold higher than an EC₅₀ of an identical IL-18 polypeptide without the polymer attached. In some embodiments, EC₅₀ of IFNγ production of the modified IL-18 polypeptide is at most about 10-fold higher than an EC₅₀ of an identical IL-18 polypeptide without the polymer attached. In some embodiments, EC₅₀ of IFNγ production of the modified IL-18 polypeptide is at most about 20-fold higher than an EC₅₀ of an identical IL-18 polypeptide without the polymer attached. In some embodiments, EC₅₀ of IFNγ production of the modified IL-18 polypeptide is at most about 30-fold higher than an EC₅₀ of an identical IL-18 polypeptide without the polymer attached. In some embodiments, EC₅₀ of IFNγ production of the modified IL-18 polypeptide is at most about 40-fold higher than an EC₅₀ of an identical IL-18 polypeptide without the polymer attached. In some embodiments, EC₅₀ of IFNγ production of the modified IL-18 polypeptide is at most about 50-fold higher than an EC₅₀ of an identical IL-18 polypeptide without the polymer attached. In some embodiments, EC₅₀ of IFNγ production of the modified IL-18 polypeptide is at most about 60-fold higher than an EC₅₀ of an identical IL-18 polypeptide without the polymer attached. In some embodiments, EC₅₀ of IFNγ production of the modified IL-18 polypeptide is at most about 70-fold higher than an EC₅₀ of an identical IL-18 polypeptide without the polymer attached. In some embodiments, EC₅₀ of IFNγ production of the modified IL-18 polypeptide is at most about 80-fold higher than an EC₅₀ of an identical IL-18 polypeptide without the polymer attached. In some embodiments, EC₅₀ of IFNγ production of the modified IL-18 polypeptide is at most about 90-fold higher than an EC₅₀ of an identical IL-18 polypeptide without the polymer attached. In some embodiments, EC₅₀ of IFNγ production of the modified IL-18 polypeptide is at most about 100-fold higher than an EC₅₀ of an identical IL-18 polypeptide without the polymer attached.

### Inhibition by IL-18BP (IC₅₀)

In some embodiments, the modified IL-18 exhibits a reduced ability to have its IFN□ induction activity inhibited by IL-18BP compared to WT IL-18. In some embodiments, the modified IL-18 displays a half-maximal inhibitory concentration (IC₅₀) by IL-18BP which is at least about 10-fold higher than, at least about 20-fold higher than, at least about 30-fold at least about 50-fold higher than, at least about 75-fold higher than, at least about 100-fold higher than, at least about 200-fold higher than, at least about 300-fold higher than, at least about 400-fold higher than, at least about 500-fold higher than, at least about 600-fold higher than, at least about 700-fold higher than, at least about 800-fold higher than, at least about 900-fold higher than, or at least about 1000-fold higher than an IC₅₀ of WT IL-18's inhibition by IL-18BP. In some embodiments, the modified IL-18 displays a half-maximal inhibitory concentration (IC₅₀) by IL-18BP which is at least about 20-fold higher than an IC₅₀ of WT IL-18's inhibition by IL-18BP. In some embodiments, the modified IL-18 displays a half-maximal inhibitory concentration (IC₅₀) by IL-18BP which is at least about 30-fold higher than an IC₅₀ of WT IL-18's inhibition by IL-18BP. In some embodiments, the modified IL-18 displays a half-maximal inhibitory concentration (IC₅₀) by IL-18BP which is at least about 50-fold higher than an IC₅₀ of WT IL-18's inhibition by IL-18BP.

Ratio of IC₅₀/EC₅₀

In some embodiments, the modified IL-18 polypeptide exhibits a favorable ratio of half-maximal inhibitory concentration (IC₅₀) by IL-18BP over a half-maximal effective concentration (EC₅₀) of IFN□ induction (IC₅₀/EC₅₀ ratio). In some embodiments, the IC₅₀/EC₅₀ ratio for the modified IL-18 polypeptide is increased compared to WT IL-18. In some embodiments, the IC₅₀/EC₅₀ ratio of the modified IL-18 polypeptide is at least about 2, at least about 5, at least about 10, at least about 50, at least about 100, at least about 250, or at least about 500.

### IV. Pharmaceutical Compositions

In one aspect, described herein is a pharmaceutical composition comprising: a modified IL-18 polypeptide described herein; and a pharmaceutically acceptable carrier or excipient. In some embodiments, the pharmaceutical composition comprises a plurality of the modified IL-18 polypeptides. In an aspect, described herein is a pharmaceutical composition comprising: a modified IL-18 polypeptide described herein; and a pharmaceutically acceptable carrier or excipient. In some embodiments, the pharmaceutical composition comprises a plurality of the modified IL-18 polypeptides. In some embodiments, the pharmaceutical compositions further comprises one or more excipient selected from a carbohydrate, an inorganic salt, an antioxidant, a surfactant, or a buffer.

In some embodiments, the pharmaceutical composition further comprises a carbohydrate. In certain embodiments, the carbohydrate is selected from the group consisting of fructose, maltose, galactose, glucose, D-mannose, sorbose, lactose, sucrose, trehalose, cellobiose raffinose, melezitose, maltodextrins, dextrans, starches, mannitol, xylitol, maltitol, lactitol, xylitol, sorbitol (glucitol), pyranosyl sorbitol, myoinositol, cyclodextrins, and combinations thereof.

In some embodiments, the pharmaceutical composition comprises an inorganic salt. In certain embodiments, the inorganic salt is selected from the group consisting of sodium chloride, potassium chloride, magnesium chloride, calcium chloride, sodium phosphate, potassium phosphate, sodium sulfate, or combinations thereof.

In certain embodiments, the pharmaceutical composition comprises an antioxidant. In certain embodiments, the antioxidant is selected from the group consisting of ascorbyl palmitate, butylated hydroxyanisole, butylated hydroxytoluene, potassium metabisulfite, propyl gallate, sodium metabisulfite, sodium thiosulfate, vitamin E, 3,4-dihydroxybenzoic acid, and combinations thereof.

In certain embodiments, the pharmaceutical composition comprises a surfactant. In certain embodiments, the surfactant is selected from the group consisting of polysorbates, sorbitan esters, lipids, phospholipids, phosphatidylethanolamines, fatty acids, fatty acid esters, steroids, EDTA, zinc, and combinations thereof.

In certain embodiments, the pharmaceutical composition comprises a buffer. In certain embodiments, the buffer is selected from the group consisting of citric acid, sodium phosphate, potassium phosphate, acetic acid, ethanolamine, histidine, amino acids, tartaric acid, succinic acid, fumaric acid, lactic acid, tris, HEPES, CHAPS, or combinations thereof.

In some embodiments, the pharmaceutical composition is formulated for parenteral or enteral administration. In some embodiments, the pharmaceutical composition is formulated for intravenous or subcutaneous administration. In some embodiments, the pharmaceutical composition is in a lyophilized form.

In one aspect, described herein is a liquid or lyophilized composition that comprises a described modified IL-18 polypeptide. In some embodiments, the modified IL-18 polypeptide is a lyophilized powder. In an aspect, described herein is a liquid or lyophilized composition that comprises a described polymer modified IL-18 polypeptide. In some embodiments, the polymer modified IL-18 polypeptide is a lyophilized powder. In some embodiments, the lyophilized powder is resuspended in a buffer solution. In some embodiments, the buffer solution comprises a buffer, a sugar, a salt, a surfactant, or any combination thereof. In some embodiments, the buffer solution comprises a phosphate salt. In some embodiments, the phosphate salt is sodium Na₂HPO₄.

In some embodiments, the salt is sodium chloride. In some embodiments, the buffer solution comprises phosphate buffered saline. In some embodiments, the buffer solution comprises mannitol. In some embodiments, the lyophilized powder is suspended in a solution comprising phosphate buffered saline solution (pH 7.4) with 50 mg/mL mannitol. In some embodiments, the pharmaceutical composition is a lyophilized composition which is reconstituted shortly before administration to a subject.

The modified IL-18 polypeptides described herein can be in a variety of dosage forms. In some embodiments, the modified IL-18 polypeptide is dosed as a lyophilized powder. In some embodiments, the modified IL-18 polypeptide is dosed as a suspension. In some embodiments, the modified IL-18 polypeptide is dosed as a solution. In some embodiments, the modified IL-18 polypeptide is dosed as an injectable solution. In some embodiments, the modified IL-18 polypeptide is dosed as an IV solution.

### V. Method of Treatment

In one aspect, described herein, is a method of treating cancer in a subject in need thereof, comprising: administering to the subject an effective amount of a modified IL-18 polypeptide as provided herein or a pharmaceutical composition as described herein. In some embodiments, the subject is a human.

In an aspect, described herein, is a modified IL-18 polypeptide as provided herein for use in treatment of cancer in a subject in need thereof. In another aspect, described herein, is a modified IL-18 polypeptide as provided herein for in the manufacture of a medicament for treatment of cancer in a subject in need thereof.

In some embodiments, the cancer is a solid cancer. In some embodiments, the solid cancer is adrenal cancer, anal cancer, bile duct cancer, bladder cancer, bone cancer, brain cancer, breast cancer, carcinoid cancer, cervical cancer, colorectal cancer, esophageal cancer, eye cancer, gallbladder cancer, gastrointestinal stromal tumor, germ cell cancer, head and neck cancer, kidney cancer, liver cancer, lung cancer, nasal cavity and paranasal sinus cancer, nasopharyngeal cancer, neuroblastoma, neuroendocrine cancer, oral cancer, oropharyngeal cancer, ovarian cancer, pancreatic cancer, pediatric cancer, penile cancer, pituitary cancer, prostate cancer, skin cancer, soft tissue cancer, spinal cord cancer, stomach cancer, testicular cancer, thymus cancer, thyroid cancer, ureteral cancer, uterine cancer, vaginal cancer, or vulvar cancer.

In some embodiments, the cancer is a blood cancer. In some embodiments, the blood cancer is leukemia, non-Hodgkin lymphoma, Hodgkin lymphoma, an AIDS-related lymphoma, multiple myeloma, plasmacytoma, post-transplantation lymphoproliferative disorder, or Waldenstrom macroglobulinemia.

A modified IL-18 polypeptide described herein can be administered to a subject in one or more doses. In some embodiments, the modified IL-18 polypeptide is administered in a single dose of the effective amount of the modified IL-18 polypeptide, including further embodiments in which (i) the modified IL-18 polypeptide is administered once a day; or (ii) the modified IL-18 polypeptide is administered to the subject multiple times over the span of one day. In some embodiments, the modified IL-18 polypeptide is administered daily, every other day, twice a week, 3 times a week, once a week, every 2 weeks, every 3 weeks, every 4 weeks, every 5 weeks, every 6 weeks, every 12 weeks, every 3 days, every 4 days, every 5 days, every 6 days, 2 times a week, 3 times a week, 4 times a week, 5 times a week, 6 times a week, once a month, twice a month, 3 times a month, 4 times a month, once every 2 months, once every 3 months, once every 4 months, once every 5 months, or once every 6 months. Administration includes, but is not limited to, injection by any suitable route (e.g., parenteral, enteral, intravenous, subcutaneous, etc.).

An effective response is achieved when the subject experiences partial or total alleviation or reduction of signs or symptoms of illness, and specifically includes, without limitation, prolongation of survival. The expected progression-free survival times may be measured in months to years, depending on prognostic factors including the number of relapses, stage of disease, and other factors. Prolonging survival includes without limitation times of at least 1 month (mo), about at least 2 mos., about at least 3 mos., about at least 4 mos., about at least 6 mos., about at least 1 year, about at least 2 years, about at least 3 years, about at least 4 years, about at least 5 years, etc. Overall or progression-free survival can be also measured in months to years. Alternatively, an effective response may be that a subject's symptoms remain static and do not worsen. Further indications of treatment of indications are described in more detail below. In some instances, a cancer or tumor is reduced by at least 2%, 5%, 10%, 15%, 20%, 25%, 30%, 35%, 40%, 45%, 50%, 55%, 60%, 65%, 70%, 75%, 80%, 85%, 90%, 95%, or 100%.

The effective amount of the modified IL-18 polypeptide administered can be 1 μg/kg/administration to about 30 mg/kg/administration. In some embodiments, the effective amount of the modified IL-18 polypeptide administered is about 1 μg/kg/administration to about 100 μg/kg/administration. In some embodiments, the effective amount of the modified IL-18 polypeptide administered is about 1 μg/kg/administration to about 100 μg/kg/administration, about 5 μg/kg/administration to about 100 μg/kg/administration, about 10 μg/kg/administration to about 20 μg/kg/administration, about 10 μg/kg/administration to about 30 μg/kg/administration, about 10 μg/kg/administration to about 40 μg/kg/administration, about 10 μg/kg/administration to about 50 μg/kg/administration, about 10 μg/kg/administration to about 100 μg/kg/administration, about 20 μg/kg/administration to about 30 μg/kg/administration, about 20 μg/kg/administration to about 40 μg/kg/administration, about 20 μg/kg/administration to about 50 μg/kg/administration, about 20 μg/kg/administration to about 100 μg/kg/administration, about 30 μg/kg/administration to about 40 μg/kg/administration, about 30 μg/kg/administration to about 50 μg/kg/administration, about 30 μg/kg/administration to about 100 μg/kg/administration, about 40 μg/kg/administration to about 50 μg/kg/administration, about 40 μg/kg/administration to about 100 μg/kg/administration, or about 50 μg/kg/administration to about 100 μg/kg/administration, about. In some embodiments, the effective amount of the modified IL-18 polypeptide administered is about 1 μg/kg/administration, about 5 μg/kg/administration, about 10 μg/kg/administration, about 20 μg/kg/administration, about 30 μg/kg/administration, about 40 μg/kg/administration, about 50 μg/kg/administration, about 60 μg/kg/administration, about 70 μg/kg/administration, about 80 μg/kg/administration, about 90 μg/kg/administration, or about 100 μg/kg/administration. In some embodiments, the effective amount of the modified IL-18 polypeptide administered is at least about 1 μg/kg/administration, about 5 μg/kg/administration, about 10 μg/kg/administration, about 20 μg/kg/administration, about 30 μg/kg/administration, about 40 μg/kg/administration, about 50 μg/kg/administration, about 60 μg/kg/administration, about 70 μg/kg/administration, about 80 μg/kg/administration, or about 90 μg/kg/administration. In some embodiments, the effective amount of the modified IL-18 polypeptide administered is at most about 5 μg/kg/administration, about 10 μg/kg/administration, about 20 μg/kg/administration, about 30 μg/kg/administration, about 40 μg/kg/administration, about 50 μg/kg/administration, about 60 μg/kg/administration, about 70 μg/kg/administration, about 80 μg/kg/administration, about 90 μg/kg/administration, or about 100 μg/kg/administration.

In some embodiments, the effective amount of the modified IL-18 polypeptide administered is about 0.1 mg/kg/administration to about 10 mg/kg/administration. In such embodiments, these higher doses are generally preferred in non-human mammalian subjects, such mice and other smaller mammals. In some embodiments, the effective amount of the modified IL-18 polypeptide administered is about 0.1 mg/kg/administration to about 0.5 mg/kg/administration, about 0.1 mg/kg/administration to about 1 mg/kg/administration, about 0.1 mg/kg/administration to about 5 mg/kg/administration, about 0.5 mg/kg/administration to about 1 mg/kg/administration, about 0.5 mg/kg/administration to about 5 mg/kg/administration, about 0.5 mg/kg/administration to about 10 mg/kg/administration, about 1 mg/kg/administration to about 2 mg/kg/administration, about 1 mg/kg/administration to about 3 mg/kg/administration, about 1 mg/kg/administration to about 5 mg/kg/administration, about 1 mg/kg/administration to about 8 mg/kg/administration, a about 1 mg/kg/administration to about 10 mg/kg/administration, about 2 mg/kg/administration to about 5 mg/kg/administration, about 2 mg/kg/administration to about 10 mg/kg/administration, about 3 mg/kg/administration to about 5 mg/kg/administration, about 3 mg/kg/administration to about 10 mg/kg/administration, about 4 mg/kg/administration to about 10 mg/kg/administration, about 5 mg/kg/administration to about 10 mg/kg/administration, about 6 mg/kg/administration to about 10 mg/kg/administration, or about 7 mg/kg/administration to about 10 mg/kg/administration. In some embodiments, the effective amount of the modified IL-18 polypeptide administered is about 0.1 mg/kg/administration, about 0.5 mg/kg/administration, about 1 mg/kg/administration, about 2 mg/kg/administration, about 3 mg/kg/administration, about 4 mg/kg/administration, about 5 mg/kg/administration, about 6 mg/kg/administration, about 7 mg/kg/administration, about 8 mg/kg/administration, about 9 mg/kg/administration, or about 10 mg/kg/administration. In some embodiments, the effective amount of the modified IL-18 polypeptide administered is at least about 0.1 mg/kg/administration, about 0.5 mg/kg/administration, about 1 mg/kg/administration, about 2 mg/kg/administration, about 3 mg/kg/administration, about 4 mg/kg/administration, about 5 mg/kg/administration, about 6 mg/kg/administration, about 7 mg/kg/administration, about 8 mg/kg/administration, or about 9 mg/kg/administration. In some embodiments, the effective amount of the modified IL-18 polypeptide administered is at most about 0.5 mg/kg/administration, about 1 mg/kg/administration, about 2 mg/kg/administration, about 3 mg/kg/administration, about 4 mg/kg/administration, about 5 mg/kg/administration, about 6 mg/kg/administration, about 7 mg/kg/administration, about 8 mg/kg/administration, about 9 mg/kg/administration, or about 10 mg/kg/administration.

In some embodiments, the method further comprises reconstituting a lyophilized form of the modified IL-18 polypeptide or the pharmaceutical composition. In some embodiments, the modified IL-18 polypeptide or the pharmaceutical composition is reconstituted before administration. In some embodiments, the composition is reconstituted immediately before administration, up to about 5 minutes before administration, up to about 20 minutes before administration, up to about 40 minutes before administration, up to an hour before administration, or up to about four hours before administration.

Therapeutic combinations of modified IL-18 polypeptides and additional agents are also contemplated within the instant disclosure. Combination therapy can involve co-administration of another therapeutic agent, either at the same time (e.g., on the same day) or on different days. The modified IL-18 polypeptide and the other therapeutic agent can be administered at the same or different intervals as part of a therapy regimen.

In some embodiments, a method of treatment as described herein with a modified IL-18 polypeptide further comprises administration of an immune checkpoint inhibitor. In some embodiments, the immune checkpoint inhibitor is an anti-PD-1 antibody, an anti-PD-L1 antibody, or an anti-CTLA4 antibody. In some embodiments, the immune checkpoint inhibitor is a PD-1 antibody. Examples of anti-PD-1 antibodies include, without limitation, Pembrolizumb, Nivolumab, LZM-009, Cemiplimab, Dostarlimab, and Retifanlimab. The antibody can also be a multi-specific antibody (e.g., a bi-specific antibody) which is specific for an immune checkpoint as described herein (e.g., PD-1) and an additional target.

### VI. Preparation of Recombinant IL-18 Polypeptides

In one aspect, described herein is a method of producing a modified IL-18 polypeptide, wherein the method comprises expressing the modified IL-18 polypeptide in a host cell. The modified IL-18 polypeptide without the polymer can be expressed in the host cell.

In one aspect, described herein is a host cell comprising a modified IL-18 polypeptide.

In some embodiments, the host cell is a prokaryotic cell or a eukaryotic cell. In some embodiments, the host cell is a mammalian cell, an avian cell, or an insect cell. In some embodiments, the host cell is a mammalian cell, an avian cell, a fungal cell, or an insect cell. In some embodiments, the host cell is a CHO cell, a COS cell, or a yeast cell.

In some embodiments, the method comprises attaching a polymer to the modified IL-18 polypeptide. In some embodiments, the polymer is directly attached to a residue of the modified IL-18 polypeptide, optionally through a linker attached to the polymer. In some embodiments, a heterobifunctional linker is first attached to the modified IL-18 polypeptide, followed by attachment of the polymer to the heterobifunctional linker.

### EXAMPLES

The present disclosure is further illustrated in the following Examples which are given for illustration purposes only and are not intended to limit the disclosure in any way.

### Example 1—Recombinant IL18 Expression and Purification

Recombinant IL-18 variants provided herein can be prepared according to the protocols provided below.

Soluble his-SUMO-IL18 Variants

Recombinant BL21 Star (DE3) containing plasmid encoding an IL-18 polypeptide provided herein (from glycerol stocks) is inoculated into LB medium containing 50 μg/mL kanamycin and cultured at 37° C. When the OD600 reaches about 0.8-1.0, cell culture is induced with 0.5 mM IPTG at 18° C./18 h. Cells are then harvested by centrifugation (4500×g, 45 min, 4° C.). Cells are pelleted and cell lysis is done by sonication in lysis buffer: PBS, pH 7.4. Soluble protein is purified via Ni-NTA beads 6FF (wash 1 with: PBS, 20 mM imidazole, pH7.4; wash 2 with PBS, 50 mM Imidazole, pH7.4; elution with PBS, 500 mM imidazole, pH7.4).

Cell pellets are resuspended with lysis buffer (50 mM NaH2PO4, 300 mM NaCl, 20 mM Imidazole, pH 7.4) followed by sonication on ice (20% amplitude, 5 seconds on/5 seconds off, 5 minutes total). The lysate is cleared by centrifugation (13500 RPM, 45 min, 4° C.) and the supernatant is kept for future purification. Target protein is obtained by three-step purification:

### Step 1: Column: Histrap FF 5 mL (Cytiva)

Buffer A: 50 mM NaH2PO4, 300 mM NaCl, 20 mM Imidazole pH 7.4/Buffer B: 50 mM NaH2PO4, 300 mM NaCl, 500 mM Imidazole, pH 7.4. Elution with gradient 0-18% B over 5 CV (in waste), then constant 18% B over another 10 CV (3 ml Fractions).

### Step 2: Column: Histrap FF 5 mL

The collected fractions are pooled into Dialysis tube (SnakeSkin, 10 K, 35 mm), then buffer-exchanged with 5 L dialysis buffer (50 mM NaH₂PO₄, 1 mM DTT, pH 7.4) over night at 4° C. to remove imidazole. SUMO protease is added into the tag-fused protein (amounts assessed by nanodrop) solution at a ratio of 1:50 (w/w), SUMO tag is fully cleaved after 1 h at 4° C. Reaction solution is then applied into Histrap FF 5 mL, flow through is collected, SUMO protease and His-SUMO tag are eluted with a gradient: 0% B over 10 CV (3 ml Fractions), 0-60% B over 5 CV (in waste), 60-100% B with 5 CV, 100% B with 5CV.

### Step 3: Column: HiLoad 16/600 Superdex 75 μg

Pooled flow through is concentrated to 6-8 mg/mL, 5 mL concentrated protein solution is applied to the column, target protein was eluted with 1.2 CV elution buffer (1×PBS, 10% glycerol, pH 7.4). Target protein is pooled and sterilized by 0.22 m filter before stored in aliquots at −80° C. The concentration is determined by nanodrop.

The protein purity and molecular weight are determined by standard SDS-PAGE, HR-MS, SEC- and RP-HPLC before undergoing endotoxin removal and filtration.

Soluble his-SUMO-IL18 Variants (Protocol 2)

IL18 candidates are produced as an N-terminal fusion to N-His-SUMO-IL18. The gene is synthesized and cloned into plasmids by a commercial service provider. Plasmids are transformed into E. coli BL21 (DE3). Transformed cells are inoculated into TB medium containing 50 μg/mL kanamycin and cultured at 37° C. When the OD600 reached about 1.2, the cell culture is induced with 0.1 mM IPTG at 18° C. for 20 h.

Cells are then harvested by centrifugation (4500×g, 45 min, 4° C.). Cells are pelleted and cell lysed with a homogenizer at 1000 bar. in lysis buffer: (20 mM Tris/HCl, pH 8.0, 0.15 M NaCl, 10 mM Imidazole, and one tablet of EDTA-free complete protease inhibitor (Roche, COEDTAF-RO) per liter original culture volume.

Lysates are clarified by centrifugation twice at 40,000 g for 45 minutes. Soluble lysates are then subsequent filtered through a 0.22 μm filter.

The soluble lysate is loaded on column containing Ni NTA resin (Cytiva, 17524802) that had been pre-equilibrated with 20 mM Tris/HCl, pH 8.0, 0.15 M NaCl, 10 mM Imidazole, at 5 mL/min and washed with the same buffer for 5 CV. To remove endotoxins, the column is washed with 20 mM Tris/HCl, pH 8.0, 0.15 M NaCl, 10 mM Imidazole, 0.1% Triton X-114 at 10 mL/min for 30 CV. The column is washed with 20 mM Tris/HCl, pH 8.0, 0.15 M NaCl, 10 mM Imidazole, for 5 CV at 5 mL/min and the protein of interest eluted by linear increase of imidazole concentration.

To cleave the SUMO tag, SUMO protease is added to the elution pool at a w/w ratio of 1:250 (protein:SUMO enzyme) and incubated for 18 hours at 4° C. At the same time, the protein is dialysed versus 20 mM Tris, pH 8.0, 150 mM NaCl to reduce the imidazole concentration.

To remove the cleaved SUMO tag and the SUMO protease, the digested protein is passed through a Ni NTA resin column pre-equilibrated with 20 mM Tris/HCl, pH 8.0, 0.15 M NaCl, 10 mM Imidazole, at 5 mL/min. The unbound protein is collected.

The purified protein is concentrated to 2.6 mg/mL and buffer exchanged into either 20 mM HEPES, 150 mM NaCl, 0.5 mM TCEP, 10% glycerol, pH7.5 or PBS, 10% glycerol, pH7.4. Proteins are frozen in liquid nitrogen and stored at −70° C.

Insoluble his-SUMO-IL18 Variants

E. coli BL21 (DE3) harboring a plasmid encoding a N-His-SUMO tagged IL-18 variant fusion are inoculated into 10 L LB culture medium and induced with 0.4 mM IPTG at 30° C. for 6h. Cells are pelleted and cell lysis is done by sonication in lysis buffer: PBS, 8 M urea, pH 7.4. Protein is purified via Nᵢ-NTA beads 6FF (wash 1 with: PBS, 8 M urea, 20 mM imidazole, pH7.4; wash 2 with PBS, 8 M urea, 50 mM Imidazole, pH7.4; elution with PBS, 8 M urea, 500 mM imidazole, pH7.4).

Fractions containing the protein are pooled, dialyzed into PBS pH 7.4 and followed by SUMO digestion. Then the protein is purified with Nᵢ-NTA beads (equilibrate column with PBS, 8 M urea, pH 7.4, wash with PBS, 8 M urea, pH 7.4, elution with PBS, 8 M urea, pH 7.4). Fractions containing the protein are pooled, dialyzed into PBS pH 7.4 and QC is performed using analytical techniques, such as SDS-PAGE and analytical SEC.

### Insoluble Tagless IL18 Variants

E. coli BL21 (DE3) harboring a plasmid encoding mIL-18 is inoculated into 2 L LB culture medium and induced with 0.4 mM IPTG at 30° C. for 6h. Cells are pelleted and cell lysis was done by sonication in lysis buffer: 110 mM Tris, 1.1 M guanidine HCl, 5 mM DTT, pH 8.9. Protein as purified via Q Sepharose FF (balance buffer 20 mM MES, pH 7.0, elution with an increasing gradient from 0 to 1 M NaCl).

Table 4 shows modified IL-18 polypeptides which may be prepared according to the methods provided herein.

| SEQ | | |
|---|---|---|
| ID | Sequence | |
| NO: | modifications* | Sequence |
| 1 | Native sequence | YFGKLESKLS VIRNLNDQVL FIDQGNRPLF |
| | | EDMTDSDCRD NAPRTIFIIS MYKDSQPRGM |
| | | AVTISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 2 | E6K, C38A, | YFGKLKSKLS VIRNLNDQVL FIDQGNRPLF |
| | K53A, C68A, | EDMTDSDARD NAPRTIFIIS MYADSQPRGM |
| | E85C | AVTISVKAEK ISTLSCENKI ISFKCMNPPD |
| | | NIKDTKSDII FFQRSVPGHD NKMQFESSSY |
| | | EGYFLACEKE RDLFKLILKK EDELGDRSIM |
| | | FTVQNED |
| 3 | E6K, V11I, | YFGKLKSKLS IIRNLNDQVL FIDQGNRPLF |
| | C38A, K53A, | EDMTDSDARD NAPRTIFIIS MYADSQPRGM |
| | T63A, C68A, | AVAISVKAEK ISTLSAENKI ISFKCMNPPD |
| | C76A, E85C, | NIKDTKSDII FFQRSVPGHD NKMQFESSSY |
| | C127A | EGYFLAAEKE RDLFKLILKK EDELGDRSIM |
| | | FTVQNED |
| 4 | E6K, C38A, | YFGKLKSKLS VIRNLNDQVL FIDQGNRPLF |
| | K53A, C68A, | EDMTDSDARD NAPRTIFIIS MYADSQPRGM |
| | M86C | AVTISVKAEK ISTLSCENKI ISFKECNPPD |
| | | NIKDTKSDII FFQRSVPGHD NKMQFESSSY |
| | | EGYFLACEKE RDLFKLILKK EDELGDRSIM |
| | | FTVQNED |
| 5 | E6K, V11I, | YFGKLKSKLS IIRNLNDQVL FIDQGNRPLF |
| | C38A, K53A, | EDMTDSDARD NAPRTIFIIS MYADSQPRGM |
| | T63A, C68A, | AVAISVKAEK ISTLSAENKI ISFKECNPPD |
| | C76A, M86C, | NIKDTKSDII FFQRSVPGHD NKMQFESSSY |
| | C127A | EGYFLAAEKE RDLFKLILKK EDELGDRSIM |
| | | FTVQNED |
| 6 | E6K, V11I, | YFGKLKSKLS VIRNLNDQVL FIDQGNRPLF |
| | C38A, K53A, | EDMTDSDARD NAPRTIFIIS MYADSQPRGM |
| | T63A, C68A, | AVAISVKAEK ISTLSAENKI ISFKEMNPPD |
| | C76A, D98C, | NIKDTKSCII FFQRSVPGHD NKMQFESSSY |
| | C127A | EGYFLAAEKE RDLFKLILKK EDELGDRSIM |
| | | FTVQNED |
| 7 | E6K, C38A, | YFGKLKSKLS VIRNLNDQVL FIDQGNRPLF |
| | K53A, C68A, | EDMTDSDARD NAPRTIFIIS MYADSQPRGM |
| | C76A, M86C, | AVTISVKAEK ISTLSAENKI ISFKECNPPD |
| | C127A | NIKDTKSDII FFQRSVPGHD NKMQFESSSY |
| | | EGYFLAAEKE RDLFKLILKK EDELGDRSIM |
| | | FTVQNED |
| 8 | E6K, V11I, | YFGKLKSKLS IIRNLNDQVL FIDQGNRPLF |
| | C38A, K53A, | EDMTDSDARD NAPRTIFIIS MYADSQPRGM |
| | T63A, C68A, | AVAISVKAEK ISTLSAENKI ISFKEMNPPD |
| | C76A, T95C, | NIKDCKSDII FFQRSVPGHD NKMQFESSSY |
| | C127A | EGYFLAAEKE RDLFKLILKK EDELGDRSIM |
| | | FTVQNED |
| 9 | E6K, C38A, | YFGKLKSKLS VIRNLNDQVL FIDQGNRPLF |
| | K53A, C68A, | EDMTDSDARD NAPRTIFIIS MYADSQPRGM |
| | D98C | AVTISVKAEK ISTLSCENKI ISFKEMNPPD |
| | | NIKDTKSCII FFQRSVPGHD NKMQFESSSY |
| | | EGYFLACEKE RDLFKLILKK EDELGDRSIM |
| | | FTVQNED |
| 10 | E6K, C38A, | YFGKLKSKLS VIRNLNDQVL FIDQGNRPLF |
| | K53A, C68A, | EDMTDSDARD NAPRTIFIIS MYADSQPRGM |
| | C76A, D98C, | AVTISVKAEK ISTLSAENKI ISFKEMNPPD |
| | C127A | NIKDTKSCII FFQRSVPGHD NKMQFESSSY |
| | | EGYFLAAEKE RDLFKLILKK EDELGDRSIM |
| | | FTVQNED |
| 11 | E6K, V11I, | YFGKLKSKLS IIRNLNDQVL FIDQGNRPLF |
| | C38A, K53A, | EDMTDSDARD NAPRTIFIIS MYADSQPRGM |
| | C76A, C127A | AVTISVKCEK ISTLSAENKI ISFKEMNPPD |
| | | NIKDTKSDII FFQRSVPGHD NKMQFESSSY |
| | | EGYFLAAEKE RDLFKLILKK EDELGDRSIM |
| | | FTVQNED |
| 12 | E6K, C38A, | YFGKLKSKLS VIRNLNDQVL FIDQGNRPLF |
| | K53A, T63A, | EDMTDSDARD NAPRTIFIIS MYADSQPRGM |
| | C76A, C127A | AVAISVKCEK ISTLSAENKI ISFKEMNPPD |
| | | NIKDTKSDII FFQRSVPGHD NKMQFESSSY |
| | | EGYFLAAEKE RDLFKLILKK EDELGDRSIM |
| | | FTVQNED |
| 13 | E6K, K53A, | YFGKLKSKLS VIRNLNDQVL FIDQGNRPLF |
| | T63N | EDMTDSDCRD NAPRTIFIIS MYADSQPRGM |
| | | AVNISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 14 | E6K, K53A, | YFGKLKSKLS VIRNLNDQVL FIDQGNRPLF |
| | S50A, T63N | EDMTDSDCRD NAPRTIFIIA MYADSQPRGM |
| | | AVNISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 15 | E6K, K53A, | YFGKLKSKLS VIRNLNDQVL FIDQGNRPLF |
| | S50H, T63N | EDMTDSDCRD NAPRTIFIIH MYADSQPRGM |
| | | AVNISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 16 | E6K, K53A, | YFGKLKSKLS VIRNLNDQVL FIDQGNRPLF |
| | T63N, S65A | EDMTDSDCRD NAPRTIFIIS MYADSQPRGM |
| | | AVNIAVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 17 | E6K, K53A, | YFGKLKSKLS VIRNLNDQVL FIDQGNRPLF |
| | S50H | EDMTDSDCRD NAPRTIFIIH MYADSQPRGM |
| | | AVTISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 18 | E6K, C38A, | YFGKLKSKLS VIRNLNDQVL FIDQGNRPLF |
| | K53A, C68A | EDMTDSDARD NAPRTIFIIS MYADSQPRGM |
| | | AVTISVKAEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 19 | E6K, K53A, | YFGKLKSKLS VIRNLNDQVL FIDQGNRPLF |
| | K79A | EDMTDSDCRD NAPRTIFIIS MYADSQPRGM |
| | | AVTISVKCEK ISTLSCENAI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 20 | E6K, K53A, | YFGKLKSKLS VIRNLNDQVL FIDQGNRPLF |
| | R104A | EDMTDSDCRD NAPRTIFIIS MYADSQPRGM |
| | | AVTISVKCEK ISTLSCENAI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 21 | E6K, K53A, | YFGKLKSKLS VIRNLNDQVL FIDQGNRPLF |
| | G108A | EDMTDSDCRD NAPRTIFIIS MYADSQPRGM |
| | | AVTISVKCEK ISTLSCENKI ISFKEMNPPD |
| | | NIKDTKSDII FFQRSVPAHD NKMQFESSSY |
| | | EGYFLACEKE RDLFKLILKK EDELGDRSIM |
| | | FTVQNED |
| 22 | E6K, K53A, | YFGKLKSKLS VIRNLNDQVL FIDQGNRPLF |
| | H109A | EDMTDSDCRD NAPRTIFIIS MYADSQPRGM |
| | | AVTISVKCEK ISTLSCENKI ISFKEMNPPD |
| | | NIKDTKSDII FFQRSVPGAD NKMQFESSSY |
| | | EGYFLACEKE RDLFKLILKK EDELGDRSIM |
| | | FTVQNED |
| 23 | E6K, K53A, | YFGKLKSKLS VIRNLNDQVL FIDQGNRPLF |
| | K112A | EDMTDSDCRD NAPRTIFIIS MYADSQPRGM |
| | | AVTISVKCEK ISTLSCENKI ISFKEMNPPD |
| | | NIKDTKSDII FFQRSVPGHD NAMQFESSSY |
| | | EGYFLACEKE RDLFKLILKK EDELGDRSIM |
| | | FTVQNED |
| 24 | E6K, C38A, | YFGKLKSKLS VIRNLNDQVL FIDQGNRPLF |
| | K53A, T63A, | EDMTDSDARD NAPRTIFIIS MYADSQPRGM |
| | C76A | AVAISVKCEK ISTLSAENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 25 | E6K, C38Q, | YFGKLKSKLS VIRNLNDQVL FIDQGNRPLF |
| | K53A, T63A, | EDMTDSDQRD NAPRTIFIIS MYADSQPRGM |
| | C76A | AVAISVKCEK ISTLSAENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 26 | E6K, C38A, | YFGKLKSKLS VIRNLNDQVL FIDQGNRPLF |
| | K53A, T63A, | EDMTDSDARD NAPRTIFIIS MYADSQPRGM |
| | C127A | AVAISVKCEK ISTLSCENKI ISFKEMNPPD |
| | | NIKDTKSDII FFQRSVPGHD NKMQFESSSY |
| | | EGYFLAAEKE RDLFKLILKK EDELGDRSIM |
| | | FTVQNED |
| 27 | E6K, C38Q, | YFGKLKSKLS VIRNLNDQVL FIDQGNRPLF |
| | K53A, T63A, | EDMTDSDQRD NAPRTIFIIS MYADSQPRGM |
| | C127A | AVAISVKCEK ISTLSCENKI ISFKEMNPPD |
| | | NIKDTKSDII FFQRSVPGHD NKMQFESSSY |
| | | EGYFLAAEKE RDLFKLILKK EDELGDRSIM |
| | | FTVQNED |
| 28 | E6K, C38A, | YFGKLKSKLS VIRNLNDQVL FIDQGNRPLF |
| | K53A, T63A, | EDMTDSDARD NAPRTIFIIS MYADSQPRGM |
| | C76A, C127A | AVAISVKCEK ISTLSAENKI ISFKEMNPPD |
| | | NIKDTKSDII FFQRSVPGHD NKMQFESSSY |
| | | EGYFLAAEKE RDLFKLILKK EDELGDRSIM |
| | | FTVQNED |
| 29 | E6K, V11I, | YFGKLKSKLS IIRNLNDQVL FIDQGNRPLF |
| | C38A, K53A, | EDMTDSDARD NAPRTIFIIS MYADSQPRGM |
| | T63A | AVAISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 30 | E6K, V11I, | YFGKLKSKLS IIRNLNDQVL FIDQGNRPLF |
| | C38A, K53A, | EDMTDSDARD NAPRTIFIIS MYADSQPRGM |
| | T63A, C76A, | AVAISVKCEK ISTLSAENKI ISFKEMNPPD |
| | C127A | NIKDTKSDII FFQRSVPGHD NKMQFESSSY |
| | | EGYFLAAEKE RDLFKLILKK EDELGDRSIM |
| | | FTVQNED |
| 31 | C38A, C76A, | YFGKLESKLS VIRNLNDQVL FIDQGNRPLF |
| | C127A | EDMTDSDARD NAPRTIFIIS MYKDSQPRGM |
| | | AVTISVKCEK ISTLSAENKI ISFKEMNPPD |
| | | NIKDTKSDII FFQRSVPGHD NKMQFESSSY |
| | | EGYFLAAEKE RDLFKLILKK EDELGDRSIM |
| | | FTVQNED |
| 32 | C38A | YFGKLESKLS VIRNLNDQVL FIDQGNRPLF |
| | | EDMTDSDARD NAPRTIFIIS MYKDSQPRGM |
| | | AVTISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 33 | E6K, C38A, | YFGKLKSKLS VIRNLNDQVL FIDQGNRPLF |
| | K53A, T63A | EDMTDSDARD NAPRTIFIIS MYADSQPRGM |
| | | AVAISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 34 | E06K, K53A, | YFGKLKSKLS VIRNLNDQVL FIDQGNRPLF |
| | S55A | EDMTDSDCRD NAPRTIFIIS MYADAQPRGM |
| | | AVTISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 35 | Y01G, F02A, | GAGKLKSKLS VIRNLNDQVL FIDQGNRPLF |
| | E06K, M51G, | EDMTDSDCRD NAPRTIFIIS GYAAAQPRGM |
| | K53A, D54A, | AVAISVKCEK ISTLSCENKI ISFKEMNPPD |
| | S55A, T63A | NIKDTKSDII FFQRSVPGHD NKMQFESSSY |
| | | EGYFLACEKE RDLFKLILKK EDELGDRSIM |
| | | FTVQNED |
| 36 | K53A | YFGKLESKLS VIRNLNDQVL FIDQGNRPLF |
| | | EDMTDSDCRD NAPRTIFIIS MYADSQPRGM |
| | | AVTISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 37 | S55A | YFGKLESKLS VIRNLNDQVL FIDQGNRPLF |
| | | EDMTDSDCRD NAPRTIFIIS MYKDAQPRGM |
| | | AVTISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 38 | E06K | YFGKLKSKLS VIRNLNDQVL FIDQGNRPLF |
| | | EDMTDSDCRD NAPRTIFIIS MYKDSQPRGM |
| | | AVTISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 39 | E06K, K53A | YFGKLKSKLS VIRNLNDQVL FIDQGNRPLF |
| | | EDMTDSDCRD NAPRTIFIIS MYADSQPRGM |
| | | AVTISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 40 | E06K, S55A | YFGKLKSKLS VIRNLNDQVL FIDQGNRPLF |
| | | EDMTDSDCRD NAPRTIFIIS MYKDAQPRGM |
| | | AVTISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 41 | K53A, S55A | YFGKLESKLS VIRNLNDQVL FIDQGNRPLF |
| | | EDMTDSDCRD NAPRTIFIIS MYADAQPRGM |
| | | AVTISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 42 | E06K, K53A, | YFGKLKSKLS VIRNLNDQVL FIDQGNRPLF |
| | S55A, T63A | EDMTDSDCRD NAPRTIFIIS MYADAQPRGM |
| | | AVAISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 43 | E06K, K53A, | GFGKLKSKLS VIRNLNDQVL FIDQGNRPLF |
| | S55A, Y01G | EDMTDSDCRD NAPRTIFIIS MYADAQPRGM |
| | | AVTISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 44 | E06K, K53A, | YAGKLKSKLS VIRNLNDQVL FIDQGNRPLF |
| | S55A, F02A | EDMTDSDCRD NAPRTIFIIS MYADAQPRGM |
| | | AVTISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 45 | E06K, K53A, | YFGKLKSKLS VIRNLNDQVL FIDQGNRPLF |
| | S55A, D54A | EDMTDSDCRD NAPRTIFIIS MYAAAQPRGM |
| | | AVTISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 46 | E06K, K53A, | YFGKLKSKLS VIRNLNDQVL FIDQGNRPLF |
| | S55A, M51G | EDMTDSDCRD NAPRTIFIIS GYADAQPRGM |
| | | AVTISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 47 | C38S, C68S, | YFGKLESKLS VIRNLNDQVL FIDQGNRPLF |
| | C76S, C127S | EDMTDSDSRD NAPRTIFIIS MYKDSQPRGM |
| | | AVTISVKSEK ISTLSSENKI ISFKEMNPPD |
| | | NIKDTKSDII FFQRSVPGHD NKMQFESSSY |
| | | EGYFLASEKE RDLFKLILKK EDELGDRSIM |
| | | FTVQNED |
| 48 | C38S, C68S, | YFGKLESKLS VIRNLNDQVL FIDQGNRPLF |
| | C76S, C127S, | EDMTDSDSRD NAPRTIFIIS MYKDSQPRGM |
| | K70C | AVTISVKSEC ISTLSSENKI ISFKEMNPPD |
| | | NIKDTKSDII FFQRSVPGHD NKMQFESSSY |
| | | EGYFLASEKE RDLFKLILKK EDELGDRSIM |
| | | FTVQNED |
| 49 | E06K, K53A, | YFGKLKSKLS VIRNLNDQVL FIDQGNRPLF |
| | S55A, C38S, | EDMTDSDSRD NAPRTIFIIS MYADAQPRGM |
| | C68S, C76S, | AVTISVKSEC ISTLSSENKI ISFKEMNPPD |
| | C127S, K70C | NIKDTKSDII FFQRSVPGHD NKMQFESSSY |
| | | EGYFLASEKE RDLFKLILKK EDELGDRSIM |
| | | FTVQNED |
| 50 | E06K, K53A, | YFGKLKSKLS VIRNLNDQVL FIDQGNRPLF |
| | T63A | EDMTDSDCRD NAPRTIFIIS MYADSQPRGM |
| | | AVAISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 51 | T63A | YFGKLESKLS VIRNLNDQVL FIDQGNRPLF |
| | | EDMTDSDCRD NAPRTIFIIS MYKDSQPRGM |
| | | AVAISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 52 | E06K, T63A | YFGKLKSKLS VIRNLNDQVL FIDQGNRPLF |
| | | EDMTDSDCRD NAPRTIFIIS MYKDSQPRGM |
| | | AVAISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 53 | K53A, T63A | YFGKLESKLS VIRNLNDQVL FIDQGNRPLF |
| | | EDMTDSDCRD NAPRTIFIIS MYADSQPRGM |
| | | AVAISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 54 | E06K, K53A, | YFGKLKSKLS VIRNLNDQVL FIDQGNRPLF |
| | C38S, C68S, | EDMTDSDSRD NAPRTIFIIS MYADSQPRGM |
| | C76S, C127S, | AVTISVKSEC ISTLSSENKI ISFKEMNPPD |
| | K70C | NIKDTKSDII FFQRSVPGHD NKMQFESSSY |
| | | EGYFLASEKE RDLFKLILKK EDELGDRSIM |
| | | FTVQNED |
| 55 | K53A, T63A, | YFGKLESKLS VIRNLNDQVL FIDQGNRPLF |
| | C38S, C68S, | EDMTDSDSRD NAPRTIFIIS MYADSQPRGM |
| | C76S, C127S, | AVAISVKSEC ISTLSSENKI ISFKEMNPPD |
| | K70C | NIKDTKSDII FFQRSVPGHD NKMQFESSSY |
| | | EGYFLASEKE RDLFKLILKK EDELGDRSIM |
| | | FTVQNED |
| 56 | E6K, K53A, | YFGKLKSKLS VIRNLNDQVL FIDQGNRPLF |
| | C38S, C76S, | EDMTDSDSRD NAPRTIFIIS MYADSQPRGM |
| | C127S | AVTISVKCEK ISTLSSENKI ISFKEMNPPD |
| | | NIKDTKSDII FFQRSVPGHD NKMQFESSSY |
| | | EGYFLASEKE RDLFKLILKK EDELGDRSIM |
| | | FTVQNED |
| 57 | E6K, C38S, | YFGKLKSKLS VIRNLNDQVL FIDQGNRPLF |
| | K53A | EDMTDSDSRD NAPRTIFIIS MYADSQPRGM |
| | | AVTISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 58 | E6K, K53A, | YFGKLKSKLS VIRNLNDQVL FIDQGNRPLF |
| | C38S, C68S, | EDMTDSDSRD NAPRTIFIIS MYADSQPRGM |
| | C76S, C127S, | AVTISVKSEC ISTLSSENKI ISFKEMNPPD |
| | K70C | NIKDTKSDII FFQRSVPGHD NKMQFESSSY |
| | | EGYFLASEKE RDLFKLILKK EDELGDRSIM |
| | | FTVQNED |
| 59 | E6K, C38A, | YFGKLKSKLS VIRNLNDQVL FIDQGNRPLF |
| | K53A | EDMTDSDARD NAPRTIFIIS MYADSQPRGM |
| | | AVTISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 60 | E6K, C38Q, | YFGKLKSKLS VIRNLNDQVL FIDQGNRPLF |
| | K53A | EDMTDSDQRD NAPRTIFIIS MYADSQPRGM |
| | | AVTISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 61 | E6K, C38A, | YFGKLKSKLS VIRNLNDQVL FIDQGNRPLF |
| | K53A, C76A | EDMTDSDARD NAPRTIFIIS MYADSQPRGM |
| | | AVTISVKCEK ISTLSAENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 62 | E6K, C38A, | YFGKLKSKLS VIRNLNDQVL FIDQGNRPLF |
| | K53A, C127A | EDMTDSDARD NAPRTIFIIS MYADSQPRGM |
| | | AVTISVKCEK ISTLSCENKI ISFKEMNPPD |
| | | NIKDTKSDII FFQRSVPGHD NKMQFESSSY |
| | | EGYFLAAEKE RDLFKLILKK EDELGDRSIM |
| | | FTVQNED |
| 63 | E6K, C38A, | YFGKLKSKLS VIRNLNDQVL FIDQGNRPLF |
| | K53A, C76A, | EDMTDSDARD NAPRTIFIIS MYADSQPRGM |
| | C127A | AVTISVKCEK ISTLSAENKI ISFKEMNPPD |
| | | NIKDTKSDII FFQRSVPGHD NKMQFESSSY |
| | | EGYFLAAEKE RDLFKLILKK EDELGDRSIM |
| | | FTVQNED |
| 64 | E6K, K53A, | YFGKLKSKLS VIRNLNDQVL FIDQGNRPLF |
| | C38A, S55A, | EDMTDSDARD NAPRTIFIIS MYADAQPRGM |
| | T63A | AVAISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 65 | E6K, C38Q, | YFGKLKSKLS VIRNLNDQVL FIDQGNRPLF |
| | K53A, S55A, | EDMTDSDQRD NAPRTIFIIS MYADAQPRGM |
| | T63A | AVAISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 66 | E6K, K53A, | YFGKLKSKLS VIRNLNDQVL FIDQGNRPLF |
| | K84A | EDMTDSDCRD NAPRTIFIIS MYADSQPRGM |
| | | AVTISVKCEK ISTLSCENKI ISFAEMNPPD |
| | | NIKDTKSDII FFQRSVPGHD NKMQFESSSY |
| | | EGYFLACEKE RDLFKLILKK EDELGDRSIM |
| | | FTVQNED |
| 67 | E6K, K53A, | YFGKLKSKLS VIRNLNDQVL FIDQGNRPLF |
| | D98A | EDMTDSDCRD NAPRTIFIIS MYADSQPRGM |
| | | AVTISVKCEK ISTLSCENKI ISFKEMNPPD |
| | | NIKDTKSAII FFQRSVPGHD NKMQFESSSY |
| | | EGYFLACEKE RDLFKLILKK EDELGDRSIM |
| | | FTVQNED |
| 204 | V11I, C38A, | YFGKLESKLSIIRNLNDQVLFIDQGNRPLFEDMTDSD |
| | K53A, C76A, | ARDNAPRTIFIISMYADSQPRGMAVTISVKCEKISTLS |
| | C127A | AENKIISFKEMNPPDNIKDTKSDIIFFQRSVPGHDNK |
| | | MQFESSSYEGYFLAAEKERDLFKLILKKEDELGDRSI |
| | | MFTVQNED |
| 205 | V11I, C38A, | YFGKLESKLSIIRNLNDQVLFIDQGNRPLFEDMTDSD |
| | K53A, T63A, | ARDNAPRTIFIISMYADSQPRGMAVAISVKCEKISTL |
| | C76A, C127A | SAENKIISFKEMNPPDNIKDTKSDIIFFQRSVPGHDNK |
| | | MQFESSSYEGYFLAAEKERDLFKLILKKEDELGDRSI |
| | | MFTVQNED |
| 206 | V11I, C38A, | YFGKLESKLSIIRNLNDQVLFIDQGNRPLFEDMTDSD |
| | K53A, S55A, | ARDNAPRTIFIISMYADAQPRGMAVTISVKCEKISTL |
| | C76A, C127A | SAENKIISFKEMNPPDNIKDTKSDIIFFQRSVPGHDNK |
| | | MQFESSSYEGYFLAAEKERDLFKLILKKEDELGDRSI |
| | | MFTVQNED |
| 207 | V11I, C38A, | YFGKLESKLSIIRNLNDQVLFIDQGNRPLFEDMTDSD |
| | M51G, K53A, | ARDNAPRTIFIISGYADSQPRGMAVTISVKCEKISTLS |
| | C76A, C127A | AENKIISFKEMNPPDNIKDTKSDIIFFQRSVPGHDNK |
| | | MQFESSSYEGYFLAAEKERDLFKLILKKEDELGDRSI |
| | | MFTVQNED |
| 208 | V11I, C38A, | YFGKLESKLSIIRNLNDQVLFIDQGNRPLFEDMTDSD |
| | K53A, D54A, | ARDNAPRTIFIISMYAASQPRGMAVTISVKCEKISTLS |
| | C76A, C127A | AENKIISFKEMNPPDNIKDTKSDIIFFQRSVPGHDNK |
| | | MQFESSSYEGYFLAAEKERDLFKLILKKEDELGDRSI |
| | | MFTVQNED |
| 209 | F2A, V11I, | YAGKLESKLSIIRNLNDQVLFIDQGNRPLFEDMTDSD |
| | C38A, K53A, | ARDNAPRTIFIISMYADSQPRGMAVTISVKCEKISTLS |
| | C76A, C127A | AENKIISFKEMNPPDNIKDTKSDIIFFQRSVPGHDNK |
| | | MQFESSSYEGYFLAAEKERDLFKLILKKEDELGDRSI |
| | | MFTVQNED |
| 210 | V11I, E31A, | YFGKLESKLSIIRNLNDQVLFIDQGNRPLFADMTDSD |
| | C38A, K53A, | ARDNAPRTIFIISMYADSQPRGMAVTISVKCEKISTLS |
| | C76A, C127A | AENKIISFKEMNPPDNIKDTKSDIIFFQRSVPGHDNK |
| | | MQFESSSYEGYFLAAEKERDLFKLILKKEDELGDRSI |
| | | MFTVQNED |
| 211 | V11I, T34A, | YFGKLESKLSIIRNLNDQVLFIDQGNRPLFEDMADSD |
| | C38A, K53A, | ARDNAPRTIFIISMYADSQPRGMAVTISVKCEKISTLS |
| | C76A, C127A | AENKIISFKEMNPPDNIKDTKSDIIFFQRSVPGHDNK |
| | | MQFESSSYEGYFLAAEKERDLFKLILKKEDELGDRSI |
| | | MFTVQNED |
| 212 | V11I, D35A, | YFGKLESKLSIIRNLNDQVLFIDQGNRPLFEDMTASD |
| | C38A, K53A, | ARDNAPRTIFIISMYADSQPRGMAVTISVKCEKISTLS |
| | C76A, C127A | AENKIISFKEMNPPDNIKDTKSDIIFFQRSVPGHDNK |
| | | MQFESSSYEGYFLAAEKERDLFKLILKKEDELGDRSI |
| | | MFTVQNED |
| 213 | V11I, S36A, | YFGKLESKLSIIRNLNDQVLFIDQGNRPLFEDMTDAD |
| | C38A, K53A, | ARDNAPRTIFIISMYADSQPRGMAVTISVKCEKISTLS |
| | C76A, C127A | AENKIISFKEMNPPDNIKDTKSDIIFFQRSVPGHDNK |
| | | MQFESSSYEGYFLAAEKERDLFKLILKKEDELGDRSI |
| | | MFTVQNED |
| 214 | V11I, D37A, | YFGKLESKLSIIRNLNDQVLFIDQGNRPLFEDMTDSA |
| | C38A, K53A, | ARDNAPRTIFIISMYADSQPRGMAVTISVKCEKISTLS |
| | C76A, C127A | AENKIISFKEMNPPDNIKDTKSDIIFFQRSVPGHDNK |
| | | MQFESSSYEGYFLAAEKERDLFKLILKKEDELGDRSI |
| | | MFTVQNED |
| 215 | V11I, E31A, | YFGKLESKLSIIRNLNDQVLFIDQGNRPLFADMTDSA |
| | D37A, C38A, | ARDNAPRTIFIISMYADSQPRGMAVTISVKCEKISTLS |
| | K53A, C76A, | AENKIISFKEMNPPDNIKDTKSDIIFFQRSVPGHDNK |
| | C127A | MQFESSSYEGYFLAAEKERDLFKLILKKEDELGDRSI |
| | | MFTVQNED |
| 216 | V11I, C38A, | YFGKLESKLSIIRNLNDQVLFIDQGNRPLFEDMTDSD |
| | D40A, K53A, | ARANAPRTIFIISMYADSQPRGMAVTISVKCEKISTLS |
| | C76A, C127A | AENKIISFKEMNPPDNIKDTKSDIIFFQRSVPGHDNK |
| | | MQFESSSYEGYFLAAEKERDLFKLILKKEDELGDRSI |
| | | MFTVQNED |
| 217 | V11I, C38A, | YFGKLESKLSIIRNLNDQVLFIDQGNRPLFEDMTDSD |
| | N41A, K53A, | ARDAAPRTIFIISMYADSQPRGMAVTISVKCEKISTLS |
| | C76A, C127A | AENKIISFKEMNPPDNIKDTKSDIIFFQRSVPGHDNK |
| | | MQFESSSYEGYFLAAEKERDLFKLILKKEDELGDRSI |
| | | MFTVQNED |
| 218 | V11I, C38A, | YFGKLESKLSIIRNLNDQVLFIDQGNRPLFEDMTDSD |
| | K53A, C76A, | ARDNAPRTIFIISMYADSQPRGMAVTISVKCEKISTLS |
| | C127A, D132A | AENKIISFKEMNPPDNIKDTKSDIIFFQRSVPGHDNK |
| | | MQFESSSYEGYFLAAEKERALFKLILKKEDELGDRSI |
| | | MFTVQNED |
| 219 | V11I, C38A, | YFGKLESKLSIIRNLNDQVLFIDQGNRPLFEDMTDSD |
| | K53A, C76A, | ARDNAPRTIFIISMYADSQPRGMAVTISVKCEKISTLS |
| | G108A, C127A | AENKIISFKEMNPPDNIKDTKSDIIFFQRSVPAHDNK |
| | | MQFESSSYEGYFLAAEKERDLFKLILKKEDELGDRSI |
| | | MFTVQNED |
| 220 | V11I, C38A, | YFGKLESKLSIIRNLNDQVLFIDQGNRPLFEDMTDSD |
| | K53A, C76A, | ARDNAPRTIFIISMYADSQPRGMAVTISVKCEKISTLS |
| | H109A, C127A | AENKIISFKEMNPPDNIKDTKSDIIFFQRSVPGADNK |
| | | MQFESSSYEGYFLAAEKERDLFKLILKKEDELGDRSI |
| | | MFTVQNED |
| 221 | V11I, C38A, | YFGKLESKLSIIRNLNDQVLFIDQGNRPLFEDMTDSD |
| | K53A, C76A, | ARDNAPRTIFIISMYADSQPRGMAVTISVKCEKISTLS |
| | D110A, C127A | AENKIISFKEMNPPDNIKDTKSDIIFFQRSVPGHANK |
| | | MQFESSSYEGYFLAAEKERDLFKLILKKEDELGDRSI |
| | | MFTVQNED |
| 222 | K8R, V11I, | YFGKLESRLSIIRNLNDQVLFIDQGNRPLFEDMTDSD |
| | C38A, C76A, | ARDNAPRTIFIISMYKDSQPRGMAVTISVKCEKISTLS |
| | Q103E, C127A | AENKIISFKEMNPPDNIKDTKSDIIFFERSVPGHDNK |
| | | MQFESSSYEGYFLAAEKERDLFKLILKKEDELGDRSI |
| | | MFTVQNED |
| 223 | K8E, V11I, | YFGKLESELSIIRNLNDQVLFIDQGNRPLFEDMTDSD |
| | C38A, C76A, | ARDNAPRTIFIISMYKDSQPRGMAVTISVKCEKISTLS |
| | Q103R, C127A | AENKIISFKEMNPPDNIKDTKSDIIFFRRSVPGHDNK |
| | | MQFESSSYEGYFLAAEKERDLFKLILKKEDELGDRSI |
| | | MFTVQNED |
| 224 | V11I, C38A, | YFGKLESKLSIIRNLNDQVLFIDQGNRPLFEDMTDSD |
| | C76A, Q103K, | ARDNAPRTIFIISMYKDSQPRGMAVTISVKCEKISTLS |
| | C127A | AENKIISFKEMNPPDNIKDTKSDIIFFKRSVPGHDNK |
| | | MQFESSSYEGYFLAAEKERDLFKLILKKEDELGDRSI |
| | | MFTVQNED |
| 225 | V11I, C38A, | YFGKLESKLSIIRNLNDQVLFIDQGNRPLFEDMTDSD |
| | S55H, C76A, | ARDNAPRTIFIISMYKDHQPRGMAVTISVKCEKISTL |
| | C127A | SAENKIISFKEMNPPDNIKDTKSDIIFFQRSVPGHDNK |
| | | MQFESSSYEGYFLAAEKERDLFKLILKKEDELGDRSI |
| | | MFTVQNED |
| 226 | V11I, C38A, | YFGKLESKLSIIRNLNDQVLFIDQGNRPLFEDMTDSD |
| | S55R, C76A, | ARDNAPRTIFIISMYKDRQPRGMAVTISVKCEKISTL |
| | C127A | SAENKIISFKEMNPPDNIKDTKSDIIFFQRSVPGHDNK |
| | | MQFESSSYEGYFLAAEKERDLFKLILKKEDELGDRSI |
| | | MFTVQNED |
| 227 | V11I, C38A, | YFGKLESKLSIIRNLNDQVLFIDQGNRPLFEDMTDSD |
| | S55T, C76A, | ARDNAPRTIFIISMYKDTQPRGMAVTISVKCEKISTLS |
| | C127A | AENKIISFKEMNPPDNIKDTKSDIIFFQRSVPGHDNK |
| | | MQFESSSYEGYFLAAEKERDLFKLILKKEDELGDRSI |
| | | MFTVQNED |
| 228 | V11I, C38A, | YFGKLESKLSIIRNLNDQVLFIDQGNRPLFEDMTDSD |
| | C76A, S105I, | ARDNAPRTIFIISMYKDSQPRGMAVTISVKCEKISTLS |
| | C127A | AENKIISFKEMNPPDNIKDTKSDIIFFQRIVPGHDNKM |
| | | QFESSSYEGYFLAAEKERDLFKLILKKEDELGDRSIM |
| | | FTVQNED |
| 229 | V11I, C38A, | YFGKLESKLSIIRNLNDQVLFIDQGNRPLFEDMTDSD |
| | C76A, S105K, | ARDNAPRTIFIISMYKDSQPRGMAVTISVKCEKISTLS |
| | C127A | AENKIISFKEMNPPDNIKDTKSDIIFFQRKVPGHDNK |
| | | MQFESSSYEGYFLAAEKERDLFKLILKKEDELGDRSI |
| | | MFTVQNED |
| 230 | K8L, E6K, V11I, | YFGKLKSLLSIIRNLNDQVLFIDQGNRPLFEDMTDSD |
| | C38A, K53A, | ARDNAPRTIFIISMYADSQPRGMAVAISVKCEKISTL |
| | T63A, C76A, | SAENKIISFKEMNPPDNIKDTKSDIIFFQRSVPGHDNK |
| | C127A | MQFESSSYEGYFLAAEKERDLFKLILKKEDELGDRSI |
| | | MFTVQNED |
| 231 | E6K, V11I, | YFGKLKSKLSIIRNLNDQVLFIDQGNRPLFEDMTDSD |
| | C38A, I49E, | ARDNAPRTIFIESMYADSQPRGMAVAISVKCEKISTL |
| | K53A, T63A, | SAENKIISFKEMNPPDNIKDTKSDIIFFQRSVPGHDNK |
| | C76A, C127A | MQFESSSYEGYFLAAEKERDLFKLILKKEDELGDRSI |
| | | MFTVQNED |
| 232 | E6K, V11I, | YFGKLKSKLSIIRNLNDQVLFIDQGNRPLFEDMTDSD |
| | C38A, I49M, | ARDNAPRTIFIMSMYADSQPRGMAVAISVKCEKIST |
| | K53A, T63A, | LSAENKIISFKEMNPPDNIKDTKSDIIFFQRSVPGHDN |
| | C76A, C127A | KMQFESSSYEGYFLAAEKERDLFKLILKKEDELGDR |
| | | SIMFTVQNED |
| 233 | E6K, V11I, | YFGKLKSKLSIIRNLNDQVLFIDQGNRPLFEDMTDSD |
| | C38A, I49R, | ARDNAPRTIFIRSMYADSQPRGMAVAISVKCEKISTL |
| | K53A, T63A, | SAENKIISFKEMNPPDNIKDTKSDIIFFQRSVPGHDNK |
| | C76A, C127A | MQFESSSYEGYFLAAEKERDLFKLILKKEDELGDRSI |
| | | MFTVQNED |
| 234 | E6K, V11I, | YFGKLKSKLSIIRNLNDQVLFIDQGNRPLFEDMTDSD |
| | C38A, K53A, | ARDNAPRTIFIISMYADSQPRGMAVAISVKCEKISTL |
| | T63A, C76A, | SAENKIISFKEMNPPDNIKDTKSDIIFFRRSVPGHDNK |
| | Q103R, C127A | MQFESSSYEGYFLAAEKERDLFKLILKKEDELGDRSI |
| | | MFTVQNED |
| 235 | E6K, K8E, V11I, | YFGKLKSELSIIRNLNDQVLFIDQGNRPLFEDMTDSD |
| | C38A, K53A, | ARDNAPRTIFIISMYADSQPRGMAVAISVKCEKISTL |
| | T63A, C76A, | SAENKIISFKEMNPPDNIKDTKSDIIFFRRSVPGHDNK |
| | Q103R, C127A | MQFESSSYEGYFLAAEKERDLFKLILKKEDELGDRSI |
| | | MFTVQNED |
| 236 | E6K, V11I, | YFGKLKSKLSIIRNLNDQVLFIDQGNRPLFEDMTDSD |
| | C38A, K53A, | ARDNAPRTIFIISMYADSQPRGMAVAISVKCEKISTL |
| | T63A, C76A, | SAENKIISFKEMNPPDNIKDTKSDIIFFQRSVPGHDNK |
| | C127A, V153R | MQFESSSYEGYFLAAEKERDLFKLILKKEDELGDRSI |
| | | MFTRQNED |
| 237 | E6K, V11I, | YFGKLKSKLSIIRNLNDQVLFIDQGNRPLFEDMTDSD |
| | C38A, K53A, | ARDNAPRTIFIISMYADSQPRGMAVAISVKCEKISTL |
| | T63A, C76A, | SAENKIISFKEMNPPDNIKDTKSDIIFFQRSVPGHDNK |
| | C127A, V153E | MQFESSSYEGYFLAAEKERDLFKLILKKEDELGDRSI |
| | | MFTEQNED |
| 238 | E6K, V11I, | YFGKLKSKLSIIRNLNDQVLFIDQGNRPLFEDMTDSD |
| | C38A, K53A, | ARDNAPRTIFIISMYADSQPRGMAVAISVKCEKISTL |
| | T63A, C76A, | SAENKIISFKEMNPPDNIKDTKSDIIFFQRSVPGHDNK |
| | C127A, V153Y | MQFESSSYEGYFLAAEKERDLFKLILKKEDELGDRSI |
| | | MFTYQNED |
| 239 | E6K, V11I, | YFGKLKSKLSIIRNLNDQVLFIDQGNRPLFEDMTDSD |
| | C38A, M51G, | ARDNAPRTIFIISGYADSQPRGMAVAISVKCEKISTLS |
| | K53A, T63A, | AENKIISFKEMNPPDNIKDTKSDIIFFQRSVPGHDNK |
| | C76A, C127A | MQFESSSYEGYFLAAEKERDLFKLILKKEDELGDRSI |
| | | MFTVQNED |
| 240 | E6R, V11I, | YFGKLRSKLSIIRNLNDQVLFIDQGNRPLFEDMTDSD |
| | C38A, K53A, | ARDNAPRTIFIISMYADSQPRGMAVAISVKCEKISTL |
| | | SAENKIISFKEMNPPDNIKDTKSDIIFFQRSVPGHDNK |
| | T63A, C76A, | MQFESSSYEGYFLAAEKERDLFKLILKKEDELGDRSI |
| | C127A | MFTVQNED |
| 241 | N-terminal 4xG, | GGGGYFGKLKSKLSIIRNLNDQVLFIDQGNRPLFED |
| | E6K, V11I, | MTDSDARDNAPRTIFIISMYADSQPRGMAVAISVKC |
| | C38A, K53A, | EKISTLSAENKIISFKEMNPPDNIKDTKSDIIFFQRSVP |
| | T63A, C76A, | GHDNKMQFESSSYEGYFLAAEKERDLFKLILKKEDE |
| | C127A | LGDRSIMFTVQNED |
| 242 | N-terminal G, | GYFGKLKSKLSIIRNLNDQVLFIDQGNRPLFEDMTDS |
| | E6K, V11I, | DARDNAPRTIFIISMYADSQPRGMAVAISVKCEKIST |
| | C38A, K53A, | LSAENKIISFKEMNPPDNIKDTKSDIIFFQRSVPGHDN |
| | T63A, C76A, | KMQFESSSYEGYFLAAEKERDLFKLILKKEDELGDR |
| | C127A | SIMFTVQNED |
| 243 | Y1M, E6K, V11I, | MFGKLKSKLSIIRNLNDQVLFIDQGNRPLFEDMTDS |
| | C38A, K53A, | DARDNAPRTIFIISMYADSQPRGMAVAISVKCEKIST |
| | T63A, C76A, | LSAENKIISFKEMNPPDNIKDTKSDIIFFQRSVPGHDN |
| | C127A | KMQFESSSYEGYFLAAEKERDLFKLILKKEDELGDR |
| | | SIMFTVQNED |
| 244 | N-terminal G, | GYFGKLKSKLSIIRNLNDQVLFIDQGNRPLFEDMTDS |
| | E6K, V11I, | DARDNAPRTIFIISGYADSQPRGMAVAISVKCEKIST |
| | C38A, M51G, | LSAENKIISFKEMNPPDNIKDTKSDIIFFQRSVPGHDN |
| | K53A, T63A, | KMQFESSSYEGYFLAAEKERDLFKLILKKEDELGDR |
| | C76A, C127A | SIMFTVQNED |

*Residue position numbering based on SEQ ID NO: 1 as a reference sequence

### Example 2—Covalently Attaching a Polymer to a IL-18 Polypeptides

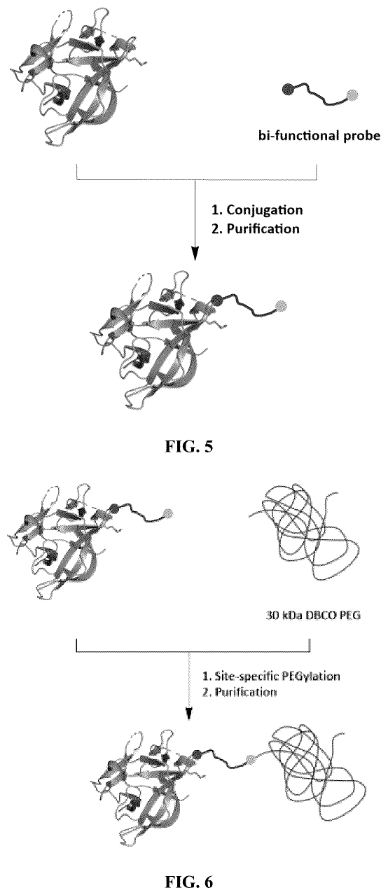

A modified IL-18 polypeptide was conjugated to a PEG functionality. In an experiment, the PEG was attached via a bifunctional linker which first attaches to a desired residue of the IL-18 polypeptide (e.g., C68 or suitable naturally occurring cysteine or a cysteine residue which has been incorporated at a desired site, such as residue 69 or 70). Once attached to the IL-18 polypeptide, the second functionality of the bifunctional linker was used to attach the PEG moiety. An exemplary schematic of such a process is shown in FIGS. 5 and 6. An exemplary protocol on a recombinant IL-18 variant provided herein is described below.

Protocol for Conjugation with Bromoacetamido-PEG5-Azide Linker

Protein was stored in PBS with 10% glycerol at pH 7.4 at −70 C and thawed on ice. Stock protein solution was diluted as necessary with PBS pH 7.4. Bromoacetamido-PEG5-azide was dissolved in water at a concentration of 40 mM. Protein stock solution at 0.2 mg/mL and the linker stock solution at (40 mM, 100 equivalents) was mixed together at 100 equivalents and the reaction was gently mixed. The reaction was allowed to proceed at 20° C. for 3 h with very gentle mixing. Purification was performed immediately after 3 h to stop the reaction. Protein was separated from excess linker on AKTA with HiTrap Desalting FF columns. The column buffer was: 25 mM Tris, pH 7.4. Fractions were analyzed with RP-HPLC and fractions containing the protein are pooled and stored at 4° C. Concentration was assessed with nanodrop based on the theoretical MW and extinction coefficient (5960 M−1 cm−1).

### Example 3—PEGylation of Modified IL-18 Polypeptides

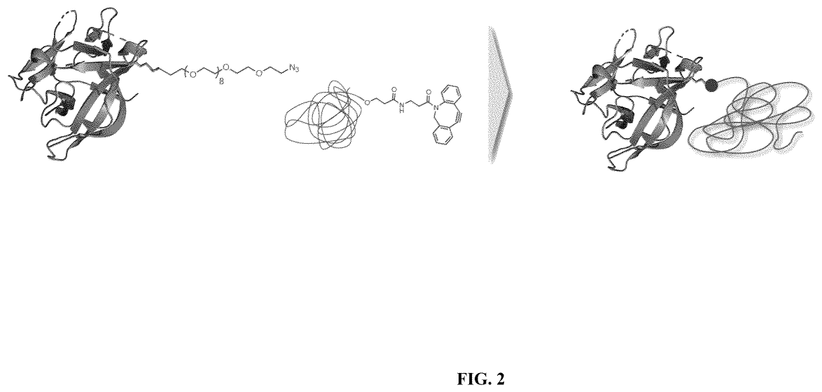

After conjugation of the bifunctional linker as described in Example 2 and as shown in FIG. 5, the modified IL-18 polypeptide is covalently linked with a PEG group. An exemplary schematic of this process is shown in FIG. 6. An exemplary protocol of the conjugation reaction between a PEG and a suitably activated IL-18 polypeptide is provided below. Additionally, the protocols below can be used to covalently link a desired PEG group to a IL-18 polypeptide which incorporates a conjugation handle directly during the preparation of the IL-18 polypeptide (e.g., during the synthesis of a synthetic IL-18 polypeptide). An exemplary schematic of such a process is shown in FIG. 2.

Conjugation—In one example, ten equivalents of 30 kDa DBCO-mPEG was added directly to purified protein (IL-18 conjugated to a linked, such as Bromoacetamido-PEG5-azide) solution. The reaction was incubated overnight at 20° C. with protection from light and with very slow mixing. After completion, the PEGylated protein was purified via anion exchange chromatography on an AKTA FPLC using a 5 ml HiTrap Capto Q ImpRes column with buffer A: 25 mM Tris, pH 7.4; and Buffer B: 25 mM Tris, 350 mM NaCl, pH 7.4. Protein was eluted from the column using a two part linear gradient: Part 1 is 0-10 CV, 0-50% buffer B; then part 2 is 10-15 CV, 50-100% buffer B. Using this protocol, the PEGylated IL-18 protein readily separates from excess PEG and pure PEGylated protein was obtained.

Characterization—The purity and identity of the recombinant protein from commercial source and the conjugated protein was confirmed by aSEC, HPLC and MALDI-TOF MS.

Conjugation—In another example, an IL-18 conjugated to a linker was stored at −80° C. in PBS (pH 7.4) containing 75 mM NaCl and 5% (v/v) glycerol. Prior to PEGylation reaction, the sample was thawed on ice yielding a clear solution. 200 μL of the protein solution (0.4 mg/mL) was mixed with 2.0 mg of 30 kDa DBCO-polyethylene glycol polymer. It was let to react overnight at 20° C. The progress of the synthesis was monitored by reverse-phase HPLC using a gradient of 5 to 30% (2.5 min) and 30 to 75% (7.5 min) CH₃CN with 0.1% TFA (v/v) on a Aeris WIDEPORE C4 200 Å column (3.6 m, 150×4.6 mm) at a flow rate of 1 mL/min at 40° C. and by MALDI-TOF MS. Purification— To remove the excess of PEG, the reaction mixture was diluted with Tris buffer (25 mM, pH 7.4) and flowed through a Hi-Trap-Q-FF column using 25 mM Tris (pH 7.4) as the buffer. The column was eluted with a linear gradient of 0-0.35 M NaCl in the same buffer. The fractions containing the target protein were gathered, buffer exchanged (25 mM Tris, pH 7.4, 75 mM NaCl, 5% glycerol) and concentrated at 0.04 mg/mL. The concentration of purified protein was determined by BCA protein assay. The protein solution was kept at −80° C. Characterization—The purity and identity of the conjugated protein is confirmed by HPLC and MALDI-TOF MS.

### Example 4—Characterization of Modified IL-18 Polypeptides

Modified IL-18 polypeptides provided herein are subject to a series of analytical experiments to characterize the compositions. The modified IL-18 polypeptides are analyzed by HPLC to determine the degree of uniformity in the compositions. The modified IL-18 polypeptide compositions are also analyzed by MALDI-MS to determine the MW and distribution of molecular weights of the compositions. The modified IL-18 polypeptide compositions are further analyzed by circular dichroism to compare the folding of the modified IL-18 polypeptide compositions compared to wild type IL-18.

### Example 5—Formulation of Modified IL-18 Polypeptides

Lyophilized modified IL-18 polypeptides are suspended in a solution comprising PBS buffer (pH 7.4) with 50 mg/mL mannitol.

### Example 6—IL-18 SPR Measurements

The interaction of the wild type and of modified IL-18 polypeptides with human IL-18 receptor subunits are measured with Surface Plasmon Resonance (SPR) technology. Anti-human IgG antibodies are bound by amine coupling onto a CM5 chip to capture 6 g/mL of Fc fused human IL-18Rα, 6 g/mL of Fc fused human IL-18Rβ, or 2 g/mL of Fc fused human IL-18BP isoform a (IL-18BPa) for 30 min before capture. In other settings, 6 g/mL of alpha and beta IL-18 receptors are mixed and pre-incubated for 30 min before capture of the alpha/beta heterodimer IL-18 receptor.

The kinetic binding of the IL-18 analytes are measured with a Biacore 8K instrument in two fold serial dilutions starting at 1 M down to 0.98 nM. Regeneration of the surface back to amine coupled anti IgG antibody is done after every concentration of analyte. To measure the protein association to the receptors, the samples are injected with a flow rate of 50 L/min for 60 s, followed by 300 s buffer only to detect the dissociation. The used running buffer is 1×PBS with 0.05% Tween20. The relative response units (RU, Y-axis) are plotted against time (s, X-axis) and analyzed in a kinetic 1:1 binding model for the monomer receptor binding and for the binding to the IL-18BP. A kinetic heterogenous ligand fit model is applied for the alpha/beta heterodimer binding.

### Example 7—IL-18BP Binding alphaLISA Assay

A human IL-18BP AlphaLISA Assay Kit is used to determine the binding affinity of each IL-18 variant for IL-18BP, which detected the presence of free form IL-18BP.

Sixteen three fold serial dilutions of IL-18 analytes are prepared in alpha-MEM medium supplemented with 20% FCS, Glutamax™, and 25 M β-mercaptoethanol in the presence of 5 ng/mL of His-tagged human IL-18BP. Final IL-18 analytes concentration range from 2778 nM to 0.2 pM.

After 1 hr incubation at room temperature, free IL-18BP levels are measured using a Human IFNγ AlphaLISA Assay Kit. In a 384 well OPTI plate, 5 μL of 5× Anti-IL-18BP acceptor beads are added to 7.5 SL of an IL-18/L-18BP mix. After 30 min incubation at room temperature with shaking, 5 μL of biotinylated Anti-IL-181BP antibodies are added to each well. The plate is incubated further for 1 hr at room temperature. Under subdued light, 12.5 μL of 2× streptavidin (SA) donor beads are pipetted into each well, and the wells are incubated with shaking for an additional 30 min at room temperature. The AlphaLisa signal is then measured on an Enspire plate reader with 680 and 615 nm as excitation and emission wavelengths, respectively. The dissociation constant (K_{D}) is calculated based on a variable slope, four parameter analysis using GraphPad PRISM software.

### Example 8—Binding of IL-18 Variants to IL-D8Rα Monomer

Table 5 shows results of the dissociation constants (K_{D}) observed for the IL-18 variants described to IL-18Rα using the protocol as set forth in Example 6.

TABLE 5
Binding of IL-18Rα monomer

| SEQ ID | | Kₒₙ | K_{off} | K_{D} |
|---|---|---|---|---|
| NO: | Sequence modifications | (1/Ms) | (1/s) | (nM) |
| 1 | Native sequence | 6.76E+05 | 4.31E−03 | 6.61 |
| 57 + | E6K, C38S, K53A, C68-30kDPEG | 3.79E+04 | 2.67E−02 | 701 |
| PEG30 | | | | |
| 57 | E6K, C38S, K53A | 9.52E+04 | 1.33E−02 | 75 |
| 59 | E6K, C38A, K53A | 1.77E+06 | 1.65E−02 | 17.8 |
| 59 + | E6K, C38A, K53A, C68-30kDPEG | 7.46E+04 | 2.64E−02 | 352 |
| PEG30 | | | | |
| 11 + | E6K, V11I, C38A, K53A, C76A, C127A, | NT | NT | NT |
| PEG | C68-30kPEG | | | |
| 11 | E6K, V11I, C38A, K53A, C76A, C127A | NT | NT | NT |
| 6 + | E6K, V11I, C38A, K53A, T63A, C68A, | NT | NT | NT |
| PEG | C76A, C127A, D98C-30kPEG | | | |
| 6 | E6K, V11I, C38A, K53A, T63A, C68A, | NT | NT | NT |
| | C76A, C127A, D98C | | | |
| 5 + | E6K, V11I, C38A, K53A, T63A, C68A, | NT | NT | NT |
| PEG | C76A, C127A, M86C-30kPEG | | | |
| 5 | E6K, V11I, C38A, K53A, T63A, C68A, | NT | NT | NT |
| | C76A, C127A, M86C | | | |
| 9 + | E6K, C38A, K53A, C68A, D98C-30kPEG | NT | NT | NT |
| PEG | | | | |
| 9 | E6K, C38A, K53A, C68A, D98C | NT | NT | NT |
| 4 + | E6K, C38A, K53A, C68A, M86C-30kPEG | NT | NT | NT |
| PEG | | | | |
| 4 | E6K, C38A, K53A, C68A, M86C | NT | NT | NT |
| 28 + | E6K, C38A, K53A, T63A, C76A, C127A, | NT | NT | NT |
| PEG | C68-30kPEG | | | |
| 28 | E6K, C38A, K53A, T63A, C76A, C127A | NT | NT | NT |
| 30 + | E6K, V11I, C38A, K53A, T63A, C76A, | 1.25E+05 | 8.42E−02 | 770 |
| PEG30 | C127A, C68-30kPEG | | | |
| 30 + | E6K, V11I, C38A, K53A, T63A, C76A, | 2.13E+05 | 8.05E−02 | 379 |
| PEG20 | C127A, C68-20kPEG | | | |
| 30 + | E6K, V11I, C38A, K53A, T63A, C76A, | 3.26E+05 | 7.79E−02 | 239 |
| PEG10 | C127A, C68-10kPEG | | | |
| 30 + | E6K, V11I, C38A, K53A, T63A, C76A, | 4.75E+05 | 7.48E−02 | 158 |
| PEG5 | C127A, C68-5kPEG | | | |
| 30 | E6K, V11I, C38A, K53A, T63A, C76A, | 1.77E+06 | 5.36E−02 | 30.3 |
| | C127A | | | |
| 62 + | E6K, C38A, K53A, C127A, C68-30kPEG | — | — | >1000 |
| PEG | | | | |
| 62 | E6K, C38A, K53A, C127A | 6.64E+05 | 4.24E−02 | 63.9 |
| 61 + | E6K, C38A, K53A, C76A, C68-30kPEG | 2.87E+04 | 2.88E−02 | ~1000 |
| PEG | | | | |
| 61 | E6K, C38A, K53A, C76A | 2.22E+06 | 6.74E−02 | 30.4 |
| 60 + | E6K, C38Q, K53A, C68-30kPEG | 6.63E+04 | 1.95E−02 | 345 |
| PEG | | | | |
| 60 | E6K, C38Q, K53A | 1.03E+06 | 1.02E−02 | 9.85 |
| 59 + | E6K, C38A, K53A, C68-30kPEG | NT | NT | NT |
| PEG | | | | |
| 59 | E6K, C38A, K53A | NT | NT | NT |
| 63 | E6K, C38A, K53A, C76A, C127A | 2.31E+07 | 9.76E−01 | 42.2 |

### Example 9—Binding of IL-18 Variants to IL-18Rα/β Heterodimer

Table 6 shows results of the dissociation constants (K_{D}) observed for the IL-18 variants described to IL-18Rα/β heterodimer using the experimental as described in Example 6.

TABLE 6
Binding of IL-18Rα/β heterodimer

| SEQ ID | | Kₒₙ | K_{off} | K_{D} |
|---|---|---|---|---|
| NO: | Sequence modifications | (1/Ms) | (1/s) | (nM) |
| 1 | Native sequence | 7.26E+05 | 4.51E−04 | 1.02 |
| 57 + | E6K, C38S, K53A, C68-30kDPEG | 3.38E+04 | 7.87E−04 | 23 |
| PEG | | | | |
| 57 | E6K, C38S, K53A | 5.35E+05 | 8.16E−04 | 2.30 |
| 59 | E6K, C38A, K53A | 1.24E+06 | 8.15E−04 | 0.66 |
| 59 + | E6K, C38A, K53A, C68-30kDPEG | 8.45E+04 | 5.80E−04 | 8.02 |
| PEG30 | | | | |
| 11 + | E6K, V11I, C38A, K53A, C76A, | NT | NT | NT |
| PEG | C127A, C68-30kPEG | | | |
| 11 | E6K, V11I, C38A, K53A, C76A, | NT | NT | NT |
| | C127A | | | |
| 6 + | E6K, V11I, C38A, K53A, T63A, C68A, | NT | NT | NT |
| PEG | C76A, C127A, D98C-30kPEG | | | |
| 6 | E6K, V11I, C38A, K53A, T63A, C68A, | NT | NT | NT |
| | C76A, C127A, D98C | | | |
| 5 + | E6K, V11I, C38A, K53A, T63A, C68A, | NT | NT | NT |
| PEG | C76A, C127A, M86C-30kPEG | | | |
| 5 | E6K, V11I, C38A, K53A, T63A, C68A, | NT | NT | NT |
| | C76A, C127A, M86C | | | |
| 9 + | E6K, C38A, K53A, C68A, D98C- | NT | NT | NT |
| PEG | 30kPEG | | | |
| 9 | E6K, C38A, K53A, C68A, D98C | NT | NT | NT |
| 4 + | E6K, C38A, K53A, C68A, M86C- | NT | NT | NT |
| PEG | 30kPEG | | | |
| 4 | E6K, C38A, K53A, C68A, M86C | NT | NT | NT |
| 28 + | E6K, C38A, K53A, T63A, C76A, | NT | NT | NT |
| PEG | C127A, C68-30kPEG | | | |
| 28 | E6K, C38A, K53A, T63A, C76A, | NT | NT | NT |
| | C127A | | | |
| 30 + | E6K, V11I, C38A, K53A, T63A, C76A, | 3.94E+04 | 6.18E−04 | 15.7 |
| PEG30 | C127A, C68-30kPEG | | | |
| 30 + | E6K, V11I, C38A, K53A, T63A, C76A, | 1.16E+05 | 6.37E−04 | 5.49 |
| PEG20 | C127A, C68-20kPEG | | | |
| 30 + | E6K, V11I, C38A, K53A, T63A, C76A, | 1.20E+05 | 1.05E−03 | 8.7 |
| PEG10 | C127A, C68-10kPEG | | | |
| 30 + | E6K, V11I, C38A, K53A, T63A, C76A, | 2.69E+05 | 5.94E−04 | 2.2 |
| PEG5 | C127A, C68-5kPEG | | | |
| 30 | E6K, V11I, C38A, K53A, T63A, C76A, | 8.66E+05 | 3.11E−04 | 0.49 |
| | C127A | | | |
| 62 + | E6K, C38A, K53A, C127A, C68- | 5.23E+04 | 7.79E−04 | 14.9 |
| PEG | 30kPEG | | | |
| 62 | E6K, C38A, K53A, C127A | 1.21E+06 | 1.49E−03 | 1.24 |
| 61 + | E6K, C38A, K53A, C76A, C68- | 3.81E+04 | 5.89E−04 | 15.5 |
| PEG | 30kPEG | | | |
| 61 | E6K, C38A, K53A, C76A | 1.09E+06 | 1.10E−03 | 1.01 |
| 60 + | E6K, C38Q, K53A, C68-30kPEG | 6.83E+04 | 7.89E−04 | 13.3 |
| PEG | | | | |
| 60 | E6K, C38Q, K53A | 1.21E+06 | 5.89E−04 | 0.490 |
| 59 + | E6K, C38A, K53A, C68-30kPEG | NT | NT | NT |
| PEG | | | | |
| 59 | E6K, C38A, K53A | NT | NT | NT |
| 63 | E6K, C38A, K53A, C76A, C127A | 1.15E+06 | 4.67E−04 | 0.41 |

### Example 10—Binding Assay to IL-18BP Monomer

Table 7 shows results of the dissociation constants (K_{D}) observed for the IL-18 variants described to IL-18BP using an analogous protocol to that described in Example 6.

TABLE 7
Binding of IL-18BP monomer determined by SPR

| SEQ ID | | Kₒₙ | K_{off} | K_{D} |
|---|---|---|---|---|
| NO: | Sequence modifications | (1/Ms) | (1/s) | (nM) |
| 1 | Native sequence | 1.28E+06 | 3.80E−04 | 0.246 |
| 57 + | E6K, C38S, K53A, C68-30kDPEG | 1.77E+05 | 3.97E−03 | 23 |
| PEG | | | | |
| 57 | E6K, C38S, K53A | 1.63E+06 | 3.11E−03 | 1.80 |
| 59 | E6K, C38A, K53A | 1.47E+06 | 3.01E−03 | 2.05 |
| 59 + | E6K, C38A, K53A, C68-30kDPEG | 1.99E+05 | 3.55E−03 | 19.4 |
| PEG30 | | | | |
| 11 + | E6K, V11I, C38A, K53A, C76A, | NT | NT | NT |
| PEG | C127A, C68-30kPEG | | | |
| 11 | E6K, V11I, C38A, K53A, C76A, | NT | NT | NT |
| | C127A | | | |
| 6 + | E6K, V11I, C38A, K53A, T63A, | NT | NT | NT |
| PEG | C68A, C76A, C127A, D98C-30kPEG | | | |
| 6 | E6K, V11I, C38A, K53A, T63A, | NT | NT | NT |
| | C68A, C76A, C127A, D98C | | | |
| 5 + | E6K, V11I, C38A, K53A, T63A, | NT | NT | NT |
| PEG | C68A, C76A, C127A, M86C- | | | |
| | 30kPEG | | | |
| 5 | E6K, V11I, C38A, K53A, T63A, | NT | NT | NT |
| | C68A, C76A, C127A, M86C | | | |
| 9 + | E6K, C38A, K53A, C68A, D98C- | NT | NT | NT |
| PEG | 30kPEG | | | |
| 9 | E6K, C38A, K53A, C68A, D98C | NT | NT | NT |
| 4 + | E6K, C38A, K53A, C68A, M86C- | NT | NT | NT |
| PEG | 30kPEG | | | |
| 4 | E6K, C38A, K53A, C68A, M86C | NT | NT | NT |
| 28 + | E6K, C38A, K53A, T63A, C76A, | NT | NT | NT |
| PEG | C127A, C68-30kPEG | | | |
| 28 | E6K, C38A, K53A, T63A, C76A, | NT | NT | NT |
| | C127A | | | |
| 30 + | E6K, V11I, C38A, K53A, T63A, | 1.06E+05 | 4.51E−03 | 45.10 |
| PEG30 | C76A, C127A, C68-30kPEG | | | |
| 30 + | E6K, V11I, C38A, K53A, T63A, | 3.30E+05 | 3.97E−03 | 12 |
| PEG20 | C76A, C127A, C68-20kPEG | | | |
| 30 + | E6K, V11I, C38A, K53A, T63A, | 5.27E+05 | 3.60E−03 | 6.83 |
| PEG10 | C76A, C127A, C68-10kPEG | | | |
| 30 + | E6K, V11I, C38A, K53A, T63A, | 8.13E+05 | 3.86E−03 | 4.75 |
| PEG5 | C76A, C127A, C68-5kPEG | | | |
| 30 | E6K, V11I, C38A, K53A, T63A, | 1.05E+06 | 3.45E−03 | 3.05 |
| | C76A, C127A | | | |
| 62 + | E6K, C38A, K53A, C127A, C68- | 1.42E+05 | 3.42E−03 | 24.0 |
| PEG | 30kPEG | | | |
| 62 | E6K, C38A, K53A, C127A | 1.61E+06 | 3.15E−03 | 1.96 |
| 61 + | E6K, C38A, K53A, C76A, C68- | 1.02E+05 | 3.82E−03 | 37.4 |
| PEG | 30kPEG | | | |
| 61 | E6K, C38A, K53A, C76A | 1.74E+06 | 3.60E−03 | 2.1 |
| 60 + | E6K, C38Q, K53A, C68-30kPEG | 1.93E+05 | 3.95E−03 | 22 |
| PEG | | | | |
| 60 | E6K, C38Q, K53A | 1.87E+06 | 3.08E−03 | 1.700 |
| 59 + | E6K, C38A, K53A, C68-30kPEG | NT | NT | NT |
| PEG | | | | |
| 59 | E6K, C38A, K53A | NT | NT | NT |
| 63 | E6K, C38A, K53A, C76A, C127A | 2.82E+06 | 4.24E−03 | 1.5 |

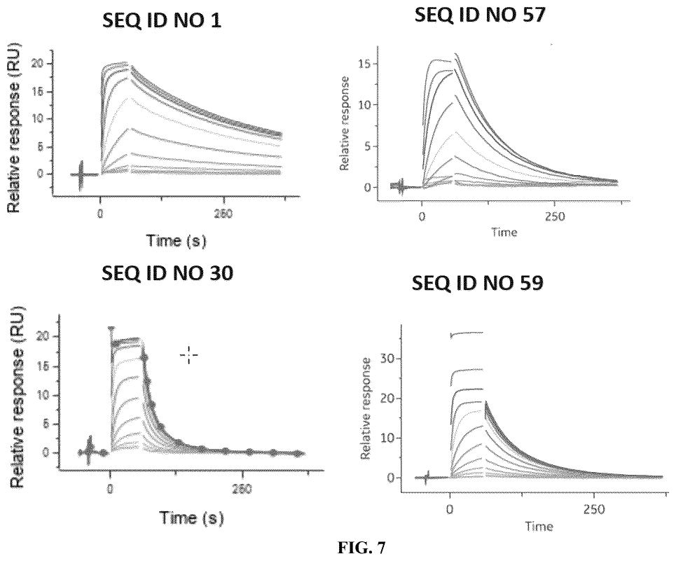

FIG. 7 shows plots measuring the binding activity of wild type IL-18 and of modified IL-18 polypeptides to human IL-18 receptor alpha (CD218a) in surface plasmon resonance experiments, where the x-axis is time, and the y-axis is relative response. The modified IL-18 polypeptides tested in these experiments are native IL-18 wild-type (SEQ ID NO: 01), SEQ ID NO: 57, SEQ ID NO: 59, and SEQ ID NO: 30.

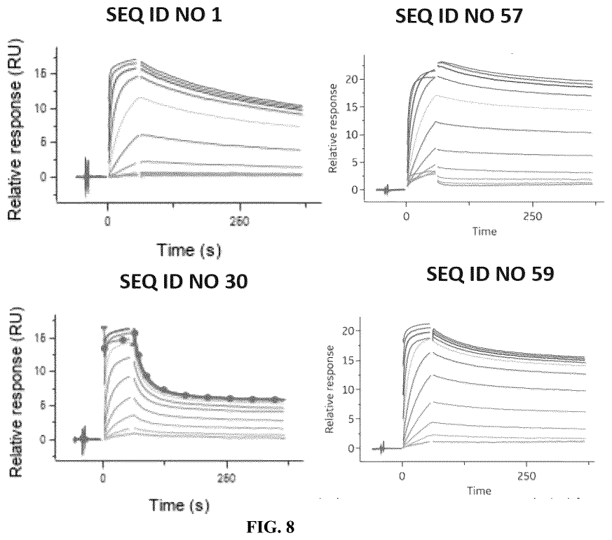

FIG. 8 shows plots measuring the binding activity of wild type IL-18 and of modified IL-18 polypeptides to heterodimeric human IL-18 receptor alpha (CD218a) and IL-18R accessory protein (IL-18RAP/CDw218b) in surface plasmon resonance experiments, where the x-axis is time, and the y-axis is relative response. The modified IL-18 polypeptides tested in these experiments are native IL-18 wild-type (SEQ ID NO: 01), SEQ ID NO: 57, SEQ ID NO: 59, and SEQ ID NO: 30.

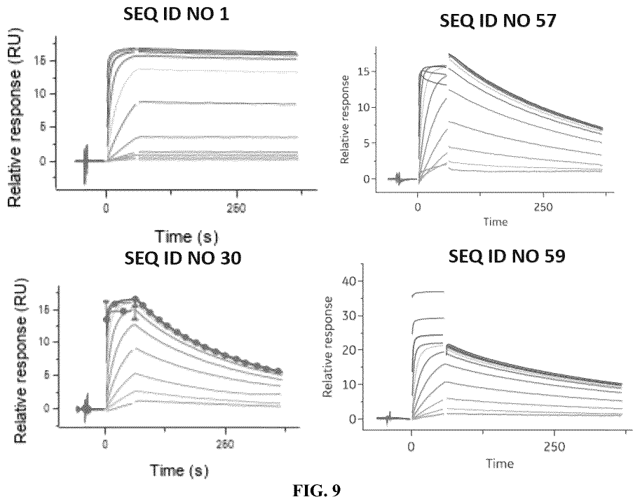

FIG. 9 shows plots measuring the binding activity of wild type IL-18 and of modified IL-18 polypeptides to human IL-18 binding protein (IL-18BP) in surface plasmon resonance experiments, where the x-axis is time, and the y-axis is relative response. The modified IL-18 polypeptides tested in these experiments are native IL-18 wild-type (SEQ ID NO: 01), SEQ ID NO: 57, SEQ ID NO: 59, and SEQ ID NO: 30.

Table 8 shows results of the dissociation constants (K_{D}) observed for the IL-18 variants described to IL-18BP as measured using the protocol described in Example 7.

TABLE 8
Binding of IL-18BP monomer determined by alphaLISA

| SEQ ID | | KD |
|---|---|---|
| NO: | Sequence modifications | (nM) |
| 1 | Native Sequence | 0.411 |
| 57 | E6K, K53A, C38S | 48.4 |
| 57 + | E6K, C38S, K53A, C68-30kDPEG | 42.5 |
| PEG | | |
| 8 | E6K, V11I, C38A, K53A, T63A, C68A, C76A, T95C, | 61.055 |
| | C127A | |
| 11 + | E6K, V11I, C38A, K53A, C76A, C127A, C68-30kPEG | NT |
| PEG | | |
| 11 | E6K, V11I, C38A, K53A, C76A, C127A | 36.800 |
| 6 + | E6K, V11I, C38A, K53A, T63A, C68A, C76A, C127A, | 20.180 |
| PEG | D98C-30kPEG | |
| 6 | E6K, V11I, C38A, K53A, T63A, C68A, C76A, C127A, | 630.900 |
| | D98C | |
| 5 + | E6K, V11I, C38A, K53A, T63A, C68A, C76A, C127A, | 31.410 |
| PEG | M86C-30kPEG | |
| 5 | E6K, V11I, C38A, K53A, T63A, C68A, C76A, C127A, | 180.680 |
| | M86C | |
| 9 + | E6K, C38A, K53A, C68A, D98C-30kPEG | 150.600 |
| PEG | | |
| 9 | E6K, C38A, K53A, C68A, D98C | 82.650 |
| 4 + | E6K, C38A, K53A, C68A, M86C-30kPEG | 128.600 |
| PEG | | |
| 4 | E6K, C38A, K53A, C68A, M86C | 55.600 |
| 28 + | E6K, C38A, K53A, T63A, C76A, C127A, C68-30kPEG | 40.675 |
| PEG | | |
| 28 | E6K, C38A, K53A, T63A, C76A, C127A | 44.410 |
| 30 + | E6K, V11I, C38A, K53A, T63A, C76A, C127A, C68- | 26.310 |
| PEG30 | 30kPEG | |
| 30 + | E6K, V11I, C38A, K53A, T63A, C76A, C127A, C68- | 19.670 |
| PEG20 | 20kPEG | |
| 30 + | E6K, V11I, C38A, K53A, T63A, C76A, C127A, C68- | 15.690 |
| PEG10 | 10kPEG | |
| 30 + | E6K, V11I, C38A, K53A, T63A, C76A, C127A, C68- | 19.700 |
| PEG5 | 5kPEG | |
| 30 | E6K, V11I, C38A, K53A, T63A, C76A, C127A | 24.600 |
| 62 + | E6K, C38A, K53A, C127A, C68-30kPEG | 29.314 |
| PEG | | |
| 62 | E6K, C38A, K53A, C127A | 69.620 |
| 61 + | E6K, C38A, K53A, C76A, C68-30kPEG | NT |
| PEG | | |
| 61 | E6K, C38A, K53A, C76A | NT |
| 60 + | E6K, C38Q, K53A, C68-30kPEG | 63.658 |
| PEG | | |
| 60 | E6K, C38Q, K53A | 24.800 |
| 59 + | E6K, C38A, K53A, C68-30kPEG | 75.220 |
| PEG30 | | |
| 59 + | E6K, C38A, K53A, C68-20kPEG | 48.460 |
| PEG20 | | |
| 59 + | E6K, C38A, K53A, C68-10kPEG | 50.710 |
| PEG10 | | |
| 59 | E6K, C38A, K53A | 36.425 |
| 63 | E6K, C38A, K53A, C76A, C127A | 20.257 |
| 204 | V11I, C38A, K53A, C76A, C127A | 8.86 |
| 205 | V11I, C38A, K53A, T63A, C76A, C127A | 0.66 |
| 206 | V11I, C38A, K53A, S55A, C76A, C127A | 9.74 |
| 207 | V11I, C38A, M51G, K53A, C76A, C127A | 373.30 |
| 208 | V11I, C38A, K53A, D54A, C76A, C127A | 25.77 |
| 209 | F2A, V11I, C38A, K53A, C76A, C127A | 57.21 |
| 210 | V11I, E31A, C38A, K53A, C76A, C127A | 0.64 |
| 211 | V11I, T34A, C38A, K53A, C76A, C127A | 1.24 |
| 212 | V11I, D35A, C38A, K53A, C76A, C127A | 2.88 |
| 213 | V11I, S36A, C38A, K53A, C76A, C127A | 1.12 |
| 214 | V11I, D37A, C38A, K53A, C76A, C127A | 4.55 |
| 215 | V11I, E31A, D37A, C38A, K53A, C76A, C127A | 2.12 |
| 216 | V11I, C38A, D40A, K53A, C76A, C127A | 0.74 |
| 217 | V11I, C38A, N41A, K53A, C76A, C127A | 18.47 |
| 218 | V11I, C38A, K53A, C76A, C127A, D132A | 13.70 |
| 219 | V11I, C38A, K53A, C76A, G108A, C127A | 1.24 |
| 220 | V11I, C38A, K53A, C76A, H109A, C127A | 0.55 |
| 221 | V11I, C38A, K53A, C76A, D110A, C127A | 0.71 |
| 222 | K8R, V11I, C38A, C76A, Q103E, C127A | 0.06 |
| 223 | K8E, V11I, C38A, C76A, Q103R, C127A | 0.85 |
| 224 | V11I, C38A, C76A, Q103K, C127A | 0.05 |
| 225 | V11I, C38A, S55H, C76A, C127A | 0.08 |
| 226 | V11I, C38A, S55R, C76A, C127A | 0.15 |
| 227 | V11I, C38A, S55T, C76A, C127A | 0.02 |
| 228 | V11I, C38A, C76A, S105I, C127A | 0.04 |
| 229 | V11I, C38A, C76A, S105K, C127A | 0.05 |
| 230 | K8L, E6K, V11I, C38A, K53A, T63A, C76A, C127A | 0.14 |
| 231 | E6K, V11I, C38A, I49E, K53A, T63A, C76A, C127A | |
| 232 | E6K, V11I, C38A, I49M, K53A, T63A, C76A, C127A | 25.84 |
| 233 | E6K, V11I, C38A, I49R, K53A, T63A, C76A, C127A | >2800 |
| 234 | E6K, V11I, C38A, K53A, T63A, C76A, Q103R, C127A | >2800 |
| 235 | E6K, K8E, V11I, C38A, K53A, T63A, C76A, Q103R, | >2800 |
| | C127A | |
| 236 | E6K, V11I, C38A, K53A, T63A, C76A, C127A, V153R | NT |
| 237 | E6K, V11I, C38A, K53A, T63A, C76A, C127A, V153E | >2800 |
| 238 | E6K, V11I, C38A, K53A, T63A, C76A, C127A, V153Y | >2800 |
| 239 | E6K, V11I, C38A, M51G, K53A, T63A, C76A, C127A | >2800 |
| 240 | E6R, V11I, C38A, K53A, T63A, C76A, C127A | 5.46 |
| 241 | E6K, V11I, C38A, K53A, T63A, C76A, C127A | >2800 |
| 242 | E6K, V11I, C38A, K53A, T63A, C76A, C127A | >2800 |
| 243 | Y1M, E6K, V11I, C38A, K53A, T63A, C76A, C127A | 2.25 |
| 244 | E6K, V11I, C38A, M51G, K53A, T63A, C76A, C127A | >2800 |

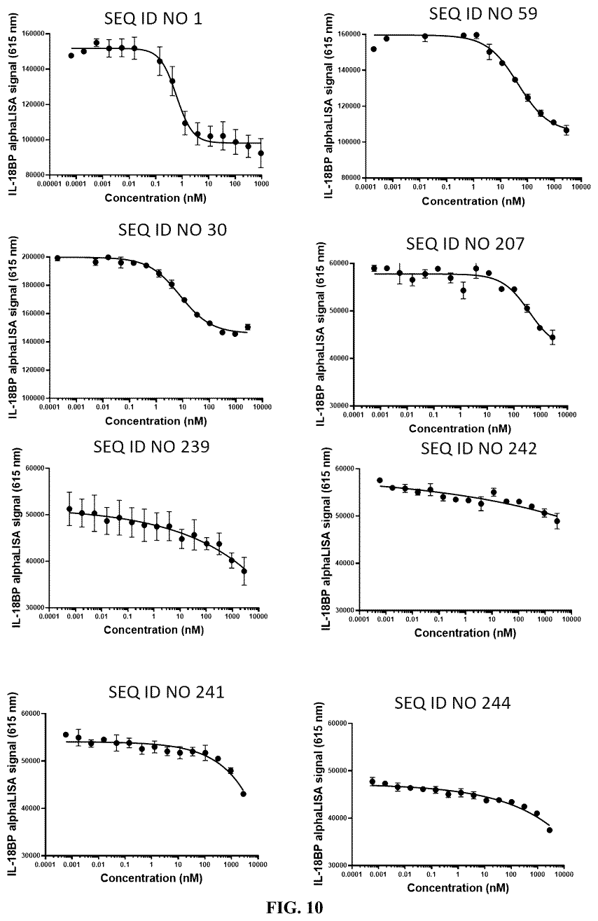

FIG. 10 shows plots measuring the ability of wild type IL-18 and of modified IL-18 polypeptides to bind to the human IL-18 Binding Protein (IL-18BP). The figure shows mean free IL-18BP AlphaLISA signal on the y-axis, and dosage of wild type IL-18 and of modified IL-18 polypeptides on the x-axis. The unconjugated IL-18 variants are native IL-18 wild-type (SEQ ID NO: 01), SEQ ID NO: 57, SEQ ID NO: 59, SEQ ID NO: 30, SEQ ID NO: 207, SEQ ID NO: 239, SEQ ID NO: 241, SEQ ID NO: 242, and SEQ ID NO: 244.

### Example 11—IFNγ Induction NK-92 Cellular Assay

The ability of IL-18 polypeptides provided herein are assessed for ability to induce IFN□ in a cellular assay according to the protocol below.

The NK cell line NK-92 derived from a patient with lymphoma (ATCC® CRL-2407™) is cultured in aMEM medium supplemented with 20% FCS, Glutamax™, 25 M B-mercaptoethanol, and 100 IU/mL of recombinant human IL-2.

On the day of experiment, cells are harvested and washed with aMEM medium without IL-2 and containing 1 ng/mL of recombinant human IL-12. After counting, cells are seeded at 100,000 cells/well in a 384 well titer plate and incubated at 37° C./5% CO₂. Sixteen 4-fold serial dilutions of IL-18 analytes are prepared in aMEM medium, and 1 ng/mL of IL-12 were added to the NK-92 cells. Final IL-18 analyte concentrations range from 56 nM to 5×10⁻⁵ pM.

After incubating the cells for 16-20 hr at 37° C./5% CO₂, 5 μL of supernatant is carefully transferred to a 384 microwell OptiPlate. IFNγ levels are measured using a human IFNγ AlphaLISA Assay Kit. Briefly, 10 μL of 2.5× AlphaLISA Anti-IFNγ acceptor beads and biotinylated antibody anti-IFNγ mix are added to the 5 μL of NK-92 supernatants. The mixtures are incubated for 1 hr at room temperature with shaking. Under subdued light, 2.5 μL of 2× streptavidin (SA) donor beads are pipetted into each well, and the wells are incubated for 30 min at room temperature with shaking. AlphaLISA signals are then measured on an EnSpire™ plate reader using 680 nm and 615 nm as excitation and emission wavelengths, respectively. Half maximal effective concentrations (EC₅₀) are calculated based on a variable slope and four parameter analysis using GraphPad PRISM software.

Results of this experiment for various IL-18 polypeptides is shown below in Table 9 (EC₅₀ data).

### Example 12—IL-18 Binding Protein-Mediated Inhibition of IFNγ Secretion in NK-92 Cellular Assay

The NK cell line NK-92 derived from a patient with lymphoma (ATCC® CRL-2407™) is cultured in aMEM medium supplemented with 20% FCS-Glutamax™, 25 M B-mercaptoethanol, and 100 IU/mL of recombinant human IL-2.

On the day of experiment, cells are harvested and washed with aMEM medium without IL-2 and containing 1 ng/mL of recombinant human IL-12. After counting, the cells are seeded at 100,000 cells/well in a 384 well titer plate and incubated at 37° C./5% CO₂. Sixteen 2-fold serial dilutions of Fc-fused human IL-18 binding protein isoform a (IL-18BPa) are prepared in aMEM medium. 1 ng/mL of IL-12 containing 2 nM of each modified IL-18 polypeptide variant is added to the NK-92 cells. The final IL-18 analyte concentration is 1 nM, and the final IL-18BPa concentration ranged from 566 nM to 17 pM.

After incubating the cells for 16-20 hr at 37° C./5% CO₂, 5 μL of the supernatant is carefully transferred to a 384 microwell OptiPlate. IFNγ levels are measured using a human IFNγ AlphaLISA Assay Kit. Briefly, 10 μL of 2.5× AlphaLISA anti-IFNγ acceptor beads and biotinylated antibody anti-IFNγ mix are added to 5 μL of NK-92 supernatants. The mixtures are incubated for 1 hr at room temperature with shaking. Under subdued light, 2.5 μL of 2× SA donor beads are pipetted in each well and incubated for 30 min at room temperature with shaking. AlphaLISA signals are then measured on an EnSpire™ plate reader using 680 nm and 615 nm as excitation and emission wavelengths, respectively. Half maximal inhibitory concentrations (IC₅₀) are calculated based on a variable slope and four parameter analysis using GraphPad PRISM software.

Modified IL-18 variants of the disclosure are active and able to induce IFNγ secretion in vitro. Table 9 shows the ability of many of the tested IL-18 variants to induce IFNγ production while some IL-18 variants are significantly less sensitive to inhibition by IL-18BP, as measured by EC₅₀ and IC₅₀, respectively.

TABLE 9
IC₅₀/EC₅₀ in NK92 IFNγ Release Assay Data

| SEQ ID | | EC₅₀ | IC₅₀ | IC₅₀/ |
|---|---|---|---|---|
| NO: | Sequence modifications | (nM) | (nM) | EC₅₀ |
| 1 | Native sequence | 0.367 | 0.781 | 2.653 |
| 8 | E6K, V11I, C38A, K53A, T63A, C68A, C76A, T95C, | 0.022 | 522 | 517879 |
| | C127A | | | |
| 11 + | E6K, V11I, C38A, K53A, C76A, C127A, C68-30kPEG | NT | NT | NT |
| PEG | | | | |
| 11 | E6K, V11I, C38A, K53A, C76A, C127A | 0.008 | 532 | 475272 |
| 6 + | E6K, V11I, C38A, K53A, T63A, C68A, C76A, C127A, | 0.272 | 35.510 | 701.1 |
| PEG | D98C-30kPEG | | | |
| 6 | E6K, V11I, C38A, K53A, T63A, C68A, C76A, C127A, | 0.631 | 25.290 | 222.232 |
| | D98C | | | |
| 5 + | E6K, V11I, C38A, K53A, T63A, C68A, C76A, C127A, | 0.262 | 32.500 | 1799.557 |
| PEG | M86C-30kPEG | | | |
| 5 | E6K, V11I, C38A, K53A, T63A, C68A, C76A, C127A, | 0.033 | 357.500 | 159030.249 |
| | M86C | | | |
| 9 + | E6K, C38A, K53A, C68A, D98C-30kPEG | 0.226 | 142.900 | 4455.878 |
| PEG | | | | |
| 9 | E6K, C38A, K53A, C68A, D98C | 0.015 | 1000.000 | 248262.165 |
| 4 + | E6K, C38A, K53A, C68A, M86C-30kPEG | 0.224 | 125.600 | 4085.882 |
| PEG | | | | |
| 4 | E6K, C38A, K53A, C68A, M86C | 0.011 | 1000.000 | 253356.980 |
| 28 + | E6K, C38A, K53A, T63A, C76A, C127A, C68-30kPEG | 0.437 | 12.243 | 122.081 |
| PEG | | | | |
| 28 | E6K, C38A, K53A, T63A, C76A, C127A | 0.005 | 233.050 | 126262.157 |
| 30 + | E6K, V11I, C38A, K53A, T63A, C76A, C127A, C68- | 0.048 | 34.295 | 574.446 |
| PEG30 | 30kPEG | | | |
| 30 + | E6K, V11I, C38A, K53A, T63A, C76A, C127A, C68- | 0.011 | 44.250 | 9890.478 |
| PEG20 | 20kPEG | | | |
| 30 + | E6K, V11I, C38A, K53A, T63A, C76A, C127A, C68- | 0.009 | 67.080 | 14759.076 |
| PEG10 | 10kPEG | | | |
| 30 + | E6K, V11I, C38A, K53A, T63A, C76A, C127A, C68- | 0.003 | 105.100 | 124526.066 |
| PEG5 | 5kPEG | | | |
| 30 | E6K, V11I, C38A, K53A, T63A, C76A, C127A | 0.0014 | 487.800 | 234185.122 |
| 62 + | E6K, C38A, K53A, C127A, C68-30kPEG | 0.795 | 4.831 | 6 |
| PEG | | | | |
| 62 | E6K, C38A, K53A, C127A | 0.026 | 283.600 | 11285 |
| 61 + | E6K, C38A, K53A, C76A, C68-30kPEG | 0.503 | 4.291 | 13 |
| PEG | | | | |
| 61 | E6K, C38A, K53A, C76A | 0.007 | 778.900 | 187617 |
| 60 + | E6K, C38Q, K53A, C68-30kPEG | 0.279 | 28.140 | 132 |
| PEG | | | | |
| 60 | E6K, C38Q, K53A | 0.008 | 828.700 | 133297.787 |
| 59 + | E6K, C38A, K53A, C68-30kPEG | 0.278 | 41.840 | 252 |
| PEG30 | | | | |
| 59 + | E6K, C38A, K53A, C68-20kPEG | 0.153 | NT | — |
| PEG20 | | | | |
| 59 + | E6K, C38A, K53A, C68-10kPEG | 0.142 | 235.900 | 2711 |
| PEG10 | | | | |
| 59 | E6K, C38A, K53A | 0.015 | 653.500 | 44066 |
| 57 + | E6K, C38S, K53A, C68-30kDPEG | 1.319 | 11.425 | 25 |
| PEG | | | | |
| 57 | E6K, C38S, K53A | 0.045 | 146.200 | 3621 |
| 63 | E6K, C38A, K53A, C76A, C127A | 0.007 | 286.000 | 35344 |
| 204 | V11I, C38A, K53A, C76A, C127A | 0.138 | 1.625 | 11.8 |
| 205 | V11I, C38A, K53A, T63A, C76A, C127A | 0.012 | 7.522 | 631.0 |
| 206 | V11I, C38A, K53A, S55A, C76A, C127A | 0.087 | 10.240 | 117.8 |
| 207 | V11I, C38A, M51G, K53A, C76A, C127A | 0.037 | 732.900 | 19633.0 |
| 208 | V11I, C38A, K53A, D54A, C76A, C127A | 0.079 | 47.630 | 604.9 |
| 209 | F2A, V11I, C38A, K53A, C76A, C127A | 0.256 | 5.055 | 19.7 |
| 210 | V11I, E31A, C38A, K53A, C76A, C127A | 0.187 | 1.167 | 6.2 |
| 211 | V11I, T34A, C38A, K53A, C76A, C127A | 0.015 | 21.270 | 1378.5 |
| 212 | V11I, D35A, C38A, K53A, C76A, C127A | 0.061 | 3.622 | 59.4 |
| 213 | V11I, S36A, C38A, K53A, C76A, C127A | 0.033 | 7.850 | 237.3 |
| 214 | V11I, D37A, C38A, K53A, C76A, C127A | 0.175 | 2.222 | 12.7 |
| 215 | V11I, E31A, D37A, C38A, K53A, C76A, C127A | 0.062 | 3.709 | 59.4 |
| 216 | V11I, C38A, D40A, K53A, C76A, C127A | 0.067 | 3.233 | 48.5 |
| 217 | V11I, C38A, N41A, K53A, C76A, C127A | 0.558 | 0.681 | 1.2 |
| 218 | V11I, C38A, K53A, C76A, C127A, D132A | 0.056 | 6.082 | 108.9 |
| 219 | V11I, C38A, K53A, C76A, G108A, C127A | 0.073 | 3.981 | 54.3 |
| 220 | V11I, C38A, K53A, C76A, H109A, C127A | 0.123 | 1.807 | 14.7 |
| 221 | V11I, C38A, K53A, C76A, D110A, C127A | 0.028 | 3.181 | 115.1 |
| 222 | K8R, V11I, C38A, C76A, Q103E, C127A | 0.057 | 1.073 | 18.7 |
| 223 | K8E, V11I, C38A, C76A, Q103R, C127A | 0.061 | 7.292 | 120.3 |
| 224 | V11I, C38A, C76A, Q103K, C127A | 0.093 | 0.823 | 8.9 |
| 225 | V11I, C38A, S55H, C76A, C127A | 0.414 | 0.456 | 1.1 |
| 226 | V11I, C38A, S55R, C76A, C127A | 0.176 | 0.885 | 5.0 |
| 227 | V11I, C38A, S55T, C76A, C127A | 0.098 | 0.440 | 4.5 |
| 228 | V11I, C38A, C76A, S105I, C127A | 0.103 | 0.809 | 7.9 |
| 229 | V11I, C38A, C76A, S105K, C127A | 0.098 | 0.176 | 1.8 |

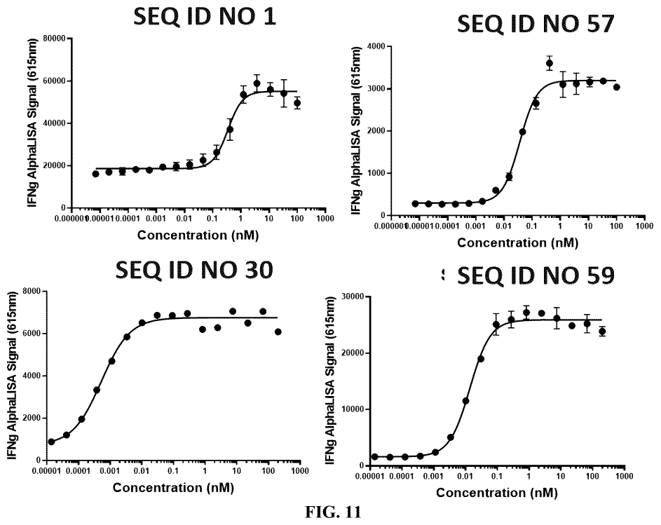

FIG. 11 shows plots measuring ability of wild type IL-18 and of modified IL-18 polypeptides to stimulate the secretion of IFNgamma by NK-92 cells. The figure shows mean IFNg alphaLISA signal on the y-axis and dosage of the IL-18 polypeptides on the x-axis. The IL-18 polypeptides are native IL-18 wild-type (SEQ ID NO: 01), SEQ ID NO: 57, SEQ ID NO: 59, and SEQ ID NO: 30.

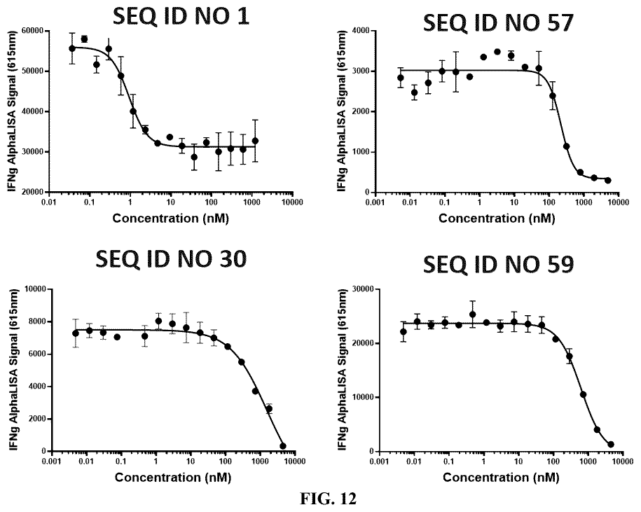

FIG. 12 shows plots measuring the ability of the human IL-18 Binding Protein to inhibit the secretion of IFNgamma by NK92 cells stimulated with 2 nM of wild type IL-18 and of modified IL-18 polypeptides. The figure shows mean IFNg alphaLISA signal on the y-axis, and dosage of the human IL-18 Binding Protein on the x-axis. The IL-18 polypeptides are native IL-18 wild-type (SEQ ID NO: 01), SEQ ID NO: 57, SEQ ID NO: 59, and SEQ ID NO: 30.

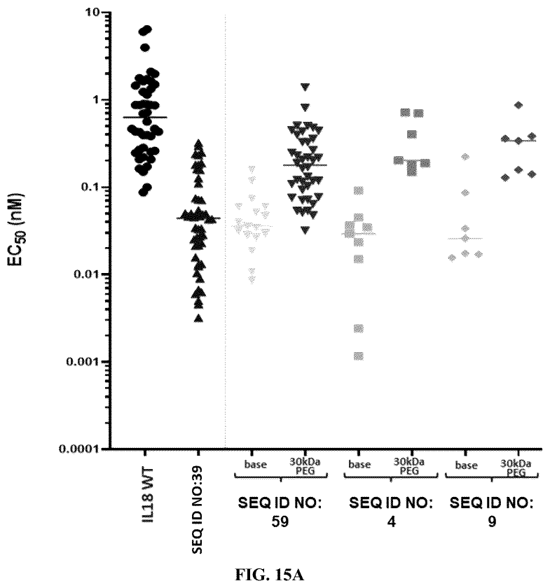

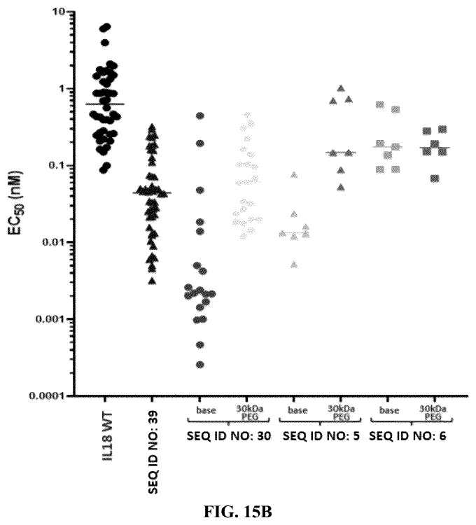

FIGS. 15A-B shows effect of attaching 30 kDa PEG to IL-18 variants on IL-18 mediated IFNγ secretion in human peripheral blood mononuclear cells. EC50 for IL-18 mediated IFNγ secretion for IL-18 of SEQ ID NO: 59, 4 and 9 is shown in FIG. 32A, and of SEQ ID NO: 30, 5 and 6 is shown in FIG. 32B.

### Example 13—HEK-Blue IL18R Reporter Assay

An IL-18R positive HEK-Blue reporter cell line is used to determine binding of IL-18 variants to IL-18R and subsequent downstream signaling. The general protocol is outlined below.

5×10⁴ cells HEK-Blue IL18R reporter cells (InvivoGen, #hkb-hmil18) are seeded into each well of a 96 well plate and stimulated with 0-100 nM of IL-18 polypeptide variants at 37° C. and 5% CO₂. After 20h incubation, 20 μL of cell culture supernatant is then taken from each well and mixed with 180 μL QUANTI-Blue media in a 96 well plate, incubated for 1 hour at 37° C. and 5% CO₂. The absorbance signal at 620 nm is then measured on an Enspire plate reader with 680 and 615 nm as excitation and emission wavelengths, respectively. Half Maximal Effective dose (EC50) is calculated based on a variable slope, four parameter analysis using GraphPad PRISM software.

Results of this experiment for select variants are shown below in Table 10.

TABLE 10
EC₅₀ in HEK-Blue IL18R Reporter Assay Data

| SEQ ID | | EC₅₀ |
|---|---|---|
| NO: | Sequence modifications | (nM) |
| 1 | Native sequence | 2.58 |
| 8 | E6K, V11I, C38A, K53A, T63A, C68A, C76A, T95C, | |
| | C127A | |
| 11 | E6K, V11I, C38A, K53A, C76A, C127A | |
| 6 + | E6K, V11I, C38A, K53A, T63A, C68A, C76A, C127A, | |
| PEG | D98C-30kPEG | |
| 6 | E6K, V11I, C38A, K53A, T63A, C68A, C76A, C127A, | |
| | D98C | |
| 5 + | E6K, V11I, C38A, K53A, T63A, C68A, C76A, C127A, | |
| PEG | M86C-30kPEG | |
| 5 | E6K, V11I, C38A, K53A, T63A, C68A, C76A, C127A, | |
| | M86C | |
| 9 + | E6K, C38A, K53A, C68A, D98C-30kPEG | |
| PEG | | |
| 9 | E6K, C38A, K53A, C68A, D98C | |
| 4 + | E6K, C38A, K53A, C68A, M86C-30kPEG | |
| PEG | | |
| 4 | E6K, C38A, K53A, C68A, M86C | |
| 28 | E6K, C38A, K53A, T63A, C76A, C127A | |
| 30 | E6K, V11I, C38A, K53A, T63A, C76A, C127A | 0.053 |
| 30 + | E6K, V11I, C38A, K53A, T63A, C76A, C127A, | 0.230 |
| PEG | C68-30 kD PEG | |
| 63 | E6K, C38A, K53A, C76A, C127A | 0.01389 |
| 62 | E6K, C38A, K53A, C127A | 0.17 |
| 61 | E6K, C38A, K53A, C76A | 0.084 |
| 60 | E6K, C38Q, K53A | 0.203 |
| 59 | E6K, C38A, K53A | 0.268 |
| 57 | E6K, C38S, K53A | 0.53 |
| 204 | V11I, C38A, K53A, C76A, C127A | 0.98 |
| 205 | V11I, C38A, K53A, T63A, C76A, C127A | 0.17 |
| 206 | V11I, C38A, K53A, S55A, C76A, C127A | 3.63 |
| 207 | V11I, C38A, M51G, K53A, C76A, C127A | 0.8 |
| 208 | V11I, C38A, K53A, D54A, C76A, C127A | 1 |
| 209 | F2A, V11I, C38A, K53A, C76A, C127A | 7.28 |
| 210 | V11I, E31A, C38A, K53A, C76A, C127A | 6.6 |
| 211 | V11I, T34A, C38A, K53A, C76A, C127A | 0.7 |
| 212 | V11I, D35A, C38A, K53A, C76A, C127A | 13.12 |
| 213 | V11I, S36A, C38A, K53A, C76A, C127A | 0.25 |
| 214 | V11I, D37A, C38A, K53A, C76A, C127A | 14.12 |
| 215 | V11I, E31A, D37A, C38A, K53A, C76A, C127A | 11.95 |
| 216 | V11I, C38A, D40A, K53A, C76A, C127A | 0.52 |
| 217 | V11I, C38A, N41A, K53A, C76A, C127A | 11.7 |
| 218 | V11I, C38A, K53A, C76A, C127A, D132A | 1.95 |
| 219 | V11I, C38A, K53A, C76A, G108A, C127A | 15.56 |
| 220 | V11I, C38A, K53A, C76A, H109A, C127A | 19.5 |
| 221 | V11I, C38A, K53A, C76A, D110A, C127A | 2.02 |
| 222 | K8R, V11I, C38A, C76A, Q103E, C127A | 2.01 |
| 223 | K8E, V11I, C38A, C76A, Q103R, C127A | 2.3 |
| 224 | V11I, C38A, C76A, Q103K, C127A | 1.5 |
| 225 | V11I, C38A, S55H, C76A, C127A | 3.14 |
| 226 | V11I, C38A, S55R, C76A, C127A | 1.91 |
| 227 | V11I, C38A, S55T, C76A, C127A | 4.73 |
| 228 | V11I, C38A, C76A, S105I, C127A | 5.37 |
| 229 | V11I, C38A, C76A, S105K, C127A | 7.73 |
| 230 | K8L, E6K, V11I, C38A, K53A, T63A, C76A, C127A | 0.29 |
| 231 | E6K, V11I, C38A, I49E, K53A, T63A, C76A, C127A | |
| 232 | E6K, V11I, C38A, I49M, K53A, T63A, C76A, C127A | 0.07 |
| 233 | E6K, V11I, C38A, I49R, K53A, T63A, C76A, C127A | 0.04 |
| 234 | E6K, V11I, C38A, K53A, T63A, C76A, Q103R, C127A | 0.26 |
| 235 | E6K, K8E, V11I, C38A, K53A, T63A, C76A, Q103R, | 0.4 |
| | C127A | |
| 236 | E6K, V11I, C38A, K53A, T63A, C76A, C127A, V153R | |
| 237 | E6K, V11I, C38A, K53A, T63A, C76A, C127A, V153E | 0.1 |
| 238 | E6K, V11I, C38A, K53A, T63A, C76A, C127A, | 0.08 |
| | V153Y | |
| 239 | E6K, V11I, C38A, M51G, K53A, T63A, C76A, C127A | 0.1 |
| 240 | E6R, V11I, C38A, K53A, T63A, C76A, C127A | 0.04 |
| 241 | E6K, V11I, C38A, K53A, T63A, C76A, C127A | 2.5 |
| 242 | E6K, V11I, C38A, K53A, T63A, C76A, C127A | 1.68 |
| 243 | Y1M, E6K, V11I, C38A, K53A, T63A, C76A, C127A | 0.02 |
| 244 | E6K, V11I, C38A, M51G, K53A, T63A, C76A, C127A | 13.99 |

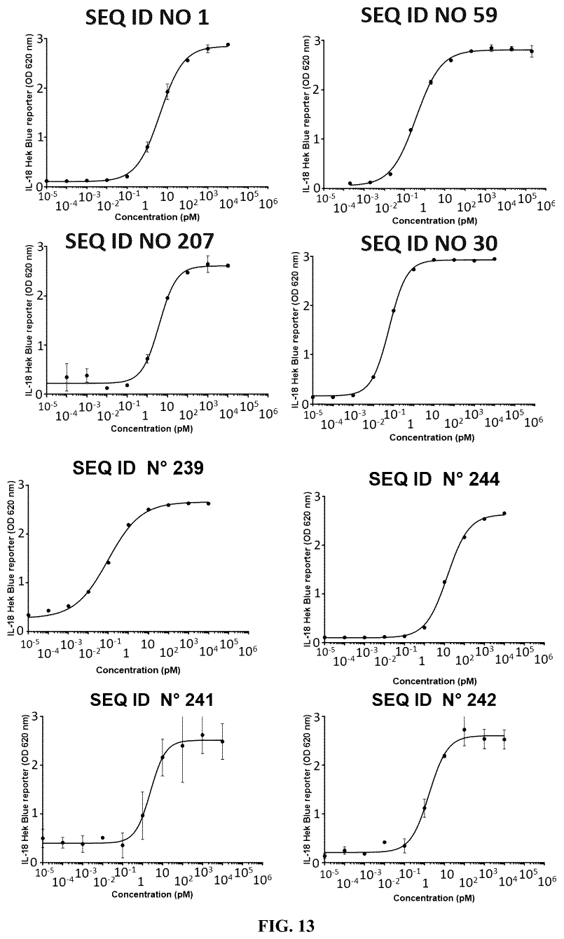

FIG. 13 shows plots measuring ability of wild type IL-18 and of modified IL-18 polypeptides to induce the NF-κB/AP-1-inducible secreted embryonic alkaline phosphatase (SLAP) reporter gene in Hek Blue cells expressing the IL-18 receptor. The figure shows mean SLAP reporter signal (GD 620 nm) on the y-axis, and dosage of the IL-18 polypeptides on the x-axis. The IL-18 polypeptides are native IL-18 wild-type (SEQ ID NO: 01), SEQ ID NO: 57, SEQ ID NO: 59, SEQ ID NO: 207, SEQ ID NO: 239, SEQ ID NO: 241, SEQ ID NO: 242, and SEQ ID NO: 244.

### Example 14—Pharmacokinetic and Pharmacodynamic Properties of Modified IL-18 Polypeptide Variants

The pharmacokinetic (PK) and pharmacodynamic (PD) properties of select IL-18 polypeptide variants are measured. Three MC38 colon carcinoma tumor bearing C57BL/6 mice are tested per group and per time point. IL-18 variants are applied via single intravenous injections at 3 mg/kg.

Study included the following time points (5 min, 1h, 2 h, 6h, 12 h, 24h, 48h, 72h, 96h, 120h, 168h, 240h) with 3 mice sampled per time point. At indicated time points, blood samples were collected in the presence of EDTA either via tail vein sampling or via cardiac puncture (end-point). Blood samples were immediately processed by centrifugation. Plasma samples were cryopreserved at −80° C. until bioanalysis.

Immune-related PD effects are determined by analyzing cytokine levels in plasma and tumors. A pre-configured kit (Th1/Th2 Cytokine & Chemokine 20-Plex Mouse ProcartaPlex™ Panel 1; EPX200-26090-901; Thermofisher) was used to analyze the following mouse cytokines: Eotaxin (CCL11), GRO alpha (CXCL1), GM-CSF, IFN gamma, IL-1 beta, IL-2, IL-4, IL-5, IL-6, IL-12p70, IL-13, IL-18, TNF alpha, IP-10 (CXCL10), MCP-1 (CCL2), MCP-3 (CCL7), MIP-1 alpha (CCL3), MIP-1 beta (CCL4), MIP-2, and RANTES (CCL5).

Each survival plasma sample were measured in singlicate and each terminal plasma sample were measured in duplicate (25 μl plasma per well, 132 wells total over two plates including standards) and expression levels determined per analyte using standard curves. The following plasma cytokines ae measured: IFNγ, and TNFα. The activation status of leukocytes was determined by monitoring surface markers: CD25, CD69, and Fas.

Bioanalysis is conducted by detecting the total amount of IL-18 variants (free and IL-18BP-complexed). Corning high-binding half-area plates (Fisher Scientific, Reinach, Switzerland) are coated overnight at 4° C. with 25 μl of anti-IL18 monoclonal antibody (MBL, cat #D043-3, Clone 25-2G) at 2 μg/ml in PBS. Plates are then washed four times with 100 μl of PBS-0.02% Tween20. Plates surfaces are blocked with 25 μl of PBS-0.02% Tween20-1% BSA at 37° C. during 1h. Plates are then washed four times with 100 μl of PBS-0.02% Tween20. Twenty-five microliters of IL-18 variants (or of mouse plasma) are added in eight-fold serial dilutions starting at 50 nM down to 0.02 nM into PBS-0.02% Tween20-0.1% BSA and incubated at 37° C. during 2h. Plates are then washed four times with 100 μl of PBS-0.02% Tween20 and 25 μl of biotinylated anti-IL18 monoclonal antibody (MBL, cat #D045-6, Clone 159-12B) at 2 μg/ml in PBS. Plates are incubated during 2 h at 37° C. and ae then washed four times with 100 μl of PBS-0.02% Tween20. Twenty-five microliters of Streptavidin- Horseradish peroxidase (#RABHRP3, Merck, Buchs, Switzerland) diluted at 1:500 into PBS-0.02% Tween20-0.1% BSA are added to each well and incubated at Room Temperature during 30 min. Plates are then washed four times with 100 μl of PBS-0.02% Tween20. Fifty microliters of TMB substrate reagent (#CL07, Merck, Buchs, Switzerland) are added to each well and incubated at 37° C. during 5 min. After 5 min at 37° C., Horseradish peroxidase reaction is stopped by adding 50 μl/well of 0.5M H2SO4 stop solution. ELISA signal is then measured at 450 nm on an EnSpire plate reader from Perkin Elmer (Schwerzenbach, Switzerland)

FIG. 16A shows a plot describing plasma exposure of modified IL-18 polypeptides in C57BL/6 mice implanted with MC38 syngeneic colon carcinoma tumors. The figure shows plasma concentrations on the y-axis, and time on the x-axis. The IL-18 polypeptides tested in this figure are SEQ ID NO: 57 and SEQ ID NO: 57 conjugated to a 30 kDa PEG tested as a single agent at 3 mg/kg as a single i.v. injection. (n=3 per time point; mean±SEM).

FIG. 16B shows a plot describing the effect of modified IL-18 polypeptides on levels of IFNgamma and TNFalpha in blood of C57BL/6 mice implanted with MC38 syngeneic colon carcinoma tumors. The figure shows plasma concentrations of IFNg and TNFα on the y-axis, and time on the x-axis. The IL-18 polypeptides tested in this figure are SEQ ID NO: 57 and SEQ ID NO: 57 conjugated to a 30 kDa PEG tested as a single agent at 3 mg/kg as a single i.v. injection. (n=3 per time point; mean±SEM).

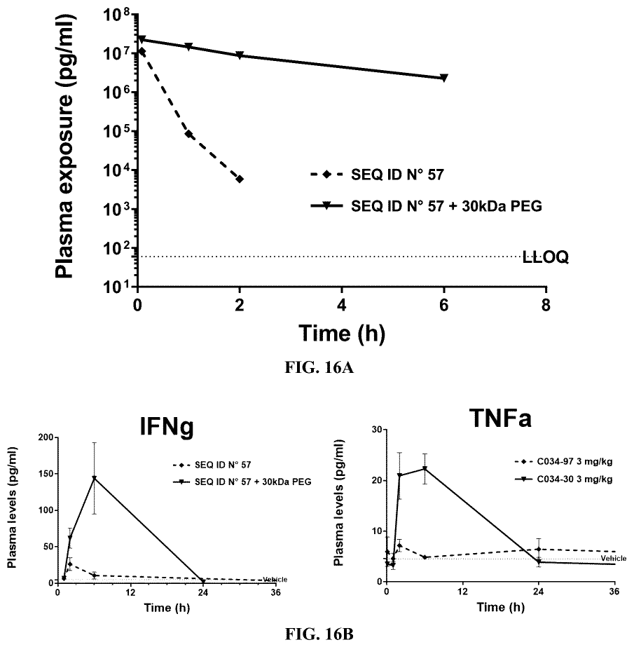

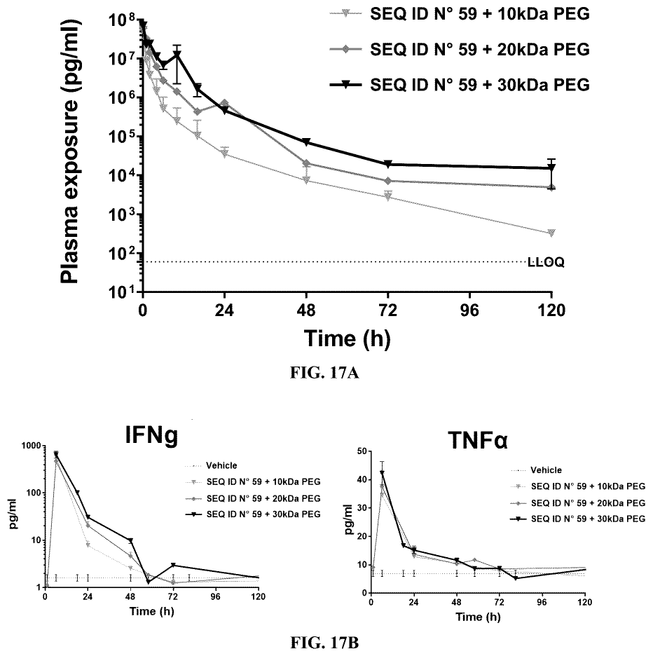

FIG. 17A shows a plot describing plasma exposure of modified IL-18 polypeptides in C57BL/6 mice implanted with MC38 syngeneic colon carcinoma tumors. The figure shows plasma concentrations on the y-axis, and time on the x-axis. The IL-18 polypeptides tested in this figure are SEQ ID NO: 59 and SEQ ID NO: 59 conjugated to a 10, 20 and 30 kDa PEG tested as a single agent at 3 mg/kg as a single i.v. injection. (n=3 per time point; mean±SEM).

FIG. 17B shows a plot describing the effect of modified IL-18 polypeptides on levels of IFNgamma and TNFalpha in blood of C57BL/6 mice implanted with MC38 syngeneic colon carcinoma tumors. The figure shows plasma concentrations of IFNg and TNFα on the y-axis, and time on the x-axis. The IL-18 polypeptides tested in this figure are SEQ ID NO: 59 and SEQ ID NO: 59 conjugated to a 10, 20 and 30 kDa tested as a single agent at 3 mg/kg as a single i.v. injection. (n=3 per time point; mean±SEM).

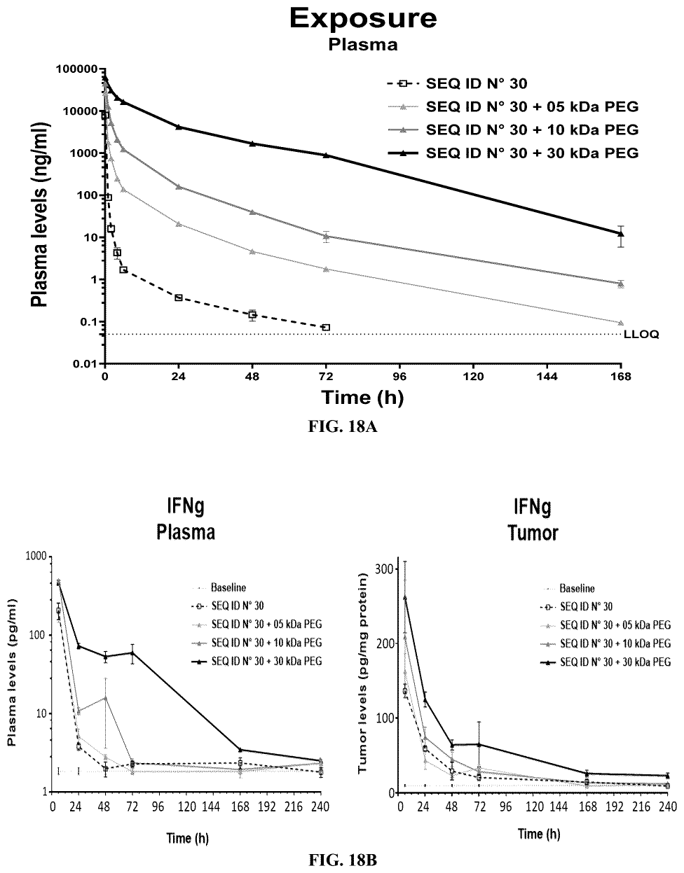

FIG. 18A shows a plot describing plasma exposure of modified IL-18 polypeptides in C57BL/6 mice implanted with MC38 syngeneic colon carcinoma tumors. The figure shows plasma concentrations on the y-axis, and time on the x-axis. The IL-18 polypeptides tested in this figure are SEQ ID NO: 30 and SEQ ID NO: 30 conjugated to a 05, 10 and 30 kDa PEG tested as a single agent at 3 mg/kg as a single i.v. injection. (n=3 per time point; mean±SEM).

FIG. 18B shows a plot describing the effect of modified IL-18 polypeptides on levels of IFNgamma in blood (left panel) and tumors (right panel) of C57BL/6 mice implanted with MC38 syngeneic colon carcinoma tumors. The figure shows plasma concentrations of IFNg and TNFα on the y-axis, and time on the x-axis. The IL-18 polypeptides tested in this figure are SEQ ID NO: 30 and SEQ ID NO: 30 conjugated to a 05, 10 and 30 kDa tested as a single agent at 3 mg/kg as a single i.v. injection. (n=3 per time point; mean±SEM).

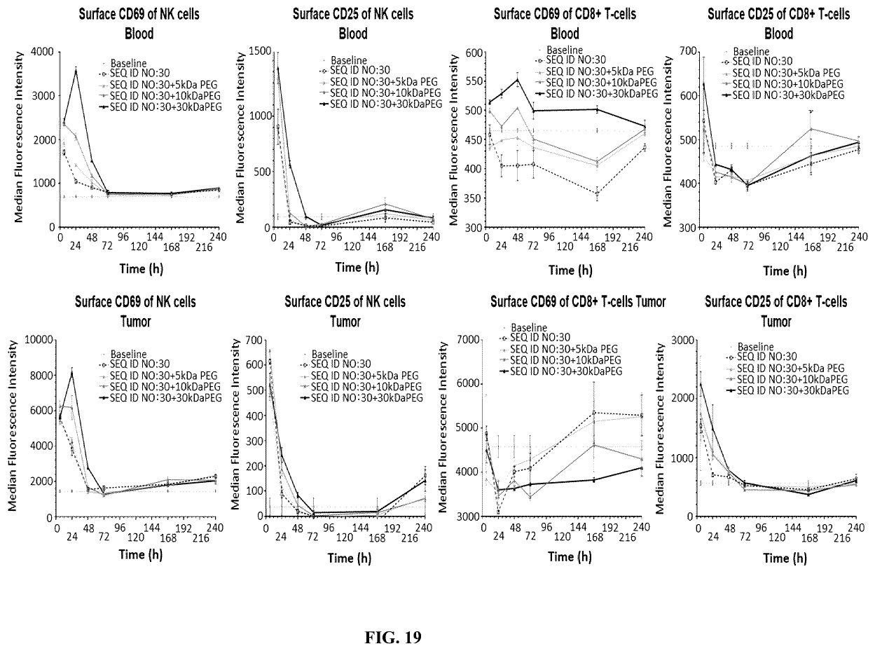

FIG. 19 shows a plot describing the effect of modified IL-18 polypeptides on activation status of NK and CD8⁺ T-cells in blood (upper panel) and tumors (bottom panel) of C57BL/6 mice implanted with MC38 syngeneic colon carcinoma tumors. The figure surface expression levels (Median Fluorescence Intensity) on the y-axis, and time on the x-axis. The IL-18 polypeptides tested in this figure are SEQ ID NO: 30 and SEQ ID NO: 30 conjugated to a 05, 10 and 30 kDa tested as a single agent at 3 mg/kg as a single i.v. injection. (n=3 per time point; mean±SEM).

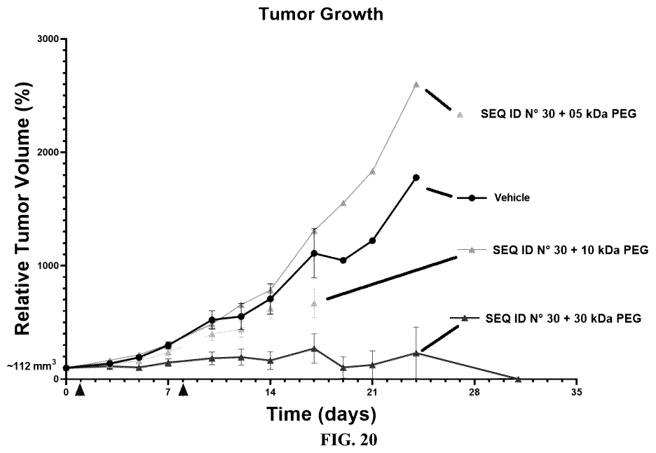

Tumor growth inhibition of IL-18 variants SEQ ID NO: 30 and SEQ ID NO: 30 conjugated to a 05, 10 and 30 kDa, were determined. The IL-18 variants were administered via single intravenous injections, at dosage 3 mg/kg to mice having MC38 tumor. FIG. 20 shows the variant with the 30 kDa PEG group shows superior tumor growth inhibition properties compared to the 5 and 10 kDa variants tested.

### Example 15—In Vivo Antitumor Activity in MC38 Colon Carcinoma Model

In vivo efficacy study were performed in mice. Naïve, 6-8 weeks old, C57BL/6 female mice were inoculated subcutaneously at the right upper flank with MC38 tumor cells (3×10⁵) in 0.1 mL of PBS for tumor development. The animals were randomized (using an Excel-based randomization software performing stratified randomization based upon tumor volumes), and treatment started when the average tumor volume reached 70-90 mm³. Animals treated with modified IL-18 polypeptides received two 10 mL/kg bolus intravenous (i.v.) injections of 0.5, 2.5, 3, 7 or 10 mg/kg of modified IL-18 polypeptides. Animals treated with anti-PD-1 antibody received 10 mL/kg bolus intraperitoneal (i.p.) injections of 2 mg/kg of InVivoMAb anti-mouse PD-1/CD279 (Clone RMP1-14; BioXcell; Cat #BE0146) twice per week over two weeks. Finally, animals treated with a combination of 30 kDa pegylated SEQ ID NO 30 and anti-PD-1 antibody received a single 10 mL/kg bolus intravenous (i.v.) injection of 2.5 mg/kg of modified IL-18 polypeptides on day 0, 7 and 10 ml/kg bolus intraperitoneal (i.p.) injections of 2 mg/kg of InVivoMAb anti-mouse PD-1/CD279 (Clone RMP1-14; BioXcell; Cat #BE0146) twice per week over two weeks. After inoculation, the animals were checked daily for morbidity and mortality. At the time, animals were checked for effects on tumor growth and normal behavior such as mobility, food and water consumption, body weight gain/loss (body weights were measured twice weekly), eye/hair matting and any other abnormal effect. The major endpoints were delayed tumor growth or complete tumor regression. Tumor sizes were measured three times a week in two dimensions using a caliper, and the volume was expressed in mm³ using the formula: V=0.5 a×b² where a and b are the long and short diameters of the tumor, respectively. Death and observed clinical signs were recorded on the basis of the numbers of animals within each subset.

A re-challenge study was performed on tumor-free animals. 77 days after start of treatment, animals that showed complete tumor regression were enrolled in a re-challenge study to probe the establishment of a long-lasting immunological memory response. Briefly, twelve naïve non-treated control animals, two animal previously treated with 30 kDa pegylated SEQ ID NO 30 at 2.5 mg/kg, five animals previously treated with 30 kDa pegylated SEQ ID NO 30 at 10 mg/kg, three animals previously treated with anti-PD1 antibody at 2 mg/kg, and twelve animals previously treated with 30 kDa pegylated SEQ ID NO 30 at 2.5 mg/kg in combination with anti-PD1 antibody at 2 mg/kg were inoculated subcutaneously at the left lower flank with MC38 tumor cells (3×10⁵) in 0.1 mL of PBS. At the time of routine monitoring, animals were checked for effects on tumor growth and normal behavior such as mobility, food and water consumption, body weight gain/loss (body weights were measured twice weekly), eye/hair matting and any other abnormal effect. The major endpoints were delayed tumor growth or tumor graft rejection. Tumor sizes were measured twice a week in two dimensions using a caliper, and the volume was expressed in mm³ using the formula: V=0.5 a×b² where a and b are the long and short diameters of the tumor, respectively. Death and observed clinical signs were recorded on the basis of the numbers of animals within each subset.

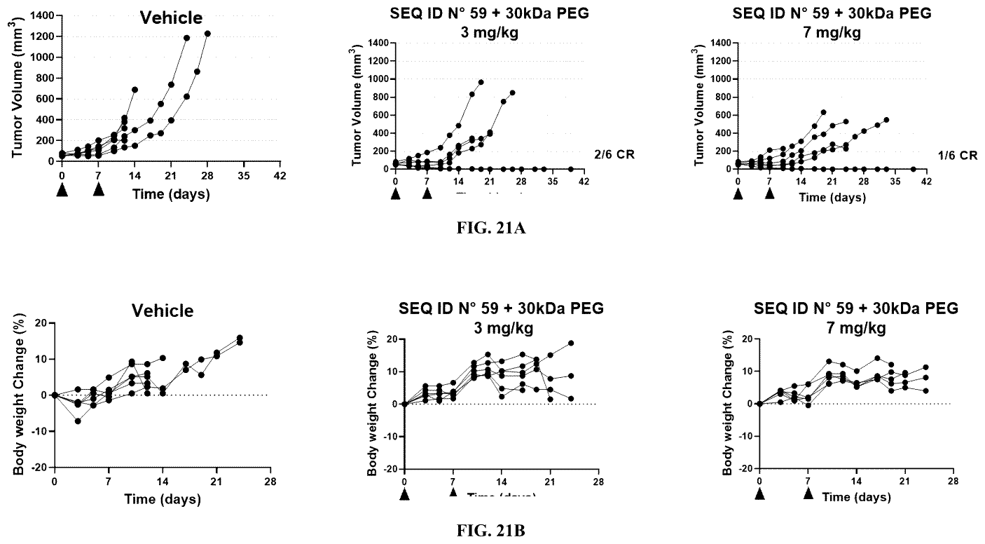

FIG. 21A shows a plot describing the effect of modified IL-18 polypeptides on the growth of MC38 syngeneic colon carcinoma tumors in C57BL/6 mice. The figure shows tumor volume of each individual mouse on the y-axis and time on the x-axis. The IL-18 polypeptides tested in this figure is SEQ ID NO: 59 conjugated to a 30 kDa PEG tested as a single agent at 3 and 7 mg/kg as two weekly i.v. injections. (n=6; mean±SEM).

FIG. 21B shows a plot describing the effect of modified IL-18 polypeptides on the weight C57BL/6 mice engrafted with MC38 syngeneic colon carcinoma tumors. The figure shows weight of each individual mouse on the y-axis and time on the x-axis. The IL-18 polypeptides tested in this figure is SEQ ID NO: 59 conjugated to a 30 kDa PEG tested as a single agent at 3 and 7 mg/kg as two weekly i.v. injections. (n=6; mean±SEM).

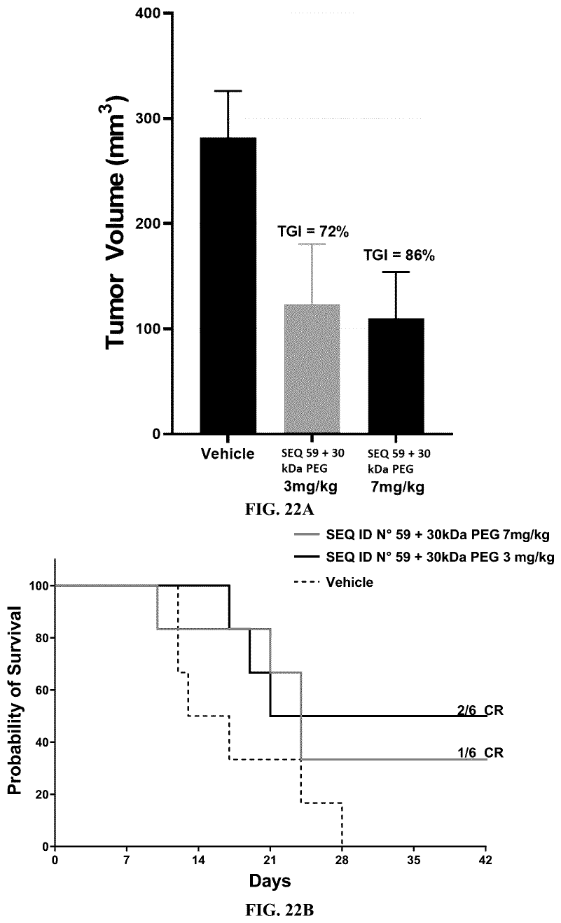

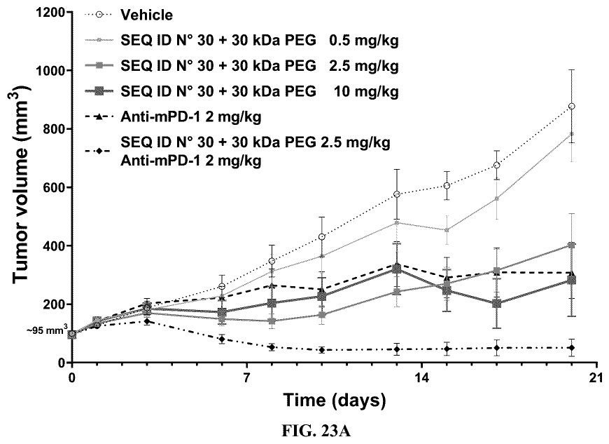

FIG. 22A shows a plot describing the effect of modified IL-18 polypeptide on the growth of MC38 syngeneic colon carcinoma tumors in C57BL/6 mice. The figure shows the mean tumor volume on day 12 post treatment initiation on the y-axis. The modified IL-18 polypeptide tested in this figure is SEQ ID NO: 59 conjugated to a 30 kDa PEG tested as a single agent at 3 and 7 mg/kg as two weekly i.v. injections. (n=6; mean±SEM; TGI: Tumor Growth Inhibition).

FIG. 22B shows a plot describing the effect of modified IL-18 polypeptide on the survival of MC38 syngeneic colon carcinoma tumor-bearing C57BL/6 mice The modified IL-18 polypeptide tested in this figure is SEQ ID NO: 59 conjugated to a 30 kDa PEG tested as a single agent at 3 and 7 mg/kg as two weekly i.v. injections. (n=6; CR: Complete response).

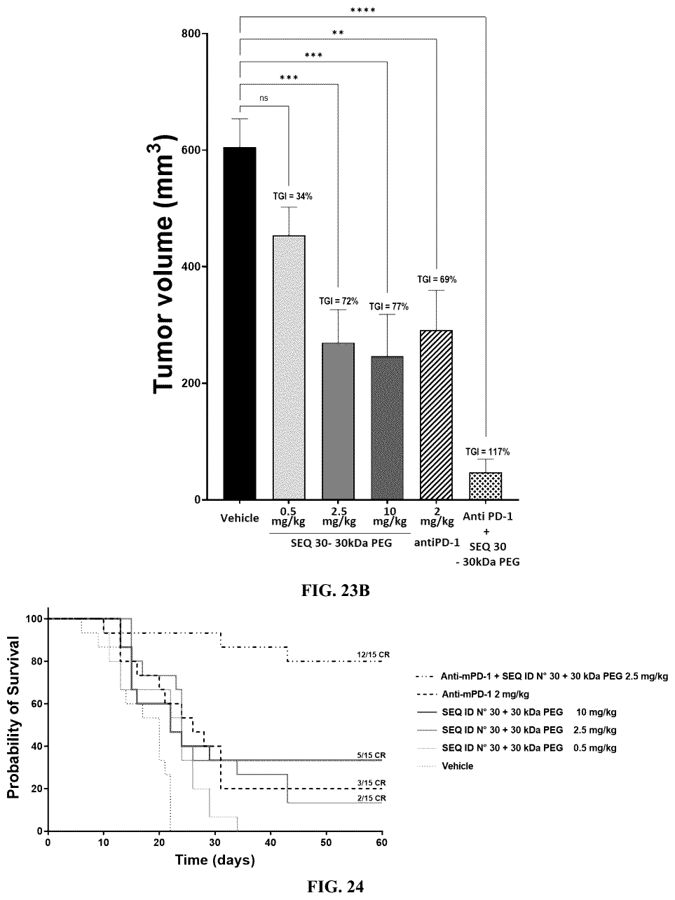

FIG. 23A shows a plot describing the effect of modified IL-18 polypeptides on the growth of MC38 syngeneic colon carcinoma tumors in C57BL/6 mice. The figure shows mean tumor volume on the y-axis and time on the x-axis. The IL-18 polypeptide tested in this figure is SEQ ID NO: 30 conjugated to a 30 kDa PEG tested as a single agent at 0.5, 2.5, and 10 mg/kg as two weekly i.v. injections. As a control, anti PD-1 was applied at 2 mg/kg as a single agent as six biweekly i.v. injections for two weeks. SEQ ID NO: 30 conjugated to a 30 kDa PEG was also tested at 2.5 mg/kg in combination with anti-PD1 antibody at 2 mg/kg (n=15; mean±SEM).

FIG. 23B shows a plot describing the effect of modified IL-18 polypeptides on the growth of MC38 syngeneic colon carcinoma tumors in C57BL/6 mice. The figure shows the mean tumor volume on day 15 post treatment initiation on the y-axis. The IL-18 polypeptide tested in this figure is SEQ ID NO: 30 conjugated to a 30 kDa PEG tested as a single agent at 0.5, 2.5, and 10 mg/kg as two weekly i.v. injections. As a control, anti PD-1 was applied at 2 mg/kg as a single agent as six biweekly i.v. injections for two weeks. SEQ ID NO: 30 conjugated to a 30 kDa PEG was also tested at 2.5 mg/kg in combination with anti-PD1 antibody at 2 mg/kg (n=15; One-way Anova test *** P-value<0.001, ** P-value<0.01, * P-value<0.1, ns non significant, TGI: Tumor Growth Inhibition).

FIG. 24 shows a plot describing the effect of modified IL-18 polypeptide on the survival of MC38 syngeneic colon carcinoma tumor-bearing C57BL/6 mice. The IL-18 polypeptide tested in this figure is SEQ ID NO: 30 conjugated to a 30 kDa PEG tested as a single agent at 0.5, 2.5, and 10 mg/kg as two weekly i.v. injections. As a control, anti PD-1 was applied at 2 mg/kg as a single agent as six biweekly i.v. injections for two weeks. SEQ ID NO: 30 conjugated to a 30 kDa PEG was also tested at 2.5 mg/kg in combination with anti-PD1 antibody at 2 mg/kg (n=15; CR: Complete response).

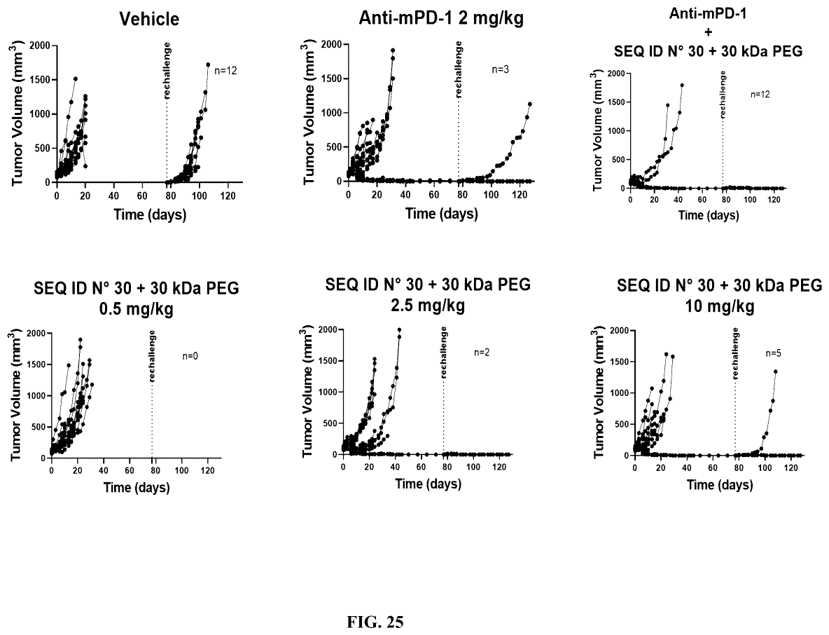

FIG. 25 shows a plot describing the effect of modified IL-18 polypeptides on the growth of MC38 syngeneic colon carcinoma tumors in C57BL/6 mice. The figure shows tumor volume of each individual mouse on the y-axis and time on the x-axis. The IL-18 polypeptide tested in this figure is SEQ ID NO: 30 conjugated to a 30 kDa PEG tested as a single agent at 0.5, 2.5, and 10 mg/kg as two weekly i.v. injections. As a control, anti PD-1 was applied at 2 mg/kg as a single agent as six biweekly i.v. injections for two weeks. SEQ ID NO: 30 conjugated to a 30 kDa PEG was also tested at 2.5 mg/kg in combination with anti-PD1 antibody at 2 mg/kg

### Example 16—In Vivo Studies in NHPs

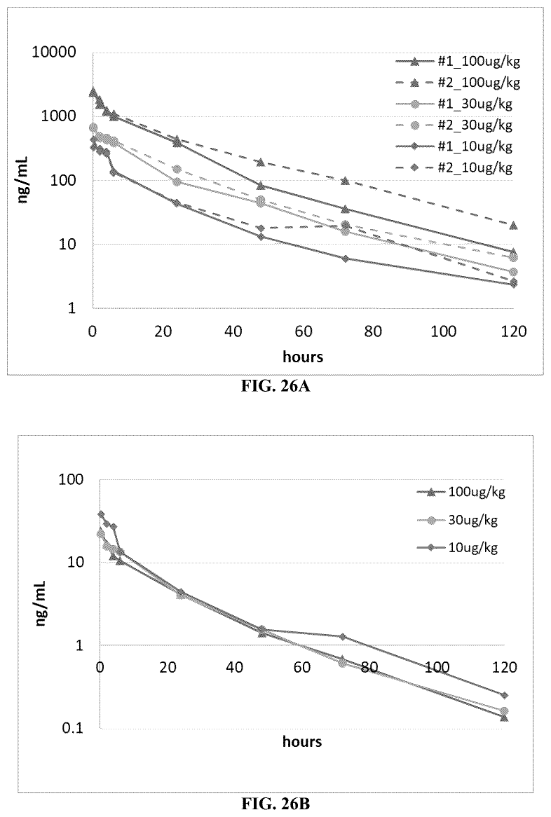

A PK/PD study was performed in non-human primates (NHPs) (2 animals per dosage) to ascertain the PK distribution and other effects of IL-18 variant of SEQ ID NO: 30+30 kDa PEG. Dose dependent plasma levels of IL-18 variant were studied. The IL-18 variant was administered to NHPs at dosage 10 μg/kg, 30 μg/kg, and 100 μg/kg via single intravenous injections. FIG. 26A shows individual PK, and FIG. 26B shows dose normalized average PK over 1 to 120 hrs post administration. The PK parameters are listed in Table 13. Low individual to individual variability was observed, with dosage correlated with linear PK levels over time. The half-life/mean residence time was observed to be approximately 1 day.

TABLE 13
PK parameters of SEQ ID NO: 30 + 30 kD PEG in NHPs

| | AUC_ | | | | | | |
|---|---|---|---|---|---|---|---|
| | INF_obs | AUC_INF_ | | Cl_obs | Vss_obs | HL_Lamb | MRT_INF_ |
| Dose | (h · ng · m | D_obs | AUC_% E | (mL · h⁻¹ | (mL · k | da_z | obs |
| (μg/kg) | L⁻¹) | (h · mL⁻¹kg⁻¹) | xtrap | kg⁻¹) | g⁻¹) | (h) | (h) |
| 10 | 4145 | 0.41 | 1% | 2.4 | 52 | 23.7 | 21.7 |
| 10 | 4690 | 0.47 | 1% | 2.1 | 58 | 24.7 | 27.3 |
| 30 | 9329 | 0.31 | 1% | 3.2 | 71 | 20.3 | 22.2 |
| 30 | 11215 | 0.37 | 0% | 2.7 | 66 | 21.2 | 24.6 |
| 100 | 27351 | 0.27 | 0% | 3.7 | 71 | 17.6 | 19.4 |
| 100 | 35629 | 0.36 | 0% | 2.8 | 76 | 23.8 | 27.1 |

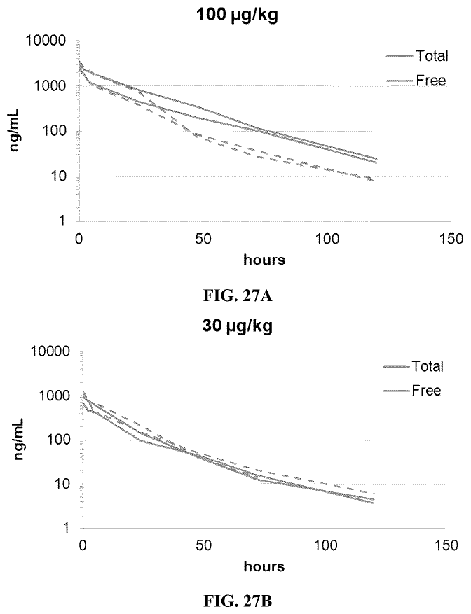

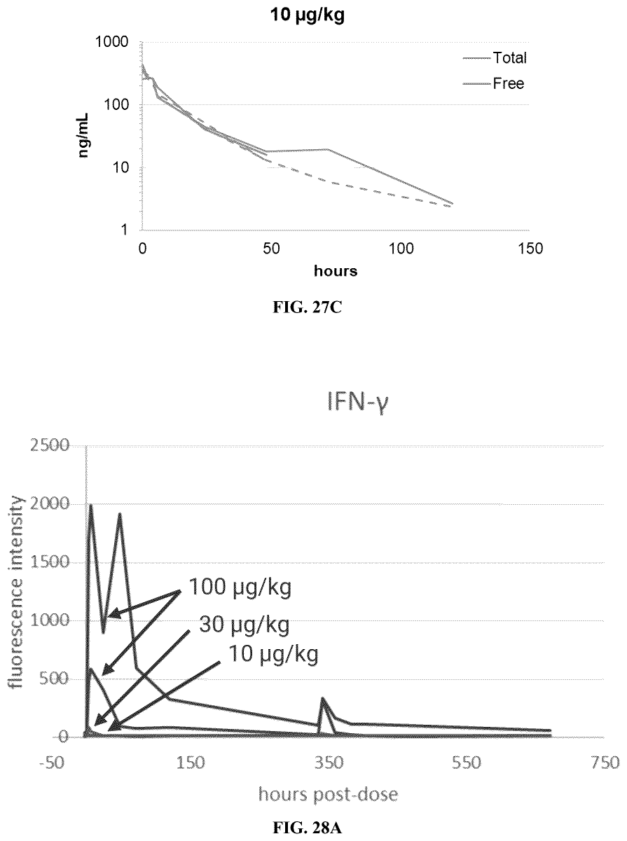

From the same study, binding to the IL-18 variant with IL-18 Binding protein in plasma were studiedPlasma levels of free IL-18 variant and total IL-18 variant (free IL-18+IL-18 complexed with IL8BP) were determined over 0 to 150 hours post administration and can bee senn in FIG. 27A (10 tag/kg), FIG. 27B (30 tag/kg), and FIG. 27C (100 rag/kg). As can be seen from FIGS. 27A-C, levels of free and total IL-18 variant are superimposable, thus indicating that the IL-18 variant tested is resistant to IL-18BP systemic levels in NHPs.

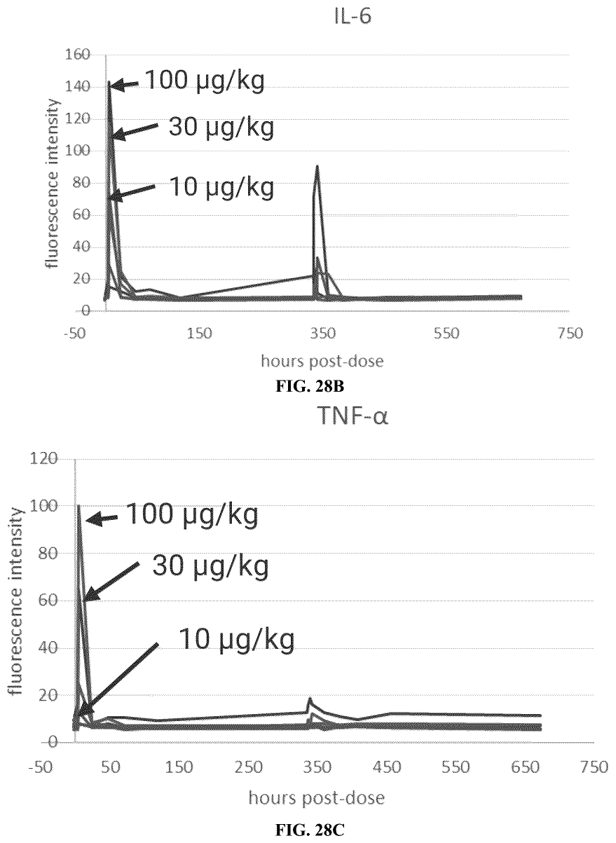

From the same study, levels of IFNγ, IL-6, and TNFα in plasma were studied. FIGS. 28A, B and C show levels of IFNγ, IL-6, and TNFα respectively in plasma, 0 to 650 hours post administration. A second dosage was administered shortly before the 350 hour time point. The IL-18 variant induces release of pro-inflammatory cytokines in blood and tumors, induces strong and sustained release of IFNγ after first administration (FIG. 28A), and measurable but less durable release of IL-6 and TNFα (FIGS. 28B and 28C). No effect on IL-12p70 and IP-10 was observed (data not shown). The effects were diminished following the second administration, suggesting a possible effect of anti-drug antibody onset.

### Example 17—PBMC Stimulation Assay

Ability of IL-18 variants to stimulate peripheral blood mononuclear cells (PBMCs) for IFNγ secretion is assessed according to the following protocol.

Isolation of lymphocytes: Blood from Buffy Coats of healthy volunteers is diluted with equal volume of PBS and slowly poured on top of SepMate tube prefilled with 15 mL Histopaque-1077. Tubes are centrifuged for 10 minutes at 1200 g, the top layer is collected and washed 3 times with PBS containing 2% of Fetal Bovine Serum. PBMCs are counted and cryopreserved as aliquots of 20×10⁶ cells.

Cryopreserved PBMCs are thawed and stimulated with gradient of human IL-18 variants ranging from 0.2 pM to 1 □M in RPMI containing 10% Fetal Bovine Serum.

Cytokine production after 24 hr stimulation is measured using Legendplex bead-based cytokine assay (Biolegend #740930) following manufacturer instructions. Half maximal effective concentrations (EC₅₀) of IFN□ released in culture supernatant are calculated based on a variable slope and four parameter analysis using GraphPad PRISM software.

Surface expression of FcγRIII on NK cells is measured by flow cytometry (Mouse IgG1 clone 3G8) after 72 hr stimulation.

TABLE 11
EC₅₀ in IFNγ secretion assay in PBMCs

| SEQ ID | | EC50 |
|---|---|---|
| NO: | Sequence modifications | (nM) |
| 1 | Native sequence | 0.698 |
| 8 | E6K, V11I, C38A, K53A, T63A, C68A, C76A, T95C, C127A | 0.0086 |
| 11 + | E6K, V11I, C38A, K53A, C76A, C127A, C68-30kPEG | NT |
| PEG | | |
| 11 | E6K, V11I, C38A, K53A, C76A, C127A | 0.0052 |
| 6 + | E6K, V11I, C38A, K53A, T63A, C68A, C76A, C127A, D98C- | 0.177 |
| PEG | 30kPEG | |
| 6 | E6K, V11I, C38A, K53A, T63A, C68A, C76A, C127A, D98C | 0.196 |
| 5 + | E6K, V11I, C38A, K53A, T63A, C68A, C76A, C127A, M86C- | 0.149 |
| PEG | 30kPEG | |
| 5 | E6K, V11I, C38A, K53A, T63A, C68A, C76A, C127A, M86C | 0.013 |
| 9 + | E6K, C38A, K53A, C68A, D98C-30kPEG | 0.340 |
| PEG | | |
| 9 | E6K, C38A, K53A, C68A, D98C | 0.027 |
| 4 + | E6K, C38A, K53A, C68A, M86C-30kPEG | 0.203 |
| PEG | | |
| 4 | E6K, C38A, K53A, C68A, M86C | 0.035 |
| 28 + | E6K, C38A, K53A, T63A, C76A, C127A, C68-30kPEG | 0.0693 |
| PEG | | |
| 28 | E6K, C38A, K53A, T63A, C76A, C127A | 0.0019 |
| 30 + | E6K, V11I, C38A, K53A, T63A, C76A, C127A, C68-30kPEG | 0.0960 |
| PEG30 kDa | | |
| 30 + | E6K, V11I, C38A, K53A, T63A, C76A, C127A, C68-20kPEG | 0.0332 |
| PEG20 kDa | | |
| 30 + | E6K, V11I, C38A, K53A, T63A, C76A, C127A, C68-10kPEG | 0.0407 |
| PEG10 kDa | | |
| 30 + | E6K, V11I, C38A, K53A, T63A, C76A, C127A, C68-5kPEG | 0.0203 |
| PEG5 kDa | | |
| 30 | E6K, V11I, C38A, K53A, T63A, C76A, C127A | 0.0021 |
| 63 | E6K, C38A, K53A, C76A, C127A | 0.034 |
| 62 + | E6K, C38A, K53A, C127A, C68-30kPEG | NT |
| PEG | | |
| 62 | E6K, C38A, K53A, C127A | 0.074 |
| 61 + | E6K, C38A, K53A, C76A, C68-30kPEG | NT |
| PEG | | |
| 61 | E6K, C38A, K53A, C76A | 0.004 |
| 60 + | E6K, C38Q, K53A, C68-30kPEG | 0.330 |
| PEG | | |
| 60 | E6K, C38Q, K53A | 0.033 |
| 59 + | E6K, C38A, K53A, C68-30kPEG | 0.196 |
| PEG30 | | |
| 59 + | E6K, C38A, K53A, C68-20kPEG | 0.220 |
| PEG20 | | |
| 59 + | E6K, C38A, K53A, C68-10kPEG | 0.111 |
| PEG10 | | |
| 59 | E6K, C38A, K53A | 0.036 |
| 57 + | E6K, C38S, K53A, C68-30kDPEG | 7.014 |
| PEG | | |
| 57 | E6K, C38S, K53A | 2.580 |

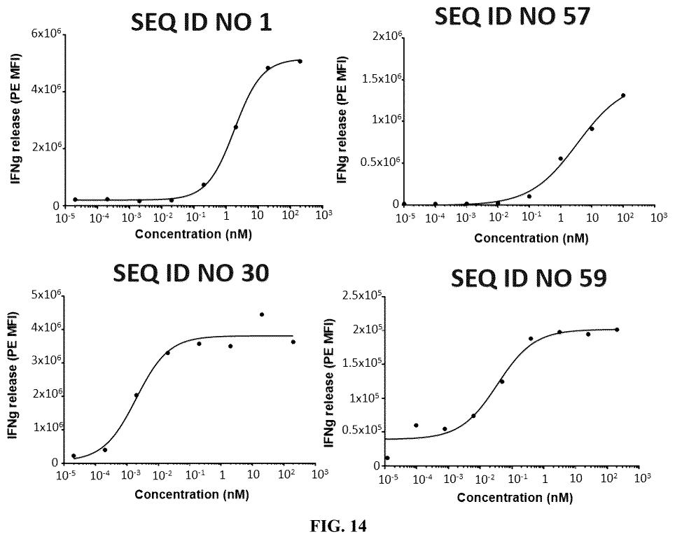

FIG. 14 shows plots measuring ability of wild type IL-18 and of modified IL-18 polypeptides to stimulate the secretion of IFNgamma by human Peripheral Blood Mononuclear Cells (PBMCs). The figure shows mean IFNγ signal on the y-axis and dosage of the IL-18 polypeptides on the x-axis. The IL-18 polypeptides are native IL-18 wild-type (SEQ ID NO: 01), SEQ ID NO: 57, 30 and 59.

FIGS. 15A-B show effect of attaching 30 kDa PEG to IL-18 variants on IL-18 mediated IFNγ secretion in human peripheral blood mononuclear cells. EC50 for IL-18 mediated IFNγ secretion for IL-18 of SEQ ID NO: 59, 4 and 9 (with and without 30 kDa PEG) is shown in FIG. 15A; and SEQ ID NO: 30, 5 and 6 (with and without 30 kDa PEG) is shown in FIG. 32B.

Although the present disclosure and its advantages have been described in detail, it should be understood that various changes, substitutions and alterations can be made herein without departing from the spirit and scope of the disclosure as defined in the appended claims.

1. A modified interleukin 18 (IL-18) polypeptide, wherein the modified IL-18 polypeptide comprises a substitution at residue V11 and a polymer covalently attached to residue 68, 69, or 70 of the modified IL-18 polypeptide, wherein residue position numbering is based on SEQ ID NO: 1 as a reference sequence.

2. The modified IL-18 polypeptide of claim 1, wherein the modified IL-18 polypeptide comprises a substitution at residue T63, K53, E6, or a combination thereof.

3. (canceled)

4. (canceled)

5. (canceled)

6. The polymer modified IL-18 polypeptide of claim 1, wherein the polymer is attached at residue 68 of the modified IL-18 polypeptide.

7. (canceled)

8. The modified IL-18 polypeptide of claim 1, wherein the modified IL-18 polypeptide exhibits a half-maximal effective concentration (EC50) to induce IFNγ production in a cell which is at least 4-fold lower than the EC50 for wild type IL-18 (WT IL-18).

9. (canceled)

10. (canceled)

11. (canceled)

12. (canceled)

13. The modified IL-18 polypeptide of claim 1, wherein the polymer has a weight average molecular weight of from about 5 kDa to about 50 kDa, from about 10 kDa to about 50 kDa, from about 10 kDa to about 40 kDa, from about 10 kDa to about 30 kDa, from about 15 kDa to about 50 kDa, from about 15 kDa to about 40 kDa, from about 15 kDa to about 30 kDa, from about 20 kDa to about 50 kDa, from about 20 kDa to about 40 kDa, from about 20 kDa to about 35 kDa, from about 20 kDa to about 30 kDa, from about 25 kDa to about 50 kDa, from about 25 kDa to about 40 kDa, from about 25 kDa to about 35 kDa, or from about 25 kDa to about 30 kDa.

14. The modified IL-18 polypeptide of claim 1, wherein the polymer is a water-soluble polymer.

15. (canceled)

16. (canceled)

17. The modified IL-18 polypeptide of claim 14, wherein the water-soluble polymer is polyethylene glycol (PEG).

18. (canceled)

19. The modified IL-18 polypeptide of claim 17, wherein the PEG has a molecular weight of from about 5 kDa to about 50 kDa, from about 10 kDa to about 50 kDa, from about 10 kDa to about 40 kDa, from about 10 kDa to about 30 kDa, from about 15 kDa to about 50 kDa, from about 15 kDa to about 40 kDa, from about 15 kDa to about 30 kDa, from about 20 kDa to about 50 kDa, from about 20 kDa to about 40 kDa, from about 20 kDa to about 35 kDa, from about 20 kDa to about 30 kDa, from about 25 kDa to about 50 kDa, from about 25 kDa to about 40 kDa, from about 25 kDa to about 35 kDa, or from about 25 kDa to about 30 kDa.

20. The modified IL-18 polypeptide of claim 17, wherein the polyethylene glycol has a weight average molecular weight of about 10 kDa, about 20 kDa, or about 30 kDa.

21. (canceled)

22. The modified IL-18 polypeptide of claim 1, wherein the modified IL-18 polypeptide comprises at least one substitution at a residue selected from Y1, F2, K8, S10, D17, T34, D35, S36, D37, D40, N41, I49, M51, D54, S55, Q103, S105, G108, H109, D110, and D132.

23. (canceled)

24. (canceled)

25. (canceled)

26. (canceled)

27. (canceled)

28. (canceled)

29. The modified IL-18 polypeptide of claim 1, wherein the modified IL-18 polypeptide comprises a substitution at residue C38, C76, and/or C127.

30. The modified IL-18 polypeptide of claim 29, wherein the modified IL-18 polypeptide comprises a C38A, C38S, C76A, C76S, C127A, and/or C127S substitution.

31. (canceled)

32. (canceled)

33. (canceled)

34. (canceled)

35. The modified IL-18 polypeptide of claim 1, wherein the modified IL-18 polypeptide comprises E6K substitution, V11I substitution, C38A substitution, K53A substitution, T63 Å substitution, C76A substitution, C127A substitution, M51G substitution, or a combination thereof.

36. (canceled)

37. (canceled)

38. (canceled)

39. (canceled)

40. (canceled)

41. (canceled)

42. (canceled)

43. The modified IL-18 polypeptide of claim 1, wherein the modified IL-18 polypeptide comprises an amino acid sequence having at least 80%, 85%, 90%, 95%, 98%, 99%, or 100% sequence identity to the sequence set forth in SEQ ID NO: 30, SEQ ID NO: 207, SEQ ID NO: 239, SEQ ID NO: 241 SEQ ID NO: 242, or SEQ ID NO: 244.

44. The modified IL-18 polypeptide of claim 1, wherein the modified IL-18 polypeptide comprises the sequence set forth in SEQ ID NO: 30.

45. (canceled)

46. (canceled)

47. (canceled)

48. (canceled)

49. (canceled)

50. (canceled)

51. (canceled)

52. (canceled)

53. (canceled)

54. (canceled)

55. The modified IL-18 polypeptide of claim 1, wherein the modified IL-18 polypeptide exhibits a half-maximal effective concentration (EC50) to induce IFNγ production in a cell which is at most 5-fold, 10-fold, 15-fold, 20-fold, 25-fold, 30-fold, 35-fold, 40-fold, 45-fold, 50-fold, 55-fold, 60-fold, 70-fold, 80-fold, 90-fold or 100-fold higher than an identical IL-18 polypeptide with no polymer attached.

56. The modified IL-18 polypeptide of claim 1, wherein the modified IL-18 polypeptide exhibits half maximal effective concentration (EC50) for signaling through the IL-18 receptor which is at least 5-fold, 6-fold, 7-fold, 8-fold, 9-fold, or 10-fold lower than the EC50 for wild type IL-18 (WT IL-18).

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. A pharmaceutical composition comprising:
a) a modified IL-18 polypeptide comprising a substitution at residue V11 and a polymer covalently attached to the modified IL-18 polypeptide; and
b) a pharmaceutically acceptable carrier or excipient.

69. A method of treating cancer in a subject in need thereof, comprising administering to the subject a pharmaceutically effective amount of a modified IL-18 polypeptide comprising a substitution at residue V11 and a polymer covalently attached to residue 68, 69, or 70 of the modified IL-18 polypeptide, wherein residue position numbering is based on SEQ ID NO: 1 as a reference sequence.

70. The method of claim 69, further comprising administering an immune checkpoint inhibitor to the subject.

71-81. (canceled)


SEQUENCE LISTING (244 sequences)

SEQ ID NO: 1 - protein, 157 aa, Homo sapiens
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 2 - protein, 157 aa, synthetic construct
YFGKLKSKLSVIRNLNDQVLFIDQGNRPLFEDMTDSDARDNAPRTIFIISMYADSQPRGMAVTISVKAEKISTLSCENKIISFKCMNPPDNIKDTKSDIIFFQRSVPGHDNKMQFESSSYEGYFLACEKERDLFKLILKKEDELGDRSIMFTVQNED

SEQ ID NO: 3 - protein, 157 aa, synthetic construct
YFGKLKSKLSIIRNLNDQVLFIDQGNRPLFEDMTDSDARDNAPRTIFIISMYADSQPRGMAVAISVKAEKISTLSAENKIISFKCMNPPDNIKDTKSDIIFFQRSVPGHDNKMQFESSSYEGYFLAAEKERDLFKLILKKEDELGDRSIMFTVQNED

SEQ ID NO: 4 - protein, 157 aa, synthetic construct
YFGKLKSKLSVIRNLNDQVLFIDQGNRPLFEDMTDSDARDNAPRTIFIISMYADSQPRGMAVTISVKAEKISTLSCENKIISFKECNPPDNIKDTKSDIIFFQRSVPGHDNKMQFESSSYEGYFLACEKERDLFKLILKKEDELGDRSIMFTVQNED

SEQ ID NO: 5 - protein, 157 aa, synthetic construct
YFGKLKSKLSIIRNLNDQVLFIDQGNRPLFEDMTDSDARDNAPRTIFIISMYADSQPRGMAVAISVKAEKISTLSAENKIISFKECNPPDNIKDTKSDIIFFQRSVPGHDNKMQFESSSYEGYFLAAEKERDLFKLILKKEDELGDRSIMFTVQNED

SEQ ID NO: 6 - protein, 157 aa, synthetic construct
YFGKLKSKLSVIRNLNDQVLFIDQGNRPLFEDMTDSDARDNAPRTIFIISMYADSQPRGMAVAISVKAEKISTLSAENKIISFKEMNPPDNIKDTKSCIIFFQRSVPGHDNKMQFESSSYEGYFLAAEKERDLFKLILKKEDELGDRSIMFTVQNED

SEQ ID NO: 7 - protein, 157 aa, synthetic construct
YFGKLKSKLSVIRNLNDQVLFIDQGNRPLFEDMTDSDARDNAPRTIFIISMYADSQPRGMAVTISVKAEKISTLSAENKIISFKECNPPDNIKDTKSDIIFFQRSVPGHDNKMQFESSSYEGYFLAAEKERDLFKLILKKEDELGDRSIMFTVQNED

SEQ ID NO: 8 - protein, 157 aa, synthetic construct
YFGKLKSKLSIIRNLNDQVLFIDQGNRPLFEDMTDSDARDNAPRTIFIISMYADSQPRGMAVAISVKAEKISTLSAENKIISFKEMNPPDNIKDCKSDIIFFQRSVPGHDNKMQFESSSYEGYFLAAEKERDLFKLILKKEDELGDRSIMFTVQNED

SEQ ID NO: 9 - protein, 157 aa, synthetic construct
YFGKLKSKLSVIRNLNDQVLFIDQGNRPLFEDMTDSDARDNAPRTIFIISMYADSQPRGMAVTISVKAEKISTLSCENKIISFKEMNPPDNIKDTKSCIIFFQRSVPGHDNKMQFESSSYEGYFLACEKERDLFKLILKKEDELGDRSIMFTVQNED

SEQ ID NO: 10 - protein, 157 aa, synthetic construct
YFGKLKSKLSVIRNLNDQVLFIDQGNRPLFEDMTDSDARDNAPRTIFIISMYADSQPRGMAVTISVKAEKISTLSAENKIISFKEMNPPDNIKDTKSCIIFFQRSVPGHDNKMQFESSSYEGYFLAAEKERDLFKLILKKEDELGDRSIMFTVQNED

SEQ ID NO: 11 - protein, 157 aa, synthetic construct
YFGKLKSKLSIIRNLNDQVLFIDQGNRPLFEDMTDSDARDNAPRTIFIISMYADSQPRGMAVTISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 12 - protein, 157 aa, synthetic construct
YFGKLKSKLSVIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 13 - protein, 157 aa, synthetic construct
YFGKLKSKLSVIRNLNDQVLFIDQGNRPLFEDMTDSDCRDNAPRTIFIISMYADSQPRGMAVNISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 14 - protein, 157 aa, synthetic construct
YFGKLKSKLSVIRNLNDQVLFIDQGNRPLFEDMTDSDCRDNAPRTIFIIAMYADSQPRGMAVNISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 15 - protein, 157 aa, synthetic construct
YFGKLKSKLSVIRNLNDQVLFIDQGNRPLFEDMTDSDCRDNAPRTIFIIHMYADSQPRGMAVNISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 16 - protein, 157 aa, synthetic construct
YFGKLKSKLSVIRNLNDQVLFIDQGNRPLFEDMTDSDCRDNAPRTIFIISMYADSQPRGMAVNIAVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 17 - protein, 157 aa, synthetic construct
YFGKLKSKLSVIRNLNDQVLFIDQGNRPLFEDMTDSDCRDNAPRTIFIIHMYADSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 18 - protein, 157 aa, synthetic construct
YFGKLKSKLSVIRNLNDQVLFIDQGNRPLFEDMTDSDARDNAPRTIFIISMYADSQPRGMAVTISVKAEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 19 - protein, 157 aa, synthetic construct
YFGKLKSKLSVIRNLNDQVLFIDQGNRPLFEDMTDSDCRDNAPRTIFIISMYADSQPRGMAVTISVKCEKISTLSCENAIISFKEMNPPDNIKDTKSDIIFFQRSVPGHDNKMQFESSSYEGYFLACEKERDLFKLILKKEDELGDRSIMFTVQNED

SEQ ID NO: 20 - protein, 157 aa, synthetic construct; identical to SEQ ID NO: 19

SEQ ID NO: 21 - protein, 157 aa, synthetic construct
YFGKLKSKLSVIRNLNDQVLFIDQGNRPLFEDMTDSDCRDNAPRTIFIISMYADSQPRGMAVTISVKCEKISTLSCENKIISFKEMNPPDNIKDTKSDIIFFQRSVPAHDNKMQFESSSYEGYFLACEKERDLFKLILKKEDELGDRSIMFTVQNED

SEQ ID NO: 22 - protein, 157 aa, synthetic construct
YFGKLKSKLSVIRNLNDQVLFIDQGNRPLFEDMTDSDCRDNAPRTIFIISMYADSQPRGMAVTISVKCEKISTLSCENKIISFKEMNPPDNIKDTKSDIIFFQRSVPGADNKMQFESSSYEGYFLACEKERDLFKLILKKEDELGDRSIMFTVQNED

SEQ ID NO: 23 - protein, 157 aa, synthetic construct
YFGKLKSKLSVIRNLNDQVLFIDQGNRPLFEDMTDSDCRDNAPRTIFIISMYADSQPRGMAVTISVKCEKISTLSCENKIISFKEMNPPDNIKDTKSDIIFFQRSVPGHDNAMQFESSSYEGYFLACEKERDLFKLILKKEDELGDRSIMFTVQNED

SEQ ID NO: 24 - protein, 157 aa, synthetic construct
YFGKLKSKLSVIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLACEKERDLFKLILKKEDELGDRSIMFTVQNED

SEQ ID NO: 25 - protein, 157 aa, synthetic construct
YFGKLKSKLSVIRNLNDQVLFIDQGNRPLFEDMTDSDQRDNAPRTIFIISMYADSQPRGMAVAISVKCEKISTLSAENKIISFKEMNPPDNIKDTKSDIIFFQRSVPGHDNKMQFESSSYEGYFLACEKERDLFKLILKKEDELGDRSIMFTVQNED

SEQ ID NO: 26 - protein, 157 aa, synthetic construct
YFGKLKSKLSVIRNLNDQVLFIDQGNRPLFEDMTDSDARDNAPRTIFIISMYADSQPRGMAVAISVKCEKISTLSCENKIISFKEMNPPDNIKDTKSDIIFFQRSVPGHDNKMQFESSSYEGYFLAAEKERDLFKLILKKEDELGDRSIMFTVQNED

SEQ ID NO: 27 - protein, 157 aa, synthetic construct
YFGKLKSKLSVIRNLNDQVLFIDQGNRPLFEDMTDSDQRDNAPRTIFIISMYADSQPRGMAVAISVKCEKISTLSCENKIISFKEMNPPDNIKDTKSDIIFFQRSVPGHDNKMQFESSSYEGYFLAAEKERDLFKLILKKEDELGDRSIMFTVQNED

SEQ ID NO: 28 - protein, 157 aa, synthetic construct; identical to SEQ ID NO: 12

SEQ ID NO: 29 - protein, 157 aa, synthetic construct
YFGKLKSKLSIIRNLNDQVLFIDQGNRPLFEDMTDSDARDNAPRTIFIISMYADSQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 30 - protein, 157 aa, synthetic construct
YFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 31 - protein, 157 aa, synthetic construct
YFGKLESKLSVIRNLNDQVLFIDQGNRPLFEDMTDSDARDNAPRTIFIISMYKDSQPRGMAVTISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 32 - protein, 157 aa, synthetic construct
YFGKLESKLSVIRNLNDQVLFIDQGNRPLFEDMTDSDARDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 33 - protein, 157 aa, synthetic construct
YFGKLKSKLSVIRNLNDQVLFIDQGNRPLFEDMTDSDARDNAPRTIFIISMYADSQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 34 - protein, 157 aa, synthetic construct
YFGKLKSKLSVIRNLNDQVLFIDQGNRPLFEDMTDSDCRDNAPRTIFIISMYADAQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 35 - protein, 157 aa, synthetic construct
GAGKLKSKLSVIRNLNDQVLFIDQGNRPLFEDMTDSDCRDNAPRTIFIISGYAAAQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 36 - protein, 157 aa, synthetic construct
YFGKLESKLSVIRNLNDQVLFIDQGNRPLFEDMTDSDCRDNAPRTIFIISMYADSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 37 - protein, 157 aa, synthetic construct
YFGKLESKLSVIRNLNDQVLFIDQGNRPLFEDMTDSDCRDNAPRTIFIISMYKDAQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 38 - protein, 157 aa, synthetic construct
YFGKLKSKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 39 - protein, 157 aa, synthetic construct
YFGKLKSKLSVIRNLNDQVLFIDQGNRPLFEDMTDSDCRDNAPRTIFIISMYADSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 40 - protein, 157 aa, synthetic construct
YFGKLKSKLSVIRNLNDQVLFIDQGNRPLFEDMTDSDCRDNAPRTIFIISMYKDAQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 41 - protein, 157 aa, synthetic construct
YFGKLESKLSVIRNLNDQVLFIDQGNRPLFEDMTDSDCRDNAPRTIFIISMYADAQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 42 - protein, 157 aa, synthetic construct
YFGKLKSKLSVIRNLNDQVLFIDQGNRPLFEDMTDSDCRDNAPRTIFIISMYADAQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 43 - protein, 157 aa, synthetic construct
GFGKLKSKLSVIRNLNDQVLFIDQGNRPLFEDMTDSDCRDNAPRTIFIISMYADAQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 44 - protein, 157 aa, synthetic construct
YAGKLKSKLSVIRNLNDQVLFIDQGNRPLFEDMTDSDCRDNAPRTIFIISMYADAQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 45 - protein, 157 aa, synthetic construct
YFGKLKSKLSVIRNLNDQVLFIDQGNRPLFEDMTDSDCRDNAPRTIFIISMYAAAQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 46 - protein, 157 aa, synthetic construct
YFGKLKSKLSVIRNLNDQVLFIDQGNRPLFEDMTDSDCRDNAPRTIFIISGYADAQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 47 - protein, 157 aa, synthetic construct
YFGKLESKLSVIRNLNDQVLFIDQGNRPLFEDMTDSDSRDNAPRTIFIISMYKDSQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 48 - protein, 157 aa, synthetic construct
YFGKLESKLSVIRNLNDQVLFIDQGNRPLFEDMTDSDSRDNAPRTIFIISMYKDSQPRGMAVTISVKSECISTLSSENKIISFKEMNPPDNIKDTKSDIIFFQRSVPGHDNKMQFESSSYEGYFLASEKERDLFKLILKKEDELGDRSIMFTVQNED

SEQ ID NO: 49 - protein, 157 aa, synthetic construct
YFGKLKSKLSVIRNLNDQVLFIDQGNRPLFEDMTDSDSRDNAPRTIFIISMYADAQPRGMAVTISVKSECISTLSSENKIISFKEMNPPDNIKDTKSDIIFFQRSVPGHDNKMQFESSSYEGYFLASEKERDLFKLILKKEDELGDRSIMFTVQNED

SEQ ID NO: 50 - protein, 157 aa, synthetic construct
YFGKLKSKLSVIRNLNDQVLFIDQGNRPLFEDMTDSDCRDNAPRTIFIISMYADSQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 51 - protein, 157 aa, synthetic construct
YFGKLESKLSVIRNLNDQVLFIDQGNRPLFEDMTDSDCRDNAPRTIFIISMYKDSQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 52 - protein, 157 aa, synthetic construct
YFGKLKSKLSVIRNLNDQVLFIDQGNRPLFEDMTDSDCRDNAPRTIFIISMYKDSQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 53 - protein, 157 aa, synthetic construct
YFGKLESKLSVIRNLNDQVLFIDQGNRPLFEDMTDSDCRDNAPRTIFIISMYADSQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 54 - protein, 157 aa, synthetic construct
YFGKLKSKLSVIRNLNDQVLFIDQGNRPLFEDMTDSDSRDNAPRTIFIISMYADSQPRGMAVTISVKSECISTLSSENKIISFKEMNPPDNIKDTKSDIIFFQRSVPGHDNKMQFESSSYEGYFLASEKERDLFKLILKKEDELGDRSIMFTVQNED

SEQ ID NO: 55 - protein, 157 aa, synthetic construct
YFGKLESKLSVIRNLNDQVLFIDQGNRPLFEDMTDSDSRDNAPRTIFIISMYADSQPRGMAVAISVKSECISTLSSENKIISFKEMNPPDNIKDTKSDIIFFQRSVPGHDNKMQFESSSYEGYFLASEKERDLFKLILKKEDELGDRSIMFTVQNED

SEQ ID NO: 56 - protein, 157 aa, synthetic construct
YFGKLKSKLSVIRNLNDQVLFIDQGNRPLFEDMTDSDSRDNAPRTIFIISMYADSQPRGMAVTISVKCEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 57 - protein, 157 aa, synthetic construct
YFGKLKSKLSVIRNLNDQVLFIDQGNRPLFEDMTDSDSRDNAPRTIFIISMYADSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 58 - protein, 157 aa, synthetic construct; identical to SEQ ID NO: 54

SEQ ID NO: 59 - protein, 157 aa, synthetic construct
YFGKLKSKLSVIRNLNDQVLFIDQGNRPLFEDMTDSDARDNAPRTIFIISMYADSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 60 - protein, 157 aa, synthetic construct
YFGKLKSKLSVIRNLNDQVLFIDQGNRPLFEDMTDSDQRDNAPRTIFIISMYADSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 61 - protein, 157 aa, synthetic construct
YFGKLKSKLSVIRNLNDQVLFIDQGNRPLFEDMTDSDARDNAPRTIFIISMYADSQPRGMAVTISVKCEKISTLSAENKIISFKEMNPPDNIKDTKSDIIFFQRSVPGHDNKMQFESSSYEGYFLACEKERDLFKLILKKEDELGDRSIMFTVQNED

SEQ ID NO: 62 - protein, 157 aa, synthetic construct
YFGKLKSKLSVIRNLNDQVLFIDQGNRPLFEDMTDSDARDNAPRTIFIISMYADSQPRGMAVTISVKCEKISTLSCENKIISFKEMNPPDNIKDTKSDIIFFQRSVPGHDNKMQFESSSYEGYFLAAEKERDLFKLILKKEDELGDRSIMFTVQNED

SEQ ID NO: 63 - protein, 157 aa, synthetic construct
YFGKLKSKLSVIRNLNDQVLFIDQGNRPLFEDMTDSDARDNAPRTIFIISMYADSQPRGMAVTISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 64 - protein, 157 aa, synthetic construct
YFGKLKSKLSVIRNLNDQVLFIDQGNRPLFEDMTDSDARDNAPRTIFIISMYADAQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 65 - protein, 157 aa, synthetic construct
YFGKLKSKLSVIRNLNDQVLFIDQGNRPLFEDMTDSDQRDNAPRTIFIISMYADAQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 66 - protein, 157 aa, synthetic construct
YFGKLKSKLSVIRNLNDQVLFIDQGNRPLFEDMTDSDCRDNAPRTIFIISMYADSQPRGMAVTISVKCEKISTLSCENKIISFAEMNPPDNIKDTKSDIIFFQRSVPGHDNKMQFESSSYEGYFLACEKERDLFKLILKKEDELGDRSIMFTVQNED

SEQ ID NO: 67 - protein, 157 aa, synthetic construct
YFGKLKSKLSVIRNLNDQVLFIDQGNRPLFEDMTDSDCRDNAPRTIFIISMYADSQPRGMAVTISVKCEKISTLSCENKIISFKEMNPPDNIKDTKSAIIFFQRSVPGHDNKMQFESSSYEGYFLACEKERDLFKLILKKEDELGDRSIMFTVQNED

SEQ ID NO: 68-203 - skipped (136 entries)

SEQ ID NO: 204 - protein, 157 aa, synthetic construct
YFGKLESKLSIIRNLNDQVLFIDQGNRPLFEDMTDSDARDNAPRTIFIISMYADSQPRGMAVTISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 205 - protein, 157 aa, synthetic construct
YFGKLESKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 206 - protein, 157 aa, synthetic construct
YFGKLESKLSIIRNLNDQVLFIDQGNRPLFEDMTDSDARDNAPRTIFIISMYADAQPRGMAVTISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 207 - protein, 157 aa, synthetic construct
YFGKLESKLSIIRNLNDQVLFIDQGNRPLFEDMTDSDARDNAPRTIFIISGYADSQPRGMAVTISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 208 - protein, 157 aa, synthetic construct
YFGKLESKLSIIRNLNDQVLFIDQGNRPLFEDMTDSDARDNAPRTIFIISMYAASQPRGMAVTISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 209 - protein, 157 aa, synthetic construct
YAGKLESKLSIIRNLNDQVLFIDQGNRPLFEDMTDSDARDNAPRTIFIISMYADSQPRGMAVTISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 210 - protein, 157 aa, synthetic construct
YFGKLESKLSIIRNLNDQVLFIDQGNRPLFADMTDSDARDNAPRTIFIISMYADSQPRGMAVTISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 211 - protein, 157 aa, synthetic construct
YFGKLESKLSIIRNLNDQVLFIDQGNRPLFEDMADSDARDNAPRTIFIISMYADSQPRGMAVTISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 212 - protein, 157 aa, synthetic construct
YFGKLESKLSIIRNLNDQVLFIDQGNRPLFEDMTASDARDNAPRTIFIISMYADSQPRGMAVTISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 213 - protein, 157 aa, synthetic construct
YFGKLESKLSIIRNLNDQVLFIDQGNRPLFEDMTDADARDNAPRTIFIISMYADSQPRGMAVTISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 214 - protein, 157 aa, synthetic construct
YFGKLESKLSIIRNLNDQVLFIDQGNRPLFEDMTDSAARDNAPRTIFIISMYADSQPRGMAVTISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 215 - protein, 157 aa, synthetic construct
YFGKLESKLSIIRNLNDQVLFIDQGNRPLFADMTDSAARDNAPRTIFIISMYADSQPRGMAVTISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 216 - protein, 157 aa, synthetic construct
YFGKLESKLSIIRNLNDQVLFIDQGNRPLFEDMTDSDARANAPRTIFIISMYADSQPRGMAVTISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 217 - protein, 157 aa, synthetic construct
YFGKLESKLSIIRNLNDQVLFIDQGNRPLFEDMTDSDARDAAPRTIFIISMYADSQPRGMAVTISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 218 - protein, 157 aa, synthetic construct
YFGKLESKLSIIRNLNDQVLFIDQGNRPLFEDMTDSDARDNAPRTIFIISMYADSQPRGMAVTISVKCEKISTLSAENKIISFKEMNPPDNIKDTKSDIIFFQRSVPGHDNKMQFESSSYEGYFLAAEKERALFKLILKKEDELGDRSIMFTVQNED

SEQ ID NO: 219 - protein, 157 aa, synthetic construct
YFGKLESKLSIIRNLNDQVLFIDQGNRPLFEDMTDSDARDNAPRTIFIISMYADSQPRGMAVTISVKCEKISTLSAENKIISFKEMNPPDNIKDTKSDIIFFQRSVPAHDNKMQFESSSYEGYFLAAEKERDLFKLILKKEDELGDRSIMFTVQNED

SEQ ID NO: 220 - protein, 157 aa, synthetic construct
YFGKLESKLSIIRNLNDQVLFIDQGNRPLFEDMTDSDARDNAPRTIFIISMYADSQPRGMAVTISVKCEKISTLSAENKIISFKEMNPPDNIKDTKSDIIFFQRSVPGADNKMQFESSSYEGYFLAAEKERDLFKLILKKEDELGDRSIMFTVQNED

SEQ ID NO: 221 - protein, 157 aa, synthetic construct
YFGKLESKLSIIRNLNDQVLFIDQGNRPLFEDMTDSDARDNAPRTIFIISMYADSQPRGMAVTISVKCEKISTLSAENKIISFKEMNPPDNIKDTKSDIIFFQRSVPGHANKMQFESSSYEGYFLAAEKERDLFKLILKKEDELGDRSIMFTVQNED

SEQ ID NO: 222 - protein, 157 aa, synthetic construct
YFGKLESRLSIIRNLNDQVLFIDQGNRPLFEDMTDSDARDNAPRTIFIISMYKDSQPRGMAVTISVKCEKISTLSAENKIISFKEMNPPDNIKDTKSDIIFFERSVPGHDNKMQFESSSYEGYFLAAEKERDLFKLILKKEDELGDRSIMFTVQNED

SEQ ID NO: 223 - protein, 157 aa, synthetic construct
YFGKLESELSIIRNLNDQVLFIDQGNRPLFEDMTDSDARDNAPRTIFIISMYKDSQPRGMAVTISVKCEKISTLSAENKIISFKEMNPPDNIKDTKSDIIFFRRSVPGHDNKMQFESSSYEGYFLAAEKERDLFKLILKKEDELGDRSIMFTVQNED

SEQ ID NO: 224 - protein, 157 aa, synthetic construct
YFGKLESKLSIIRNLNDQVLFIDQGNRPLFEDMTDSDARDNAPRTIFIISMYKDSQPRGMAVTISVKCEKISTLSAENKIISFKEMNPPDNIKDTKSDIIFFKRSVPGHDNKMQFESSSYEGYFLAAEKERDLFKLILKKEDELGDRSIMFTVQNED

SEQ ID NO: 225 - protein, 157 aa, synthetic construct
YFGKLESKLSIIRNLNDQVLFIDQGNRPLFEDMTDSDARDNAPRTIFIISMYKDHQPRGMAVTISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 226 - protein, 157 aa, synthetic construct
YFGKLESKLSIIRNLNDQVLFIDQGNRPLFEDMTDSDARDNAPRTIFIISMYKDRQPRGMAVTISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 227 - protein, 157 aa, synthetic construct
YFGKLESKLSIIRNLNDQVLFIDQGNRPLFEDMTDSDARDNAPRTIFIISMYKDTQPRGMAVTISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 228 - protein, 157 aa, synthetic construct
YFGKLESKLSIIRNLNDQVLFIDQGNRPLFEDMTDSDARDNAPRTIFIISMYKDSQPRGMAVTISVKCEKISTLSAENKIISFKEMNPPDNIKDTKSDIIFFQRIVPGHDNKMQFESSSYEGYFLAAEKERDLFKLILKKEDELGDRSIMFTVQNED

SEQ ID NO: 229 - protein, 157 aa, synthetic construct
YFGKLESKLSIIRNLNDQVLFIDQGNRPLFEDMTDSDARDNAPRTIFIISMYKDSQPRGMAVTISVKCEKISTLSAENKIISFKEMNPPDNIKDTKSDIIFFQRKVPGHDNKMQFESSSYEGYFLAAEKERDLFKLILKKEDELGDRSIMFTVQNED

SEQ ID NO: 230 - protein, 157 aa, synthetic construct
YFGKLKSLLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 231 - protein, 157 aa, synthetic construct
YFGKLKSKLSIIRNLNDQVLFIDQGNRPLFEDMTDSDARDNAPRTIFIESMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 232 - protein, 157 aa, synthetic construct
YFGKLKSKLSIIRNLNDQVLFIDQGNRPLFEDMTDSDARDNAPRTIFIMSMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 233 - protein, 157 aa, synthetic construct
YFGKLKSKLSIIRNLNDQVLFIDQGNRPLFEDMTDSDARDNAPRTIFIRSMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 234 - protein, 157 aa, synthetic construct
YFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFRRSVPGHDNKMQFESSSYEGYFLAAEKERDLFKLILKKEDELGDRSIMFTVQNED

SEQ ID NO: 235 - protein, 157 aa, synthetic construct
YFGKLKSELSIIRNLNDQVLFIDQGNRPLFEDMTDSDARDNAPRTIFIISMYADSQPRGMAVAISVKCEKISTLSAENKIISFKEMNPPDNIKDTKSDIIFFRRSVPGHDNKMQFESSSYEGYFLAAEKERDLFKLILKKEDELGDRSIMFTVQNED

SEQ ID NO: 236 - protein, 157 aa, synthetic construct
YFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTRQNED

SEQ ID NO: 237 - protein, 157 aa, synthetic construct
YFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTEQNED

SEQ ID NO: 238 - protein, 157 aa, synthetic construct
YFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTYQNED

SEQ ID NO: 239 - protein, 157 aa, synthetic construct
YFGKLKSKLSIIRNLNDQVLFIDQGNRPLFEDMTDSDARDNAPRTIFIISGYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 240 - protein, 157 aa, synthetic construct
YFGKLRSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 241 - protein, 161 aa, synthetic construct
GGGGYFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 242 - protein, 158 aa, synthetic construct
GYFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 243 - protein, 157 aa, synthetic construct
MFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 244 - protein, 158 aa, synthetic construct
GYFGKLKSKLSIIRNLNDQVLFIDQGNRPLFEDMTDSDARDNAPRTIFIISGYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED